(19) United States
(12) Patent Application Publication
(10) Pub. No.: US 2003/0131386 A1
(43) Pub. Date: Jul. 10, 2003

(54) Stress-induced polynucleotides

(76) Inventors: Raymond Samaha, Capitola, CA (US); Jacqueline Heard, San Mateo, CA (US); Cai-Zhong Jiang, Fremont, CA (US); Omaira Pineda, Castro Valley, CA (US); Lynne Reuber, San Mateo, CA (US); Jose Luis Riechmann, Oakland, CA (US); Guo-Liang Yu, Berkeley, CA (US); James Keddie, San Mateo, CA (US); Oliver Ratcliffe, Oakland, CA (US); Marsha Pilgrim, Palo Alto, CA (US); Luc Adam, Hayward, CA (US); Pierre Broun, San Mateo, CA (US); James Zhang, Palo Alto, CA (US)

Correspondence Address: MATTHEW KASER, Mendel Biotechnology, Inc., 21375 Cabot Blvd., Hayward, CA, 94545, US

(21) Appl. No.: 10/278,536

(22) Filed: Oct. 22, 2002

Related U.S. Application Data
(62) Division of application No. 09/532,591, filed on Mar. 22, 2000, now abandoned.
(60) Provisional application No. 60/125,814, filed on Mar. 23, 1999.

(51) Int. Cl. A01H 1/00; C12N 15/82

(52) U.S. Cl. 800/289; 435/468

(57) ABSTRACT

Recombinant polynucleotides and methods for altering the regulation of gene expression in plants are provided to modify a plant's traits.

The present invention claims priority in part from U.S. Provisional Application Serial No. 60/125,814 filed Mar. 23, 1999.

### FIELD OF THE INVENTION

This invention is in the field of plant molecular biology and relates to compositions and methods for modifying a plant's traits, in particular with respect to environmental stress responses.

### BACKGROUND OF THE INVENTION

Gene expression levels are controlled in part at the level of transcription, and transcription is affected by transcription factors. Transcription factors regulate gene expression throughout the life cycle of an organism and so are responsible for differential levels of gene expression at various developmental stages, in different tissue and cell types, and in response to different stimuli. Transcription factors may interact with other proteins or with specific sites on a target gene sequence to activate, suppress or otherwise regulate transcription. In addition, the transcription of the transcription factors themselves may be regulated.

Because transcription factors are key controlling elements for biological pathways, altering the expression levels of one or more transcription factors may change entire biological pathways in an organism. For example, manipulation of the levels of selected transcription factors may result in increased expression of economically useful proteins or metabolic chemicals in plants or to improve other agriculturally relevant characteristics. Conversely, blocked or reduced expression of a transcription factor may reduce biosynthesis of unwanted compounds or remove an undesirable trait. Therefore, manipulating transcription factor levels in a plant offers tremendous potential in agricultural biotechnology for modifying a plant's traits.

The present invention provides transcription factors for use in modifying a plant's response to environmental stresses.

### SUMMARY OF THE INVENTION

In one aspect, the present invention relates to a transgenic plant comprising a recombinant polynucleotide. The recombinant polynucleotide comprises a nucleotide sequence encoding a polypeptide comprising at least 6 consecutive amino acids of a sequence selected from the group consisting of SEQ ID Nos. 2N, where N=1-111. And the presence of the recombinant polynucleotide alters the environmental stress tolerance of the transgenic plant when compared with the same trait of another plant lacking the recombinant polynucleotide.

In one embodiment, the nucleotide sequence encodes a polypeptide comprising a conserved domain such as 1) a localization domain, 2) an activation domain, 3) a repression domain, 4) an oligomerization domain or 5) a DNA binding domain. In a further embodiment, the nucleotide sequence further comprises a promoter operably linked to the nucleotide sequence. The promoter may be a constitutive or inducible or tissue-active.

In a second aspect, the present invention relates to a method for altering a plant's tolerance to stress. The method comprises (a) transforming a plant with a recombinant polynucleotide comprising a nucleotide sequence encoding a polypeptide comprising at least 6 consecutive amino acids of a sequence selected from the group consisting of SEQ ID Nos. 2N, where N=1-111 (b) selecting transformed plants; and (c) identifying a transformed plant with roots having an altered trait.

In one embodiment, the nucleotide sequence encodes a polypeptide comprising a conserved domain such as 1) a localization domain, 2) an activation domain, 3) a repression domain, 4) an oligomerization domain or 5) a DNA binding domain. In a further embodiment, the nucleotide sequence further comprises a promoter operably linked to the nucleotide sequence. The promoter may be a constitutive or inducible or tissue-active.

In a third aspect, the present invention relates to a method for altering the expression levels of at least one gene in a plant. The method comprises (a) transforming the plant with a recombinant polynucleotide comprising a nucleotide sequence encoding a polypeptide comprising at least 6 consecutive amino acids of a sequence selected from the group consisting of SEQ ID Nos. 2N, where N=1-111; and (b) selecting said transformed plant.

In one, embodiment, the nucleotide sequence encodes a polypeptide comprising a conserved domain such as 1) a localization domain, 2) an activation domain, 3) a repression domain, 4) an oligomerization domain or 5) a DNA binding domain. In a further embodiment, the nucleotide sequence further comprises a promoter operably linked to the nucleotide sequence. The promoter may be a constitutive or inducible or tissue-active.

In a fourth aspect, the present invention relates to another method for altering the environmental stress tolerance of a plant. The method comprises (a) transforming the plant with a recombinant polynucleotide comprising a nucleotide sequence comprising at least 18 consecutive nucleotides of a sequence selected from the group consisting of SEQ ID Nos. 2−N1, where N=1-111, and SEQ ID Nos. 223-238; and (b) selecting said transformed plant.

In yet another aspect, the present invention is yet another method for altering a plant's trait. The method comprises (a) providing a database sequence; (b) comparing the database sequence with a polypeptide selected from SEQ ID Nos. 2N, where N=1-111; (c) selecting a database sequence that meets selected sequence criteria; and (d) transforming said database sequence in the plant. Alternatively, the database sequence can be compared with a polynucleotide selected from SEQ ID Nos. 2N−1, where N=1-111 or SEQ ID Nos. 223-238.

In a further aspect, the present invention is method for altering a plant's trait, and the method entails (a) providing a test polynucleotide; (b) hybridizing the test polynucleotide with a polynucleotide selected from SEQ ID Nos. 2N−1, where N=1-111 or SEQ ID Nos. 223-238 at low stringency; and (c) transforming the hybridizing test polynucleotide in a plant to alter a trait of the plant.

In a further aspect, the present invention is method for altering a plant's trait, and the method entails (a) providing a test polynucleotide; (b) hybridizing the test polynucleotide with a polynucleotide selected from SEQ ID Nos. 2N-1, where N=1-111 or SEQ ID Nos. 223-238 at low stringency; and (c) transforming the hybridizing test polynucleotide in a plant to alter a trait of the plant.

### BRIEF DESCRIPTION OF THE FIGURES

FIGS. 1a-1h provide a table of exemplary polynucleotide and polypeptide sequences of the invention. The table includes from left to right for each sequence: the SEQ ID No., the internal code reference number, the transcription factor family of the sequence, particular DNA or protein fragments for each sequence, whether the sequence is a polynucleotide or polypeptide sequence, identification of the coding sequence for each full length and identification of any conserved domains for the polypeptide sequences.

### DETAILED DESCRIPTION OF THE INVENTION

Definitions

A “recombinant polynucleotide” is a nucleotide sequence comprising a gene coding sequence or a fragment thereof (comprising at least 18 consecutive nucleotides, preferably at least 30 consecutive nucleotides, and more preferably at least 50 consecutive nucleotides). Additionally, the polynucleotide may comprise a promoter, an intron, an enhancer region, a polyadenylation site, a translation initiation site, 5′ or 3′ untranslated regions, a reporter gene, a selectable marker or the like. The polynucleotide may comprise single stranded or double stranded DNA or RNA. The polynucleotide may comprise modified bases or a modified backbone. The polynucleotide may be genomic, a transcript (such as an mRNA) or a processed nucleotide sequence (such as a cDNA). The polynucleotide may comprise a sequence in either sense or antisense orientations.

“recombinant polynucleotide” is a polynucleotide that is not in its native state, e.g., the polynucleotide is comprised of a nucleotide sequence not found in nature or the polynucleotide is separated from nucleotide sequences with which it typically is in proximity or is next to nucleotide sequences with which it typically is not in proximity.

An “recombinant polypeptide” is a polypeptide derived from the translation of a recombinant polynucleotide or is more enriched in a cell than the polypeptide in its natural state in a wild type cell, e.g. more than 5% enriched, more than 10% enriched or more than 20% enriched and is not the result of a natural response of a wild type plant or is separated from other components with which it is typically associated with in a cell.

A “transgenic plant” may refer to a plant that contains genetic material not normally found in a wild type plant of the same species, or in a naturally occurring variety or in a cultivar, and which has been introduced into the plant by human manipulation. A transgenic plant is a plant that may contain an expression vector or cassette. The expression cassette comprises a gene coding sequence and allows for the expression of the gene coding sequence. The expression cassette may be introduced into a plant by transformation or by breeding after transformation of a parent plant.

A transgenic plant refers to a whole plant as well as to a plant part, such as seed, fruit, leaf, or root, plant tissue, plant cells or any other plant material, and progeny thereof.

The phrase “altered expression” in reference to polynucleotide or polypeptide expression refers to an expression pattern in the transgenic plant that is different from the expression pattern in the wild type plant or a reference; for example, by expression in a cell type other than a cell type in which the sequence is expressed in the wild type plant, or by expression at a time other than at the time the sequence is expressed in the wild type plant, or by a response to different inducible agents, such as hormones or environmental signals, or at different expression levels (either higher or lower) compared with those found in a wild type plant. The term also refers to lowering the levels of expression to below the detection level or completely abolishing expression. The resulting expression pattern may be transient or stable, constitutive or inducible.

A “transcription factor” (TF) refers to a polynucleotide or polypeptide that controls the expression of a gene or genes either directly by binding to one or more nucleotide sequences associated with a gene coding sequence or indirectly by affecting the level or activity of other polypeptides that do bind directly or indirectly to one or more nucleotide sequences associated with a gene coding sequence. A TF, in this definition, includes any polypeptide that can activate or repress transcription of a single gene or a number of genes. This polypeptide group includes, but is not limited to, DNA binding proteins, protein kinases, protein phosphatases, GTP-binding proteins and receptors.

The transcription factor sequence may comprise a whole coding sequence or a fragment or domain of a coding sequence. A “fragment or domain”, as referred to polypeptides, may be a portion of a polypeptide which performs at least one biological function of the intact polypeptide in substantially the same manner or to a similar extent as does the intact polypeptide. A fragment may comprise, for example, a DNA binding domain that binds to a specific DNA promoter region, an activation domain or a domain for protein-protein interactions. Fragments may vary in size from as few as 6 amino acids to the length of the intact polypeptide, but are preferably at least 30 amino acids in length and more preferably at least 60 amino acids in length. In reference to a nucleotide sequence “a fragment” refers to any sequence of at least consecutive 18 nucleotides, preferably at least 30 nucleotides, more preferably at least 50, of any of the sequences provided herein.

Exemplary polynucleotides and polypeptides comprise a sequence provided in the Sequence Listing as SEQ ID No.1 (G1426), SEQ ID No.2 (G1426 protein), SEQ ID No.3 (G578), SEQ ID No.4 (G578 protein), SEQ ID No.5 (G598), SEQ ID No.6 (G598 protein), SEQ ID No.7 (G610), SEQ ID No.8 (G610 protein), SEQ ID No.9 (G620), SEQ ID No.10 (G620 protein), SEQ ID No.11 (G634), SEQ ID No.12 (G634 protein), SEQ ID No.13 (G659), SEQ ID No.14 (G659 protein), SEQ ID No.15 (G759), SEQ ID No.16 (G759 protein), SEQ ID No.17 (G764), SEQ ID No.18 (G764 protein), SEQ ID No.19 (G765), SEQ ID No.20 (G765 protein), SEQ ID No.21 (G777), SEQ ID No.22 (G777 protein), SEQ ID No.23 (G867), SEQ ID No.24 (G867 protein), SEQ ID No.25 (G503), SEQ ID No.26 (G503 protein), SEQ ID No.27 (G975), SEQ ID No.28 (G975 protein), SEQ ID No.29 (G562), SEQ ID No.30 (G562 protein), SEQ ID No.31 (G1508), SEQ ID No.32 (G1508 protein), SEQ ID No.33 (G176), SEQ ID No.34 (G176 protein), SEQ ID No.35 (G197), SEQ ID No.36 (G197 protein), SEQ ID No.37 (G222), SEQ ID No.38 (G222 protein), SEQ ID No.39 (G229), SEQ ID No.40 (G229 protein), SEQ ID No.41 (G24), SEQ ID No.42 (G24 protein), SEQ ID No.43 (G10), SEQ ID No.44 (G10 protein), SEQ ID No.45 (G308), SEQ ID No.46 (G308 protein), SEQ ID No.47 (G928), SEQ ID No.48 (G928 protein), SEQ ID No.49 (G404), SEQ ID No.50 (G404 protein), SEQ ID No.51 (G438), SEQ ID No.52 (G438 protein), SEQ ID No.53 (G46), SEQ ID No.54 (G46 protein), SEQ ID No.55 (G921), SEQ ID No.56 (G921 protein), SEQ ID No.57 (G233), SEQ ID No.58 (G233 protein), SEQ ID No.59 (G1131), SEQ ID No.60 (G1131 protein), SEQ ID No.61 (G1141), SEQ ID No.62 (G1141 protein), SEQ ID No.63 (G1181), SEQ ID No.64 (G1181 protein), SEQ ID No.65 (G1319), SEQ ID No.66 (G1319 protein), SEQ ID No.67 (G1328), SEQ ID No.68 (G1328 protein), SEQ ID No.69 (G1334), SEQ ID No.70 (G1334 protein), SEQ ID No.71 (G14), SEQ ID No.72 (G14 protein), SEQ ID No.73 (G1412), SEQ ID No.74 (G1412 protein), SEQ ID No.75 (G16), SEQ ID No.76 (G16 protein), SEQ ID No.77 (G184), SEQ ID No.78 (G184 protein), SEQ ID No.79 (G198), SEQ ID No.80 (G198 protein), SEQ ID No.81 (G20), SEQ ID No.82 (G20 protein), SEQ ID No.83 (G571), SEQ ID No.84 (G571 protein), SEQ ID No.85 (G221), SEQ ID No.86 (G221 protein), SEQ ID No.87 (G570), SEQ ID No.88 (G570 protein), SEQ ID No.89 (G25), SEQ ID No.90 (G25 protein), SEQ ID No.91 (G258), SEQ ID No.92 (G258 protein), SEQ ID No.93 (G38), SEQ ID No.94 (G38 protein), SEQ ID No.95 (G389), SEQ ID No.96 (G389 protein), SEQ ID No.97 (G395), SEQ ID No.98 (G395 protein), SEQ ID No.99 (G471), SEQ ID No.100 (G471 protein), SEQ ID No.101 (G486), SEQ ID No.102 (G486 protein), SEQ ID No.103 (G511), SEQ ID No.104 (G511 protein), SEQ ID No.105 (G512), SEQ ID No.106 (G512 protein), SEQ ID No.107 (G525), SEQ ID No.108 (G525 protein), SEQ ID No.109 (G555), SEQ ID No.110 (G555 protein), SEQ ID No.111 (G350), SEQ ID No.112 (G350 protein), SEQ ID No.113 (G220), SEQ ID No.114 (G220 protein), SEQ ID No.115 (G472), SEQ ID No.116 (G472 protein), SEQ ID No.117 (G227), SEQ ID No.118 (G227 protein), SEQ ID No.119 (G234), SEQ ID No.120 (G234 protein), SEQ ID No.121 (G515), SEQ ID No.122 (G515 protein), SEQ ID No.123 (G274), SEQ ID No.124 (G274 protein), SEQ ID No.125 (G3), SEQ ID No.126 (G3 protein), SEQ ID No.127 (G1004), SEQ ID No.128 (G1004 protein), SEQ ID No.129 (G1043), SEQ ID No.130 (G1043 protein), SEQ ID No.131 (G291), SEQ ID No.132 (G291 protein), SEQ ID No.133 (G315), SEQ ID No.134 (G315 protein), SEQ ID No.135 (G398), SEQ ID No.136 (G398 protein), SEQ ID No.137 (G4), SEQ ID No.138 (G4 protein), SEQ ID No.139 (G428), SEQ ID No.140 (G428 protein), SEQ ID No.141 (G226), SEQ ID No.142 (G226 protein), SEQ ID No.143 (G464), SEQ ID No.144 (G464 protein), SEQ ID No.145 (G263), SEQ ID No.146 (G263 protein), SEQ ID No.147 (G502), SEQ ID No.148 (G502 protein), SEQ ID No.149 (G509), SEQ ID No.150 (G509 protein), SEQ ID No.151 (G514), SEQ ID No.152 (G514 protein), SEQ ID No.153 (G553), SEQ ID No.154 (G553 protein), SEQ ID No.155 (G572), SEQ ID No.156 (G572 protein), SEQ ID No.157 (G632), SEQ ID No.158 (G632 protein), SEQ ID No.159 (G7), SEQ ID No.160 (G7 protein), SEQ ID No.161 (G725), SEQ ID No.162 (G725 protein), SEQ ID No.163 (G760), SEQ ID No.164 (G760 protein), SEQ ID No.165 (G770), SEQ ID No.166 (G770 protein), SEQ ID No.167 (G778), SEQ ID No.168 (G778 protein), SEQ ID No.169 (G881), SEQ ID No.170 (G881 protein), SEQ ID No.171 (G9), SEQ ID No.172 (G9 protein), SEQ ID No.173 (G437), SEQ ID No.174 (G437 protein), SEQ ID No.175 (G773), SEQ ID No.176 (G773 protein), SEQ ID No.177 (G1005), SEQ ID No.178 (G1005 protein), SEQ ID No.179 (G1232), SEQ ID No.180 (G1232 protein), SEQ ID No.181 (G915), SEQ ID No.182 (G915 protein), SEQ ID No.183 (G21), SEQ ID No.184 (G21 protein), SEQ ID No.185 (G8), SEQ ID No.186 (G8 protein), SEQ ID No.187 (G1362), SEQ ID No.188 (G1362 protein), SEQ ID No.189 (G142), SEQ ID No.190 (G142 protein), SEQ ID No.191 (G1425), SEQ ID No.192 (G1425 protein), SEQ ID No.193 (G1448), SEQ ID No.194 (G1448 protein), SEQ ID No.195 (G145), SEQ ID No.196 (G145 protein), SEQ ID No.197 (G162), SEQ ID No.198 (G162 protein), SEQ ID No.199 (G680), SEQ ID No.200 (G680 protein), SEQ ID No.201 (G6), SEQ ID No.202 (G6 protein), SEQ ID No.203 (G801), SEQ ID No.204 (G801 protein), SEQ ID No.205 (G216), SEQ ID No.206 (G216 protein), SEQ ID No.207 (G230), SEQ ID No.208 (G230 protein), SEQ ID No.209 (G528), SEQ ID No.210 (G528 protein), SEQ ID No.211 (G228), SEQ ID No.212 (G228 protein), SEQ ID No.213 (G241), SEQ ID No.214 (G241 protein), SEQ ID No.215 (G681), SEQ ID No.216 (G681 protein), SEQ ID No.217 (G1261), SEQ ID No.218 (G1261 protein), SEQ ID No.219 (G313), SEQ ID No.220 (G313 protein), SEQ ID No.221 (G335), SEQ ID No.222 (G335 protein), SEQ ID No.223 (G624), SEQ ID No.224 (G501), SEQ ID No.225 (G682), SEQ ID No.226 (G1274), SEQ ID No.227 (G767), SEQ ID No.228 (G961), SEQ ID No.229 (G206), SEQ ID No.230 (G907), SEQ ID No.231 (G171), SEQ ID No.232 (G348), SEQ ID No.233 (G1237), SEQ ID No.234 (G516), SEQ ID No.235 (G1222), SEQ ID No.236 (G1218), SEQ ID No.237 (G251) and SEQ ID No.238 (G 134).

A “conserved domain” refers to a polynucleotide or polypeptide fragment that is more conserved at a sequence level than other fragments when the polynucleotide or polypeptide is compared with homologous genes or proteins from other plants. The conserved domain may be 1) a localization domain, 2) an activation domain, 3) a repression domain, 4) an oligomerization domain or 5) a DNA binding domain.

A nucleotide sequence is “operably linked” when it is placed into a functional relationship with another nucleotide sequence. For example, a promoter or enhancer is operably linked to a gene coding sequence if the presence of the promoter or enhancer increases the level of expression of the gene coding sequence.

“Trait” refers to a physiological, morphological, biochemical or physical characteristic of a plant or particular plant material or cell. This characteristic may be visible to the human eye, such as seed or plant size, or be measured by biochemical techniques, such as the protein, starch or oil content of seed or leaves or by the observation of the expression level of genes by employing Northerns, RT PCR, microarray gene expression assays or reporter gene expression systems or be measured by agricultural observations such as stress tolerance, yield or disease resistance.

“Trait modification” refers to a detectable difference in a characteristic in a transgenic plant expressing a polynucleotide or polypeptide of the present invention relative to a plant not doing so, such as a wild type plant. The trait modification may entail at least a 5% increase or decrease in an observed trait (difference), at least a 10% difference, at least a 20% difference, at least a 30%, at least a 50%, at least a 70%, at least a 100% or a greater difference. It is known that there may be a natural variation in the modified trait. Therefore, the trait modification observed entails a change in the normal distribution of the trait in transgenic plants compared with the distribution observed in wild type plant.

Trait modifications of particular interest include those to seed (embryo), fruit, root, flower, leaf, stem, shoot, seedling or the like, including: enhanced tolerance to environmental conditions including freezing, chilling, heat, drought, water saturation, radiation and ozone; enhanced resistance to microbial, fungal or viral diseases; resistance to nematodes, decreased herbicide sensitivity, enhanced tolerance of heavy metals (or enhanced ability to take up heavy metals), enhanced growth under poor photoconditions (e.g., low light and/or short day length), or changes in expression levels of genes of interest. Other phenotypes that may be modified relate to the production of plant metabolites, such as variations in the production of taxol, tocopherol, tocotrienol, sterols, phytosterols, vitamins, wax monomers, anti-oxidants, amino acids, lignins, cellulose, tannins, prenyllipids (such as chlorophylls and carotenoids), glucosinolates, and terpenoids, enhanced or compositionally altered protein or oil production (especially in seeds), or modified sugar (insoluble or soluble) and/or starch composition. Physical plant characteristics that may be modified include cell development (such as the number of trichomes), fruit and seed size and number, yields of plant parts such as stems, leaves and roots, the stability of the seeds during storage, characteristics of the seed pod (e.g., susceptibility to shattering), root hair length and quantity, internode distances, or the quality of seed coat. Plant growth characteristics that may be modified include growth rate, germination rate of seeds, vigor of plants and seedlings, leaf and flower senescence, male sterility, apomixis, flowering time, flower abscission, rate of nitrogen uptake, biomass or transpiration characteristics, as well as plant architecture characteristics such as apical dominance, branching patterns, number of organs, organ identity, organ shape or size.

Of particular interest are traits relating to improved tolerance to environmental stresses, such heat, drought, osmotic stress, cold, freezing, salt, pathogen or nutrient stress or the like. Another desirable phenotype is a change in the overall gene expression pattern of the plant.

1. The Sequences

We have discovered particular plant transcription factors (TFs) that are induced when plants are exposed to environmental stresses, such as heat, drought, osmotic stress, cold, salt stress, nutrient stress or the like. These transcription factors can be used to modulate a plant's response to environmental stress. The plant transcription factors may belong to one of the following transcription factor families: the AP2 (APETALA2) domain transcription factor family (Riechmann and Meyerowitz (1998) J. Biol. Chem. 379:633-646); the MYB transcription factor family (Martin and Paz-Ares, (1997) Trends Genet. 13:67-73); the MADS domain transcription factor family (Riechmann and Meyerowitz (1997) J. Biol. Chem. 378:1079-1101); the WRKY protein family (Ishiguro and Nakamura (1994) Mol. Gen. Genet. 244:563-571); the ankyrin-repeat protein family (Zhang et al. (1992) Plant Cell 4:1575-1588); the zinc finger protein (Z) family (Klug and Schwabe (1995) FASEB J. 9: 597-604); the homeobox (HB) protein family (Duboule (1994) Guidebook to the Homeobox Genes, Oxford University Press); the CAAT-element binding proteins (Forsburg and Guarente (1989) Genes Dev. 3:1166-1178); the squamosa promoter binding proteins (SPB) (Klein et al. (1996) Mol. Gen. Genet. 1996 250:7-16); the NAM protein family (Souer et al. (1996) Cell 85:159-170); the IAA/AUX proteins (Rouse et al. (1998) Science 279:1371-1373); the HLH/MYC protein family (Littlewood et al. (1994) Prot. Profile 1:639-709); the DNA-binding protein (DBP) family (Tucker et al. (1994) EMBO J. 13:2994-3002); the bZIP family of transcription factors (Foster et al. (1994) FASEB J. 8:192-200); the Box P-binding protein (the BPF-1) family (da Costa e Silva et al. (1993) Plant J. 4:125-135); the high mobility group (HMG) family (Bustin and Reeves (1996) Prog. Nucl. Acids Res. MoL Biol. 54:35-100); the scarecrow (SCR) family (Di Laurenzio et al. (1996) Cell 86:423-433); the GF14 family (Wu et al. (1997) Plant Physiol. 114:1421-1431); the polycomb (PCOMB) family (Kennison (1995) Annu. Rev. Genet. 29:289303); the teosinte branched (TEO) family (Luo et al. (1996) Nature 383:794-799; the ABI3 family (Giraudat et al. (1992) Plant Cell 4:1251-1261); the triple helix (TH) family (Dehesh et al. (1990) Science 250:1397-1399); the EIL family (Chao et al. (1997) Cell 89:1133-44); the AT-HOOK family (Reeves and Nissen (1990) Journal of Biological Chemistry 265:8573-8582); the S1 FA family (Zhou et al. (1995) Nucleic Acids Res. 23:1165-1169); the bZIPT2 family (Lu and Ferl (1995) Plant Physiol. 109:723); the YABBY family (Bowman et al. (1999) Development 126:2387-96); the PAZ family (Bohmert et al. (1998) EMBO J. 17:170-80); a family of miscellaneous (MISC) transcription factors including the DPBF family (Kim et al. (1997) Plant J. 11:1237-1251) and the SPF1 family (Ishiguro and Nakamura (1994) Mol. Gen. Genet. 244:563-571); the golden (GLD) family (Hall et al. (1998) Plant Cell 10:925-936).

Producing transgenic plants with modified expression levels of one or more of these transcription factors compared with those levels found in a wild type or reference plant may be used to modify a plant's traits. The effect of modifying the expression levels of a particular transcription factor on the traits of a transgenic plant is described further in the Examples.

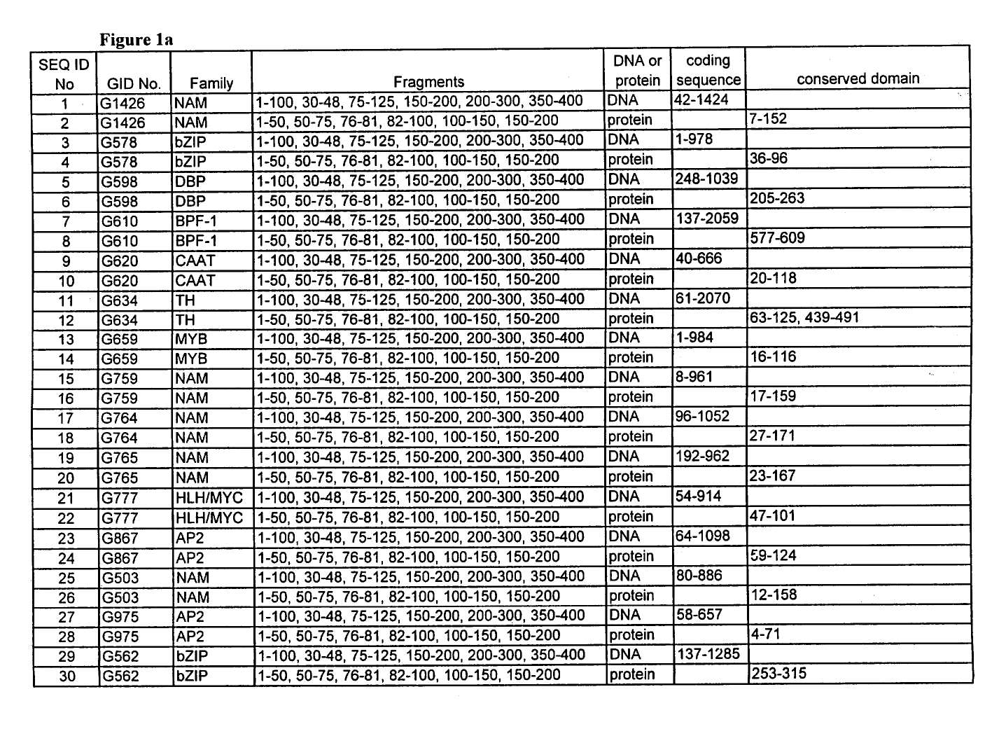

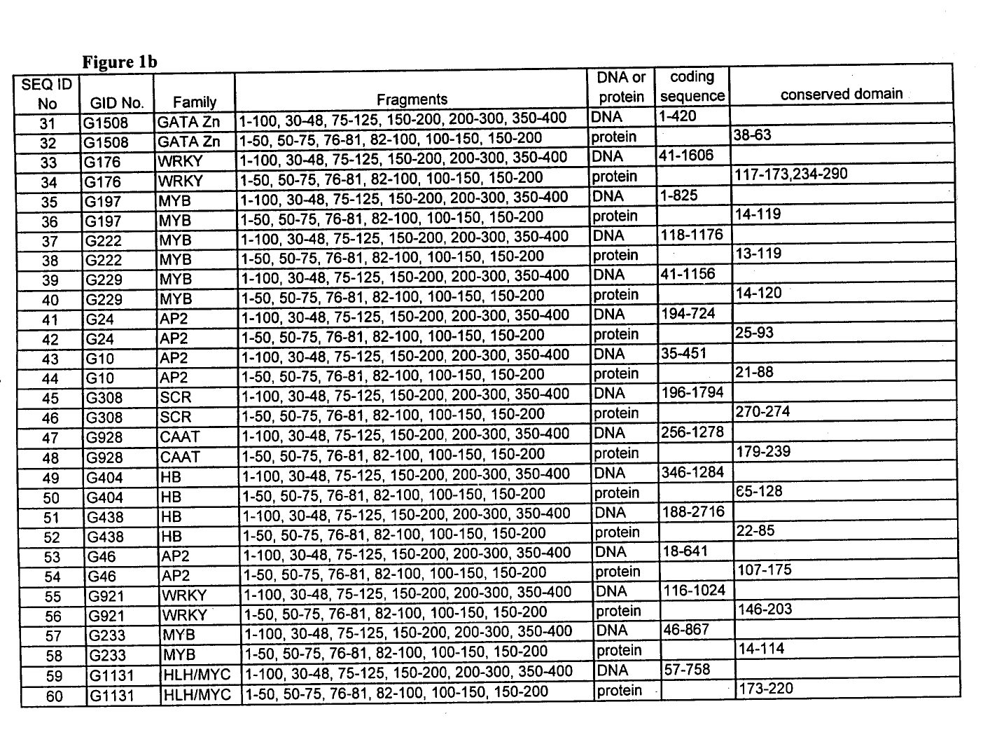

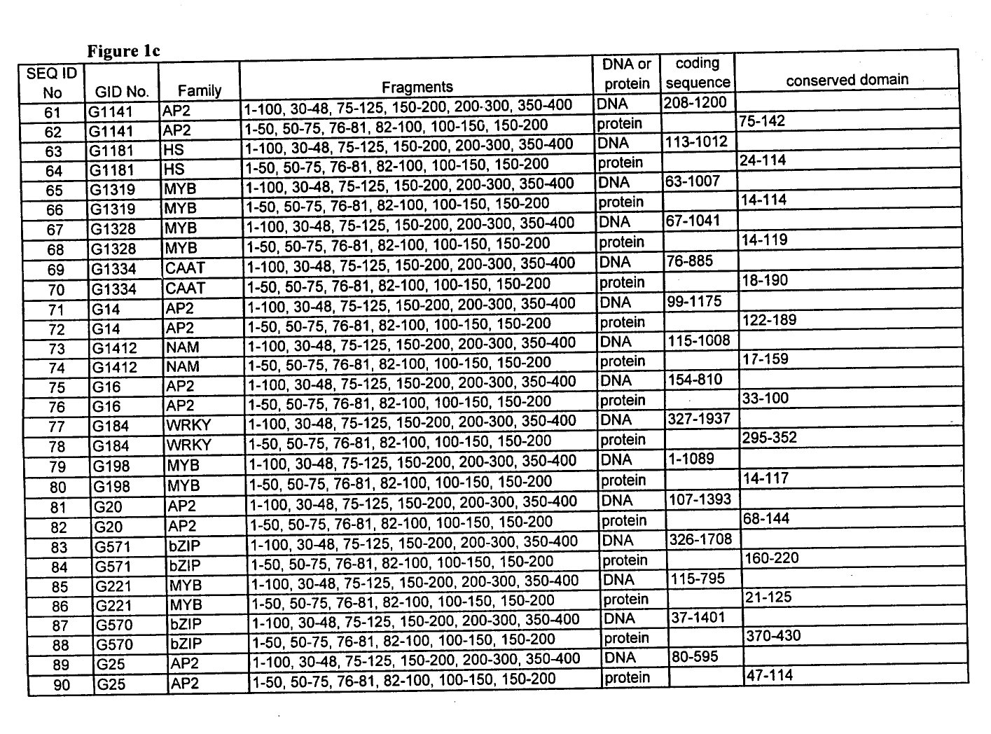

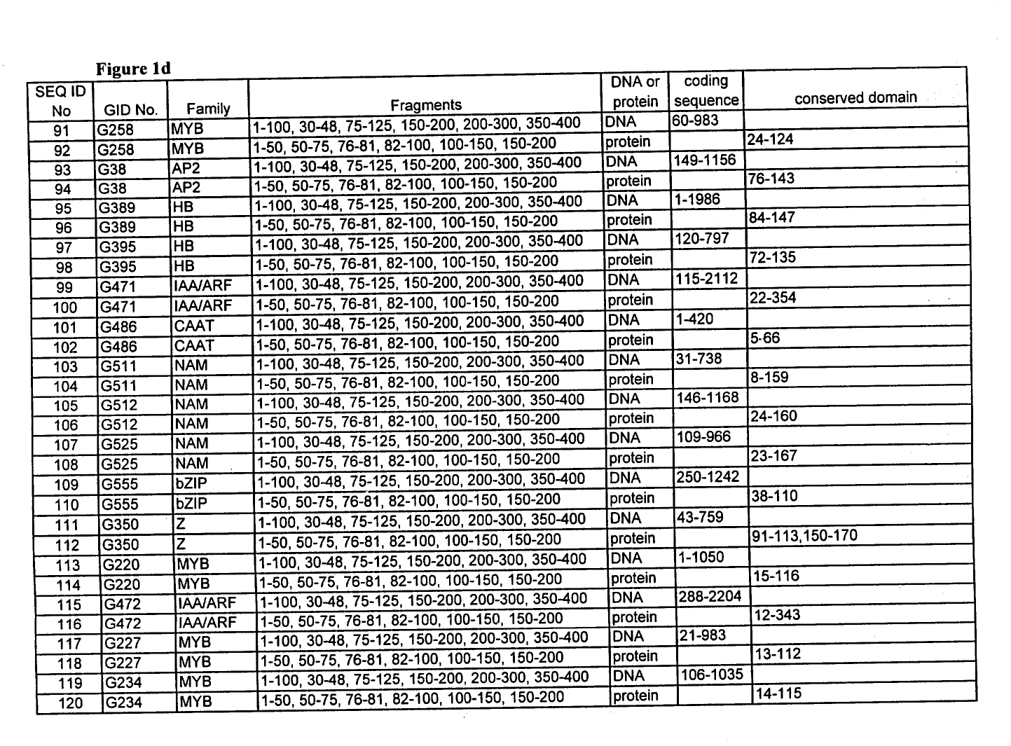

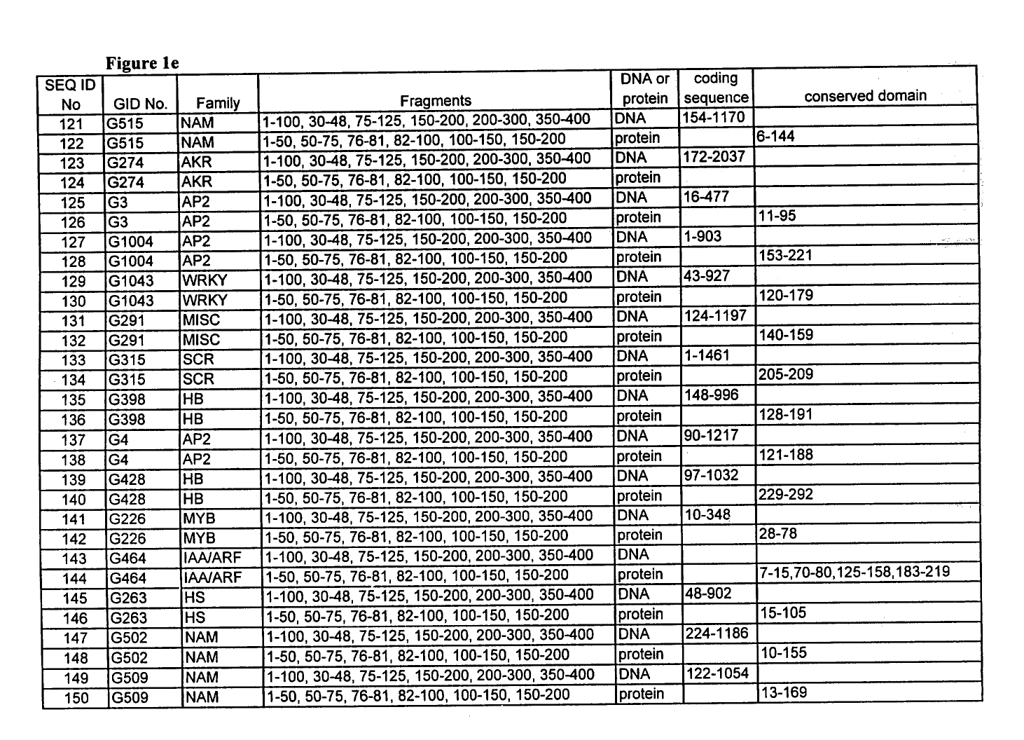

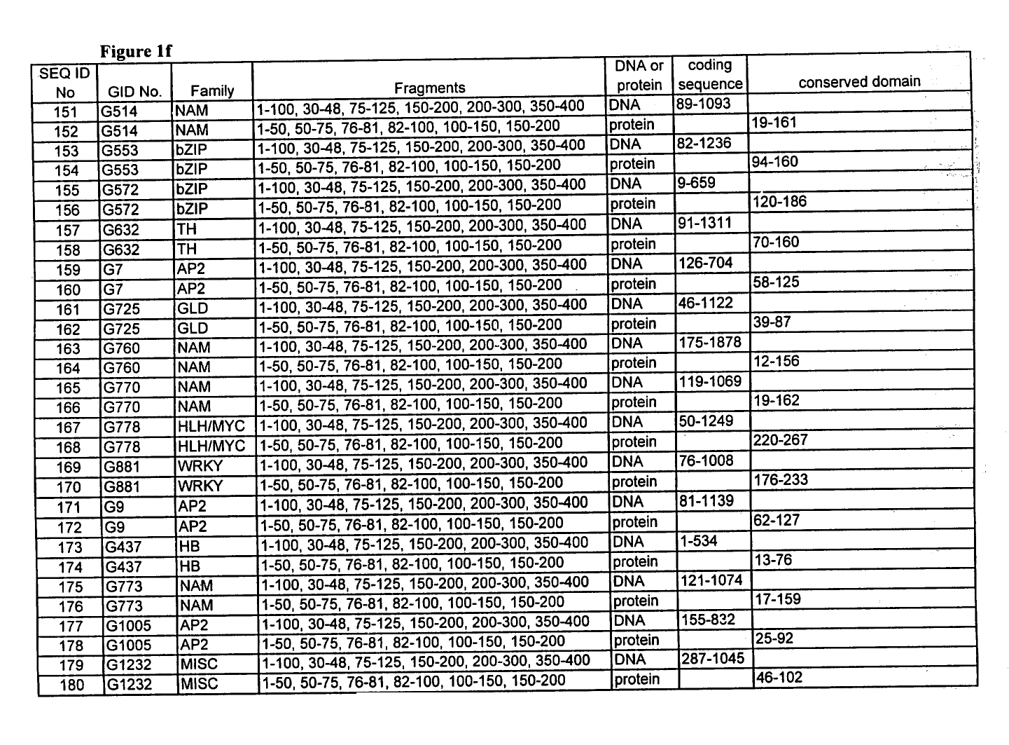

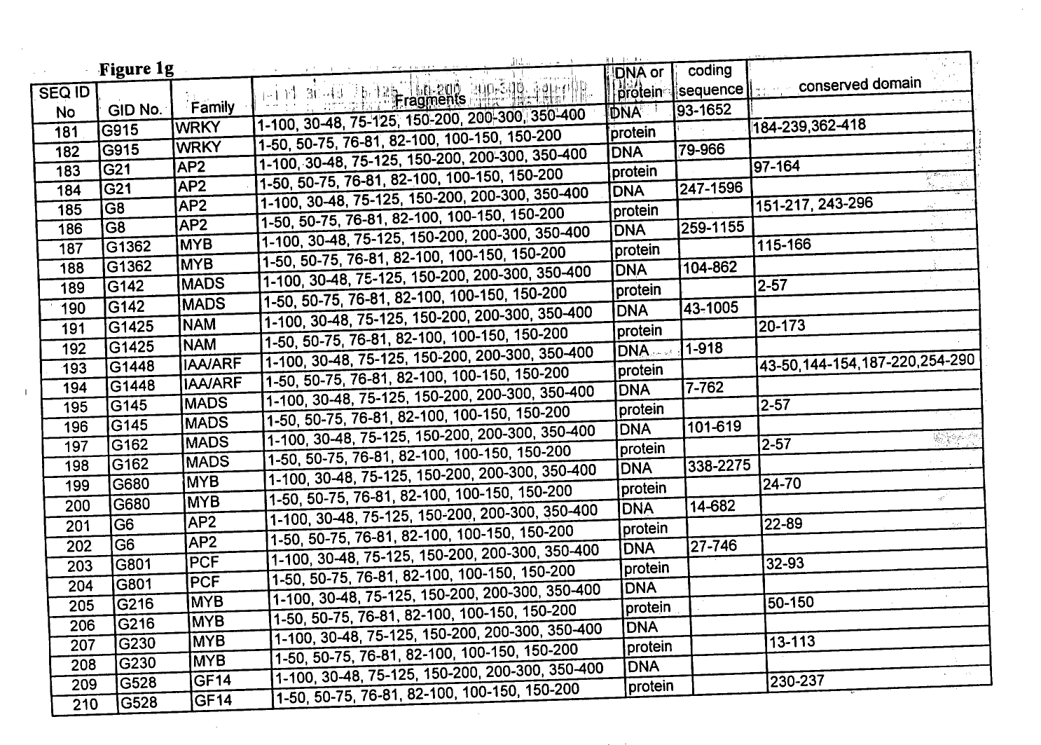

The polynucleotides and polypeptides are provided in the Sequence Listing and are tabulated in FIG. 1. FIG. 1 identifies a SEQ ID No., its corresponding GID number, the transcription factor family to which the sequence belongs, fragments derived from the sequences, whether the sequence is a polynucleotide or a polypeptide sequence, the full length coding sequences and conserved domains. We have also identified domains or fragments derived from the sequences. The numbers indicating the fragment location for the DNA sequences may be from either 5′ or 3′ end of the DNA. For the protein sequences the fragment location is determined from the N-terminus of the protein and may include adjacent amino acid sequences, such as for example for SEQ ID No. 2 an additional 10, 20, 40, 60 or 100 amino acids in either N-terminal or C-terminal direction of the described fragments.

The identified polypeptide fragments may be linked to fragments or sequences derived from other transcription factors so as to generate additional novel sequences, such as by employing the methods described in Short, PCT publication WO9827230, entitled “Methods and Compositions for Polypeptide Engineering” or in Patten et al., PCT publication WO9923236, entitled “Method of DNA Shuffling”. Alternatively, the identified fragment may be linked to a transcription activation domain. A transcription activation domain assists in initiating transcription from a DNA binding site. A common feature of some activation domains is that they are designed to form amphiphilic alpha helices with excess positive or negative charge (Giniger and Ptashne (1987) Nature 330:670-672, Gill and Ptashne (1987) Cell 51:121-126, Estruch et al (1994) Nucl. Acids Res. 22:3983-3989). Examples include the transcription activation region of VP16 or GAL4 (Moore et al. (1998) Proc. Natl. Acad. Sci. USA 95: 376-381; and Aoyama et al. (1995) Plant Cell 7:1773-1785), peptides derived from bacterial sequences (Ma and Ptashne (1987) Cell 51; 113-119) and synthetic peptides (Giniger and Ptashne, supra).

The isolated polynucleotides and polypeptides may be used to modify plant development, physiology or biochemistry such that the modified plants have a trait advantage over wild type plants. The identified polynucleotide fragments are also useful as nucleic acid probes and primers. A nucleic acid probe is useful in hybridization protocols, including protocols for microarray experiments. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, e.g., by the polymerase chain reaction (PCR) or other nucleic-acid amplification methods. See Sambrook et al., Molecular Cloning. A Laboratory Manual, Ed. 2, Cold Spring Harbor Laboratory Press, New York (1989) and Ausubel et al. (eds) Current Protocols in Molecular Biology, John Wiley & Sons (1998).

2. Identification of Homologous Sequences (Homologs)

Homologous sequences to those provided in the Sequence Listing derived from Arabidopsis thaliana or from other plants may be used to modify a plant trait. Homologous sequences may be derived from any plant including monocots and dicots and in particular agriculturally important plant species, including but not limited to, crops such as soybean, wheat, corn, potato, cotton, rice, oilseed rape (including canola), sunflower, alfalfa, sugarcane and turf; or fruits and vegetables, such as banana, blackberry, blueberry, strawberry, and raspberry, cantaloupe, carrot, cauliflower, coffee, cucumber, eggplant, grapes, honeydew, lettuce, mango, melon, onion, papaya, peas, peppers, pineapple, spinach, squash, sweet corn, tobacco, tomato, watermelon, rosaceous fruits (such as apple, peach, pear, cherry and plum) and vegetable brassicas (such as broccoli, cabbage, cauliflower, brussel sprouts and kohlrabi). Other crops, fruits and vegetables whose phenotype may be changed include barley, currant, avocado, citrus fruits such as oranges, lemons, grapefruit and tangerines, artichoke, cherries, nuts such as the walnut and peanut, endive, leek, roots, such as arrowroot, beet, cassaya, turnip, radish, yam, sweet potato and beans. The homologs may also be derived from woody species, such pine, poplar and eucalyptus.

Substitutions, deletions and insertions introduced into the sequences provided in the Sequence Listing are also envisioned by the invention. Such sequence modifications can be engineered into a sequence by site-directed mutagenesis (Wu (ed.) Meth. Enzymol. (1993) vol. 217, Academic Press). Amino acid substitutions are typically of single residues; insertions usually will be on the order of about from 1 to 10 amino acid residues; and deletions will range about from 1 to 30 residues. In preferred embodiments, deletions or insertions are made in adjacent pairs, e.g., a deletion of two residues or insertion of two residues. Substitutions, deletions, insertions or any combination thereof may be combined to arrive at a sequence. The mutations that are made in the polynucleotide encoding the transcription factor should not place the sequence out of reading frame and should not create complementary regions that could produce secondary mRNA structure.

Substitutions are those in which at least one residue in the amino acid sequence has been removed and a different residue inserted in its place. Such substitutions may be conservative with little effect on the function of the gene, for example by substituting alanines for serines, arginines for lysines, glutamate for aspartate and the like. The substitutions which are not conservative are expected to produce the greatest changes in protein properties will be those in which (a) a hydrophilic residue, e.g., seryl or threonyl, is substituted for (or by) a hydrophobic residue, e.g., leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine.

Additionally, the term “homologous sequence” may encompass a polypeptide sequence that is modified by chemical or enzymatic means. The homologous sequence may be a sequence modified by lipids, sugars, peptides, organic or inorganic compounds, by the use of modified amino acids or the like. Protein modification techniques are illustrated in Ausubel et al. (eds) Current Protocols in Molecular Biology, John Wiley & Sons (1998).

Homologous sequences also may mean two sequences having a substantial percentage of sequence identity after alignment as determined by using sequence analysis programs for database searching and sequence alignment and comparison available, for example, from the Wisconsin Package Version 10.0, such as BLAST, FASTA, PILEUP, FINDPATTERNS or the like (GCG, Madision, Wis.). Public sequence databases such as GenBank, EMBL, Swiss-Prot and PIR or private sequence databases such as PhytoSeq (Incyte Pharmaceuticals, Palo Alto, Calif.) may be searched. Alignment of sequences for comparison may be conducted by the local homology algorithm of Smith and Waterman (1981) Adv. Appl. Math. 2:482, by the homology alignment algorithm of Needleman and Wunsch (1970) J. Mol. Biol. 48:443, by the search for similarity method of Pearson and Lipman (1988) Proc. Natl. Acad. Sci. U.S.A. 85: 2444, by computerized implementations of these algorithms. After alignment, sequence comparisons between two (or more) polynucleotides or polypeptides are typically performed by comparing sequences of the two sequences over a comparison window to identify and compare local regions of sequence similarity. The comparison window may be a segment of at least about 20 contiguous positions, usually aboit 50 to about 200, more usually about 100 to about 150 contiguous positions. A description of the method is provided in Ausubel et al. (eds) (1999) Current Protocols in Molecular Biology, John Wiley & Sons.

Transcription factors that are homologs of the disclosed sequences will typically share at least 40% amino acid sequence identity. More closely related TFs may share at least 50%, 60%, 65%, 70%, 75% or 80% sequence identity with the disclosed sequences. Factors that are most closely related to the disclosed sequences share at least 85%, 90% or 95% sequence identity. At the nucleotide level, the sequences will typically share at least 40% nucleotide sequence identity, preferably at least 50%, 60%, 70% or 80% sequence identity, and more preferably 85%, 90%, 95% or 97% sequence identity. The degeneracy of the genetic code enables major variations in the nucleotide sequence of a polynucleotide while maintaining the amino acid sequence of the encoded protein.

One way to identify whether two nucleic acid molecules are closely related is that the two molecules hybridize to each other under stringent conditions. Generally, stringent conditions are selected to be about 5° C. to 20° C. lower than the thermal melting point (Tm) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (1989) Molecular Cloning. A Laboratory Manual, Ed. 2, Cold Spring Harbor Laboratory Press, New York and Tijssen (1993) Laboratory Techniques in Biochemistry and Molecular Biology—Hybridization with Nucleic Acid Probes Part I, Elsevier, New York. Nucleic acid molecules that hybridize under stringent conditions will typically hybridize to a probe based on either the entire cDNA or selected portions of the cDNA under wash conditions of 0.2×SSC to 2.0×SSC, 0.1% SDS at 50-65° C., for example 0.2×SSC, 0.1% SDS at 65° C. For detecting less closely related homologs washes may be performed at 50° C.

For conventional hybridization the hybridization probe is conjugated with a detectable label such as a radioactive label, and the probe is preferably of at least 20 nucleotides in length. As is well known in the art, increasing the length of hybridization probes tends to give enhanced specificity. The labeled probe derived from the Arabidopsis nucleotide sequence may be hybridized to a plant cDNA or genomic library and the hybridization signal detected using means known in the art. The hybridizing colony or plaque (depending on the type of library used) is then purified and the cloned sequence contained in that colony or plaque isolated and characterized. Homologs may also be identified by PCR-based techniques, such as inverse PCR or RACE, or hybridizing to DNA microarrays using degenerate primers. See Ausubel et al. (eds) (1998) Current Protocols in Molecular Biology, John Wiley & Sons.

TF homologs may alternatively be obtained by immunoscreening an expression library. With the provision herein of the disclosed TF nucleic acid sequences, the polypeptide may be expressed and purified in a heterologous expression system (e.g., E. coli) and used to raise antibodies (monoclonal or polyclonal) specific for the TF. Antibodies may also be raised against synthetic peptides derived from TF amino acid sequences. Methods of raising antibodies are well known in the art and are described in Harlow and Lane (1988) Antibodies: A Laboratory Manual, Cold Spring Harbor Laboratory, New York. Such antibodies can then be used to screen an expression library produced from the plant from which it is desired to clone the TF homolog, using the methods described above. The selected cDNAs may be confirmed by sequencing and enzymatic activity.

3. Altered Expression of Transcription Factors

Any of the identified sequences may be incorporated into a cassette or vector for expression in plants. A number of expression vectors suitable for stable transformation of plant cells or for the establishment of transgenic plants have been described including those described in Weissbach and Weissbach, (1989) Methods for Plant Molecular Biology, Academic Press, and Gelvin et al., (1990) Plant Molecular Biology Manualx, Kluwer Academic Publishers. Specific examples include those derived from a Ti plasmid of Agrobacterium tumefaciens, as well as those disclosed by Herrera-Estrella, L., et al., (1983) Nature 303: 209, Bevan, M., Nucl. Acids Res. (1984) 12: 8711-8721, Klee, H. J., (1985) Bio/Technology 3: 637642, for dicotyledonous plants. Ti-derived plasmids can be transferred into both monocotonous and docotyledonous species using Agrobacterium-mediated transformation (Ishida et al (1996) Nat. Biotechnol. 14:745-50; Barton et al. (1983) Cell 32:1033-1043).

Alternatively, non-Ti vectors can be used to transfer the DNA into plants and cells by using free DNA delivery techniques. Such methods may involve, for example, the use of liposomes, electroporation, microprojectile bombardment, silicon carbide wiskers, and viruses. By using these methods transgenic plants such as wheat, rice (Christou, P., (1991) Bio/Technology 9: 957-962) and corn (Gordon-Kamm, W., (1990) Plant Cell 2: 603-618) can be produced. An immature embryo can also be a good target tissue for monocots for direct DNA delivery techniques by using the particle gun (Weeks, T. et al., (1993) Plant Physiol. 102: 1077-1084; Vasil, V., (1993) Bio/Technology 10: 667-674; Wan, Y. and Lemeaux, P., (1994) Plant Physiol. 104: 37-48, and for Agrobacterium-mediated DNA transfer (Ishida et al., (1996) Nature Biotech. 14: 745-750).

Typically, plant transformation vectors include one or more cloned plant coding sequences (genomic or cDNA) under the transcriptional control of 5′ and 3′ regulatory sequences and a dominant selectable marker. Such plant transformation vectors typically also contain a promoter (e.g., a regulatory region controlling inducible or constitutive, environmentally-or developmentally-regulated, or cell- or tissue-specific expression), a transcription initiation start site, an RNA processing signal (such as intron splice sites), a transcription termination site, and/or a polyadenylation signal.

Examples of constitutive plant promoters which may be useful for expressing the TF sequence include: the cauliflower mosaic virus (CaMV) 35S promoter, which confers constitutive, high-level expression in most plant tissues (see, e.g., Odel et al., (1985) Nature 313:810); the nopaline synthase promoter (An et al., (1988) Plant Physiol. 88:547); and the octopine synthase promoter (Fromm et al., (1989) Plant Cell 1: 977).

A variety of plant gene promoters that regulate gene expression in response to environmental, hormonal, chemical, developmental signals, and in a tissue-active manner can be used for expression of the TFs in plants, as illustrated by seed-specific promoters (such as the napin, phaseolin or DC3 promoter described in U.S. Pat. No. 5,773,697), root-specific promoters, such as those disclosed in U.S. Pat. Nos. 5,618,988, 5,837,848 and 5,905,186; fruit-specific promoters that are active during fruit ripening (such as the dru 1 promoter (U.S. Pat. No. 5,783,393), or the 2A11 promoter (U.S. Pat. No. 4,943,674) and the tomato polygalacturonase promoter (Bird et al. (1988) Plant Mol. Biol. 11:651), root-specific promoters, such as those disclosed in U.S. Pat. Nos. 5,618,988, 5,837,848 and 5,905,186, pollen-active promoters such as PTA29, PTA26 and PTA13 (U.S. Pat. No. 5,792,929), promoters active in vascular tissue (Ringli and Keller (1998) Plant Mol. Biol. 37:977-988), flower-specific (Kaiser et al, (1995) Plant Mol. Biol. 28:231-243), pollen (Baerson et al. (1994) Plant Mol. Biol. 26:1947-1959), carpels (Ohl et al. (1990) Plant Cell 2:837-848), pollen and ovules (Baerson et al. (1993) Plant Mol. Biol. 22:255-267) auxin-inducible promoters (such as that described in van der Kop et al (1999) Plant Mol. Biol 39:979-990 or Baumann et al. (1999) Plant Cell 11:323-334), cytokinin-inducible promoter (Guevara-Garcia (1998) Plant Mol. Biol. 38:743-753), promoters responsive to gibberellin (Shi et al. (1998) Plant Mol. Biol 38:1053-1060, Willmott et al. (1998) 38:817-825) and the like. Additional promoters are those that elicit expression in response to heat (Ainley, et al. (1993) Plant Mol. Biol. 22: 13-23), light (e.g., the pea rbcS-3A promoter, Kuhlemeier et al., (1989) Plant Cell 1:471, and the maize rbcS promoter, Schaffner and Sheen, (1991) Plant Cell 3: 997); wounding (e.g., wunl, Siebertz et al., (1989) Plant Cell 1: 961); pathogen resistance, and chemicals such as methyl jasmonate or salicylic acid (Gatz et al., (1997) Plant Mol. Biol 48: 89-108). In addition, the timing of the expression can be controlled by using promoters such as those acting at late seed development (Odell et al. (1994) Plant Physiol. 106:447-458).

Plant expression vectors may also include RNA processing signals that may be positioned within, upstream or downstream of the coding sequence. In addition, the expression vectors may include additional regulatory sequences from the 3′-untranslated region of plant genes, e.g., a 3′ terminator region to increase mRNA stability of the mRNA, such as the PI-II terminator region of potato or the octopine or nopaline synthase 3′ terminator regions.

Finally, as noted above, plant expression vectors may also include dominant selectable marker genes to allow for the ready selection of transformants. Such genes include those encoding antibiotic resistance genes (e.g., resistance to hygromycin, kanamycin, bleomycin, G418, streptomycin or spectinomycin) and herbicide resistance genes (e.g., phosphinothricin acetyltransferase).

A reduction of TF expression in a transgenic plant to modifiy a plant trait may be obtained by introducing into plants antisense constructs based on the TF cDNA. For antisense suppression, the TF cDNA is arranged in reverse orientation relative to the promoter sequence in the expression vector. The introduced sequence need not be the full length TF cDNA or gene, and need not be identical to the TF cDNA or a gene found in the plant type to be transformed. Generally, however, where the introduced sequence is of shorter length, a higher degree of homology to the native TF sequence will be needed for effective antisense suppression. Preferably, the introduced antisense sequence in the vector will be at least 30 nucleotides in length, and improved antisense suppression will typically be observed as the length of the antisense sequence increases. Preferably, the length of the antisense sequence in the vector will be greater than 100 nucleotides. Transcription of an antisense construct as described results in the production of RNA molecules that are the reverse complement of mRNA molecules transcribed from the endogenous TF gene in the plant cell. Suppression of endogenous TF gene expression can also be achieved using a ribozyme. Ribozymes are synthetic RNA molecules that possess highly specific endoribonuclease activity. The production and use of ribozymes are disclosed in U.S. Pat. No. 4,987,071 to Cech and U.S. Pat. No. 5,543,508 to Haselhoff. The inclusion of ribozyme sequences within antisense RNAs may be used to confer RNA cleaving activity on the antisense RNA, such that endogenous mRNA molecules that bind to the antisense RNA are cleaved, which in turn leads to an enhanced antisense inhibition of endogenous gene expression.

Vectors in which RNA encoded by the TF cDNA (or variants thereof) is over-expressed may also be used to obtain co-suppression of the endogenous TF gene in the manner described in U.S. Pat. No. 5,231,020 to Jorgensen. Such co-suppression (also termed sense suppression) does not require that the entire TF cDNA be introduced into the plant cells, nor does it require that the introduced sequence be exactly identical to the endogenous TF gene. However, as with antisense suppression, the suppressive efficiency will be enhanced as (1) the introduced sequence is lengthened and (2) the sequence similarity between the introduced sequence and the endogenous TF gene is increased.

Vectors expressing an untranslatable form of the TF mRNA may also be used to suppress the expression of endogenous TF activity to modify a trait. Methods for producing such constructs are described in U.S. Pat. No. 5,583,021 to Dougherty et al. Preferably, such constructs are made by introducing a premature stop codon into the TF gene. Alternatively, a plant trait may be modified by gene silencing using double-strand RNA (Sharp (1999) Genes and Development 13:139-141).

Another method for abolishing the expression of a gene is by insertion mutagenesis using the T-DNA of Agrobacterium tumefaciens. After generating the insertion mutants, the mutants can be screened to identify those containing the insertion in a TF gene. Mutants containing a single mutation event at the desired gene may be crossed to generate homozygous plants for the mutation (Koncz et al. (1992) Methods in Arabidopsis Research. World Scientific).

A plant trait may also be modified by using the cre-lox system (for example, as described in U.S. Pat. No. 5,658,772). A plant genome may be modified to include first and second lox sites that are then contacted with a Cre recombinase. If the lox sites are in the same orientation, the intervening DNA sequence between the two sites is excised. If the lox sites are in the opposite orientation, the intervening sequence is inverted.

The polynucleotides and polypeptides of this invention may also be expressed in a plant in the absence of an expression cassette by manipulating the activity or expression level of the endogenous gene by other means. For example, by ectopically expressing a gene by T-DNA activation tagging (Ichikawa et al., (1997) Nature 390 698-701, Kakimoto et al., (1996) Science 274: 982-985). This method entails transforming a plant with a gene tag containing multiple transcriptional enhancers and once the tag has inserted into the genome, expression of a flanking gene coding sequence becomes deregulated. In another example, the transcriptional machinery in a plant may be modified so as to increase transcription levels of a polynucleotide of the invention (See PCT Publications WO9606166 and WO 9853057 which describe the modification of the DNA binding specificity of zinc finger proteins by changing particular amino acids in the DNA binding motif).

The transgenic plant may also comprise the machinery necessary for expressing or altering the activity of a polypeptide encoded by an endogenous gene, for example by altering the phosphorylation state of the polypeptide to maintain it in an activated state.

4. Transgenic Plants with Modified TF Expression

Once an expression cassette comprising a polynucleotide encoding a TF gene of this invention has been constructed, standard techniques may be used to introduce the polynucleotide into a plant in order to modify a trait of the plant. The plant may be any higher plant, including gymnosperms, monocotyledonous and dicotyledenous plants. Suitable protocols are available for Leguminosae (alfalfa, soybean, clover, etc.), Umbelliferae (carrot, celery, parsnip), Cruciferae (cabbage, radish, rapeseed, broccoli, etc.), Curcurbitaceae (melons and cucumber), Gramineae (wheat, corn, rice, barley, millet, etc.), Solanaceae (potato, tomato, tobacco, peppers, etc.), and various other crops. See protocols described in Ammirato et al. (1984) Handbook of Plant Cell Culture—Crop Species. Macmillan Publ. Co. Shimamoto et al. (1989) Nature 338:274-276; Fromm et al. (1990) Bio/Technology 8:833839; and Vasil et al. (1990) Bio/Technology 8:429-434.

Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods may include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium tumeficiens mediated transformation. Transformation means introducing a nucleotide sequence in a plant in a manner to cause stable or transient expression of the sequence.

Successful examples of the modification of plant characteristics by transformation with cloned sequences which serve to illustrate the current knowledge in this field of technology, and which are herein incorporated by reference, include: U.S. Pat. Nos. 5,571,706; 5,677,175; 5,510,471; 5,750,386; 5,597,945; 5,589,615; 5,750,871; 5,268,526; 5,780,708; 5,538,880; 5,773,269; 5,736,369 and 5,610,042.

Following transformation, plants are preferably selected using a dominant selectable marker incorporated into the transformation vector. Typically, such a marker will confer antibiotic or herbicide resistance on the transformed plants, and selection of transformants can be accomplished by exposing the plants to appropriate concentrations of the antibiotic or herbicide.

After transformed plants are selected and grown to maturity, those plants showing a modified trait are identified. The modifed trait may be any of those traits described above. Additionally, to confirm that the modified trait is due to changes in expression levels or activity of the polypeptide or polynucleotide of the invention may be determined by analyzing mRNA expression using Northern blots, RT-PCR or microarrays, or protein expression using immunoblots or Western blots or gel shift assays.

5. Other Utility of the Polypeptide and Polynucleotide Sequences

A transcription factor provided by the present invention may also be used to identify exogenous or endogenous molecules that may affect expression of the transcription factors and may affect any of the traits described herein. These molecules may include organic or inorganic compounds.

For example, the method may entail first placing the molecule in contact with a plant or plant cell. The molecule may be introduced by topical administration, such as spraying or soaking of a plant, and then the molecule's effect on the expression or activity of the TF polypeptide or the expression of the polynucleotide monitored. Changes in the expression of the TF polypeptide may be monitored by use of polyclonal or monoclonal antibodies, gel electrophoresis or the like. Changes in the expression of the corresponding polynucleotide sequence may be detected by use of microarrays, Northerns or any other technique for monitoring changes in mRNA expression. These techniques are exemplified in Ausubel et al. (eds) Current Protocols in Molecular Biology, John Wiley & Sons (1998). Such changes in the expression levels may be correlated with modified plant traits and thus identified molecules may be useful for soaking or spraying on fruit, vegetable and grain crops to modify traits in plants.

The transcription factors may also be employed to identify promoter sequences with which they may interact. After identifying a promoter sequence, interactions between the transcription factor and the promoter sequence may be modified by changing specific nucleotides in the promoter sequence or specific amino acids in the transcription factor that interact with the promoter sequence to alter a plant trait. Typically, transcription factor DNA binding sites are identified by gel shift assays. After identifying the promoter regions, the promoter region sequences may be employed in double-stranded DNA arrays to identify molecules that affect the interactions of the TFs with their promoters (Bulyk et al. (1999) Nature Biotechnology 17:573-577).

The identified transcription factors are also useful to identify proteins that modify the activity of the transcription factor. Such modification may occur by covalent modification, such as by phosphorylation, or by protein-protein (homo or-heteropolymer) interactions. Any method suitable for detecting protein-protein interactions may be employed. Among the methods that may be employed are co-immunoprecipitation, cross-linking and co-purification through gradients or chromatographic columns, and the two-hybrid yeast system.

The two-hybrid system detects protein interactions in vivo and is described in Chien, et al., (1991), Proc. Natl. Acad. Sci. USA, 88, 9578-9582 and is commercially available from Clontech (Palo Alto, Calif.). In such a system, plasmids are constructed that encode two hybrid proteins: one consists of the DNA-binding domain of a transcription activator protein fused to the TF polypeptide and the other consists of the transcription activator protein's activation domain fused to an unknown protein that is encoded by a cDNA that has been recombined into the plasmid as part of a cDNA library. The DNA-binding domain fusion plasmid and the cDNA library are transformed into a strain of the yeast Saccharomyces cerevisiae that contains a reporter gene (e.g., lacZ) whose regulatory region contains the transcription activator's binding site. Either hybrid protein alone cannot activate transcription of the reporter gene. Interaction of the two hybrid proteins reconstitutes the functional activator protein and results in expression of the reporter gene, which is detected by an assay for the reporter gene product. Then, the library plasmids responsible for reporter gene expression are isolated and sequenced to identify the proteins encoded by the library plasmids. After identifying proteins that interact with the transcription factors, assays for compounds that interfere with the TF protein-protein interactions may be preformed.

The following examples are intended to illustrate but not limit the present invention.

### EXAMPLE I

### Full Length Gene Identification and Cloning

Putative transcription factor sequences (genomic or ESTs) related to known transcription factors were identified in the Arabidopsis thaliana GenBank database using the tblastn sequence analysis program using default parameters and a P-value cutoff threshold of −4 or −5 or lower, depending on the length of the query sequence. Putative transcription factor sequence hits were then screened to identify those containing particular sequence strings. If the sequence hits contained such sequence strings, the sequences were confirmed as transcription factors.

Alternatively, Arabidopsis thaliana cDNA libraries derived from different tissues or treatments, or genomic libraries were screened to identify novel members of a transcription family using a low stringency hybridization approach. Probes were synthesized using gene specific primers in a standard PCR reaction (annealing temperature 60° C.) and labeled with ³²P dCTP using the High Prime DNA Labeling Kit (Boehringer Mannheim). Purified radiolabelled probes were added to filters immersed in Church hybridization medium (0.5 M NaPO₄ pH 7.0, 7% SDS, 1% w/v bovine serum albumin) and hybridized overnight at 60° C. with shaking. Filters were washed two times for 45 to 60 minutes with 1×SCC, 1% SDS at 60° C.

To identify additional sequence 5′ or 3′ of a partial cDNA sequence in a cDNA library, 5′ and 3′ rapid amplification of cDNA ends (RACE) was performed using the Marathon™ cDNA amplification kit (Clontech, Palo Alto, Calif.). Generally, the method entailed first isolating poly(A) mRNA, performing first and second strand cDNA synthesis to generate double stranded cDNA, blunting cDNA ends, followed by ligation of the Marathon™ Adaptor to the cDNA to form a library of adaptor-ligated ds cDNA. Gene-specific primers were designed to be used along with adaptor specific primers for both 5′ and 3′ RACE reactions. Nested primers, rather than single primers, were used to increase PCRspecificity. Using 5′ and 3′ RACE reactions, 5′ and 3′ RACE fragments were obtained, sequenced and cloned. The process may be repeated until 5′ and 3′ ends of the full-length gene were identified. Then the full-length cDNA was generated by PCR using primers specific to 5′ and 3′ ends of the gene by end-to-end PCR.

### EXAMPLE II

### Environmental Stress Genes

RT-PCR experiments using treated rosette leaf tissue were performed as described above to identify those genes induced after exposure of the plants or seedlings to chilling stress (6 hour exposure to 4° C.), heat stress (6 hour exposure to 37° C.), high salt stress (6 hour exposure to 200 mM NaCl), drought stress (168 hours after removing water from trays), osmotic stress (6 hour exposure to 3 M mannitol), hormones (6 hours after spraying plants with 1 uM indole acetic acid (2,4-D) or 50 uM abcissic acid (ABA)). The gene expression patterns from ground plant leaf tissue was investigated as described above.

Reverse transcriptase PCR was done using gene specific primers within the coding region for each sequence identified. The primers were designed near the 3′ region of each coding sequence initially identified.

Total RNA from these tissues were isolated using the CTAB extraction protocol. Once extracted total RNA was normalized in concentration across all the tissue types to ensure that the PCR reaction for each tissue received the same amount of cDNA template using the 28S band as reference. Poly A+ was purified using a modified protocol from the Qiagen Oligotex kit batch protocol. cDNA was synthesized using standard protocols. After the first strand cDNA synthesis, primers for Actin 2 were used to normalize the concentration of cDNA across the tissue types. Actin 2 is found to be constitutively expressed in fairly equal levels across the tissue types we are investigating.

For RT PCR, cDNA template was mixed with corresponding primers and Taq polymerase. Each reaction consisted of 0.2 ul cDNA template, 2 ul 10×Tricine buffer, 2 ul 10×Tricine buffer and 16.8 ul water, 0.05 ul Primer 1, 0.05 ul, Primer 2, 0.3 ul Taq polymerase and 8.6 ul water.

The 96 well plate was covered with microfilm and set in the Thermocycler to start the following reaction cycle. Step 1 93° C. for 3 mins, Step 2 93° C. for 30 sec, Step 3 65° C. for 1 min, Step 4 72° C. for 2 mins, Steps 2, 3 and 4 were repeated for 28 cycles, Step 5 72° C. for 5 mins and Step 6 4° C. The PCR plate was placed back in the thermocycler to amplify more products at 8 more cycles to identify genes that have very low expression. The reaction cycle was as follows: Step 2 93° C. for 30 sec, Step 3 65° C. for 1 min, and Step 4 72° C. for 2 ins, repeated for 8 cycles, and Step 4 4° C.

8 ul of PCR product and 1.5 ul of loading dye were loaded on a 1.2% agarose gel for analysis after 28 cycles and 36 cycles. Expression levels of specific transcripts were considered low if they were only detectable after 36 cycles of PCR. Expression levels were considered medium or high depending on the levels of transcript compared with observed transcript levels for actin2.

Microarray experiments were also performed after plants were treated at 4° C. for 2, 4, 8, 12 or 24 hours, after plants were grown on 500 mM mannitol for 24, 48, or 72 hours For measuring the effect of drought, plants were grown on MS plates for two weeks, then pulled from the media and placed in a laminar flow hood for 30 minutes. The plants were subsequently transplanted on MS plates and allowed to recover for 3 hours after which time leaf tissue was harvested for analysis.

Expression patterns of the transcription factors was monitored by microarray experiments. cDNAs were generated by PCR and resuspended at a final concentration of ˜100 ng/ul in 3×SSC or 150 mM Na-phosphate (Eisen and Brown (1999) Meth. in Enzymol. 303:179-205). The cDNAs were spotted on microscope glass slides coated with polylysine. The prepared cDNAs were aliquoted into 384 well plates and spotted on the slides using an x-y-z gantry (OmniGrid) purchased from GeneMachines (Menlo Park, Calif.) outfitted with quill type pins purchased from Telechem International (Sunnyvale, Calif.). After spotting, the arrays were cured for a minimum of one week at room temperature, rehydrated and blocked following the protocol recommended by Eisen and Brown (1999).

Sample total RNA (10 ug) samples were labeled using fluorescent Cy3 and Cy5 dyes. Labeled samples were resuspended in 4×SSC/0.03% SDS/4 ug salmon sperm DNA/2 ug tRNA/50 mM Na-pyrophosphate, heated for 95° C. for 2.5 minutes, spun down and placed on the array. The array was then covered with a glass coverslip and placed in a sealed chamber. The chamber was then kept in a water bath at 62° C. overnight.

The arrays were washed as described in Eisen and Brown (1999) and scanned on a General Scanning 3000 laser scanner. The resulting files are subsequently quantified using Imagene a software purchased from BioDiscovery (Los Angeles, Calif.).

Transcript levels of the genes described in the invention were more than 2-fold compared with control samples.

### EXAMPLE III

### Construction of Expression Vectors

The sequence was amplified from a genomic or cDNA library using primers specific to sequences upstream and downstream of the coding region. The expression vector was pMEN20, which is derived from pMON316 (Sanders et al, (1987) Nucleic Acids Research 15:1543-58). To clone the sequence into the vector, both pMEN20 and the amplified DNA fragment were digested separately with SalI and NotI restriction enzymes at 37° C. for 2 hours. The digestion products were subject to electrophoresis in a 0.8% agarose gel and visualized by ethidium bromide staining. The DNA fragments containing the sequence and the linearized plasmid were excised and purified by using a Qiaquick gel extraction kit (Qiagen, CA). The fragments of interest were ligated at a ratio of 3:1 (vector to insert). Ligation reactions using T4 DNA ligase (New England Biolabs, MA) were carried out at 16° C. for 16 hours. The ligated DNAs were transformed into competent cells of the E. coli strain DH5alpha by using the heat shock method. The transformations were plated on LB plates containing 50 mg/l spectinomycin (Sigma).

Individual colonies were grown overnight in five milliliters of LB broth containing 50 mg/l spectinomycin at 37° C. Plasmid DNA was purified by using Qiaquick Mini Prep kits (Qiagen, CA).

### EXAMPLE IV

### Transformation of Agrobacterium with the Expression Vector

After the plasmid vector containing the gene was constructed, the vector was used to transform Agrobacterium tumefaciens cells expressing the gene products. The stock of Agrobacterium tumefaciens cells for transformation were made as described by Nagel et al. FEMS Microbiol Letts 67: 325-328 (1990). Agrobacterium strain GV3101 was grown in 250 ml LB medium (Sigma) overnight at 28° C. with shaking until an absorbance (A₆₀₀) of 0.5-1.0 was reached. Cells were harvested by centrifugation at 4,000×g for 15 min at 4° C. Cells were then resuspended in 250 μl chilled buffer (1 mM HEPES, pH adjusted to 7.0 with KOH). Cells were centrifuged again as described above and resuspended in 125 μl chilled buffer. Cells were then centrifuged and resuspended two more times in the same HEPES buffer as described above at a volume of 100 μl and 750 μl, respectively. Resuspended cells were then distributed into 40 μl aliquots, quickly frozen in liquid nitrogen, and stored at −80° C.

Agrobacterium cells were transformed with plasmids prepared as described above following the protocol described by Nagel et al. FEMS Microbiol Letts 67: 325-328 (1990). For each DNA construct to be transformed, 50-100 ng DNA (generally resuspended in 10 mM Tris-HCl, 1 mM EDTA, pH 8.0) was mixed with 40 μl of Agrobacterium cells. The DNA/cell mixture was then transferred to a chilled cuvette with a 2 mm electrode gap and subject to a 2.5 kV charge dissipated at 25 μF and 200 μF using a Gene Pulser II apparatus (Bio-Rad). After electroporation, cells were immediately resuspended in 1.0 ml LB and allowed to recover without antibiotic selection for 2-4 hours at 28° C. in a shaking incubator. After recovery, cells were plated onto selective medium of LB broth containing 100 pg/ml spectinomycin (Sigma) and incubated for 24-48 hours at 28° C. Single colonies were then picked and inoculated in fresh medium. The presence of the plasmid construct was verified by PCR amplification and sequence analysis.

### EXAMPLE V

### Transformation of Arabidopsis Plants with Agrobacterium tumefaciens with Expression Vector

After transformation of Agrobacterium tumefaciens with plasmid vectors containing the gene, single Agrobacterium colonies were identified, propagated, and used to transform Arabidopsis plants. Briefly, 500 ml cultures of LB medium containing 50 mg/l spectinomycin were inoculated with the colonies and grown at 28° C. with shaking for 2 days until an absorbance (A₆₀₀) of >2.0 is reached. Cells were then harvested by centrifugation at 4,000×g for 10 min, and resuspended in infiltration medium (½×Murashige and Skoog salts (Sigma), 1×Gamborg's B-5 vitamins (Sigma), 5.0% (w/v) sucrose (Sigma), 0.044 μM benzylamino purine (Sigma), 200 μl/L Silwet L-77 (Lehle Seeds) until an absorbance (A₆₀₀) of 0.8 was reached.

Prior to transformation, Arabidopsis thaliana seeds (ecotype Columbia) were sown at a density of ˜10 plants per 4″ pot onto Pro-Mix BX potting medium (Hummert International) covered with fiberglass mesh (18 mm×16 mm). Plants were grown under continuous illumination (50-75 μE/m²/sec) at 22-23° C. with 65-70% relative humidity. After about 4 weeks, primary inflorescence stems (bolts) are cut off to encourage growth of multiple secondary bolts. After flowering of the mature secondary bolts, plants were prepared for transformation by removal of all siliques and opened flowers.

The pots were then immersed upside down in the mixture of Agrobacterium infiltration medium as described above for 30 sec, and placed on their sides to allow draining into a 1′×2′ flat surface covered with plastic wrap. After 24 h, the plastic wrap was removed and pots are turned upright. The immersion procedure was repeated one week later, for a total of two immersions per pot. Seeds were then collected from each transformation pot and analyzed following the protocol described below.

### EXAMPLE VI

### Identification of Arabidopsis Primary Transformants

Seeds collected from the transformation pots were sterilized essentially as follows. Seeds were dispersed into in a solution containing 0.1% (v/v) Triton X-100 (Sigma) and sterile H₂O and washed by shaking the suspension for 20 min. The wash solution was then drained and replaced with fresh wash solution to wash the seeds for 20 min with shaking. After removal of the second wash solution, a solution containing 0.1% (v/v) Triton X-100 and 70% ethanol (Equistar) was added to the seeds and the suspension was shaken for 5 min. After removal of the ethanol/detergent solution, a solution containing 0.1% (v/v) Triton X-100 and 30% (v/v) bleach (Clorox) was added to the seeds, and the suspension was shaken for 10 min. After removal of the bleach/detergent solution, seeds were then washed five times in sterile distilled H₂O. The seeds were stored in the last wash water at 4° C. for 2 days in the dark before being plated onto antibiotic selection medium (1×Murashige and Skoog salts (pH adjusted to 5.7 with 1 M KOH), 1×Gamborg's B-5 vitamins, 0.9% phytagar (Life Technologies), and 50 mg/l kanamycin). Seeds were germinated under continuous illumination (50-75 μE/m²/sec) at 22-23° C. After 7-10 days of growth under these conditions, kanamycin resistant primary transformants (T₁ generation) were visible and obtained. These seedlings were transferred first to fresh selection plates where the seedlings continued to grow for 3-5 more days, and then to soil (Pro-Mix BX potting medium).

Primary transformants are self-crossed and progeny seeds (T2) collected.

### EXAMPLE VII

### Analysis of Arabidopsis T2 Progeny Plants for Stress Resistance or Stress Tolerance

T2 progeny seeds were germinated on kanamycin as described above and kanamycin resistant seedlings were selected, transferred to soil and analyzed. Environmental stress tolerance in a transgenic Arabidopsis plant was compared with that of a wild type plant.

In one analysis to identify plants with superior germination or seedling growth in high salt-stress conditions, T2 progeny seed were tested for their ability to germinate in 80% Murashige and Skoog media plus vitamins (Sigma) containing 150 mM sodium chloride in 24 hour light (120-130 uEin s⁻¹ m⁻²) in a growth chamber. 150 mM sodium chloride is usually a restrictive germination temperature for Arabidopsis. Plants containing the G482 and G226 constructs showed more vigorous seedling growth than controls with 150 mM sodium chloride, so G482 and G226 could be used for conferring salt tolerance to germinating seedlings.

In another analysis to identify plants with superior germination or seedling growth under temperature stress conditions, T2 progeny seed were tested for their ability to germinate in 80% Murashige and Skoog media plus vitamins (Sigma) at 32° C. under 24-hour light (120-130 uEin s⁻¹ m⁻²) in a growth chamber. 32° C. is usually a restrictive germination temperature for Arabidopsis. Plants containing the G682 construct showed more vigorous seedling growth than controls at 32° C., so G682 could be used for conferring heat tolerance to germinating seedlings.

In another analysis to identify plants more resistant to pathogens than controls, 4week old T2 progeny plants were by hand innoculated with the bacterial pathogen Pseudomonas syringae pv maculicola (OD=0.005) using a 1 ml syringe. Plants containing the G525 construct showed fewer disease symptoms three days after innoculation with the bacterial pathogen than controls. Therefore G525 can be used to manipulate the plant defense, wound, or insect response in order to generate pathogen resistant plants.

### EXAMPLE VIII

### Transformation of Cereal Plants with the Expression Vector

A cereal plant, such as corn, wheat, rice, sorghum or barley, can also be transformed with the plasmid vectors containing the sequence and constitutive or inducible promoters to modify a trait. In these cases, a cloning vector, pMEN020, is modified to replace the NptII coding region with the BAR gene of Streptomyces hygroscopicus that confers resistance to phosphinothricin. The KpnI and BgIII sites of the Bar gene are removed by site-directed mutagenesis with silent codon changes.

Plasmids according to the present invention may be transformed into corn ernbryogenic cells derived from immature scutellar tissue by using microprojectile bombardment, with the A188XB73 genotype as the preferred genotype (Fromm et al., Bio/Technology 8: 833-839 (1990); Gordon-Kamm et al., Plant Cell 2: 603-618 (1990)). After microprojectile bombardment the tissues are selected on phosphinothricin to identify the transgenic embryogenic cells (Gordon-Kamm et al., Plant Cell 2: 603-618 (1990)). Transgenic plants are regenerated by standard corn regeneration techniques (Fromm, et al., Bio/Technology 8: 833-839 (1990); Gordon-Kamm et al., Plant Cell 2: 603-618 (1990)).

### EXAMPLE IX

### Identification of Homologous Sequences

Homologs from the same plant, different plant species or other organisms were identified using database sequence search tools, such as the Basic Local Alignment Search Tool (BLAST) (Altschul et al. (1990) J. Mol. Biol. 215:403-410; and Altschul et al. (i997) Nucl. Acid Res. 25: 3389-3402). The tblastn or blastn sequence analysis programs were employed using the BLOSUM-62 scoring matrix (Henikoff, S. and Henikoff, J. G. (1992) Proc. Natl. Acad. Sci. USA 89: 10915-10919). The output of a BLAST report provides a score that takes into account the alignment of similar or identical residues and any gaps needed in order to align the sequences. The scoring matrix assigns a score for aligning any possible pair of sequences. The P values reflect how many times one expects to see a score occur by chance. Higher scores are preferred and a low threshold P value threshold is preferred. These are the sequence identity criteria. The tblastn sequence analysis program was used to query a polypeptide sequence against six-way translations of sequences in a nucleotide database. Hits with a P value less than −25, preferably less than −70, and more preferably less than −100, were identified as homologous sequences (exemplary selected sequence criteria). The blastn sequence analysis program was used to query a nucleotide sequence against a nucleotide sequence database. In this case too, higher scores were preferred and a preferred threshold P value was less than −13, preferably less than −50, and more preferably less than 100.

Alternatively, a fragment of a sequence from FIG. 1 is ³²P-radiolabeled by random priming (Sambrook et al., (1989) Molecular Cloning. A Laboratory Manual, 2^{nd} Ed., Cold Spring Harbor Laboratory Press, New York) and used to screen a plant genomic library (the exemplary test polynucleotides). As an example, total plant DNA from Arabidopsis thaliana, Nicotiana tabacum, Lycopersicon pimpinellfolium, Prunus avium, Prunus cerasus, Cucumis sativus, or Oryza saliva are isolated according to Stockinger al (Stockinger, E. J., et al., (1996), J. Heredity, 87:214-218). Approximately 2 to 10 μg of each DNA sample are restriction digested, transferred to nylon membrane (Micron Separations, Westboro, Mass.) and hybridized. Hybridization conditions are: 42° C. in 50% formamide, 5×SSC, 20 mM phosphate buffer 1×Denhardt's, 10% dextran sulfate, and 100 μg/ml herring sperm DNA. Four low stringency washes at RT in 2×SSC, 0.05% sodium sarcosyl and 0.02% sodium pyrophosphate are performed prior to high stringency washes at 55° C. in 0.2×SSC, 0.05% sodium sarcosyl and 0.01% sodium pyrophosphate. High stringency washes are performed until no counts are detected in the washout according to Walling et al. (Walling, L. L., et al., (1988) Nucl. Acids Res. 16:10477-10492).

All references (publications and patents) are incorporated herein by reference in their entirety for all purposes.

Although the invention has been described with reference to the embodiments and examples above, it should be understood that various modifications can be made without departing from the spirit of the invention. Accordingly, the invention is limited only by the following claims.

We claim:

1. A transgenic plant comprising a recombinant polynucleotide comprising a nucleotide sequence encoding a polypeptide comprising at least 6 consecutive amino acids of a sequence selected from the group consisting of SEQ ID Nos. 2N, where N=1-111, wherein the recombinant polynucleotide alters the plant's environmental stress tolerance when compared with the same trait of another plant lacking the recombinant polynucleotide.

2. The transgenic plant of claim 1, wherein the nucleotide sequence encodes a polypeptide comprising a conserved domain selected from the group consisting of SEQ ID Nos. 2N, where N=1-111

3. The transgenic plant of claim 1, wherein the recombinant polynucleotide further comprises a promoter operably linked to said nucleotide sequence.

4. The transgenic plant of claim 3, wherein said promoter is constitutive or inducible or tissue-active.

5. A method for altering the environmental stress response of a plant, said method comprising (a) transforming a plant with a recombinant polynucleotide comprising a nucleotide sequence encoding a polypeptide comprising at least 6 consecutive amino acids of a sequence selected from the group consisting of SEQ ID Nos. 2N, where N=1-111, (b) selecting said transformed plants; and (c) identifying a transformed plant having an altered environmental stress tolerance.

6. The method of claim 5, wherein the nucleotide sequence encodes a polypeptide comprising a conserved domain selected from the group consisting of SEQ ID Nos. 2N, where N=1-111.

8. The method of claim 5, wherein the recombinant polynucleotide further comprises a promoter operably linked to said nucleotide sequence.

9. The method of claim 8, wherein said promoter is constitutive or inducible or tissue-active.

10. A method for altering the expression levels of at least one gene in a plant, said method comprising (a) transforming the plant with a recombinant polynucleotide comprising a nucleotide sequence encoding a polypeptide comprising at least 6 consecutive amino acids of a sequence selected from the group consisting of SEQ ID Nos. 2N, where N=1-111; and (b) selecting said transformed plant.

11. The method of claim 10, wherein said recombinant polynucleotide encodes a polypeptide comprising a conserved domain selected from the group consisting of SEQ ID Nos. 2N, where N=1-111.

12. The method of claim 10, wherein the nucleotide sequence further comprises a promoter operably linked to said nucleotide sequence.

13. The method of claim 10, wherein said promoter is constitutive or inducible or tissue active.

14. A method for altering the environmental stress tolerance of a plant, said method comprising (a) transforming the plant with a recombinant polynucleotide comprising a nucleotide sequence comprising at least 18 consecutive nucleotides of a sequence selected from the group consisting of SEQ ID Nos. 2N−1, where N=1-111, and SEQ ID Nos. 223-238; and (b) selecting said transformed plant.

15. A method for altering a plant's trait, said method comprising (a) providing a database sequence; (b) comparing said database sequence with a polypeptide selected from SEQ ID Nos. 2N, where N=1-111; (c) selecting a database sequence that meets selected sequence criteria; and (d) transforming said selected database sequence in the plant.

16. A method for altering a plant's trait, said method comprising (a) providing a database sequence; (b) comparing said database sequence with a polynucleotide selected from SEQ ID Nos. 2N−1, where N=1-111, and SEQ ID Nos. 223-238; (c) selecting a database sequence that meets selected sequence criteria; and (d) transforming said selected database sequence in the plant.

17. A method for altering a plant's trait, said method comprising (a) providing a test polynucleotide; (b) hybridizing said test polynucleotide with a polynucleotide selected from SEQ ID Nos. 2N−1, where N=1-111, and SEQ ID Nos. 223-238 at low stringency; and (c) transforming said hybridizing test polynucleotide in a plant to alter a trait of the plant.


SEQUENCE LISTING (238 sequences)

SEQ ID NO: 1 - DNA, 1624 bp, Arabidopsis thaliana
  G1426
gcaccagcggcaccaggaatcagaatcttcatgtattatcaatggattcgttaccagttggattaagattccgtccaacagacgaggagctaatccgttactatctccgtaggaaaatcaacggtcacgatgacgacgtcaaagctatccgtgagatcgatatttgcaaatgggaaccttgggatttacctgatttttctgtgatcaaaactaaagactcagagtggctctacttctgtccattggatcggaagtatccgagtggaagtagacagaaccgggcaacagttgcagggtactggaaagccacaggaaaagaccggaagataaaatccggtaagactaacattattggtgtgaagagaactctagttttccacgcgggtagagctcctagggggacacgaaccaattggattattcatgagtatcgtgccacggaggatgatcttagtggtaccaatcctggccagagtccgtttgttatatgcaaattgttcaagaaagaagaactggttttaggggaagaagattcgaagtcagatgaagttgaagaacctgctgtctcgtctccaactgtcgaagtgactaagtcagaagtatctgaggtaattaaaacagaagacgtgaagcgtcatgacatagcagaatcttctcttgtaatctctggagattctcatagtgatgcttgtgatgaggctacaaccgcagagcttgtagattttaaatggtatccggaattggagtccttagatttcacgctgttctctccattacactctcaagtccaatctgagcttggatcctcttacaacacattccagcctggctcgagtaatttttcagggaacaacaacaacagcttccaaatccagactcagtatggtacaaatgaagtagatacgtatatatctgattttcttgattcgattctcaagagcccagacgaggatccagagaagcacaagtatgttttgcaaagtggttttgatgttgtagcaccagatcagattgcgcaagtgtgtcaacagggctcagcagttgacatgagcaatgatgtttcagtgacgggtatccagattaagagtcgccaggcacaaccctcaggttataccaatgactatatagcgcagggaaatggtccaagaaggctgcgtctgcaaagtaactttaatggcattaacacgaagaatcctgagcttcaagctataaagagagaggctgaggacacggttggagaaagtataaagaaaaggtgtggaaagctaatgagatcaaagaacgtgaccggttttgtgtttaaaaagattacgtcggtgaaatgttcatatggaggactctttagggcggcagtagttgcagttgtgttcttgatgtcagtctgcagtctaaccgtcgacttcagagcatctgcagtgtcctgaaaactgaaactttttgtaaatatagtgcttgaaccggcccgtgtttttgcttctgttattggtttcaggctttcgccagtttataagatatatttgtttgtagtttttacatgtagttaggctgcttatgtaaacgttatgatgacagagaggtcaagactctatttacttctttgactaaaaaaaaaaaaaaaaaaaaa

SEQ ID NO: 2 - protein, 460 aa, Arabidopsis thaliana
  G1426
MDSLPVGLRFRPTDEELIRYYLRRKINGHDDDVKAIREIDICKWEPWDLPDFSVIKTKDSEWLYFCPLDRKYPSGSRQNRATVAGYWKATGKDRKIKSGKTNIIGVKRTLVFHAGRAPRGTRTNWIIHEYRATEDDLSGTNPGQSPFVICKLFKKEELVLGEEDSKSDEVEEPAVSSPTVEVTKSEVSEVIKTEDVKRHDIAESSLVISGDSHSDACDEATTAELVDFKWYPELESLDFTLFSPLHSQVQSELGSSYNTFQPGSSNFSGNNNNSFQIQTQYGTNEVDTYISDFLDSILKSPDEDPEKHKYVLQSGFDVVAPDQIAQVCQQGSAVDMSNDVSVTGIQIKSRQAQPSGYTNDYIAQGNGPRRLRLQSNFNGINTKNPELQAIKREAEDTVGESIKKRCGKLMRSKNVTGFVFKKITSVKCSYGGLFRAAVVAVVFLMSVCSLTVDFRASAVS

SEQ ID NO: 3 - DNA, 978 bp, Arabidopsis thaliana
  G578
atgcatagtttgaatgaaacagtaattcctgatgttgattacatgcagtctgatagagggcatatgcatgctgctgcctctgattccagtgatcgatcaaaggataagttggatcaaaagacccttcgtaggcttgctcaaaatcgtgaggcagcaagaaaaagcagattgaggaagaaggcgtatgttcagcagctggaagatagtcgattaaagctgactcaagttgagcaggagctgcaaagagcaagacagcagggagttttcatctcaagttcaggagaccaagctcattctactggtggcaatggtggggctttggcatttgatgcagaacactcacgatggcttgaagaaaagaacaggcaaatgaacgagctgagatctgccctgaatgctcatgcaggtgatactgagctccggataattgtggatggagtgatggctcactatgaggagcttttcaggattaagagcaatgcatctaagaatgatgtcttccacttgttatctggaatgtggaaaacaccagctgagcgatgtttcttgtggcttggcgggttcccgtcatccgaacttctcaagcttcttgcgaatcagctagagcccatgacagaacgacaggtaatgggcatcaatagcttgcagcagacgtcgcagcaggcagaagatgctttatctcaagggatggagagtttacagcaatccctagctgatactttatccagtggaactcttggttccagttcatcggataatgtcgcgagctacatgggtcagatggccatggcaatgggcaagttaggcaccctcgaaggattcatacgccaggctgataacttgaggctgcaaacactacaacagatgcttcgagtattaacaacacgtcagtcagctcgtgctcttcttgctatacacgattattcatctcgattacgtgctcttagttccttgtggcttgcccggccaagagagtga

SEQ ID NO: 4 - protein, 325 aa, Arabidopsis thaliana
  G578
MHSLNETVIPDVDYMQSDRGHMHAAASDSSDRSKDKLDQKTLRRLAQNREAARKSRLRKKAYVQQLEDSRLKLTQVEQELQRARQQGVFISSSGDQAHSTGGNGGALAFDAEHSRWLEEKNRQMNELRSALNAHAGDTELRIIVDGVMAHYEELFRIKSNASKNDVFHLLSGMWKTPAERCFLWLGGFPSSELLKLLANQLEPMTERQVMGINSLQQTSQQAEDALSQGMESLQQSLADTLSSGTLGSSSSDNVASYMGQMAMAMGKLGTLEGFIRQADNLRLQTLQQMLRVLTTRQSARALLAIHDYSSRLRALSSLWLARPRE

SEQ ID NO: 5 - DNA, 1226 bp, Arabidopsis thaliana
  G598
gtccgttgtcatattttaaatttatcaccttcttgagaattccacatttttatcctttttgtcatgtagtgtatattttttcctctaacctaattaaaatcaaaacaaaatcctttgacccaattagcttcgcgatatatcagaagagatcaaactactttgatcagaccatgatcttcttcttcttcttcttcttcttcttcttctttttagacgatcacaattcctaaaccctatttctcagattatgctgactctttaccatcaagaaaggtcaccggacgccacaagtaatgatcgcgatgagacgccagagactgtggttagagaagtccacgcgctaactccagcgccggaggataattcccggacgatgacggcgacgctacctccaccgcctgctttccgaggctatttttctcctccaaggtcagcgacgacgatgagcgaaggagagaacttcacaactataagcagagagttcaacgctctagtcatcgccggatcctccatggagaacaacgaactaatgactcgtgacgtcacgcagcgtgaagatgagagacaagacgagttgatgagaatccacgaggacacggatcatgaagaggaaacgaatcctttagcaatcgtgccggatcagtatcctggttcgggtttggatcctggaagtgataatgggccgggtcagagtcgggttgggtcgacggtgcaaagagttaagagggaagaggtggaagcgaagataacggcgtggcagacggcaaaactggctaagattaataacaggtttaagagggaagacgccgttattaacggttggtttaatgaacaagttaacaaggccaactcttggatgaagaaaattgagtataatgtaggttcattcaacaatcgtctaaatgaggaagctagaggagagaaaagcaaaagcgatggagaaaacgcaaaacaatgtggcgaaagcgcagaggaaagcggaggagagaagagcgacggcagaggcaaagagagggacagaggttgcaaaagtagttgaagttgctaatctcatgagagcccttggacgtcctcctgccaaacgctccttcttctctttctcctaatttttagttatatcaaaccattaaattaaacagtactcgttatatatctagttagtaaacaaaggggcagttttatagctcatgtacacataattgagagtgtagtactgttgtgtcaaa

SEQ ID NO: 6 - protein, 263 aa, Arabidopsis thaliana
  G598
MLTLYHQERSPDATSNDRDETPETVVREVHALTPAPEDNSRTMTATLPPPPAFRGYFSPPRSATTMSEGENFTTISREFNALVIAGSSMENNELMTRDVTQREDERQDELMRIHEDTDHEEETNPLAIVPDQYPGSGLDPGSDNGPGQSRVGSTVQRVKREEVEAKITAWQTAKLAKINNRFKREDAVINGWFNEQVNKANSWMKKIEYNVGSFNNRLNEEARGEKSKSDGENAKQCGESAEESGGEKSDGRGKERDRGCKSS

SEQ ID NO: 7 - DNA, 2311 bp, Arabidopsis thaliana
  G610
tcttaatcagagattcgtgagaggtaaaggtagcgtaattattcagtttgcgtgatctataatttaactggcgccagattgtgatccaggaatcgttggcttgttaggttgttgttttgttatgcataattgagagatggtggtcaaaaggaagttaaattgtggtggctctgatggttttgatttccccaatattcccaaggctcctcgttcaagcaggaggaaggtctcaggtaagagatctgatgatgaaagtgagatctgtgcaattgatttgctagcttctcttgctggaaagttgttggaagaaagtgaaagttcctcaacgtctacctatgcatctgaagctgataatcttgatcatttgggtggactgattaagcaagaacttgaagatggctatactactaagccttgtaaatccgagtttttcgatccaggaaaccctgcttcaaagtccactagtgaaaatactagcgtgacttgtttgccattttcgtctttcgaaaatgattgcattttggagcaaacaccagtttctgattgtaagagggcatctggtttgaagtccctggtagggagcatcactgaggagacatgtgttgttaatgaggatgccggatctgaacaaggtgctaatactttcagcttaaaggatccaagtcaattacattcgcagtctccagaatcggtccttctggatggcgatgtgaaattagcaccatgcacggatcaagtccctaatgattcttttaaaggatataggaatcattctaagttagtttgcagagatgatgacgaaaactattgtaagtattataaatttagtgacaaatgtaagtcatataggcctctctcccgggttggcaatagaagaataatgcagtcggtgagagcaatctccaagttgaagtgttttgaagacactagaacagatggtcgtttgaaggctctctaccgcaagagaaaattatgttatggttacaacccatggaagcgtgagaccattcataggaagagaagattgtctgacaaaggtttggtcgtaaattatgatggtgggctcagtagtgaaagtgtttccaattcacctgaaaagggagaatcagaaaatggtgatttctctgctgcaaaaataggtcttctttcgaaagactcccgtgtaaagttcagcatcaagtcccttaggattccggagcttgtaattgaagttccagaaacagcaacagtaggcttactgaagaggacggtgaaggaggcggttactgctttactcggtggtggaatacgtattggggtgttagtccaagggaaaaaagttagagatgacaacaacactctatcacagactggtctttcgtgtagagaaaatcttggcaaccttggcttcaccttagagcctggtttggaaacactgcctgtacctctttgttctgaaactcctgtcctttctctgccaactgactctacaaagttgtcagaaaggtccgcagcttctccagcgttagagactggaattcctctccctccccaagatgaagattacttgattaatttgggaaatagtgtggagaacaatgatgaattagtcccacatctgagtgacataccagctgatgaacaaccttcatcagattcaagagcgctggttccagttttggccttggagtccgacgctcttgcacttgttccagttaacgagaaacctaagcgtacagagctttcacaacgcagaaccaggagactattctctgttacagaggtagaagctctagtaagcgcagttgaagaagttgggactggaagatggcgtgatgtgaagttgcgttcttttgagaatgcaagtcatcgaacctatgtggacttgaaggacaaatggaaaacgttggttcacacagcaagtatatcaccacagcaacgaagaggagaaccagtgcctcaagaactgctagacagagtcttaggagcacataggtactggacacagcaccaaatgaaacagaacgggaaacatcaggtggctacaacaatggtggttgaagcaggttcgtccatgtaaagaaggagaatggtagtaacaataactttcacttgacgactaaggaaccaaagtgggcaactgtacaaagggaaacaacaaaatacagaaacatacttaatttctgaaaagaagagtctatatttttattttttttaaatcatagccggtagaaacaagacgttccttgacacttttggttacttttatggtaggtctgttcattccaaatttctaattgatttgattatgtaatttggtggtaggaccatg

SEQ ID NO: 8 - protein, 640 aa, Arabidopsis thaliana
  G610
MVVKRKLNCGGSDGFDFPNIPKAPRSSRRKVSGKRSDDESEICAIDLLASLAGKLLEESESSSTSTYASEADNLDHLGGLIKQELEDGYTTKPCKSEFFDPGNPASKSTSENTSVTCLPFSSFENDCILEQTPVSDCKRASGLKSLVGSITEETCVVNEDAGSEQGANTFSLKDPSQLHSQSPESVLLDGDVKLAPCTDQVPNDSFKGYRNHSKLVCRDDDENYCKYYKFSDKCKSYRPLSRVGNRRIMQSVRAISKLKCFEDTRTDGRLKALYRKRKLCYGYNPWKRETIHRKRRLSDKGLVVNYDGGLSSESVSNSPEKGESENGDFSAAKIGLLSKDSRVKFSIKSLRIPELVIEVPETATVGLLKRTVKEAVTALLGGGIRIGVLVQGKKVRDDNNTLSQTGLSCRENLGNLGFTLEPGLETLPVPLCSETPVLSLPTDSTKLSERSAASPALETGIPLPPQDEDYLINLGNSVENNDELVPHLSDIPADEQPSSDSRALVPVLALESDALALVPVNEKPKRTELSQRRTRRLFSVTEVEALVSAVEEVGTGRWRDVKLRSFENASHRTYVDLKDKWKTLVHTASISPQQRRGEPVPQELLDRVLGAHRYWTQHQMKQNGKHQVATTMVVEAGSSM

SEQ ID NO: 9 - DNA, 826 bp, Arabidopsis thaliana
  G620
gaattgaacttggaccagcacagcaacaacccaaccccaatgaccagctcagtcatagtagccggcgccggtgacaagaacaatggtatcgtggtccagcagcaaccaccatgtgtggctcgtgagcaagaccaatacatgccaatcgcaaacgtcataagaatcatgcgtaaaaccttaccgtctcacgccaaaatctctgacgacgccaaagaaacgattcaagaatgtgtctccgagtacatcagcttcgtgaccggtgaagccaacgagcgttgccaacgtgagcaacgtaagaccataactgctgaagatatcctttgggctatgagcaagcttgggttcgataactacgtggaccccctcaccgtgttcattaaccggtaccgtgagatagagaccgatcgtggttctgcacttagaggtgagccaccgtcgttgagacaaacctatggaggaaatggtattgggtttcacggcccatctcatggcctacctcctccgggtccttatggttatggtatgttggaccaatccatggttatgggaggtggtcggtactaccaaaacgggtcgtcgggtcaagatgaatccagtgttggtggtggctcttcgtcttccattaacggaatgccggcttttgaccattatggtcagtataagtgaagaaggagttattcttcatttttatatctattcaaaacatgtgtttcgatagatattttatttttatgtcttatcaataacatttctatataatgttgcttctttaaggaaaagtgttgtatgtcaatactttatgagaaactgatttatatatgcaaat

SEQ ID NO: 10 - protein, 208 aa, Arabidopsis thaliana
  G620
MTSSVIVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 11 - DNA, 2121 bp, Arabidopsis thaliana
  G634
ggagctagagagaaagagagagtgtgtgtgtagaaaaagattgaaacatcatcaagattgatggagcaaggaggaggtggtggtggtaatgaagttgtggaggaagcttcacctattagttcaagacctcctgctaacaacttagaagagcttatgagattctcagccgccgcggatgacggtggattaggaggtggaggtggaggaggaggaggaggaagtgcttcttcttcatcgggaaatcgatggccgagagaagaaactttagctcttcttcggatccgatccgatatggattctacttttcgtgatgctactctcaaagctcctctttgggaacatgtttccaggaagctattggagttaggttacaaacgaagttcaaagaaatgcaaagagaaattcgaaaacgttcagaaatattacaaacgtactaaagaaactcgcggtggtcgtcatgatggtaaagcttacaagttcttctctcagcttgaagctctcaacactactcctccttcatcttccctcgacgttactcctctctccgtcgctaatcccattctcatgccttcttcttcttcttctccatttcccgtattctctcaaccgcaaccgcaaacgcaaacgcaaccgcctcaaacgcataatgtctcttttactcctactccaccacctcttccacttccttcaatgggtccgatatttaccggtgttactttctcgtctcatagctcatcgacggcttcaggaatggggtctgatgatgatgacgacgatatggacgttgatcaggctaacattgcgggttctagtagccgaaaacgcaaacgtggaaaccgcggtggaggcggtaaaatgatggaattgtttgaaggtttggtgagacaagtaatgcaaaagcaagcggctatgcaaaggagtttcttggaagctcttgagaagagagagcaagaacgtcttgatcgtgaagaagcttggaaacgtcaagaaatggctcggttagctcgagaacacgaggtcatgtctcaagaacgagccgcctctgcttctcgtgacgccgcaatcatttcattgattcagaaaattactggccataccattcagttacctccttctttgtcatctcaaccgcctccaccgtatcaaccgccacccgcggtcactaaacgtgtggcggaaccaccattatcaacagctcaatctcaatcacaacaaccaataatggcgattccacaacaacaaattcttcctcctcctcctccttctcatcctcacgctcatcaaccagaacagaaacaacaacaacaaccacaacaagagatggtcatgagctcggaacaatcatcattaccatcatcatcaagatggccaaaggcagagattctagcgcttataaacctgagaagtggaatggaaccaaggtaccaagataatgtacctaaaggacttctatgggaagagatctcaacttcaatgaagagaatgggatacaacagaaacgctaagagatgtaaagagaaatgggaaaacataaacaaatactacaagaaagttaaagaaagcaacaagaaacgtcctcaagatgctaagacttgtccttactttcaccgcctcgatcttctttaccgcaacaaagtactcggtagtggcggtggttctagcacttctggtctacctcaagaccaaaaacagagtccggtcactgcgatgaaaccgccacaagaaggacttgttaatgttcaacaaactcatgggtcagcttcaactgaggaagaagagcctatagaggaaagtccacaaggaacagaaaagccagaagaccttgtgatgagagagctgattcaacaacaacagcaactacaacaacaagaatcaatgataggtgagtatgaaaagattgaagagtctcacaattataataacatggaggaagaggaagatcaggaaatggatgaggaagaactagacgaggatgagaagtccgcggctttcgagattgcgtttcaaagccctgcaaacagaggaggcaatggccatacggaaccacctttcttgacaatggttcagtaaaatcagaatcattgtttcaagaaaatgtacttatgtgtgcatagttttcta

SEQ ID NO: 12 - protein, 669 aa, Arabidopsis thaliana
  G634
MEQGGGGGGNEVVEEASPISSRPPANNLEELMRFSAAADDGGLGGGGGGGGGGSASSSSGNRWPREETLALLRIRSDMDSTFRDATLKAPLWEHVSRKLLELGYKRSSKKCKEKFENVQKYYKRTKETRGGRHDGKAYKFFSQLEALNTTPPSSSLDVTPLSVANPILMPSSSSSPFPVFSQPQPQTQTQPPQTHNVSFTPTPPPLPLPSMGPIFTGVTFSSHSSSTASGMGSDDDDDDMDVDQANIAGSSSRKRKRGNRGGGGKMMELFEGLVRQVMQKQAAMQRSFLEALEKREQERLDREEAWKRQEMARLAREHEVMSQERAASASRDAAIISLIQKITGHTIQLPPSLSSQPPPPYQPPPAVTKRVAEPPLSTAQSQSQQPIMAIPQQQILPPPPPSHPHAHQPEQKQQQQPQQEMVMSSEQSSLPSSSRWPKAEILALINLRSGMEPRYQDNVPKGLLWEEISTSMKRMGYNRNAKRCKEKWENINKYYKKVKESNKKRPQDAKTCPYFHRLDLLYRNKVLGSGGGSSTSGLPQDQKQSPVTAMKPPQEGLVNVQQTHGSASTEEEEPIEESPQGTEKPEDLVMRELIQQQQQLQQQESMIGEYEKIEESHNYNNMEEEEDQEMDEEELDEDEKSAAFEIAFQSPANRGGNGHTEPPFLTMVQ

SEQ ID NO: 13 - DNA, 984 bp, Arabidopsis thaliana
  G659
atggggaagggaagagcaccttgttgtgacaagaccaaagtgaagagaggtccatggagcccagaagaagacattaaactcatctctttcattcaaaagtttggtcatgagaactggagatctctccccaaacaatctggtatgtcattgcttttgtcatcacaatcaaagcaaaagcctcttcaattgttttttcttttctttatgattctgaatgtatatatatgcaaaaatgaagggctattgaggtgtgggaagagttgtcgtctaaggtggattaactatcttaggccagatctgaagcgtggcaacttcacttcagaggaggaagaaacaatcattaagcttcaccacaactatgggaacaagtggtcgaaaatcgcttctcaacttccaggtagaacagataacgagatcaagaatgtgtggcacactcatctaaagaaaagactggctcagagctcaggaactgcagatgaaccggcctcgccttgttcgagtgattctgtttctcgtgggaaagatgataagtcatctcacgtagaagattctttgaacagagagactaatcataggaatgagttgtctacatctatgtcttctgggggttccaaccaacaagatgatccaaagatagacgaactcaggtttgagtatatagaagaagcttatagcgagtttaacgacattattattcaagaggtagacaaacccgatctgctggagataccatttgattcagatcctgacatttggagtttcttagatacttcaaactcatttcaacaatccactgcaaatgagaacagctcaggctcaagagcaacaacagaagaagagtctgatgaggatgaggttaagaaatggttcaagcacctagaaagcgaactcgggttagaagaagacgataatcaacaacaatacaaagaagaagaatcatcatcatcatcactcttgaagaactacgagctcatgatacattga

SEQ ID NO: 14 - protein, 327 aa, Arabidopsis thaliana
  G659
MGKGRAPCCDKTKVKRGPWSPEEDIKLISFIQKFGHENWRSLPKQSGMSLLLSSQSKQKPLQLFFLFFMILNVYICKNEGLLRCGKSCRLRWINYLRPDLKRGNFTSEEEETIIKLHHNYGNKWSKIASQLPGRTDNEIKNVWHTHLKKRLAQSSGTADEPASPCSSDSVSRGKDDKSSHVEDSLNRETNHRNELSTSMSSGGSNQQDDPKIDELRFEYIEEAYSEFNDIIIQEVDKPDLLEIPFDSDPDIWSFLDTSNSFQQSTANENSSGSRATTEEESDEDEVKKWFKHLESELGLEEDDNQQQYKEEESSSSSLLKNYELMIH

SEQ ID NO: 15 - DNA, 1212 bp, Arabidopsis thaliana
  G759
aaaaaatatgggtatccaagaaactgacccgttaacgcaattgagtttaccaccgggtttccgattttacccgaccgatgaagagcttatggttcaatatctctgtagaaaagcagctggttacgatttctctcttcagctcatcgccgaaatagatctttacaaattcgatccatgggttttaccaaataaagcattatttggagaaaaagaatggtatttttttagtcctagggatagaaaatatccaaacgggtcaagacctaaccgggttgccggatcgggttattggaaagctacgggtacggataaaataatctcgacggaaggacaaagagttggtattaaaaaagctttggtgttttacatcggaaaagctcctaaaggtactaaaaccaattggatcatgcatgagtatcgtctcattgaaccttctcgtagaaacggaagcactaagttggatgattgggttctatgtcgaatatacaagaagcaatcaagtgcacaaaaacaagtttacgataatggaatcgcgaatgctagagaattcagcaacaacggtacttcgtccacgacgtcgtcttcttctcactttgaagacgttcttgattcgtttcatcaagagatcgacaacagaaatttccagttttctaacccaaaccgcatctcgtcgctcagaccggacttaaccgaacagaaaaccgggttccacggtcttgcggatacttctaacttcgattgggctagttttgccggtaatgttgagcataataactcggtaccggaactcggaatgagtcatgttgttcctaatctcgagtacaactgtggctacctgaagacggaggaggaagtcgagagcagtcacgggtttaataactcgggcgagttagctcaaaagggttatggtgttgactcgtttgggtattcggggcaagttggtgggtttgggtttatgtgatgatgaaatgctgacgcaataaaaataagtcgttaatttttgtcccgtggcaaatcttatatgtatttgaatttcaattcttttgggttaagagggagactcatagatttagatgtagatttgtaatctttcatgcatagaaaatttgacgaatagatttcgtaactttattttgttgctgtttggttatctttgtattggtataaatttagtggattgaaattgcatattgaaaaaaaaaaaaaaaaaaa

SEQ ID NO: 16 - protein, 317 aa, Arabidopsis thaliana
  G759
MGIQETDPLTQLSLPPGFRFYPTDEELMVQYLCRKAAGYDFSLQLIAEIDLYKFDPWVLPNKALFGEKEWYFFSPRDRKYPNGSRPNRVAGSGYWKATGTDKIISTEGQRVGIKKALVFYIGKAPKGTKTNWIMHEYRLIEPSRRNGSTKLDDWVLCRIYKKQSSAQKQVYDNGIANAREFSNNGTSSTTSSSSHFEDVLDSFHQEIDNRNFQFSNPNRISSLRPDLTEQKTGFHGLADTSNFDWASFAGNVEHNNSVPELGMSHVVPNLEYNCGYLKTEEEVESSHGFNNSGELAQKGYGVDSFGYSGQVGGFGFM

SEQ ID NO: 17 - DNA, 1198 bp, Arabidopsis thaliana
  G764
atcgaattcgcggccgctcgatatctttacaaccattaaacaaaaaatttggccactacaagttgaaaaagttttgattatatctaatcgctgaaatggattacaaggtatcaagaagtggggagatagtagaaggagaagtagaagattcagaaaagattgatttaccacctggtttcagatttcacccaactgatgaagaacttataacacactatctaagaccaaaggttgtaaactcttttttctctgctatagctattggtgaagttgatctcaacaaagtcgagccttgggacttgccttggaaggctaagcttggggaaaaagagtggtacttcttttgcgtaagagaccgaaaatacccgactggtttaagaacgaatcgtgctactaaagccggttattggaaagctacagggaaagataaagagatcttcaaagggaaatctcttgttggtatgaagaaaacattggttttctacaaaggaagagctcctaaaggagtaaaaacaaattgggtcatgcatgagtatcgattagaaggcaaattcgctatcgataatctctctaaaaccgctaagaacgaatgtgttattagtcgtgtttttcatacacggactgatggtacgaaggagcatatgtccgttggtttacctccgctgatggattcttctccatatctaaagagtagaggacaagactctttagccgggaccacccttggtgggttgttgtctcacgttacctacttctccgaccaaacaaccgatgacaagagtcttgtggccgattttaaaactaccatgtttggttccggatcgactaactttttaccaaacataggttctctactagacttcgatcctctgtttctacaaaacaattcttcagtactgaagatgttgcttgacaatgaagaaacccaatttaagaagaatcttcacaattcaggttcatcagagagtgaactaacagcgagttcttggcaaggtcacaattcttatggttccactggtccagtgaatcttgattgcgtttggaaattctgaatttggaaaatcgaaaatttggatgttaactagggggtatatagggtttttaaaaacagtgtatatatgcgttatgtgttagctttagattctaggatatacaaagatgacactaatagattcttataacattttgtaaaaaaaaa

SEQ ID NO: 18 - protein, 318 aa, Arabidopsis thaliana
  G764
MDYKVSRSGEIVEGEVEDSEKIDLPPGFRFHPTDEELITHYLRPKVVNSFFSAIAIGEVDLNKVEPWDLPWKAKLGEKEWYFFCVRDRKYPTGLRTNRATKAGYWKATGKDKEIFKGKSLVGMKKTLVFYKGRAPKGVKTNWVMHEYRLEGKFAIDNLSKTAKNECVISRVFHTRTDGTKEHMSVGLPPLMDSSPYLKSRGQDSLAGTTLGGLLSHVTYFSDQTTDDKSLVADFKTTMFGSGSTNFLPNIGSLLDFDPLFLQNNSSVLKMLLDNEETQFKKNLHNSGSSESELTASSWQGHNSYGSTGPVNLDCVWKF

SEQ ID NO: 19 - DNA, 1479 bp, Arabidopsis thaliana
  G765
tttaactgcatagttcttcctcaaattgctcatacctttgcttctctttctttctttattcaacctcttatttcttcttcagcaccaacgtgtatctgcagtaaacacatatccatcgatcgatctcctcaaaaagttattgttttcttgaaggatttttcttgttcttgatcaagcatacatatatatagatggtggaagaaggcggcgtagttgtgaatcaaggaggagatcaggaggtggtggatttgcctccggggtttcggtttcaccctactgatgaagagataataactcactacctcaaagagaaggtcttcaacatccgatttaccgcggcagcgattggtcaagccgacctcaacaagaacgagccatgggatctaccaaagattgcaaagatgggggagaaggagttttactttttctgccagagggatcggaagtatccgaccgggatgaggacgaaccgtgcgaccgtgtctggttattggaaggcgaccgggaaggacaaggagatctttagaggcaaaggttgtcttgttgggatgaagaaaacacttgttttctatacaggaagagctcctaaaggtgaaaagaccaattgggttatgcatgaatatcgtcttgatggaaaatattcttatcataacctccccaaaaccgcaagggatgaatgggtggtgtgtagggtttttcacaagaacgctcctagtactacaatcactactacaaaacaactctcaaggattgattctcttgataacattgatcatctcttagacttctcatctctccctcctctcatagatccgggtttcttgggtcaacccgcccaagcttctccggtgcccgtcaacaacacgatctcaaacctgtctccaccatcctacaaccgcaccagtcgacaacacttaccttcctacccaagctctcaatttcccttaccactcggtccctaattccggatctgatttcggctacggggcaggttcaggcaataataacaaaggtatgatcaagttggagcattctcttgtgagtgtgtctcaagaaaccggtttgagttccgatgtgaacacaaccgcaacgccagagatatcttcttatccaatgatgatgaatccggcaatgatggatggtagcaagtcagcgtgtgatggtcttgatgacttgatcttctgggaagatttatatactagctaaatttgggaaaaggttatttgttaattgtgattgaagagtggcatattgattactcgtctagtgtttttaatcgtgtaattagttcgtatataatatacatgtacataagatcattaggtttattaggcattggactttagttcggtgattgcttacctagtttttagcttgagaaaaaaggctgtcattggggttatgtttctttgtgattaacttgtacatatatacatttaaattaaacgtatggtttaaatcgtttaaaaaaaaaaaaaa

SEQ ID NO: 20 - protein, 256 aa, Arabidopsis thaliana
  G765
MVEEGGVVVNQGGDQEVVDLPPGFRFHPTDEEIITHYLKEKVFNIRFTAAAIGQADLNKNEPWDLPKIAKMGEKEFYFFCQRDRKYPTGMRTNRATVSGYWKATGKDKEIFRGKGCLVGMKKTLVFYTGRAPKGEKTNWVMHEYRLDGKYSYHNLPKTARDEWVVCRVFHKNAPSTTITTTKQLSRIDSLDNIDHLLDFSSLPPLIDPGFLGQPAQASPVPVNNTISNLSPPSYNRTSRQHLPSYPSSQFPLPLGP

SEQ ID NO: 21 - DNA, 1057 bp, Arabidopsis thaliana
  G777
gtggctctctctttatctttcttggagtttagttagagattttaacgttgcaaatggatcaaccaatgaaaccaaaaacttgctctgaatctgattttgctgatgattcctctgcttcttcttcttcttcttcgggacaaaatctcagaggagctgagatggtggtggaagtgaagaaggaagcagtttgttcccagaaagcagagcgagagaagcttcgtagagataagcttaaggaacagtttcttgagcttggaaatgcacttgatccgaataggcctaagagtgacaaagcctcagttctcactgatacaatacaaatgctcaaggatgtaatgaaccaagttgatagactaaaagctgagtatgaaacactatctcaagagtctcgtgagctaattcaagagaagagtgagctgagagaggagaaagcgactttaaagtctgatatcgagattcttaatgctcaatatcagcatagaatcaaaaccatggttccatgggtacctcattacagttatcatatccccttcgtagccataactcagggtcagtccagttttataccttattcagcctctgtcaatcctctaaccgaacaacaagcatcggttcagcagcattcttcttcttctgccgatgcttcaatgaaacaagattccaaaatcaagccgttagatttggatctgatgatgaacagtaaccattcaggtcaaggaaatgatcaaaaagatgatgttcgtttaaagctcgagcttaaaatccatgcctcttctttagctcaacaggatgtttctggaaaagagaagaaagtaagcttgacaaccactgcaagctcatcgaatagttactcattatctcaagctgttcaagatagttcccccggtaccgtaaatgacatgttgaagccataaaccaataaacatattcccctgaacttgtgtttaataccgtgattgagaaggtaccatgattaaacttgttgtagattatccacatgattaacgatgtattcttatcacaagcaaataaaacacaaaagcatttgcttaaaaaa

SEQ ID NO: 22 - protein, 286 aa, Arabidopsis thaliana
  G777
MDQPMKPKTCSESDFADDSSASSSSSSGQNLRGAEMVVEVKKEAVCSQKAEREKLRRDKLKEQFLELGNALDPNRPKSDKASVLTDTIQMLKDVMNQVDRLKAEYETLSQESRELIQEKSELREEKATLKSDIEILNAQYQHRIKTMVPWVPHYSYHIPFVAITQGQSSFIPYSASVNPLTEQQASVQQHSSSSADASMKQDSKIKPLDLDLMMNSNHSGQGNDQKDDVRLKLELKIHASSLAQQDVSGKEKKVSLTTTASSSNSYSLSQAVQDSSPGTVNDMLKP

SEQ ID NO: 23 - DNA, 1281 bp, Arabidopsis thaliana
  G867
cacaacacaaacacatttctgttttctccattgtttcaaaccataaaaaaaaacacagattaaatggaatcgagtagcgttgatgagagtactacaagtacaggttccatctgtgaaaccccggcgataactccggcgaaaaagtcgtcggtaggtaacttatacaggatgggaagcggatcaagcgttgtgttagattcagagaacggcgtagaagctgaatctaggaagcttccgtcgtcaaaatacaaaggtgtggtgccacaaccaaacggaagatggggagctcagatttacgagaaacaccagcgcgtgtggctcgggacattcaacgaagaagacgaagccgctcgtgcctacgacgtcgcggttcacaggttccgtcgccgtgacgccgtcacaaatttcaaagacgtgaagatggacgaagacgaggtcgatttcttgaattctcattcgaaatctgagatcgttgatatgttgaggaaacatacttataacgaagagttagagcagagtaaacggcgtcgtaatggtaacggaaacatgactaggacgttgttaacgtcggggttgagtaatgatggtgtttctacgacggggtttagatcggcggaggcactgtttgagaaagcggtaacgccaagcgacgttgggaagctaaaccgtttggttataccgaaacatcacgcagagaaacattttccgttaccgtcaagtaacgtttccgtgaaaggagtgttgttgaactttgaggacgttaacgggaaagtgtggaggttccgttactcgtattggaacagtagtcagagttatgttttgactaaaggttggagcaggttcgttaaggagaagaatctacgtgctggtgacgtggttagtttcagtagatctaacggtcaggatcaacagttgtacattgggtggaagtcgagatccgggtcagatttagatgcgggtcgggttttgagattgttcggagttaacatttcaccggagagttcaagaaacgacgtcgtaggaaacaaaagagtgaacgatactgagatgttatcgttggtgtgtagcaagaagcaacgcatctttcacgcctcgtaacaactcttcttctttttttttcttttgttgttttaataatttttaaaaactccattttcgttttctttatttgcatcggtttctttcttcttgtttaccaaaggttcatgagttgtttttgttgtattgatgaactgtaaattttatttataggataaattttaaaaaaaaaaaaaaaaaaaa

SEQ ID NO: 24 - protein, 344 aa, Arabidopsis thaliana
  G867
MESSSVDESTTSTGSICETPAITPAKKSSVGNLYRMGSGSSVVLDSENGVEAESRKLPSSKYKGVVPQPNGRWGAQIYEKHQRVWLGTFNEEDEAARAYDVAVHRFRRRDAVTNFKDVKMDEDEVDFLNSHSKSEIVDMLRKHTYNEELEQSKRRRNGNGNMTRTLLTSGLSNDGVSTTGFRSAEALFEKAVTPSDVGKLNRLVIPKHHAEKHFPLPSSNVSVKGVLLNFEDVNGKVWRFRYSYWNSSQSYVLTKGWSRFVKEKNLRAGDVVSFSRSNGQDQQLYIGWKSRSGSDLDAGRVLRLFGVNISPESSRNDVVGNKRVNDTEMLSLVCSKKQRIFHAS

SEQ ID NO: 25 - DNA, 914 bp, Arabidopsis thaliana
  G503
gaacatcaaaaactaacacacagaaagaaaaaaaacagttcctgttccattagattcttttctaaattgtctgaaaatcatggaagtaacttcccaatctaccctccctccagggttccgatttcatcctaccgacgaagaactcatcgtttactatctccgaaaccagaccatgtctaaaccatgccctgtctccatcatcccagaagttgatatctacaaattcgacccatggcaattacccgagaaaacagagtttggagaaaatgagtggtatttcttcagccctagagaaagaaaatatccaaacggagtcagaccaaaccgggcagctgtttccggttattggaaagcaaccggtacagacaaagccattcacagcggttcgagtaacgtaggtgtcaagaaagctctcgtcttctacaaaggtagacctcctaaaggaatcaaaactgactggatcatgcatgagtatcgtctccatgattcacgtaaagcatcaacgaaacgtagcggatctatgaggttagatgaatgggtactatgtaggatatacaagaagagaggagcaagtaagcttctgaatgagcaagagggtttcatggacgaagtactaatggaggatgagaccaaagttgttattaacgaagcagagagaagaaatgatgaagagataatgatgatgacgtcgatgaaacttccaaggacgtgttcgctggctcatttgttggaaatggattacatgggacccgtctctcacattgataattttagtcagttcgatcatcttcatcaacctgattcggagtctagttggttcggggatctacagtttaaccaagacgagatcttaaaccatcatcgtcaagctatgtttaagttttagtgatggggtcagtaaaaaaaaaaaaaaa

SEQ ID NO: 26 - protein, 268 aa, Arabidopsis thaliana
  G503
MEVTSQSTLPPGFRFHPTDEELIVYYLRNQTMSKPCPVSIIPEVDIYKFDPWQLPEKTEFGENEWYFFSPRERKYPNGVRPNRAAVSGYWKATGTDKAIHSGSSNVGVKKALVFYKGRPPKGIKTDWIMHEYRLHDSRKASTKRSGSMRLDEWVLCRIYKKRGASKLLNEQEGFMDEVLMEDETKVVINEAERRNDEEIMMMTSMKLPRTCSLAHLLEMDYMGPVSHIDNFSQFDHLHQPDSESSWFGDLQFNQDEILNHHRQAMFKF

SEQ ID NO: 27 - DNA, 768 bp, Arabidopsis thaliana
  G975
attactcatcatcaagttcctactttctctctgacaaacatcacagagtaagtaagaatggtacagacgaagaagttcagaggtgtcaggcaacgccattggggttcttgggtcgctgagattcgtcatcctctcttgaaacggaggatttggctagggacgttcgagaccgcagaggaggcagcaagagcatacgacgaggccgccgttttaatgagcggccgcaacgccaaaaccaactttcccctcaacaacaacaacaccggagaaacttccgagggcaaaaccgatatttcagcttcgtccacaatgtcatcctcaacatcatcttcatcgctctcttccatcctcagcgccaaactgaggaaatgctgcaagtctccttccccatccctcacctgcctccgtcttgacacagccagctcccatatcggcgtctggcagaaacgggccggttcaaagtctgactccagctgggtcatgacggtggagctaggtcccgcaagctcctcccaagagactactagtaaagcttcacaagacgctattcttgctccgaccactgaagttgaaattggtggcagcagagaagaagtattggatgaggaagaaaaggttgctttgcaaatgatagaggagcttctcaatacaaactaaatcttatttgcttatatatatgtacctattttcattgctgatttacagccaaaataatcaattataccgtgtattttatagatgttttatattaaaaggttgttagatata

SEQ ID NO: 28 - protein, 199 aa, Arabidopsis thaliana
  G975
MVQTKKFRGVRQRHWGSWVAEIRHPLLKRRIWLGTFETAEEAARAYDEAAVLMSGRNAKTNFPLNNNNTGETSEGKTDISASSTMSSSTSSSSLSSILSAKLRKCCKSPSPSLTCLRLDTASSHIGVWQKRAGSKSDSSWVMTVELGPASSSQETTSKASQDAILAPTTEVEIGGSREEVLDEEEKVALQMIEELLNTN

SEQ ID NO: 29 - DNA, 1554 bp, Arabidopsis thaliana
  G562
atttgaatttctgggtttctctctgtttaagcttcttcttcttcatcttctgcttacgtttcttcttcaaggagctttcggattcttgtagaaagagtcattgttctcttgagtgggaaaccttgaaaccattcctatgggaaatagcagcgaggaaccaaagcctcctaccaaatcagataaaccatcttcacccccggtggatcaaacaaatgttcatgtctaccctgattgggcagctatgcaggcatattatggtccaagagtagcaatgcctccttattacaattcagctatggctgcatctggtcatcctcctcctccttacatgtggaatcctcagcatatgatgtcaccatctggagcaccctatgctgctgtttatcctcatggaggaggagtttacgctcatcccggtattcccatgggatcactgcctcaaggtcaaaaggatccacctttaacaactccggggacgcttttgagcatcgacactcctactaaatctacagggaacacagacaatggattgatgaagaagctgaaagagtttgatgggcttgctatgtctctaggaaatgggaatcctgaaaatggtgcagatgaacataaacgatcacggaacagctcagaaactgatggttctactgatggaagtgatgggaatacaactggggcagatgaaccgaaacttaaaagaagtcgagagggaactccaacaaaagatgggaaacaattggttcaagctagctcatttcattctgtttctccgtcaagtggtgataccggcgtaaaactcattcaaggatctggagctatactctctcctggtgtaagtgcaaattccaaccccttcatgtcacaatctttagccatggttcctcctgaaacttggcttcagaacgagagagaactgaaacgggagcgaaggaaacagtctaatagagaatctgctagaaggtcaagattaaggaaacaggccgagacagaagaacttgctaggaaagtggaagccttgacagccgaaaacatggcattaagatctgaactaaaccaacttaatgagaaatctgataaactaagaggagcaaatgcaaccttgttggacaaactgaaatgctcggaacccgaaaagagagtccccgcaaatatgttgtctagagttaagaactcaggagctggagataagaacaagaaccaaggagacaatgattctaactctacaagcaaattccatcaactgctcgatacgaagcctcgagctaaagcagtagctgcaggctgaatcgatggtaattcatgtcgatttctacttaatttgtcgacataaacaaagaaaataagtgctactaatttcagaaaaacttgatagatagatagtatagtagagagagagagagagagagaggtgtgatgattattgatctataaattttcggagagagagagggagaaagagaaacttttcctccagatgaaaatttggtgttatggtttgttactgttaatatagagaggcttttctttttttataaaatggcttcctttgttgca

SEQ ID NO: 30 - protein, 382 aa, Arabidopsis thaliana
  G562
MGNSSEEPKPPTKSDKPSSPPVDQTNVHVYPDWAAMQAYYGPRVAMPPYYNSAMAASGHPPPPYMWNPQHMMSPSGAPYAAVYPHGGGVYAHPGIPMGSLPQGQKDPPLTTPGTLLSIDTPTKSTGNTDNGLMKKLKEFDGLAMSLGNGNPENGADEHKRSRNSSETDGSTDGSDGNTTGADEPKLKRSREGTPTKDGKQLVQASSFHSVSPSSGDTGVKLIQGSGAILSPGVSANSNPFMSQSLAMVPPETWLQNERELKRERRKQSNRESARRSRLRKQAETEELARKVEALTAENMALRSELNQLNEKSDKLRGANATLLDKLKCSEPEKRVPANMLSRVKNSGAGDKNKNQGDNDSNSTSKFHQLLDTKPRAKAVAAG

SEQ ID NO: 31 - DNA, 420 bp, Arabidopsis thaliana
  G1508
atgctagatcacagtgaaaaggtcttattggttgattcagaaaccatgaaaacaagagctgaagatatgatcgaacagaacaacactagtgttaacgacaagaagaagacttgtgctgattgtggaaccagtaaaactcctctttggcgtggtggtcctgttggtccaaagtcgttgtgtaacgcgtgtgggatcagaaacagaaagaagagaagaggaggaacagaagataataagaaattaaagaaatcgagttctggcggcggaaaccgtaaatttggtgaatcgttaaaacagagtttgatggatttggggataaggaagagatcaacggtggagaagcaacgacagaagcttggtgaagaagaacaagccgctgtgttactcatggctctttcttatggctctgtttacgcttag

SEQ ID NO: 32 - protein, 139 aa, Arabidopsis thaliana
  G1508
MLDHSEKVLLVDSETMKTRAEDMIEQNNTSVNDKKKTCADCGTSKTPLWRGGPVGPKSLCNACGIRNRKKRRGGTEDNKKLKKSSSGGGNRKFGESLKQSLMDLGIRKRSTVEKQRQKLGEEEQAAVLLMALSYGSVYA

SEQ ID NO: 33 - DNA, 1674 bp, Arabidopsis thaliana
  G176
agaagaagaagaagaagagtacctcatacgtaaaccattgatgggctcttttgatcgccaaagagctgttccgaaattcaaaacagcaacaccgtcaccgctccctctttctccttcgccttacttcactatgcctcctggccttactcccgccgactttctcgactctcctcttctcttcacttcctccaacattttgccgtctcctacgacaggcacatttccagcgcaatctctgaactataacaataacggtttgctcattgacaaaaatgaaatcaaatatgaagacacaactcctcccttgttcctaccatctatggtaactcagcctttacctcaactggatttattcaaatccgaaatcatgtcgagtaacaaaacctctgatgacggctacaattggcgcaaatacgggcagaagcaagtcaaaggaagcgaaaacccgaggagttacttcaaatgcacgtatccaaattgtctcacaaagaagaaagtagagacgtctcttgtgaagggtcagatgattgagattgtctataaaggaagccacaatcatcccaagccccaatccacgaagcgatcatcttccaccgctatagcagcacatcagaacagcagtaatggagacggtaaagacattggtgaagatgaaacagaggccaagagatggaaaagagaagagaatgtgaaggagccaagagtggtggttcagacaacaagtgatatagacattcttgacgatggctacagatggagaaagtatggtcagaaagtcgtcaagggtaatccaaatccaaggagctattacaagtgcacatttacaggatgttttgtaaggaaacacgttgaaagagcatttcaagatcccaagtcagtgatcacaacttacgaaggaaaacacaaacaccaaatcccgaccccaagaagaggtccagttttaagatctgctgcaatggcttctcctcttctcccaacttcgactactcctgatcaacttcccggcggcgatccacagttgctgagctctctacgcgtcctcttgtcccgcgttctagccaccgtccgtcacgcttctgcagatgccagaccctgggcagagctcgttgaccggtcagcgttttcccggccaccatcgctctcggaggcaacgtcacgagtaaggaagaacttttcctatttccgagccaattacataaccttagtggcaatcttactcgccgcgtctctgctcacgcaccctttcgctctcttcctcctcgcatcgctggccgcttcttggcttttcctctactttttccgtccggcggatcagccgttggtcattggaggacgcacgttctccgatcttgagacgctagggatactctgcctgtccactgtggtggtgatgttcatgaccagcgttggatcgctcttgatgtccactctagcagttgggatcatgggcgtggccatccacggagcgtttcgtgctcccgaagacctgtttcttgaagaacaagaagccattggatctggacttttcgcattcttcaacaacaatgcctctaatgcagctgccgctgccatagccacctcagcaatgtcacgcgttcgagtctgagattgttgaagagactacattcctacaccgcatttccaaagtgtgatatttattcatattgaattgtt

SEQ ID NO: 34 - protein, 521 aa, Arabidopsis thaliana
  G176
MGSFDRQRAVPKFKTATPSPLPLSPSPYFTMPPGLTPADFLDSPLLFTSSNILPSPTTGTFPAQSLNYNNNGLLIDKNEIKYEDTTPPLFLPSMVTQPLPQLDLFKSEIMSSNKTSDDGYNWRKYGQKQVKGSENPRSYFKCTYPNCLTKKKVETSLVKGQMIEIVYKGSHNHPKPQSTKRSSSTAIAAHQNSSNGDGKDIGEDETEAKRWKREENVKEPRVVVQTTSDIDILDDGYRWRKYGQKVVKGNPNPRSYYKCTFTGCFVRKHVERAFQDPKSVITTYEGKHKHQIPTPRRGPVLRSAAMASPLLPTSTTPDQLPGGDPQLLSSLRVLLSRVLATVRHASADARPWAELVDRSAFSRPPSLSEATSRVRKNFSYFRANYITLVAILLAASLLTHPFALFLLASLAASWLFLYFFRPADQPLVIGGRTFSDLETLGILCLSTVVVMFMTSVGSLLMSTLAVGIMGVAIHGAFRAPEDLFLEEQEAIGSGLFAFFNNNASNAAAAAIATSAMSRVRV

SEQ ID NO: 35 - DNA, 825 bp, Arabidopsis thaliana
  G197
atgggaaggtctccttgctgtgagaaagaccacacaaacaaaggagcttggactaaggaagaagacgataagctcatctcttacatcaaagctcacggtgaaggttgttggcgttctcttcctagatccgccggtcttcaacgttgcggaaaaagctgtcgtctccgatggattaactatctccgacctgatctcaagaggggtaacttcaccctcgaagaagatgatctcatcatcaaactacatagccttctcggtaacaagtggtctcttattgcgacgagattaccaggaagaacagataacgagattaagaattactggaacacacatgttaagaggaagctattaagaaaagggattgatccggcgactcatcgacctatcaacgagaccaaaacttctcaagattcgtctgattctagtaaaacagaggaccctcttgtcaagattctctcttttggtcctcagctggagaaaatagcaaatttcggggacgagagaattcaaaagagagttgagtactcagttgttgaagaaagatgtctggacttgaatcttgagcttaggatcagtccaccatggcaagacaagctccatgatgagaggaacctaaggtttgggagagtgaagtataggtgcagtgcgtgccgttttggattcgggaacggcaaggagtgtagctgtaataatgtgaaatgtcaaacagaggacagtagtagcagcagttattcttcaaccgacattagtagtagcattggttatgacttcttgggtctaaacaacactagggttttggattttagcactttggaaatgaaatga

SEQ ID NO: 36 - protein, 274 aa, Arabidopsis thaliana
  G197
MGRSPCCEKDHTNKGAWTKEEDDKLISYIKAHGEGCWRSLPRSAGLQRCGKSCRLRWINYLRPDLKRGNFTLEEDDLIIKLHSLLGNKWSLIATRLPGRTDNEIKNYWNTHVKRKLLRKGIDPATHRPINETKTSQDSSDSSKTEDPLVKILSFGPQLEKIANFGDERIQKRVEYSVVEERCLDLNLELRISPPWQDKLHDERNLRFGRVKYRCSACRFGFGNGKECSCNNVKCQTEDSSSSSYSSTDISSSIGYDFLGLNNTRVLDFSTLEMK

SEQ ID NO: 37 - DNA, 1364 bp, Arabidopsis thaliana
  G222
ctcattttataataattttctccatcttcttccttcctcttcaagcttatagaacctccaagaaccataaaactctctctgcaacaatctatactttcttcattacttttataaaaaatgggaagacattcttgttgttttaagcagaagctaagaaaaggcctttggtctcctgaagaagatgagaaacttctcaattacatcactagacatggtcatggctgttggagttctgtccctaaactcgcaggtttgcaaagatgtggaaagagttgtagacttaggtggataaattatttgagaccagatttaaagagaggagctttctctcaagacgaagaaagcttgatcattgagctccatgctgcattaggcaacagatggtctcaaatcgcaacgcggttaccgggaagaacagacaacgagatcaaaaacttttggaactcatgtcttaagaagaagctgagaagaaaaggcattgacccaacaacacataaacccttaataacaaacgagcttcaatctcttaacgtcatagatcagaaactgacgtcatcagaagtagtaaagtcaacgggttcgataaacaacctacatgatcagtcaatggtcgtctcatcgcaacaaggtccatggtggttcccggcgaatacaactacgactaatcaaaactctgcgttttgctttagttcaagtaatactacaacggtttcagaccagatcgtatctttaatctcttcaatgtctacgtcatcatctccgacaccaatgacttcaaacttcagtcctgctccaaacaactgggaacaactcaactactgcaacacagtaccaagtcagagcaacagtatcttcagtgccttctttggtaatcaatacacagaagctagccaaaccatgaacaataataatccactagtagatcaacatcatcatcatcaagacatgaagtcatgggcatcagagattcttcattacacagaacacaaccaaagctcagaaactgttatagaagcagaagtgaagccagacattgccaactactactggagatcagcatcatcatcgtcgtcaccaaaccaagaagctgcaacattactacacgatgctaacgtggaagtgtacggtaaaaatctacaaaagcttaataacacggtgtttgatcagagcctttagattattaacatgtgttatagagagtttcattttctattcatattcatagacctgagtttattgattgatccatacatttgttatgcttgtgtgtaaagtatgtatatggtttttgatttatgtaaattttctgctcgagtgaatttttttggtgctaagatgtagttctttattccaaaaaaaaaaaa

SEQ ID NO: 38 - protein, 352 aa, Arabidopsis thaliana
  G222
MGRHSCCFKQKLRKGLWSPEEDEKLLNYITRHGHGCWSSVPKLAGLQRCGKSCRLRWINYLRPDLKRGAFSQDEESLIIELHAALGNRWSQIATRLPGRTDNEIKNFWNSCLKKKLRRKGIDPTTHKPLITNELQSLNVIDQKLTSSEVVKSTGSINNLHDQSMVVSSQQGPWWFPANTTTTNQNSAFCFSSSNTTTVSDQIVSLISSMSTSSSPTPMTSNFSPAPNNWEQLNYCNTVPSQSNSIFSAFFGNQYTEASQTMNNNNPLVDQHHHHQDMKSWASEILHYTEHNQSSETVIEAEVKPDIANYYWRSASSSSSPNQEAATLLHDANVEVYGKNLQKLNNTVFDQSL

SEQ ID NO: 39 - DNA, 1209 bp, Arabidopsis thaliana
  G229
ttgtggtcagtggaataaacacatataaccgccggagaaaatgggaagagcgccatgttgcgagaaggtcggtatcaagagagggcggtggacggcggaggaggaccagattctctccaactacattcaatccaatggtgaaggttcttggagatctctccccaaaaatgccggattaaaaaggtgtggaaagagctgtagattgagatggataaactatctaagatcagacctcaagcgtggaaacataactccagaagaagaagaactcgttgttaaattgcattccactttgggaaacaggtggtcactaatcgcgggtcatctaccagggagaacagacaacgaaataaaaaattattggaactctcatctcagccgtaaactccacaacttcattaggaagccatccatctctcaagacgtctccgccgtaatcatggcgaacgcttcttcagcgccaccgccgccgcaggcaaaacgcagacttgggagaacgagtaggtccgctatgaaaccaaaaatccgcagaacaaaaactcgtaaaacgaagaaaacgtctgcaccaccggagcctaacgccgatgtagctggggctgataaagaagcattaatggtggagtcaagtggagccgaggctgagctaggacgaccatgtgactactatggagatgattgtaacaaaaatctcatgagcattaatggcgataatggagttttaacgtttgatgatgatatcatcgatcttttgttggacgagtcagatcctggccacttgtacacaaacacaacgtgcggtggtggtggggagttgcataacataagagactctgaaggagccagagggttctcggatacttggaaccaagggaatctcgactgtcttcttcagtcttgtccatctgtggagtcgtttctcaactacgaccaccaagttaacgacgcgtcgacggatgagtttatcgattgggattgtgtttggcaagaaggtagtgataataatctttggcatgagaaagagaatcccgactcaatggtctcgtggcttttagacggtgatgatgaggccacgatcgggaatagtaattgtgagaactttggagaaccgttagatcatgacgacgaaagcgctttggtcgcttggcttctgtcatgatgatattgattgatccgttatgtaatcttttttgtgcattcacagtttgaatc

SEQ ID NO: 40 - protein, 371 aa, Arabidopsis thaliana
  G229
MGRAPCCEKVGIKRGRWTAEEDQILSNYIQSNGEGSWRSLPKNAGLKRCGKSCRLRWINYLRSDLKRGNITPEEEELVVKLHSTLGNRWSLIAGHLPGRTDNEIKNYWNSHLSRKLHNFIRKPSISQDVSAVIMANASSAPPPPQAKRRLGRTSRSAMKPKIRRTKTRKTKKTSAPPEPNADVAGADKEALMVESSGAEAELGRPCDYYGDDCNKNLMSINGDNGVLTFDDDIIDLLLDESDPGHLYTNTTCGGGGELHNIRDSEGARGFSDTWNQGNLDCLLQSCPSVESFLNYDHQVNDASTDEFIDWDCVWQEGSDNNLWHEKENPDSMVSWLLDGDDEATIGNSNCENFGEPLDHDDESALVAWLLS

SEQ ID NO: 41 - DNA, 957 bp, Arabidopsis thaliana
  G24
cggacgcgtgggcaaatattaaaataaaaagtgtcggtgaattctcaatctttgtcttctttcgtcgtctctttaaaactcctccgtccctccttattatgtaaccgtctcgccgtcaaattttcaaaatctctccctccgttcataaacccagatcgaaatttatggttttgtaatttttttaccggcggttatggagacggaagcggcggtgacagcgacggttacggcggcgacgatggggattgggacgaggaagagagatctgaaaccgtataaaggaatacgaatgaggaaatgggggaaatgggtggcggagatacgggaaccgaataagagatcaaggatctggttaggttcttatgcgacgcctgaagcggcggcgagagcttacgacactgctgttttttacctccgtggtccttcagcgaggcttaattttccggagcttttggctggacttactgtttctaacggcggaggaagaggtggtgatttatcggcggcgtatattaggagaaaagcggcggaggttggtgctcaggttgatgcgcttggagcgacggtggttgtgaataccggcggcgagaatcgcggtgattacgagaagattgagaattgtcgtaagagcggtaacgggtcattggaacgggtcgatttgaataaattacccgacccggaaaattcggatggtgatgatgacgaatgtgtgaaaagaagatagaaaaaataaaaagtagttgtagaaggagagacgagaatgtttgtctttaagatgcgctgttgccgctaacatgcgctttcgattttagtgttaaacatgcgcctccattgtttttgggttttgttttcgtcgtcgataatcaaagattttaaaacacaattctcaaatttttcacttgttacaaactagatttgcatgatctttgtattaacgaataacgattaagtcctaaa

SEQ ID NO: 42 - protein, 176 aa, Arabidopsis thaliana
  G24
METEAAVTATVTAATMGIGTRKRDLKPYKGIRMRKWGKWVAEIREPNKRSRIWLGSYATPEAAARAYDTAVFYLRGPSARLNFPELLAGLTVSNGGGRGGDLSAAYIRRKAAEVGAQVDALGATVVVNTGGENRGDYEKIENCRKSGNGSLERVDLNKLPDPENSDGDDDECVKRR

SEQ ID NO: 43 - DNA, 583 bp, Arabidopsis thaliana
  G10
ggtgattcaatacaaacgaaaacaagagtttcctatggtgaaagaaggaatggtcatgaccgagaagccaaagaggaatctcataagctctaatgagaagcgatacaaaggaataaggatgaggaagtggggcaagtgggtggctgagataagagagcctaataaacgatcacggatctggcttggttcatacaaaaccgccgttgccgcggcacgggcctacgataccgctgtgttttacttacgtggtccttcggcgagactcaatttccctgaagaggtctttaaggatggaaacggcggtgaaggcttaggaggagatatgtctccgacgttgatacggaagaaggcggctgaggtgggagctagagtcgacgcagagttgcggttagagaataggatggttgagaacttagacatgaataagttgccggaggcatatggattgtaatttatagtttggtagtttataggttggagattgcccggagacagagtcaaacagaggttctctgactcatatgaggcataatatagttaatatagtaatttttgttttgagcatagtaattatgtcataacc

SEQ ID NO: 44 - protein, 138 aa, Arabidopsis thaliana
  G10
MVKEGMVMTEKPKRNLISSNEKRYKGIRMRKWGKWVAEIREPNKRSRIWLGSYKTAVAAARAYDTAVFYLRGPSARLNFPEEVFKDGNGGEGLGGDMSPTLIRKKAAEVGARVDAELRLENRMVENLDMNKLPEAYGL

SEQ ID NO: 45 - DNA, 1951 bp, Arabidopsis thaliana
  G308
agtaatttagttttttttttttttttttacaatttattttgttattagaagtggtagtggagtgaaaaaacaaatcctaagcagtcctaaccgatccccgaagctaaagattcttcaccttcccaaataaagcaaaacctagatccgacattgaaggaaaaaccttttagatccatctctgaaaaaaacccaaccatgaagagagatcatcatcatcatcatcaagataagaagactatgatgatgaatgaagaagacgacggtaacggcatggatgagcttctagctgttcttggttacaaggttaggtcatcggaaatggctgatgttgctcagaaactcgagcagcttgaagttatgatgtctaatgttcaagaagacgatctttctcaactcgctactgagactgttcactataatccggcggagctttacacgtggcttgattctatgctcaccgaccttaatcctccgtcgtctaacgccgagtacgatcttaaagctattcccggtgacgcgattctcaatcagttcgctatcgattcggcttcttcgtctaaccaaggcggcggaggagatacgtatactacaaacaagcggttgaaatgctcaaacggcgtcgtggaaaccaccacagcgacggctgagtcaactcggcatgttgtcctggttgactcgcaggagaacggtgtgcgtctcgttcacgcgcttttggcttgcgctgaagctgttcagaaggagaatctgactgtggcggaagctctggtgaagcaaatcggattcttagctgtttctcaaatcggagctatgagacaagtcgctacttacttcgccgaagctctcgcgcggcggatttaccgtctctctccgtcgcagagtccaatcgaccactctctctccgatactcttcagatgcacttctacgagacttgtccttatctcaagttcgctcacttcacggcgaatcaagcgattctcgaagcttttcaagggaagaaaagagttcatgtcattgatttctctatgagtcaaggtcttcaatggccggcgcttatgcaggctcttgcgcttcgacctggtggtcctcctgttttccggttaaccggaattggtccaccggcaccggataatttcgattatcttcatgaagttgggtgtaagctggctcatttagctgaggcgattcacgttgagtttgagtacagaggatttgtggctaacactttagctgatcttgatgcttcgatgcttgagcttagaccaagtgagattgaatctgttgcggttaactctgttttcgagcttcacaagctcttgggacgacctggtgcgatcgataaggttcttggtgtggtgaatcagattaaaccggagattttcactgtggttgagcaggaatcgaaccataatagtccgattttcttagatcggtttactgagtcgttgcattattactcgacgttgtttgactcgttggaaggtgtaccgagtggtcaagacaaggtcatgtcggaggtttacttgggtaaacagatctgcaacgttgtggcttgtgatggacctgaccgagttgagcgtcatgaaacgttgagtcagtggaggaaccggttcgggtctgctgggtttgcggctgcacatattggttcgaatgcgtttaagcaagcgagtatgcttttggctctgttcaacggcggtgagggttatcgggtggaggagagtgacggctgtctcatgttgggttggcacacacgaccgctcatagccacctcggcttggaaactctccaccaattagatggtggctcaatgaattgatctgttgaaccggttatgatgatagatttccgaccgaagccaaactaaatcctactgtttttccctttgtcacttgttaagatcttatctttcattatattaggtaattgaaaaattttaatctcgcctaaattact

SEQ ID NO: 46 - protein, 532 aa, Arabidopsis thaliana
  G308
MKRDHHHHHQDKKTMMMNEEDDGNGMDELLAVLGYKVRSSEMADVAQKLEQLEVMMSNVQEDDLSQLATETVHYNPAELYTWLDSMLTDLNPPSSNAEYDLKAIPGDAILNQFAIDSASSSNQGGGGDTYTTNKRLKCSNGVVETTTATAESTRHVVLVDSQENGVRLVHALLACAEAVQKENLTVAEALVKQIGFLAVSQIGAMRQVATYFAEALARRIYRLSPSQSPIDHSLSDTLQMHFYETCPYLKFAHFTANQAILEAFQGKKRVHVIDFSMSQGLQWPALMQALALRPGGPPVFRLTGIGPPAPDNFDYLHEVGCKLAHLAEAIHVEFEYRGFVANTLADLDASMLELRPSEIESVAVNSVFELHKLLGRPGAIDKVLGVVNQIKPEIFTVVEQESNHNSPIFLDRFTESLHYYSTLFDSLEGVPSGQDKVMSEVYLGKQICNVVACDGPDRVERHETLSQWRNRFGSAGFAAAHIGSNAFKQASMLLALFNGGEGYRVEESDGCLMLGWHTRPLIATSAWKLSTN

SEQ ID NO: 47 - DNA, 1516 bp, Arabidopsis thaliana
  G928
ctcatggcgatgttggtttcccaggaaaggtaaaagagacggagacgaaccaaaacaaggaagaaagaagaagatcttacatacgaagatcactctctgattcactctgagagacaaactggtttactttggttctgtttgacaaaaggagacatgcaaaaataaatctctatcccttgtttttcttcttcgcttcatcgattactcaaagaggttgttggttgtgagaataattagcttgttaaggaagacgttatgatgcatcagatgttgaataagaaagattcagctactcattccactttgccataccttaatactagcatctcttggggagtggttccaactgattccgttgctaatcgtcgcggtcctgctgaatcactaagcttgaaggttgattcaagacctgggcatatacaaactacaaagcaaatcagttttcaggaccaagattcatcttcaacacagtccactggtcaatcttatactgaagttgctagtagtggtgatgataatccttccagacaaatctccttttcggctaaatcaggatctgaaataactcaacggaaggggtttgcaagtaatcctaaacaaggctcgatgactggatttccgaatattcactttgctcctgcacaggctaatttctcatttcactatgctgatccacattatggtggtttattagctgcaacttacctaccacaggcaccaacatgcaatcctcaaatggtgagtatgattcctggtcgtgttcctttaccagcagagctcacagaaactgatccagtctttgtcaatgcgaagcaataccacgcaattatgaggaggagacagcaacgtgctaagcttgaggctcaaaacaaactaatcagagcccgtaagccctatcttcatgagtctcgacatgttcatgctcttaaaaggccaagaggatctggtggaagattcctaaacaccaaaaaacttcttcaagaatccgaacaggctgctgctagagaacaagaacaggacaagttaggccaacaggtaaacagaaagaccaacatgtctagattcgaagctcatatgctgcagaacaacaaagaccgcagctcaaccacttctggctcagacatcacctctgtttccgacggtgctgatatctttggacacactgaattccagttttcaggtttcccaactccgataaaccgagccatgcttgttcatggtcagtctaatgacatgcatggaggtggagacatgcaccatttctctgtccatatctgagacagtggatcttggtgctgtgttcatgttcccaccaagaaggggaagtcatccttggctactactagttctttcgcttgttgtaacttcagtgtttttatttcatattatgtctgtgttagacatcacaagaacgaccaagatcttcactttgaaacactctattaccttttcatcttctgttaccatggatctcttgtctaaactagtgatatgattcttctgataaaaaaaaaaa

SEQ ID NO: 48 - protein, 340 aa, Arabidopsis thaliana
  G928
MMHQMLNKKDSATHSTLPYLNTSISWGVVPTDSVANRRGPAESLSLKVDSRPGHIQTTKQISFQDQDSSSTQSTGQSYTEVASSGDDNPSRQISFSAKSGSEITQRKGFASNPKQGSMTGFPNIHFAPAQANFSFHYADPHYGGLLAATYLPQAPTCNPQMVSMIPGRVPLPAELTETDPVFVNAKQYHAIMRRRQQRAKLEAQNKLIRARKPYLHESRHVHALKRPRGSGGRFLNTKKLLQESEQAAAREQEQDKLGQQVNRKTNMSRFEAHMLQNNKDRSSTTSGSDITSVSDGADIFGHTEFQFSGFPTPINRAMLVHGQSNDMHGGGDMHHFSVHI

SEQ ID NO: 49 - DNA, 1553 bp, Arabidopsis thaliana
  G404
ccgggattccgggaaaataataatacgacttttgaaatctctcactaatatttcccaagaaaacctgatgaattaaaagttcatattttcttggtgttgattggtattagatgactatttcttagtattagtcaattctctgtttccatcttatatggatagtctagacacagagcaaagatccttctttggagaaaaagaagatgagagacgagagaggatcgtttcttcacgtattggtcgtagatagatcttaatcgataaccatctaggccttgatctaaccattcaatataaactttatttccttcttcttttcgatctcaattgttccgaccaaacaagatgaagagatcacgtggaagctccgattctttatccggtttcttaccaattcgccattctacaacagacaaacaaataagcccaagaccaacaactaccggctttctctattccggcgccggagactactcccagatgtttgacgcattagaagacgacggaagtctagaggacctcggcggcgttggacacgcgtcttctacggcggcggagaaaaaacggcggttgggtgtagagcaagtgaaagcgttagagaagaatttcgagattgataacaagttagagccagagaggaaagtgaagctggctcaagagcttgggttgcagccacgccaagtcgcgatctggtttcaaaaccgccgtgctcggtggaagacaaagcagcttgaacgtgattacggcgttcttaagtcaaactttgatgcactcaaacgcaaccgcgactcgcttcaacgcgataacgattcactccttggacagattaaagagctgaaagcaaaacttaacgtggaaggggttaaaggtatagaagagaacggcgctttaaaagcagtggaagcaaatcaatcggtgatggctaataatgaagtcttagagctaagccaccgttctccatcgccaccaccgcatattcctacggacgctccgacatcggagctcgcattcgaaatgtttagcatttttccacgcacggaaaacttcagagacgatcctgccgatagcagcgactcaagcgcggttttgaacgaagagtatagtcccaatacggttgaagcagctggcgcagtggcggccacgactgtagaaatgtcgacgatgggttgttttagccaattcgtgaaaatggaagagcatgaagatctgtttagtggagaggaagcttgcaagttgtttgcggacaatgagcaatggtattgctccgatcagtggaattcgtaaaatgtgggggcagaattaaaagcatagaaaagctgaagggggtatgtggaaatattactgaagtcatattgggtcggaaaatgcattactcaaaatattggggtttttcttgagaaaatggaatatgacgcgagagggtagtaaaaacgagcgtgtggcgttccatgcacgtgtggccgtaatcttttcaaggttgaatgagagaggacaaaaaagtctgaaatcatcgggaataaaaaaaatatgtgacgaagaaaaaaaaaaaaaaa

SEQ ID NO: 50 - protein, 312 aa, Arabidopsis thaliana
  G404
MKRSRGSSDSLSGFLPIRHSTTDKQISPRPTTTGFLYSGAGDYSQMFDALEDDGSLEDLGGVGHASSTAAEKKRRLGVEQVKALEKNFEIDNKLEPERKVKLAQELGLQPRQVAIWFQNRRARWKTKQLERDYGVLKSNFDALKRNRDSLQRDNDSLLGQIKELKAKLNVEGVKGIEENGALKAVEANQSVMANNEVLELSHRSPSPPPHIPTDAPTSELAFEMFSIFPRTENFRDDPADSSDSSAVLNEEYSPNTVEAAGAVAATTVEMSTMGCFSQFVKMEEHEDLFSGEEACKLFADNEQWYCSDQWNS

SEQ ID NO: 51 - DNA, 2821 bp, Arabidopsis thaliana
  G438
cggggtacccaagccacgaccgtagaatcttcttttgtctgaaaagaattacaatttacgtttctcttacgatacgacggactttccgaagaaattaatttaaagagaaaagaagaagaagccaaagaagaagaagaagctagaagaaacagtaaagtttgagactttttttgagggtcgagctaaaatggagatggcggtggctaaccaccgtgagagaagcagtgacagtatgaatagacatttagatagtagcggtaagtacgttaggtacacagctgagcaagtcgaggctcttgagcgtgtctacgctgagtgtcctaagcctagctctctccgtcgacaacaattgatccgtgaatgttccattttggccaatattgagcctaagcagatcaaagtctggtttcagaaccgcaggtgtcgagataagcagaggaaagaggcgtcgaggctccagagcgtaaaccggaagctctctgcgatgaataaactgttgatggaggagaatgataggttgcagaagcaggtttctcagcttgtctgcgaaaatggatatatgaaacagcagctaactactgttgttaacgatccaagctgtgaatctgtggtcacaactcctcagcattcgcttagagatgcgaatagtcctgctggattgctctcaatcgcagaggagactttggcagagttcctatccaaggctacaggaactgctgttgattgggttcagatgcctgggatgaagcctggtccggattcggttggcatctttgccatttcgcaaagatgcaatggagtggcagctcgagcctgtggtcttgttagcttagaacctatgaagattgcagagatcctcaaagatcggccatcttggttccgtgactgtaggagccttgaagttttcactatgttcccggctggtaatggtggcacaatcgagcttgtttatatgcagacgtatgcaccaacgactctggctcctgcccgcgatttctggaccctgagatacacaacgagcctcgacaatgggagttttgtggtttgtgagaggtcgctatctggctctggagctgggcctaatgctgcttcagcttctcagtttgtgagagcagaaatgctttctagtgggtatttaataaggccttgtgatggtggtggttctattattcacattgtcgatcaccttaatcttgaggcttggagtgttccggatgtgcttcgacccctttatgagtcatccaaagtcgttgcacaaaaaatgaccatttccgcgttgcggtatatcaggcaattagcccaagagtctaatggtgaagtagtgtatggattaggaaggcagcctgctgttcttagaacctttagccaaagattaagcaggggcttcaatgatgcggttaatgggtttggtgacgacgggtggtctacgatgcattgtgatggagcggaagatattatcgttgctattaactctacaaagcatttgaataatatttctaattctctttcgttccttggaggcgtgctctgtgccaaggcttcaatgcttctccaaaatgttcctcctgcggttttgatccggttccttagagagcatcgatctgagtgggctgatttcaatgttgatgcatattccgctgctacacttaaagctggtagctttgcttatccgggaatgagaccaacaagattcactgggagtcagatcataatgccactaggacatacaattgaacacgaagaaatgctagaagttgttagactggaaggtcattctcttgctcaagaagatgcatttatgtcacgggatgtccatctccttcagatttgtaccgggattgacgagaatgccgttggagcttgttctgaactgatatttgctccgattaatgagatgttcccggatgatgctccacttgttccctctggattccgagtcatacccgttgatgctaaaacgggagatgtacaagatctgttaaccgctaatcaccgtacactagacttaacttctagccttgaagtcggtccatcacctgagaatgcttctggaaactctttttctagctcaagctcgagatgtattctcactatcgcgtttcaattcccttttgaaaacaacttgcaagaaaatgttgctggtatggcttgtcagtatgtgaggagcgtgatctcatcagttcaacgtgttgcaatggcgatctcaccgtctgggataagcccgagtctgggctccaaattgtccccaggatctcctgaagctgttactcttgctcagtggatctctcaaagttacagtcatcacttaggctcggagttgctgacgattgattcacttggaagcgacgactcggtactaaaacttctatgggatcaccaagatgccatcctgtgttgctcattaaagccacagccagtgttcatgtttgcgaaccaagctggtctagacatgctagagacaacacttgtagccttacaagatataacactcgaaaagatattcgatgaatcgggtcgtaaggctatctgttcggacttcgccaagctaatgcaacagggatttgcttgcttgccttcaggaatctgtgtgtcaacgatgggaagacatgtgagttatgaacaagctgttgcttggaaagtgtttgctgcatctgaagaaaacaacaacaatctgcattgtcttgccttctcctttgtaaactggtcttttgtgtgattcgattgacagaaaaagactaatttaaatttacgttagagaactcaaatttttggttgttgtttaggtgtctctgttttgttttttaaaattattttgatcaaa

SEQ ID NO: 52 - protein, 842 aa, Arabidopsis thaliana
  G438
MEMAVANHRERSSDSMNRHLDSSGKYVRYTAEQVEALERVYAECPKPSSLRRQQLIRECSILANIEPKQIKVWFQNRRCRDKQRKEASRLQSVNRKLSAMNKLLMEENDRLQKQVSQLVCENGYMKQQLTTVVNDPSCESVVTTPQHSLRDANSPAGLLSIAEETLAEFLSKATGTAVDWVQMPGMKPGPDSVGIFAISQRCNGVAARACGLVSLEPMKIAEILKDRPSWFRDCRSLEVFTMFPAGNGGTIELVYMQTYAPTTLAPARDFWTLRYTTSLDNGSFVVCERSLSGSGAGPNAASASQFVRAEMLSSGYLIRPCDGGGSIIHIVDHLNLEAWSVPDVLRPLYESSKVVAQKMTISALRYIRQLAQESNGEVVYGLGRQPAVLRTFSQRLSRGFNDAVNGFGDDGWSTMHCDGAEDIIVAINSTKHLNNISNSLSFLGGVLCAKASMLLQNVPPAVLIRFLREHRSEWADFNVDAYSAATLKAGSFAYPGMRPTRFTGSQIIMPLGHTIEHEEMLEVVRLEGHSLAQEDAFMSRDVHLLQICTGIDENAVGACSELIFAPINEMFPDDAPLVPSGFRVIPVDAKTGDVQDLLTANHRTLDLTSSLEVGPSPENASGNSFSSSSSRCILTIAFQFPFENNLQENVAGMACQYVRSVISSVQRVAMAISPSGISPSLGSKLSPGSPEAVTLAQWISQSYSHHLGSELLTIDSLGSDDSVLKLLWDHQDAILCCSLKPQPVFMFANQAGLDMLETTLVALQDITLEKIFDESGRKAICSDFAKLMQQGFACLPSGICVSTMGRHVSYEQAVAWKVFAASEENNNNLHCLAFSFVNWSFV

SEQ ID NO: 53 - DNA, 818 bp, Arabidopsis thaliana
  G46
ttctctctttcgtaaaaatggcgagttttgaggaaagctctgatttggaagctatacagagccatctcttagaagacttgttggtttgtgatggtttcatgggagattttgacttcgatgcttcttttgtctcaggactttggtgtatagaaccacacgttcctaaacaagaacctgattctccagttcttgatccggattctttcgtcaacgagttcttgcaagtggaaggggaatcatcatcatcatcatcaccagagctgaattcatcgtcatcaacatatgagactgatcagagtgtgaaaaaggcagagaggttcgaagaagaagtagatgctagacattaccgaggagtgaggcgaaggccgtgggggaaatttgcagcagagattcgagatccagcaaagaaaggatcaagaatctggctaggaacatttgagagtgatgttgatgctgcaagagcctatgactgtgcagctttcaagctccggggaagaaaagccgtgctcaacttccctcttgacgccgggaaatatgaagctccagcgaattcaggaaggaaaaggaagagaagtgatgtgcatgaagagcttcaaagaactcagagcaattcatcttcatcttcctgtgatgcattttagcatattaagagtgtgagcagtttccttaagttgtataaagtaattgtacagaggaaacgaattgtgtaggtttagtgtgcttgcaagttgcaacaaatgtgtatggatgttctgtttcttcatgtccctaagatttagaaacatcttcttatttccaagaaaaaaaaaaaaaaaaaa

SEQ ID NO: 54 - protein, 207 aa, Arabidopsis thaliana
  G46
MASFEESSDLEAIQSHLLEDLLVCDGFMGDFDFDASFVSGLWCIEPHVPKQEPDSPVLDPDSFVNEFLQVEGESSSSSSPELNSSSSTYETDQSVKKAERFEEEVDARHYRGVRRRPWGKFAAEIRDPAKKGSRIWLGTFESDVDAARAYDCAAFKLRGRKAVLNFPLDAGKYEAPANSGRKRKRSDVHEELQRTQSNSSSSSCDAF

SEQ ID NO: 55 - DNA, 1130 bp, Arabidopsis thaliana
  G921
ccaagatcgactcttacttcgaatctctctcaactttcttcctcagcttacgggaacttccacacatatacatccacaagaacccatatcgaagattcatcctacatatatttacatggatcagtactcatcctctttggtcgatacttcattagatctcactattggcgttactcgtatgcgagttgaagaagatccaccgacaagtgctttggtggaagaattaaaccgagttagtgctgagaacaagaagctctcggagatgctaactttgatgtgtgacaactacaacgtcttgaggaagcaacttatggaatatgttaacaagagcaacataaccgagagggatcaaatcagccctcccaagaaacgcaaatccccggcgagagaggacgcattcagctgcgcggttattggcggagtgtcggagagtagctcaacggatcaagatgagtatttgtgtaagaagcagagagaagagactgtcgtgaaggagaaagtctcaagggtctattacaagaccgaagcttctgacactaccctcgttgtgaaagatgggtatcaatggaggaaatatggacagaaagtgactagagacaatccatctccaagagcttacttcaaatgtgcttgtgctccaagctgttctgtcaaaaagaaggttcagagaagtgtggaggatcagtccgtgttagttgcaacttatgagggtgaacacaaccatccaatgccatcgcagatcgattcaaacaatggcttaaaccgccacatctctcatggtggttcagcttcaacacccgttgcagcaaacagaagaagtagcttgactgtgccggtgactaccgtagatatgattgaatcgaagaaagtgacgagcccaacgtcaagaatcgattttccccaagttcagaaacttttggtggagcaaatggcttcttccttaaccaaagatcctaactttacagcagctttagcagcagctgttaccggaaaattgtatcaacagaatcataccgagaaatagtttagcttcaaattccgttagagtttttagatttgaatttgtcatgagtaagagaaagagagtagattataatccnttgtgatactgaaaaaaaaaaaaaaaaaaa

SEQ ID NO: 56 - protein, 302 aa, Arabidopsis thaliana
  G921
MDQYSSSLVDTSLDLTIGVTRMRVEEDPPTSALVEELNRVSAENKKLSEMLTLMCDNYNVLRKQLMEYVNKSNITERDQISPPKKRKSPAREDAFSCAVIGGVSESSSTDQDEYLCKKQREETVVKEKVSRVYYKTEASDTTLVVKDGYQWRKYGQKVTRDNPSPRAYFKCACAPSCSVKKKVQRSVEDQSVLVATYEGEHNHPMPSQIDSNNGLNRHISHGGSASTPVAANRRSSLTVPVTTVDMIESKKVTSPTSRIDFPQVQKLLVEQMASSLTKDPNFTAALAAAVTGKLYQQNHTEK

SEQ ID NO: 57 - DNA, 1046 bp, Arabidopsis thaliana
  G233
gaaaaacatttcaacttcttttatcagcaatcacaaatcaaagagatgggaagagctccatgctgtgagaagatggggttgaagagaggaccatggacacctgaagaagatcaaatcttggtctcttttatcctcaaccatggacatagtaactggcgagccctccctaagcaagctggtcttttgagatgtggaaaaagctgtagacttaggtggatgaactatttaaagcctgatattaaacgtggcaatttcaccaaagaagaggaagatgctatcatcagcttacaccaaatacttggcaatagatggtcagcgattgcagcaaaactgcctggaagaaccgataacgagatcaagaacgtatggcacactcacttgaagaagagactcgaagattatcaaccagctaaacctaagaccagcaacaaaaagaagggtactaaaccaaaatctgaatccgtaataacgagctcgaacagtactagaagcgaatcggagctagcagattcatcaaacccttctggagaaagcttattttcgacatcgccttcgacaagtgaggtttcttcgatgacactcataagccacgacggctatagcaacgagattaatatggataacaaaccgggagatatcagtactatcgatcaagaatgtgtttctttcgaaacttttggtgcggatatcgatgaaagcttctggaaagagacactgtatagccaagatgaacacaactacgtatcgaatgacctagaagtcgctggtttagttgagatacaacaagagtttcaaaacttgggctccgctaataatgagatgatttttgacagtgagatggaacttctggttcgatgtattggctagaaccggcggggaacaagatctcttagccgggctctagttaacatgtttgaggagtaaagtgaaatggtgcaaattagttaaggctaagaaattcaaaagcttttgtttaccgagaaaaaaacacactctaactcttgatgtgatgtagttagtgtattaattagaggctgcgttttcaa

SEQ ID NO: 58 - protein, 273 aa, Arabidopsis thaliana
  G233
MGRAPCCEKMGLKRGPWTPEEDQILVSFILNHGHSNWRALPKQAGLLRCGKSCRLRWMNYLKPDIKRGNFTKEEEDAIISLHQILGNRWSAIAAKLPGRTDNEIKNVWHTHLKKRLEDYQPAKPKTSNKKKGTKPKSESVITSSNSTRSESELADSSNPSGESLFSTSPSTSEVSSMTLISHDGYSNEINMDNKPGDISTIDQECVSFETFGADIDESFWKETLYSQDEHNYVSNDLEVAGLVEIQQEFQNLGSANNEMIFDSEMELLVRCIG

SEQ ID NO: 59 - DNA, 1150 bp, Arabidopsis thaliana
  G1131
tcgactcctctcctgattgcttcaccttcttctttactacaggtttcagctcctcaatgtccatggattgcttaagctacttctttaactacgatcctcctgtccagctccaggattgctttattcccgagatggatatgattatccctgaaaccgatagtttcttcttccaatctcaaccgcaactggagtttcatcagccattgtttcaagaagaagctccttcacagacccactttgaccctttctgcgaccagtttctttctccgcaagaaatctttctccctaaccctaaaaacgaaatcttcaacgaaacacacgacctcgatttctttctccccacgccaaaacgccagagacttgttaactccagctacaattgtaacactcaaaaccatttccagagccgtaacccgaatttcttcgaccctttcggcgacactgatttcgttccagaatcttgtaccttccaggagtttcgagttccggatttctctttagctttcaaggtaggccggggagatcaagatgactcaaagaaaccgacgctttcatctcagagcatcgcggctagagggaggagaagaagaattgcagagaagactcacgagctcggaaaactcatccccggtggcaataaacttaacaccgccgagatgttccaagccgccgctaagtatgtcaagtttttgcagagtcaagttgggattctccaactgatgcagaccacaaagaaggtaataaccaaccccaaataagaactttatcatccaattgaaactctaatcgtgttttctcacaagcttcttaatttgtttacgcagggtagctctaatgtgcaaatggaaactcagtatttgcttgaatcgcaagcaatccaggagaagttatcaacagaggaagtgtgtttggtaccgtgtgaaatggttcaagatctaacaactgaagaaaccatttgcagaaccccgaatatttctcgagaaatcaacaagttactgtctaaacatctggctaactagttttagtttcaagcctgaagttctctatgcctaaatttgtgtctgttatcgttgttttgtcttcttagttagtgttttgtcttgttgatttaggggctaattatcctggttaatctcctcttaactgggaa

SEQ ID NO: 60 - protein, 233 aa, Arabidopsis thaliana
  G1131
MSMDCLSYFFNYDPPVQLQDCFIPEMDMIIPETDSFFFQSQPQLEFHQPLFQEEAPSQTHFDPFCDQFLSPQEIFLPNPKNEIFNETHDLDFFLPTPKRQRLVNSSYNCNTQNHFQSRNPNFFDPFGDTDFVPESCTFQEFRVPDFSLAFKVGRGDQDDSKKPTLSSQSIAARGRRRRIAEKTHELGKLIPGGNKLNTAEMFQAAAKYVKFLQSQVGILQLMQTTKKVITNPK

SEQ ID NO: 61 - DNA, 1413 bp, Arabidopsis thaliana
  G1141
gtctttacaaaaagaaaagaaaaaagagacgctagaaagaacgcgaaagcttgcgaagaagatttgcttttgatcgacttaacacgaacaacaaacaacatctgcgtgataaagaagagatttttgcctaaataaagaagagattcgactctaatcctggagttatcattcacgatagattcttagattgcgactataaagaagaagatggctgtatatgaacaaaccggaaccgagcagccgaagaaaaggaaatctagggctcgagcaggtggtttaacggtggctgataggctaaagaagtggaaagagtacaacgagattgttgaagcttcggctgttaaagaaggagagaaaccgaaacgcaaagttcctgcgaaagggtcgaagaaaggttgtatgaagggtaaaggaggaccagataattctcactgtagttttagaggagttagacaaaggatttggggtaaatgggttgcagagattcgagaaccgaaaataggaactagactttggcttggtacttttcctaccgcggaaaaagctgcttccgcttatgatgaagcggctaccgctatgtacggttcattggctcgtcttaacttccctcagtctgttgggtctgagtttactagtacgtctagtcaatctgaggtgtgtacggttgaaaataaggcggttgtttgtggtgatgtttgtgtgaagcatgaagatactgattgtgaatctaatccatttagtcagattttagatgttagagaagagtcttgtggaaccaggccggacagttgcacggttggacatcaagatatgaattcttcgctgaattacgatttgctgttagagtttgagcagcagtattggggccaagttttgcaggagaaagagaaaccgaagcaggaagaagaggagatacagcaacagcaacaggaacagcaacagcaacagctgcaaccggatttgcttactgttgcagattacggttggccttggtctaatgatattgtaaatgatcagacttcttgggatcctaatgagtgctttgatattaatgaactccttggagatttgaatgaacctggtccccatcagagccaagaccaaaaccacgtaaattctggtagttatgatttgcatccgcttcatctcgagccacacgatggtcacgagttcaatggtttgagttctctggatatttgagagttctgaggcaatggtcctacaagactacaacataatctttggattgatcataggagaaacaagaaataggtgttaatgatctgattcacaatgaaaaaatatttaataactctatagtttttgttctttccttggatcatgaactgttgcttctcatctattgagttaatatagcgaatagcagagtttctctcataaaaaaaaaaaaaa

SEQ ID NO: 62 - protein, 330 aa, Arabidopsis thaliana
  G1141
MAVYEQTGTEQPKKRKSRARAGGLTVADRLKKWKEYNEIVEASAVKEGEKPKRKVPAKGSKKGCMKGKGGPDNSHCSFRGVRQRIWGKWVAEIREPKIGTRLWLGTFPTAEKAASAYDEAATAMYGSLARLNFPQSVGSEFTSTSSQSEVCTVENKAVVCGDVCVKHEDTDCESNPFSQILDVREESCGTRPDSCTVGHQDMNSSLNYDLLLEFEQQYWGQVLQEKEKPKQEEEEIQQQQQEQQQQQLQPDLLTVADYGWPWSNDIVNDQTSWDPNECFDINELLGDLNEPGPHQSQDQNHVNSGSYDLHPLHLEPHDGHEFNGLSSLDI

SEQ ID NO: 63 - DNA, 1247 bp, Arabidopsis thaliana
  G1181
ctcgatcttttaacccccattattacatattactccttcctacattattcttcttctgctttcgtgactttcaggggacacttttgtttttataacttacgcttaaaatcctatgaattcgccgccggttgacgcaatgattaccggagaatcatcgtcacaaagatctatcccaacgccgtttctcacaaaaacgtttaacctcgttgaagatagttccatcgacgatgttatctcatggaacgaagatggttcctctttcatcgtatggaatccgacagatttcgctaaagatttgcttcctaaacacttcaaacacaacaatttctctagtttcgttcgtcagctcaacacttacggattcaaaaaagttgtaccggatcgatgggagttttcaaacgatttctttaagagaggagaaaaacgtcttctccgtgagatccaacgtcggaaaataacaacgacgcatcaaacagttgttgctccttcgtcggaacaacgaaaccagacgatggttgtatcaccgtcaaattccggggaagataataataataatcaggtgatgtcttcgtctccgtcgtcgtggtattgtcatcaaacgaagacgactgggaatggtggtttatcagtggagttattggaagagaacgagaagcttcggagtcaaaacattcagctaaaccgtgagcttactcagatgaaatctatctgcgataatatctatagtctcatgtcgaattacgtcggatctcagcccactgatcggagttattctcccggaggtagtagtagtcaaccgatggagtttttaccggcgaagcggttttcggagatggagattgaagaagaagaagaagcgagtccgaggttgtttggtgttccgattgggttaaaacggacgagaagtgaaggtgttcaggtgaagacgacggcggtggttggggaaaattccgatgaggagacgccgtggttgagacattataatcgaaccaatcagagagtttgtaattaaaaacgaacggtttagatttgtggtgtagatatgtgcgcgaagtagacgattacagctttttaagacaagcagagcacgtgtcccatctgtttcaagaagtttctgcaatcttgacttcttcttttaacactttgtgttttttattatttaattaataacaataaatgttctttttcagttttgttttcttcaaaaatagttcggctgtttctagactttccttttttgaccaaaa

SEQ ID NO: 64 - protein, 299 aa, Arabidopsis thaliana
  G1181
MNSPPVDAMITGESSSQRSIPTPFLTKTFNLVEDSSIDDVISWNEDGSSFIVWNPTDFAKDLLPKHFKHNNFSSFVRQLNTYGFKKVVPDRWEFSNDFFKRGEKRLLREIQRRKITTTHQTVVAPSSEQRNQTMVVSPSNSGEDNNNNQVMSSSPSSWYCHQTKTTGNGGLSVELLEENEKLRSQNIQLNRELTQMKSICDNIYSLMSNYVGSQPTDRSYSPGGSSSQPMEFLPAKRFSEMEIEEEEEASPRLFGVPIGLKRTRSEGVQVKTTAVVGENSDEETPWLRHYNRTNQRVCN

SEQ ID NO: 65 - DNA, 1142 bp, Arabidopsis thaliana
  G1319
catcaatacattaccaagaaaccagagtttttcttcattgttcttttccacagcattgcaacatgaagagacattcttgttgttacaaacaaaagctgagaaaaggtctttggtctcctgaagaagatgagaaacttctcaattacattaccaaacatggccatggttgctggagttctgtccctaaactcgcaggtctcgagagatgtggaaagagctgtagactcagatggatcaattacttaagacctgatttaaagagaggagctttctcttcagaggaacagaatctcattgtcgagcttcatgctgttcttggaaacagatggtcgcaaattgctgcgaggcttcccgggagaaccgacaacgagataaagaacttgtggaattcgtgcattaagaagaagctgatgaagaaaggcattgaccctattacacataaacccctctccgaggttggtaaagaaacaaacagaagcgacaataacaattccacaagtttttcctcagaaactaatcaagacttgtttgtcaagaaaacgtctgattttgccgagtattctgcgtttcagaaagaagaatccaactctgtttcactcagaaattcgctctcttccatgatcccaacgcaattcaacatcgacgatggttctgtctcaaatgcgggttttgatacacaagtatgcgtgaaaccctcgattattcttcttcctcctccaaacaacacttcaagcactgtctctggacaggatcatgtaaacgtgtcagagcctaattgtgaatcaaacagtggaaccacaagccacctcaacaatcccggtatggaagaaatgaaatggtccgaggagtacctaaacgaatcgttattctctacccaagtttacgtgaaatcagagacggatttcaactccaacattgcctttccttggagccaaagccaagcttgtgacgtattccccaaggatcttcagagaatggccttctcttttggtggtcagaccctttagtttctttttcttatcagatttagacatattgatacgtgtaatgaatggatcaaatgttctatttggccatacgttaaaaaaataaaaaagcacaatcctttcctgaaaaaaaaaaaaaaaaaaaaaaaaaaaaaa

SEQ ID NO: 66 - protein, 314 aa, Arabidopsis thaliana
  G1319
MKRHSCCYKQKLRKGLWSPEEDEKLLNYITKHGHGCWSSVPKLAGLERCGKSCRLRWINYLRPDLKRGAFSSEEQNLIVELHAVLGNRWSQIAARLPGRTDNEIKNLWNSCIKKKLMKKGIDPITHKPLSEVGKETNRSDNNNSTSFSSETNQDLFVKKTSDFAEYSAFQKEESNSVSLRNSLSSMIPTQFNIDDGSVSNAGFDTQVCVKPSIILLPPPNNTSSTVSGQDHVNVSEPNCESNSGTTSHLNNPGMEEMKWSEEYLNESLFSTQVYVKSETDFNSNIAFPWSQSQACDVFPKDLQRMAFSFGGQTL

SEQ ID NO: 67 - DNA, 1161 bp, Arabidopsis thaliana
  G1328
aattcaatcactatatttttttaaaaacatttgacttcatcgatcggttaacaattaatcaaaaagatgggacgatcaccatgttgtgagaagaagaatggtctcaagaaaggaccatggactcctgaggaggatcaaaagctcattgattatatcaatatacatggttatggaaattggagaactcttcccaagaatgctgggttacaaagatgtggtaagagttgtcgtctccggtggaccaactatctccgaccagatattaagcgtggaagattctcttttgaagaagaagaaaccattattcaacttcacagcatcatgggaaacaagtggtctgcgattgcggctcgtttgcctggaagaacagacaacgagatcaaaaactattggaacactcacatcagaaaaagacttctaaagatgggaatcgacccggttacacacactccacgtcttgatcttctcgatatctcctccattctcagctcatctatctacaactcttcgcatcatcatcatcatcatcatcaacaacatatgaacatgtcgaggctcatgatgagtgatggtaatcatcaaccattggttaaccccgagatactcaaactcgcaacctctctcttttcaaaccaaaaccaccccaacaacacacacgagaacaacacggttaaccaaaccgaagtaaaccaataccaaaccggttacaacatgcctggtaatgaagaattacaatcttggttccctatcatggatcaattcacgaatttccaagacctcatgccaatgaagacgacggtccaaaattcattgtcatacgatgatgattgttcgaagtccaattttgtattagaaccttattactccgactttgcttcagtcttgaccacaccttcttcaagcccgactccgttaaactcaagttcctcaacttacatcaatagtagcacttgcagcaccgaggatgaaaaagagagttattacagtgataatatcactaattattcgtttgatgttaatggttttctccaattccaataaacaaaacgccattggaatagagttatgtaaacatgcaatcattgtatttgttatatagattttgttacatatccaaaatccaaaatactatagttttaaaataaaaaaaaaaaaaaaaaa

SEQ ID NO: 68 - protein, 324 aa, Arabidopsis thaliana
  G1328
MGRSPCCEKKNGLKKGPWTPEEDQKLIDYINIHGYGNWRTLPKNAGLQRCGKSCRLRWTNYLRPDIKRGRFSFEEEETIIQLHSIMGNKWSAIAARLPGRTDNEIKNYWNTHIRKRLLKMGIDPVTHTPRLDLLDISSILSSSIYNSSHHHHHHHQQHMNMSRLMMSDGNHQPLVNPEILKLATSLFSNQNHPNNTHENNTVNQTEVNQYQTGYNMPGNEELQSWFPIMDQFTNFQDLMPMKTTVQNSLSYDDDCSKSNFVLEPYYSDFASVLTTPSSSPTPLNSSSSTYINSSTCSTEDEKESYYSDNITNYSFDVNGFLQFQ

SEQ ID NO: 69 - DNA, 1274 bp, Arabidopsis thaliana
  G1334
atagctcccaactaataggaatctcaagcttctcactctctcttgtttttccattggacttttggaacataagctatgcaaactgaggagcttttgtcgccaccacagactccttggtggaatgcttttggatctcagccgttgactacagagagcctttccggcgaagcttctgattcattcaccggagttaaggcagttactacggaggcagaacaaggtgtggtggataaacaaacttctacaactctcttcactttctcacctggtggtgaaaagagttcaagagatgtgccaaagcctcatgttgctttcgcgatgcaatcagcttgcttcgagtttggatttgctcagccaatgatgtacacaaagcatcctcatgttgaacaatactatggagttgtttcagcatacggatctcagaggtcttcgggccgagtaatgattccactgaagatggagacagaagaagatggtaccatctatgtgaactcaaagcagtaccatggaattatcaggcgacgccagtcccgagcaaaggctgaaaaactgagtagatgccgtaagccatatatgcatcactcacgccatctccatgctatgcgccgtcctagaggatctggcgggcgtttcttgaacaccaagacagctgatgcggctaagcagtctaagccgagtaattctcagagttctgaagtctttcatccggaaaatgagaccataaactcatcgagggaagcaaatgagtcaaatctctcggattctgcagttacaagtatggattactttctaagttcgtcggcttattctcctggtggcatggtcatgcctatcaagtggaatgcagcagcaatggatattggctgctgcaaacttaatatatgatcagcagataggggacaagacatgattggtcaccagtccttttgtcttgtcccttatctttcagccaaacggaaagagaacttgtgtcttggaaaaaagacattgagtttccttggtttataagattggtccttttaccatccgtttggctgtaaacaggcaaatcatctttggctcatgcttcatcaagttcttatcttcgtctgttttcttctacgcatcttcataagatctctgaactagtgaataacatttcctagcatcatgtttcaactagtgtgtgttgtaagaaactctgccttatttccagatgatgtattgtgtgtaacgtgtttatgaaacaaacgtaagactttcaagttaaaaaaaaaaaaaaaaaaaaaaaaaaa

SEQ ID NO: 70 - protein, 269 aa, Arabidopsis thaliana
  G1334
MQTEELLSPPQTPWWNAFGSQPLTTESLSGEASDSFTGVKAVTTEAEQGVVDKQTSTTLFTFSPGGEKSSRDVPKPHVAFAMQSACFEFGFAQPMMYTKHPHVEQYYGVVSAYGSQRSSGRVMIPLKMETEEDGTIYVNSKQYHGIIRRRQSRAKAEKLSRCRKPYMHHSRHLHAMRRPRGSGGRFLNTKTADAAKQSKPSNSQSSEVFHPENETINSSREANESNLSDSAVTSMDYFLSSSAYSPGGMVMPIKWNAAAMDIGCCKLNI

SEQ ID NO: 71 - DNA, 1498 bp, Arabidopsis thaliana
  G14
gaagaagcaaagatataacccccaaaagtatcaattagtttccattttcgccgctaagattctgttttcgaacatttacaccctcaagaatcgccgccatgtgtggaggagctataatatccgatttcattccaccgccgaggtctcgccgtgttactagcgagtttatttggccggatctgaagaagaatttgaaaggatcgaagaaaagctcgaagaatcgttcgaatttcttcgattttgacgctgagttcgaagctgatttccaaggtttcaaagatgattcgtctatcgattgcgatgatgatttcgacgtcggtgatgttttcgccgatgtgaaaccattcgttttcacttcgactccaaaacccgccgtctccgccgctgcggaaggttcagtttttggtaagaaagttactggcttggatggggacgctgagaaatctgcaaataggaagaggaagaatcagtaccgagggattaggcaacgtccttggggaaaatgggctgctgagatacgtgatccaagggaaggtgctagaatctggcttggaacgttcaagacagctgaggaagctgctagagcttacgatgctgcagcgcggagaatccgtggatctaaagctaaggtgaatttccctgaagaaaacatgaaggctaattctcagaaacgctctgtgaaggctaatcttcagaaaccagtggctaaacctaaccctaacccaagtccagctttggttcagaactcgaacatctcctttgaaaatatgtgtttcatggaggagaaacaccaagtgagcaacaacaacaacaaccagtttgggatgacaaactccgttgatgctggatgtaatgggtatcagtatttcagctctgaccagggtagtaattctttcgattgttcggagtttggttggagcgatcaagctccgataactcccgacatctcttctgcggttatcaacaacaacaactcagctctgttctttgaggaagccaatccagctaagaagctcaagtctatggatttcgagacaccttacaacaacactgaatgggacgcttcactggatttcctcaacgaagatgctgtaacgactcaggacaatggtgcaaaccctatggacctatggagtattgatgaaattcattccatgattggaggagtcttctgaagagatccagtttcatgtaaataaggctgcatgtttgtgagtttcccgcatcgttcgtttatcaacctccaaaactttctaatgtctgttacttgcatcttcttctgctgtctctgtctgtctctctcaggagttcctgtttgcattgcgagaagccatgagcctctatcttgagggtagttgtgatgaagttaagtagaggcttatttttaggggttgtggtagtttttgttttagtgaatcttttgaattcgtttgtgttttgtttttgttactttatgccccaaaactcctttaacatttgtcataatgtgtttgaacct

SEQ ID NO: 72 - protein, 358 aa, Arabidopsis thaliana
  G14
MCGGAIISDFIPPPRSRRVTSEFIWPDLKKNLKGSKKSSKNRSNFFDFDAEFEADFQGFKDDSSIDCDDDFDVGDVFADVKPFVFTSTPKPAVSAAAEGSVFGKKVTGLDGDAEKSANRKRKNQYRGIRQRPWGKWAAEIRDPREGARIWLGTFKTAEEAARAYDAAARRIRGSKAKVNFPEENMKANSQKRSVKANLQKPVAKPNPNPSPALVQNSNISFENMCFMEEKHQVSNNNNNQFGMTNSVDAGCNGYQYFSSDQGSNSFDCSEFGWSDQAPITPDISSAVINNNNSALFFEEANPAKKLKSMDFETPYNNTEWDASLDFLNEDAVTTQDNGANPMDLWSIDEIHSMIGGVF

SEQ ID NO: 73 - DNA, 1260 bp, Arabidopsis thaliana
  G1412
cccacgcgtccgcccacgcgtccgaaacaaaaacatataatttgggtttttagagttcgaaacttgaaatctttttttttttggttgctgaggaatcgaagtagaagagtataaatgggtgttagagagaaagatccgttagcccagttgagtttgccaccaggttttagattttatccgacagatgaagagcttcttgttcagtatctatgtcggaaagttgcaggctatcatttctctctccaggtcatcggagacatcgatctctacaagttcgatccttgggatttgccaagtaaggctttgtttggagagaaggaatggtatttctttagcccaagagatcggaaatatccgaacgggtcaagacccaatagagtagccgggtcgggttattggaaagcaacgggtactgacaaaattatcacggcggatggtcgtcgtgtcgggattaaaaaagctctggtcttttacgccggaaaagctcccaaaggcactaaaaccaactggattatgcacgagtatcgcttaatagaacattctcgtagccatggaagctccaagttggatgattgggtgttgtgtcgaatttacaagaaaacatctggatctcagagacaagctgttactcctgttcaagcttgtcgtgaagagcatagcacgaatgggtcgtcatcgtcttcttcatcacagcttgacgacgttcttgattcgttcccggagataaaagaccagtcttttaatcttcctcggatgaattcgctcaggacgattcttaacgggaactttgattgggctagcttggcaggtcttaatccaattccagagctagctccgaccaatggattaccgagttacggtggttacgatgcgtttcgagcggcggaaggtgaggcggagagtgggcatgtgaatcggcagcagaactcgagcgggttgactcagagtttcgggtacagctcgagtgggtttggtgtttcgggtcaaacattcgagtttaggcaatgagagagatgtgaagttactgatgggtgaaaaaagtaaaaaaaaaacttggagatagtagagtggcaattgatgtaaataatagggatttatatggggcttttaccgattcggtgaggcttaggattccccaaaggaaaaaggctcgactggggactagtttgatccaacttgacggcccccaaatgtgtaatgtttctcaacggagagaaaaataaatggttaccaatatttttccaaaaaaaaaaaaaaaaa

SEQ ID NO: 74 - protein, 297 aa, Arabidopsis thaliana
  G1412
MGVREKDPLAQLSLPPGFRFYPTDEELLVQYLCRKVAGYHFSLQVIGDIDLYKFDPWDLPSKALFGEKEWYFFSPRDRKYPNGSRPNRVAGSGYWKATGTDKIITADGRRVGIKKALVFYAGKAPKGTKTNWIMHEYRLIEHSRSHGSSKLDDWVLCRIYKKTSGSQRQAVTPVQACREEHSTNGSSSSSSSQLDDVLDSFPEIKDQSFNLPRMNSLRTILNGNFDWASLAGLNPIPELAPTNGLPSYGGYDAFRAAEGEAESGHVNRQQNSSGLTQSFGYSSSGFGVSGQTFEFRQ

SEQ ID NO: 75 - DNA, 1015 bp, Arabidopsis thaliana
  G16
ctgtctctcactttccttcattatttactcattgcccaaaataattcatttatcatatcctcaaacttttttttttctatacctcgaaatccaaaaaaataaatacattggtttagtgtggcattgatgtttttggtataaagctcttcaccaatgatagcttcagagagtaccaagagctgggaagctagcgcagtcagacaagagaatgaagaagagaagaagaaaccggttaaagattccggtaagcatccggtttatcggggtgtccgaaagaggaactggggaaaatgggtgtccgagatacgtgaacctaggaaaaaatcccgaatatggctaggaacgtttccttccccggagatggcggcgcgtgcacacgacgtagccgctcttagcatcaaaggagcctccgctatactcaatttccctgacctagccggctctttcccacgccctagctcgcttagccctcgagacatccaggtcgcggctctcaaagccgcacacatggagacctcacagtctttttcttcttcttcttctttaacgttttcatcttcacagtcttcttcttcgctagagtctctcgtgtcttcctccgcgaccggctccgaggagctaggggagattgtagagctcccaagtttgggatcgagctatgatggtttgactcagctaggtaacgagtttatattctctgactccgcagacttatggccttatccaccgcaatggtcagaaggtgattaccaaatgattcctgcctcgttatcacaagattgggatcttcaaggactgtataattattaagtcataatcataaaaatatactagtaatgctgtgtttaataattactttttaatgacccctttattttgtatagggcggctttaggtttatttgtctttgaatcttcttttctcaatttgtgggtgtgagagagctcgatatatgaatacttgtacatacttctcataacatgtgttttttttttcttaataaatgtggaaactt

SEQ ID NO: 76 - protein, 218 aa, Arabidopsis thaliana
  G16
MIASESTKSWEASAVRQENEEEKKKPVKDSGKHPVYRGVRKRNWGKWVSEIREPRKKSRIWLGTFPSPEMAARAHDVAALSIKGASAILNFPDLAGSFPRPSSLSPRDIQVAALKAAHMETSQSFSSSSSLTFSSSQSSSSLESLVSSSATGSEELGEIVELPSLGSSYDGLTQLGNEFIFSDSADLWPYPPQWSEGDYQMIPASLSQDWDLQGLYNY

SEQ ID NO: 77 - DNA, 1974 bp, Arabidopsis thaliana
  G184
tgaattctagcctttttgtaggcgaatcatctggaccggtaagagactctctcatcgataataaccacataatttaatcaaactctttctctctctttctaagatcttttgctttgctcttttcctttttgatcttcctatatatggagaagcaccaaaacggtacttactatacgatactgtacggatccatcaaactggattaattatcaaaacgtacatttttatcttacctggcaagttacattcctagggttttggagaatccaatcaacaacaaagaaaataatcatcgttacaataatcagtatcacgcacagacttagatgttccggtttccagtgagtctaggcggttcacgtgacgaagaccgtcacgatcagatcacaccgttggatgaccatcgtgtggtggttgatgaggttgacttcttctcagagaagagagatagggtttcacgtgagaacatcaacgacgacgacgacgaaggcaataaggttctcatcaaaatggagggttcacgagttgaagaaaacgatcgttccagagatgtcaatatcggtctgaatcttctgaccgcgaatacgggaagcgatgagtcaacggtggatgatggactatcaatggatatggaagataaacgtgcaaagattgagaacgcacaactacaagaagagctcaagaagatgaaaatagagaatcaaaggctaagagatatgttgagccaagcgacgaccaacttcaatgccttacaaatgcaacttgttgccgtcatgaggcaacaagaacaacgtaactcttcacaagatcatctcctggagagcaaagcagaaggaaggaaacggcaggaactgcaaatcatggtgccaaggcagttcatggaccttgggccgtcgtctggagcagcagagcatggagccgaagtgtcatctgaagagaggacaacggttcgttcaggttctcctccttcgcttctagaaagttccaatccccgagagaacggaaagaggttgcttggaagagaagaaagctcagaggaatcagagtctaacgcctggggaaaccctaacaaagtccccaaacataatccatcctctagcaatagcaatggaaacagaaacggaaatgttattgatcagtcggccgcagaagccaccatgcggaaagcccgtgtctcagttcgtgcccgatctgaagctgccatgataagcgatggatgtcaatggagaaagtacggacaaaaaatggctaaaggaaacccgtgtccgcgggcttattatcgttgcacaatggccggtggatgtccagttcgcaagcaagtgcagcgttgcgcagaagacagatctattctcataaccacctacgaaggaaaccacaaccatccactcccaccagccgctacggccatggcctcaacaaccaccgcagctgcaagcatgctcctctcgggctcaatgtcgagtcaagacggtttaatgaacccaacaaacctcctagctcgagctatcttgccttgctcctcaagcatggctacaatctcagcctccgcaccattcccaaccatcacattggacctcaccaattcacccaacggtaacaaccctaatatgaccactaataacccgttgatgcagttcgctcaacggcccggtttcaacccggcagttttgcctcaagtggttggtcaagctatgtacaataaccaacaacagtccaagttttctggtttacagttaccggctcagccactgcagatcgcggccacttcctcggtggccgagagcgttagtgctgccagtgcagcaattgcgtccgatccaaactttgcggcggctctagcggcagcgatcacgtccattatgaacggttccagtcatcaaaataataacaccaataataataatgtggctacgagcaayaatgacagtaggcaataagagttttcattttgatggtcgatttttttttttgggg

SEQ ID NO: 78 - protein, 536 aa, Arabidopsis thaliana
  G184
MFRFPVSLGGSRDEDRHDQITPLDDHRVVVDEVDFFSEKRDRVSRENINDDDDEGNKVLIKMEGSRVEENDRSRDVNIGLNLLTANTGSDESTVDDGLSMDMEDKRAKIENAQLQEELKKMKIENQRLRDMLSQATTNFNALQMQLVAVMRQQEQRNSSQDHLLESKAEGRKRQELQIMVPRQFMDLGPSSGAAEHGAEVSSEERTTVRSGSPPSLLESSNPRENGKRLLGREESSEESESNAWGNPNKVPKHNPSSSNSNGNRNGNVIDQSAAEATMRKARVSVRARSEAAMISDGCQWRKYGQKMAKGNPCPRAYYRCTMAGGCPVRKQVQRCAEDRSILITTYEGNHNHPLPPAATAMASTTTAAASMLLSGSMSSQDGLMNPTNLLARAILPCSSSMATISASAPFPTITLDLTNSPNGNNPNMTTNNPLMQFAQRPGFNPAVLPQVVGQAMYNNQQQSKFSGLQLPAQPLQIAATSSVAESVSAASAAIASDPNFAAALAAAITSIMNGSSHQNNNTNNNNVATSNNDSRQ

SEQ ID NO: 79 - DNA, 1089 bp, Arabidopsis thaliana
  G198
atggcaaggtcaccttgttgcgagaagaacggactcaagaaagggccatggacatctgaagaagaccagaagcttgttgactatatccagaaacatggttatggtaactggagaaccctccccaaaaatgccggtacgtgtttgcaaagatgtggcaaaagttgtaggttaaggtggactaattatctccgaccagatataaaacgaggaagattctcttttgaggaagaagaagccattattcagcttcatagcttcttaggaaacaagtggtctgcgattgcggcgcgtttgccaggaagaacagataatgagatcaagaacttttggaacactcatataagaaagaagctacttagaatggggattgatccagtgactcacagtccacgactcgatctcctcgatatctcatccatcttagcttcatctctatacaattcatcttcacatcacatgaacatgtcaagactcatgatggatactaatcgtcgtcatcagcaacaacatccattggttaaccccgagatactcaagcttgcgacctctatattctctcaaaaccaaaaccaaaaccacaaccaaaatcaaaaccaaaaccaaaacctcgtggtggatcatgagaagcaaacagtttatcatcatcatgatgttaaccaaaccggagtaaaccaataccaaaccgaccaatatttcgagaacgcgattactcaagaactccaatcttccatgccaccattccccaatgaagctcatcagtttaacgacatggatcatcacttcaatggttttggagaacaaaatcttgtttcaacttctactacgtcagtccaagattgctataatccgtcattcaacgattattcaagttcaaattttgtcttagatcattcttattcggatcagagcttcaacttcgcaaattcggtcttaaacacgccatcctcgagcccgagcccgactacgttaaactcgagttacatcaatagtagcagttgcagcactgaggatgaaatagaaagctattgcagtaatctcatgaagtttgatattcccgatttcttggacgttaatggttttattatataa

SEQ ID NO: 80 - protein, 362 aa, Arabidopsis thaliana
  G198
MARSPCCEKNGLKKGPWTSEEDQKLVDYIQKHGYGNWRTLPKNAGTCLQRCGKSCRLRWTNYLRPDIKRGRFSFEEEEAIIQLHSFLGNKWSAIAARLPGRTDNEIKNFWNTHIRKKLLRMGIDPVTHSPRLDLLDISSILASSLYNSSSHHMNMSRLMMDTNRRHQQQHPLVNPEILKLATSIFSQNQNQNHNQNQNQNQNLVVDHEKQTVYHHHDVNQTGVNQYQTDQYFENAITQELQSSMPPFPNEAHQFNDMDHHFNGFGEQNLVSTSTTSVQDCYNPSFNDYSSSNFVLDHSYSDQSFNFANSVLNTPSSSPSPTTLNSSYINSSSCSTEDEIESYCSNLMKFDIPDFLDVNGFII

SEQ ID NO: 81 - DNA, 1650 bp, Arabidopsis thaliana
  G20
ctctcactctctttttctctctctttacttctactgtatcaagaagctccgttttgcttagccatagtgcgtctagggtttggttggtgggaagaaggttccgatcatggcgtcggtgtcgtcgtcggatcaaggacctaagacagaagcaggatgtagcggcggaggaggaggagagagctcggagacagtggcggcgagtgatcagatgttgttgtatagaggttttaagaaggcgaagaaggagagaggttgtacagctaaggagcgtattagtaaaatgcctccgtgcactgctgggaaaaggagttccatataccggggagtcaccagacatagatggacaggtcgttatgaagctcacctttgggataagagtacctggaaccaaaaccagaacaagaagggaaaacaagtttatctaggagcatatgatgatgaagaggctgctgctagagcttacgaccttgctgccttaaaatattggggtcctgggacacttataaattttccggtgactgattataccagggatttagaagaaatgcaaaatctctcaagggaagaataccttgcatctttacgtagatatccctttggcagaaaaagcagcggtttctctaggggaatagcgaaatatcgtggacttcaaagccgatgggacgcatcagccagtcgtatgcctggacctgaatacttcagtaacattcattacggggcaggtgatgatcgtggaacagaaggtgactttctaggtagcttttgtctggaaagaaagattgatctaacaggatacataaagtggtggggagccaacaagaaccgtcaaccagaatcttcatcaaaagcatcagaggatgcaaacgtcgaagatgctggtactgagcttaaaacactggaacacacatcccatgcaacagaaccatacaaggcgccaaaccttggcgtcctttgtggaactcagagaaaagaaaaagaaatatcatcaccatcaagctcttctgctttaagcatcttgtctcagtcgcctgccttcaagagcctagaggagaaagtgttgaagatccaagaaagctgcaataatgaaaacgatgagaatgcaaaccgtaacatcatcaatatggagaagaataacggcaaggcaatagagaaaccagttgtgagtcatggagttgctttaggcggtgctgctgctttgtctcttcagaaaagcatgtacccacttacctctctcttaacggctccattgctcaccaactacaatacattggatcctcttgcagaccctattctctggacaccatttcttccttcaggatcctctcttacttcagaggtgacaaagacagagaccagctgttccacgtacagctacctcccacaagagaaatgagccgttccctttagactttatgtatgtcagattctccttttttgagatgaattcgtcgacttgacatctctttgtctcttttatggagaaaaagttgggaaaagtgtgacaatggtctgaagcaggaatgtacaggttttgttagtggttgtgtttttttttttccagtgtggaatatagaatcatgatattttgtgtaaaacagaaaaaagttatcattatagtatagaagtttgctcttaaaaaaaaaaaaaaaa

SEQ ID NO: 82 - protein, 428 aa, Arabidopsis thaliana
  G20
MASVSSSDQGPKTEAGCSGGGGGESSETVAASDQMLLYRGFKKAKKERGCTAKERISKMPPCTAGKRSSIYRGVTRHRWTGRYEAHLWDKSTWNQNQNKKGKQVYLGAYDDEEAAARAYDLAALKYWGPGTLINFPVTDYTRDLEEMQNLSREEYLASLRRYPFGRKSSGFSRGIAKYRGLQSRWDASASRMPGPEYFSNIHYGAGDDRGTEGDFLGSFCLERKIDLTGYIKWWGANKNRQPESSSKASEDANVEDAGTELKTLEHTSHATEPYKAPNLGVLCGTQRKEKEISSPSSSSALSILSQSPAFKSLEEKVLKIQESCNNENDENANRNIINMEKNNGKAIEKPVVSHGVALGGAAALSLQKSMYPLTSLLTAPLLTNYNTLDPLADPILWTPFLPSGSSLTSEVTKTETSCSTYSYLPQEK

SEQ ID NO: 83 - DNA, 1888 bp, Arabidopsis thaliana
  G571
tagccgacctctcttctctcttctgaaaaaaacaccaaaggagctttaaatgctccgttacataatctctatctctttccaagaatatagagaaaggaaaataatatacaagaattaaaagaaggtatatcatcatctctctagctagtgatcaaagcaccgtcatcatcatcatatatcatcagcttgcctcagaggagaagaccaacataagagagatcgaagatcaaaatctatctctcttcatcatcttctgctgttactatcatatcacacgctctctcaaacatcatcctatatatagacttctcttcatcatcatcaaatgcaaggtcatcaccagaatcatcatcaacacttatcatcatcctccgccacgtcttcccatggaaacttcatgaacaaagatgggtatgatattggagagatagacccatcactcttcctctatcttgatggacaaggacatcatgatcctccatcaactgctccttctcctttacatcatcatcacacaactcagaatttggcgatgagacctccaacatcgacgctcaacatctttccatctcagcctatgcacatagagccacctccttcttctacacacaataccgataatacaagattagttccggctgctcaacctagtggttccactcgaccagcttctgacccgtccatggacttgaccaatcattctcagtttcatcaacctcctcaaggttctaaatccatcaagaaggaagggaaccgcaagggtcttgcctcatcggaccatgacatacctaaatcgtcagaccctaaaacattgagaagactagcacaaaacagagaagcagcaagaaaaagcagattacgtaaaaaggcttatgttcagcaactcgagtcatgtaggatcaaactgacccaactagaacaagagattcaacgggccagatcccaaggcgtattctttggagggtctcttataggaggagatcaacagcaaggtggactacccattggccctggcaacatcagctctgaagcagcggtgttcgatatggaatatgcgaggtggctggaggagcagcagaggctattaaacgaactaagggtggcaacacaagaacacttgtccgagaacgagcttaggatgtttgtggacacatgtttagctcattatgaccatttgattaacctcaaggctatggtcgctaagaccgatgtcttccacctcatttctggagcatggaaaactccagctgaacgttgcttcttgtggatgggtggtttccgtccatcggagatcattaaggtgattgtgaaccagatagaaccattgacggagcaacagatagttgggatatgtgggctgcaacagtccacacaagaggccgaggaggctctctcgcaaggcctcgaggcgttgaatcaatcactttccgatagcattgtctctgactccctcccgcctgcctccgcaccacttcctcctcatctatccaatttcatgtcacacatgtccttagctctcaacaagctctctgctctcgagggcttcgttctccaggcggataatttgaggcaccaaacgatccataggctgaaccaattgttgacgacccgtcaagaagcacggtgtcttctagccgttgcggagtacttccaccgtcttcaagctctaagttctctctggctagcccgtcctcggcaagatggataatactaaaacaactgatgaaggaaaccaaaaacaaaaacaagagaataggttgattagttagccgccagcttgacctctttatcatatatatcgtctctctactcaaatacagtgcaattagggaaaattgtttggcttctttttggtatatgattcttactattatgtttttaatcaaga

SEQ ID NO: 84 - protein, 460 aa, Arabidopsis thaliana
  G571
MQGHHQNHHQHLSSSSATSSHGNFMNKDGYDIGEIDPSLFLYLDGQGHHDPPSTAPSPLHHHHTTQNLAMRPPTSTLNIFPSQPMHIEPPPSSTHNTDNTRLVPAAQPSGSTRPASDPSMDLTNHSQFHQPPQGSKSIKKEGNRKGLASSDHDIPKSSDPKTLRRLAQNREAARKSRLRKKAYVQQLESCRIKLTQLEQEIQRARSQGVFFGGSLIGGDQQQGGLPIGPGNISSEAAVFDMEYARWLEEQQRLLNELRVATQEHLSENELRMFVDTCLAHYDHLINLKAMVAKTDVFHLISGAWKTPAERCFLWMGGFRPSEIIKVIVNQIEPLTEQQIVGICGLQQSTQEAEEALSQGLEALNQSLSDSIVSDSLPPASAPLPPHLSNFMSHMSLALNKLSALEGFVLQADNLRHQTIHRLNQLLTTRQEARCLLAVAEYFHRLQALSSLWLARPRQDG

SEQ ID NO: 85 - DNA, 1033 bp, Arabidopsis thaliana
  G221
ctctcttattctctcactcttttttttttatattcctctctctctaaatctataaaatatatttaaaaacttgatcgtatataataaagtaaataaagaataataacaaaaaaaatggagaaaagaggaggaggaagtagtggaggttcgggatcatcagcagaagcagaagtgagaaaaggaccatggacgatggaagaagatcttattcttatcaactatatcgccaaccacggcgatggtgtttggaattctctcgccaaatctgcaggtctaaaacgaaccgggaaaagttgccggctccggtggctgaactatctccgccccgacgtacgacggggaaacatcactccagaagagcaacttatcatcatggaacttcatgctaagtggggaaacaggtggtcgaaaatcgccaaacatcttccaggaagaacggacaacgagatcaaaaatttctgtaggacaagaattcaaaaatacatcaagcaatcggatgtaacaacaacatcgtccgttggatctcatcatagctcagagatcaacgatcaagctgcaagcacgtcgagccataatgtcttttgtacacaagatcaagcgatggagacttattctcctacaccgacatcatatcaacataccaatatggaattcaactatggtaactattcggccgcggcagtgacggcaaccgtggattatccagtaccgatgaccgttgatgatcaaaccggtgaaaactattggggcatggatgatatttggtcatcaatgcatttattgaatggtaattgattgatcggtggacaaaacatggaatattaattgagtattatatatgatttttaggagtactattattagtacgtgacatgtatatgtttttgcctcgttgtagaggtttggggttataattaatatataatgttatctaatatgcaaccttgatacatatttggatctttattgaacccatgttatacataaataaaattgttgaaggggtcataaaaaaaaaaaaaaaaaaaaaaaa

SEQ ID NO: 86 - protein, 226 aa, Arabidopsis thaliana
  G221
MEKRGGGSSGGSGSSAEAEVRKGPWTMEEDLILINYIANHGDGVWNSLAKSAGLKRTGKSCRLRWLNYLRPDVRRGNITPEEQLIIMELHAKWGNRWSKIAKHLPGRTDNEIKNFCRTRIQKYIKQSDVTTTSSVGSHHSSEINDQAASTSSHNVFCTQDQAMETYSPTPTSYQHTNMEFNYGNYSAAAVTATVDYPVPMTVDDQTGENYWGMDDIWSSMHLLNGN

SEQ ID NO: 87 - DNA, 1662 bp, Arabidopsis thaliana
  G570
aagcttgatcctcctagttgtacgaaagcttgagtaatggggtctagattaaacttcaagagctttgttgatggtgtgagtgagcagcagccaacggtggggactagtcttccattgactaggcagaactctgtgttctcgttaacctttgatgagtttcagaactcatggggtggtggaattgggaaagattttgggtctatgaacatggatgagctcttgaagaacatttggactgcagaggaaagtcattcaatgatgggaaacaataccagttacaccaacatcagcaatggtaatagtggaaacactgttattaacggcggtggtaacaacattggtgggttagctgttggtgtgggaggagaaagtggtggttttttcactggtgggagtttgcagagacaaggttcacttaccttgcctcggacgattagtcagaaaagggttgatgatgtctggaaggagctgatgaaggaggatgacattggaaatggtgttgttaatggtgggacaagcggaattccgcagaggcaacaaacgctgggagagatgactttggaggagtttttggtcagggctggtgtggttagggaagaacctcaaccggtggagagtgtaactaacttcaatggcggattctatggatttggcagtaatggaggtcttgggacagctagtaatgggtttgttgcaaaccaacctcaagatttgtcaggaaatggagtagcggtgagacaggatctgctgactgctcaaactcagccactacagatgcagcagccacagatggtgcagcagccacagatggtgcagcagccgcaacaactgatacagacgcaggagaggccttttcccaaacagaccactatagcattttccaacactgttgatgtggttaaccgttctcaacctgcaacacagtgccaggaagtgaagccttcaatacttggaattcataaccatcctatgaacaacaatctactgcaagctgtcgattttaaaacaggagtaacggttgcagcagtatctcctggaagccagatgtcacctgatctgactccaaagagcgccctggatgcatctttgtcccctgttccttacatgtttgggcgagtgagaaaaacaggtgcagttctggagaaagtgattgagagaaggcaaaaaaggatgataaagaatagggaatcagctgcaagatcccgcgctcgcaagcaagcttatacgatggaactggaagcagaaattgcgcaactcaaagaattgaatgaagagttgcagaagaaacaagttgaaatcatggaaaagcagaaaaatcagcttctggagcctctgcgccagccatggggaatgggatgcaaaaggcaatgcttgcgaaggacattgacgggtccctggtagagcttataatggcgtctaaggaacccaacaaagcgccgaagttatagaacaactcagaagatagaaagctagctttgtacgtagtttaggcaggttctgtgggtgattgtaaatcttgaagtgtggcggatttgacagagatagataaacacatatctgttctattttcctaaatcttttggttttatcttcctgatgtaatggatctttatcatttgtcttgaacatctttgtgacttaaccagagtgaatttatcttgt

SEQ ID NO: 88 - protein, 454 aa, Arabidopsis thaliana
  G570
MGSRLNFKSFVDGVSEQQPTVGTSLPLTRQNSVFSLTFDEFQNSWGGGIGKDFGSMNMDELLKNIWTAEESHSMMGNNTSYTNISNGNSGNTVINGGGNNIGGLAVGVGGESGGFFTGGSLQRQGSLTLPRTISQKRVDDVWKELMKEDDIGNGVVNGGTSGIPQRQQTLGEMTLEEFLVRAGVVREEPQPVESVTNFNGGFYGFGSNGGLGTASNGFVANQPQDLSGNGVAVRQDLLTAQTQPLQMQQPQMVQQPQMVQQPQQLIQTQERPFPKQTTIAFSNTVDVVNRSQPATQCQEVKPSILGIHNHPMNNNLLQAVDFKTGVTVAAVSPGSQMSPDLTPKSALDASLSPVPYMFGRVRKTGAVLEKVIERRQKRMIKNRESAARSRARKQAYTMELEAEIAQLKELNEELQKKQVEIMEKQKNQLLEPLRQPWGMGCKRQCLRRTLTGPW

SEQ ID NO: 89 - DNA, 751 bp, Arabidopsis thaliana
  G25
aggactcggttcaaaaacgaaaacgcaaatacgtcttgttctagtttggagttggaagcgtaaaaaacaaaaaacaaaaatgtgtgggggagctatcatttctgatttcatctggtcgaaatctgagtcagaaccgagtcaactcggctctgttagcagcaggaagaagcgtaaacccgtctcagtgagtgaagaaagagatgggaaacgagagaggaagaatctgtacagagggataaggcagaggccatggggcaaatgggcagcggagattcgtgacccgagcaaaggtgtacgtgtctggcttggcacattcaaaaccgccgacgaagctgctcgagcctacgacgttgctgccatcaaaatccgtggccggaaagccaaactgaatttcccaaacactcaagtagaagaagaagccgatactaaaccaggggggaatcaaaatgagctgatttcggaaaaccaagtagagagcttatcggaggacctgatggcattggaggattacatgagattctatcagattccggttgccgacgaccaatcggcgaccgatattggaaatttatggagctatcaagactccaattaaatctcttatttcccggccggtttgctcactcattaatatgctgctaatttacttgttttttacttaacaatcaagtctaatttgtttccatcaatatttcagataagagtaaagcttcaattgtctttaaaaaaaaaaaaaaaaaaaaaaaaaaaa

SEQ ID NO: 90 - protein, 171 aa, Arabidopsis thaliana
  G25
MCGGAIISDFIWSKSESEPSQLGSVSSRKKRKPVSVSEERDGKRERKNLYRGIRQRPWGKWAAEIRDPSKGVRVWLGTFKTADEAARAYDVAAIKIRGRKAKLNFPNTQVEEEADTKPGGNQNELISENQVESLSEDLMALEDYMRFYQIPVADDQSATDIGNLWSYQDSN

SEQ ID NO: 91 - DNA, 998 bp, Arabidopsis thaliana
  G258
agtgaccaccctgctggttaatcaacaccaagagaccttgtaatatataagttaggaagatgagagagaagtgggaaatgaaaagagatgaaatgggacatcgatgttgtggaaaacacaaagtgaagagaggtctttggtctccagaggaagacgagaagcttcttcgttatatcaccactcatggtcatcctagttggagttccgttccaaagcttgccgggttgcagagatgtgggaagagttgcagattaaggtggataaactatctaaggcctgatctgaggagaggttcgtttaatgaggaagaagagcagattatcatcgacgtacatcgtattcttggtaacaaatgggctcagattgctaagcacttacctggacgcactgataatgaagtcaagaacttttggaactcatgcattaagaagaaacttctttctcaaggcttagatccttctacacataatcttatgccttcacacaaaagatcttcttcttcaaacaataataatatccccaagccaaacaaaacgacgtccatcatgaagaaccctactgatcttgatcaatcaaccactgctttttcaatcacaaacatcaatccacccacttccactaaaccaaacaaacttaaatctcctaaccagactacaatcccatctcaaaccgtgatccctatcaatgataacatgtcaagtactcaaaccatgatccctatcaatgatcccatgtcaagtcttttagatgatgagaatatgattcctcactggtcagatgttgatggaatggcgatccacgaagctccgatgttgcctagtgataaggcagtagtgggagtggatgatgatgatctcaacatggacattttgtttaacactccttcttcttctgcttttgatcctgattttgcttccattttctcctctgcaatgtctatcgatttcaatcccatggatgatcttggcagctggaccttttagcttttactctacagc

SEQ ID NO: 92 - protein, 307 aa, Arabidopsis thaliana
  G258
MREKWEMKRDEMGHRCCGKHKVKRGLWSPEEDEKLLRYITTHGHPSWSSVPKLAGLQRCGKSCRLRWINYLRPDLRRGSFNEEEEQIIIDVHRILGNKWAQIAKHLPGRTDNEVKNFWNSCIKKKLLSQGLDPSTHNLMPSHKRSSSSNNNNIPKPNKTTSIMKNPTDLDQSTTAFSITNINPPTSTKPNKLKSPNQTTIPSQTVIPINDNMSSTQTMIPINDPMSSLLDDENMIPHWSDVDGMAIHEAPMLPSDKAVVGVDDDDLNMDILFNTPSSSAFDPDFASIFSSAMSIDFNPMDDLGSWTF

SEQ ID NO: 93 - DNA, 1440 bp, Arabidopsis thaliana
  G38
gaggaaaactcgaaaaagctacacacaagaagaagaagaaaagatacgagcaagaagactaaacacgaaagcgatttatcaactcgaaggaagagactttgattttcaaatttcgtcccctatagattgtgttgtttctgggaaggagatggcagtttatgatcagagtggagatagaaacagaacacaaattgatacatcgaggaaaaggaaatctagaagtagaggtgacggtactactgtggctgagagattaaagagatggaaagagtataacgagaccgtagaagaagtttctaccaagaagaggaaagtacctgcgaaagggtcgaagaagggttgtatgaaaggtaaaggaggaccagagaatagccgatgtagtttcagaggagttaggcaaaggatttggggtaaatgggttgctgagatcagagagcctaatcgaggtagcaggctttggcttggtactttccctactgctcaagaagctgcttctgcttatgatgaggctgctaaagctatgtatggtcctttggctcgtcttaatttccctcggtctgatgcgtctgaggttacgagtacctcaagtcagtctgaggtgtgtactgttgagactcctggttgtgttcatgtgaaaacagaggatccagattgtgaatctaaacccttctccggtggagtggagccgatgtattgtctggagaatggtgcggaagagatgaagagaggtgttaaagcggataagcattggctgagcgagtttgaacataactattggagtgatattctgaaagagaaagagaaacagaaggagcaagggattgtagaaacctgtcagcaacaacagcaggattcgctatctgttgcagactatggttggcccaatgatgtggatcagagtcacttggattcttcagacatgtttgatgtcgatgagcttctacgtgacctaaatggcgacgatgtgtttgcaggcttaaatcaggaccggtacccggggaacagtgttgccaacggttcatacaggcccgagagtcaacaaagtggttttgatccgctacaaagcctcaactacggaatacctccgtttcagctcgagggaaaggatggtaatggattcttcgacgacttgagttacttggatctggagaactaaacaaaacaatatgaagctttttggatttgatatttgccttaatcccacaacgactgttgattctctatccgagttttagtgatatagagaactacagaacacgttttttcttgttataaaggtgaactgtatatatcgaaacagtgatatgacaatagagaagacaactatagtttgttagtctgcttctcttaagttgttctttagatatgttttatgttttgtaacaacaggaatgaataatacacacttgtgaagcttttaaaaaaaaaaaaaaaaaaaaa

SEQ ID NO: 94 - protein, 335 aa, Arabidopsis thaliana
  G38
MAVYDQSGDRNRTQIDTSRKRKSRSRGDGTTVAERLKRWKEYNETVEEVSTKKRKVPAKGSKKGCMKGKGGPENSRCSFRGVRQRIWGKWVAEIREPNRGSRLWLGTFPTAQEAASAYDEAAKAMYGPLARLNFPRSDASEVTSTSSQSEVCTVETPGCVHVKTEDPDCESKPFSGGVEPMYCLENGAEEMKRGVKADKHWLSEFEHNYWSDILKEKEKQKEQGIVETCQQQQQDSLSVADYGWPNDVDQSHLDSSDMFDVDELLRDLNGDDVFAGLNQDRYPGNSVANGSYRPESQQSGFDPLQSLNYGIPPFQLEGKDGNGFFDDLSYLDLEN

SEQ ID NO: 95 - DNA, 1986 bp, Arabidopsis thaliana
  G389
atggaaacgaaagacaagaaagaaaaaggacatatggttttaaattccgacaacgttttcggttcggtgtcttcgtctccgacaacaacaatccaaaaccctaattactttacctccttcgagaaccctaatttcccttacatttttccgaaagaagagtatgaggtgatgagcaagatcgaaagcgggtcgggtaagtcaaccgggtcgggtcatgacccagtggagaatactgcaatcgaacaagagcctcctgcggcgaagaagaaacgttaccatagacatactgctagtcagattcaacaaatggaagcattgtttaaggaaaatgctcatccggataccaaaacaagattgagattaagtaaaaaacttggtctctcgccgatacaagtcaagttttggtttcaaaacaaacgtacccaaatcaaggcacaacaaagtagaagcgataatgcaaagctaaaagcagagaatgagactctaaagacagagagtcaaaatattcagtccaattttcaatgccttttttgctctacttgtggccacaatctccgcctcgaaaacgctcgcctccgccaagagcttgatcgtttacgcagcattgtttccatgaggaatccttctccttcacaagagattaccccggagactaataagaacaacaacgataatatgttgattgcggaagaagaaaaggcgattgatatggaacttgcagtttcttgtgctcgagaattagcaaagatgtgtgacataaatgagcccttgtggaataagaagagactagacaatgagagtgtgtgtctgaatgaggaagagtataagaagatgttcttatggcctctaatgaatgatgatgatcgttttcgcagagaagcttcaagagctaatgcagtcatcatgttgaactgcataaccctcgtcaaagcattccttgatgctgataaatggtcggaaatgttcttccccatagtctcaagcgccaaaacggctcagatcatttcttctggagcttctggaccaagtggtactcttcttctgcaaaatgctgaagaaggaaaatggatggttgtagatttcccgatcgataggatcaaaccagcatcagctactactactgatcaataccggagaaaaccttctggttgcatcattcaagcaatgcgtaacggatactctcaggtcacatgggtagagcacgtagaagtagaagagaagcacgtgcaggacgaggttgttagagagtttgtggagagcggtgtggcctttggcgctgaacggtggctatctgtgttgaagagacaatgtgaaaggatggctagtctcatggctacaaacatcactgaccttggaggtatatatataacttgtttcacaatgataccttctgtagaagcgaggaagaacttgatgaagctgtcacagagaatggtgaaaactttctgtctgaacataatcaattcacacggacaagcaccaacaaaggacacagttaaaatcgtaagcaggaaagtttgcggtggtcttgttccttgtgccgtgtcagtcacgcttctcccttattctcatcaacaagtctttgatcttctccgcgacaatcaacgtctctctcaggtggagagcaattcatcgcataatgtagagttgatgcttcaagaaacatgcaccgataactccgggagtctcctggtgtactctaccgtcgaccccgtcgctgttcagctcgccatgaacggcgaagatccttctgaaataccacttttgcccgttggattctctgttgttccagtgaatccatccgatggtgtcgagggcagttccgtcagttcgccttcgtgcttgctcactgttgcaatacaggtcttggggagcaacgtcaccaccgaaagactcgatctttccaccgtctccgtcatcaatcaccgtatttgcgccactgtcaaccgtatcacttccgcgctcgtcaacgacgtcggtaattaa

SEQ ID NO: 96 - protein, 661 aa, Arabidopsis thaliana
  G389
METKDKKEKGHMVLNSDNVFGSVSSSPTTTIQNPNYFTSFENPNFPYIFPKEEYEVMSKIESGSGKSTGSGHDPVENTAIEQEPPAAKKKRYHRHTASQIQQMEALFKENAHPDTKTRLRLSKKLGLSPIQVKFWFQNKRTQIKAQQSRSDNAKLKAENETLKTESQNIQSNFQCLFCSTCGHNLRLENARLRQELDRLRSIVSMRNPSPSQEITPETNKNNNDNMLIAEEEKAIDMELAVSCARELAKMCDINEPLWNKKRLDNESVCLNEEEYKKMFLWPLMNDDDRFRREASRANAVIMLNCITLVKAFLDADKWSEMFFPIVSSAKTAQIISSGASGPSGTLLLQNAEEGKWMVVDFPIDRIKPASATTTDQYRRKPSGCIIQAMRNGYSQVTWVEHVEVEEKHVQDEVVREFVESGVAFGAERWLSVLKRQCERMASLMATNITDLGGIYITCFTMIPSVEARKNLMKLSQRMVKTFCLNIINSHGQAPTKDTVKIVSRKVCGGLVPCAVSVTLLPYSHQQVFDLLRDNQRLSQVESNSSHNVELMLQETCTDNSGSLLVYSTVDPVAVQLAMNGEDPSEIPLLPVGFSVVPVNPSDGVEGSSVSSPSCLLTVAIQVLGSNVTTERLDLSTVSVINHRICATVNRITSALVNDVGN

SEQ ID NO: 97 - DNA, 937 bp, Arabidopsis thaliana
  G395
gcacaatcccactatccttcgcaagacccttcctctatataaggaagttcatttcatttggagaggacacgctgacaagctgactctagcagatctggtaccgtcgacaaggaggaagaatgcccttaggagcagctacggttgtggaggaggaagaggaggaggaggaagcggtgcctagtatgtcagtatcgccgccggatagtgtaacgtcgtcgtttcaattggactttgggattaaaagttatggttatgagagaagaagcaataagagagatattgatgatgaagtggagagatcagcctcaagagccagcaacgaagacaacgatgacgagaatggatccactaggaagaaacttagactctccaaagaccaatctgcttttcttgaagacagcttcaaagaacacagtacccttaatcctaaacagaagattgcattggcgaagcagttgaatcttcgtcctcgtcaggttgaagtctggtttcaaaacagacgagccaggacaaagctgaagcaaacggaagtggactgtgaatacctaaagagatgctgtgagtcactaaccgaagaaaaccggaggcttcaaaaagaggttaaagaattgagaaccttgaagacttccacacccttttacatgcaacttccggccactactctcactatgtgcccttcttgtgaacgtgttgccacttcagcagcacagccctccacgtcagctgcccacaacctctgtttgtccacgtcatcattgattccggttaagcctcggccggccaaacaagtttcatgaaagcacctgcgaaatacagtttgagcaaacgggcggccgctctagacaggcctcgtaccggatcctctagctagagctttcgttcgtatcatcggtttcgacaacgttcgtcaagttcaatgacatcagtttgattgcgc

SEQ ID NO: 98 - protein, 225 aa, Arabidopsis thaliana
  G395
MPLGAATVVEEEEEEEEAVPSMSVSPPDSVTSSFQLDFGIKSYGYERRSNKRDIDDEVERSASRASNEDNDDENGSTRKKLRLSKDQSAFLEDSFKEHSTLNPKQKIALAKQLNLRPRQVEVWFQNRRARTKLKQTEVDCEYLKRCCESLTEENRRLQKEVKELRTLKTSTPFYMQLPATTLTMCPSCERVATSAAQPSTSAAHNLCLSTSSLIPVKPRPAKQVS

SEQ ID NO: 99 - DNA, 2332 bp, Arabidopsis thaliana
  G471
gcaattcttagtttctcttctatcccctttctgggttttgtttgaaccctagctcgctttggatccagtggttttaagttaaaggtaaatttatcgaaagtaagtagattcctaatggcagcttccaatcattcatctggtaaacctggaggagttttaagtgatgctttatgtagggagctctggcatgcctgtgctggacctcttgtaaccctacctcgtgaaggggaacgagtttattatttccctgaaggccacatggagcagctcgaggcatcaatgcaccaaggtttggagcaacagatgccttccttcaacctcccatctaagatcctctgtaaagttatcaacatccagcgcagggcagagcccgagactgacgaagtatatgcgcaaataaccttattgccagaactggatcaaagcgaacccactagcccagatgcccctgttcaagaacctgaaaagtgcaccgtacattcattttgcaagacactaactgcttcagacacaagcacacatggtggcttctcggtgctacggagacatgcagatgattgtctcccacccttggatatgtcccaacaaccaccgtggcaagaattggttgcaactgatttgcacaatagtgaatggcattttaggcacattttccgaggccaaccaaggcgtcatttgctaacaactggatggagtgtttttgttagctcgaagaaactagtggctggtgatgctttcatattcttaaggggtgagaatgaagagctccgagtaggtgttaggcggcacatgagacaacagactaatatcccgtcatctgtcatttcaagtcatagcatgcatattggggtccttgcaacagcagctcatgccattacaacaggaacaatcttttctgtcttctacaagccaaggacaagtaggtcagagtttattgtgagcgtcaataggtatctcgaagctaagacccagaagctgtctgtaggcatgcgtttcaagatgagattcgagggggaagaagctcccgagaaaaggttcagtggcacaatagttggtgttcaggaaaataagtcttcggtctggcatgattctgaatggagatcgctaaaggttcaatgggacgaaccctcatctgtatttcgtcctgaaagagtttcaccttgggaacttgagcccctagttgcaaatagtactccgtcttcacaacctcagcctccgcaaaggaacaaacgaccaagacctcctggtttaccttcaccagccactggtccatctggtcctgttactccagatggtgtgtggaaatccccggcagacactccttcctcagtgccattattctctcctcctgccaaagctgctacgtttggtcatggtgggaacaaatcatttggagtatctattggatcagccttttggcccaccaatgcagatagtgcagctgaatcctttgcttcagcgtttaacaatgaatctactgaaaagaaacaaactaatggaaatgtctgtaggctttttgggtttgagctagttgaaaatgttaatgtggatgaatgtttctctgctgcctctgtgtctggtgctgtcgctgtagatcaacctgtcccatccaacgagtttgactctggccagcaatctgagccattaaacatcaaccaatctgatattccttcggggagtggtgaccctgagaaatcctctttgaggtctcctcaagaatcacaaagtagacagatacgtagctgcacaaaggtgcacatgcaaggcagtgcagtaggcagagctattgatttgacaaggtcagagtgttatgaagatctgttcaagaagctggaagagatgtttgatatcaagggtgaactcttagaatctaccaaaaaatggcaagtcgtttacaccgatgatgaagatgacatgatgatggttggtgatgatccatggaatgagttctgtggaatggtgaggaagatattcatctacacacctgaggaagtgaagaaactttcaccgaagaacaaactcgcagtcaatgcaaggatgcagctcaaagctgatgcagaggaaaatgggaatacagagggcagatcatcatctatggcgggatcaagatgagtatatcactgtgttatgttttaaatgtacttgccacgtaggaaatatgaaagcagaagcaagagatcgttagacaatatgaaagttgagatgtctgtgtatagcaatgaagttttatgtcttcaagtcttatgaattcacttagatgcaatgtgttttgaggagttgtgtagcttttgtacgggaaatatggaaattaagtttcacgtcttgttctacc

SEQ ID NO: 100 - protein, 665 aa, Arabidopsis thaliana
  G471
MAASNHSSGKPGGVLSDALCRELWHACAGPLVTLPREGERVYYFPEGHMEQLEASMHQGLEQQMPSFNLPSKILCKVINIQRRAEPETDEVYAQITLLPELDQSEPTSPDAPVQEPEKCTVHSFCKTLTASDTSTHGGFSVLRRHADDCLPPLDMSQQPPWQELVATDLHNSEWHFRHIFRGQPRRHLLTTGWSVFVSSKKLVAGDAFIFLRGENEELRVGVRRHMRQQTNIPSSVISSHSMHIGVLATAAHAITTGTIFSVFYKPRTSRSEFIVSVNRYLEAKTQKLSVGMRFKMRFEGEEAPEKRFSGTIVGVQENKSSVWHDSEWRSLKVQWDEPSSVFRPERVSPWELEPLVANSTPSSQPQPPQRNKRPRPPGLPSPATGPSGPVTPDGVWKSPADTPSSVPLFSPPAKAATFGHGGNKSFGVSIGSAFWPTNADSAAESFASAFNNESTEKKQTNGNVCRLFGFELVENVNVDECFSAASVSGAVAVDQPVPSNEFDSGQQSEPLNINQSDIPSGSGDPEKSSLRSPQESQSRQIRSCTKVHMQGSAVGRAIDLTRSECYEDLFKKLEEMFDIKGELLESTKKWQVVYTDDEDDMMMVGDDPWNEFCGMVRKIFIYTPEEVKKLSPKNKLAVNARMQLKADAEENGNTEGRSSSMAGSR

SEQ ID NO: 101 - DNA, 420 bp, Arabidopsis thaliana
  G486
atgacagacgaagatagattgttgccaatagccaatgtagggagacttatgaagcaaatcctaccatcaaatgcaaagatctcaaaagaagcaaaacaaacagttcaagaatgtgcaacagagttcataagctttgttacatgcgaagcatcagagaagtgccacagggagaatcggaagacggtgaatggagacgacatctggtgggctctcagcactctcggcctcgataactatgctgacgccgtgggtaggcatcttcacaagtaccgtgaagccgagagagaaagaactgagcacaacaaaggtagcaatgatagtgggaatgagaaagaaaccaacactagaagtgatgtacagaaccaatcgacaaaatttattagagttgttgagaagggaagcagctcctcggcccgttga

SEQ ID NO: 102 - protein, 139 aa, Arabidopsis thaliana
  G486
MTDEDRLLPIANVGRLMKQILPSNAKISKEAKQTVQECATEFISFVTCEASEKCHRENRKTVNGDDIWWALSTLGLDNYADAVGRHLHKYREAERERTEHNKGSNDSGNEKETNTRSDVQNQSTKFIRVVEKGSSSSAR

SEQ ID NO: 103 - DNA, 913 bp, Arabidopsis thaliana
  G511
gtttcttgttgttaaaaatatcgtacaaaaatggccgatgaggtcacaatcgggtttcgcttctatcccacggaagaagaactggtttcgttctacctacgaaaccagctcgaaggaaggagtgatgactcaatgcatcgtgtcattcccgtacttgacgtctttgaggtcgagcctagtcatcttccaaatgttgctggagtgagatgtcgaggagacgctgagcaatggttcttcttcgtgccacgacaagaacgcgaagcaagaggaggcagaccgagtagaactactggttcaggatactggaaagcaactggatcacctggtccagtcttttccaaagacaacaaaatgattggagcaaagaaaactatggttttctacactggaaaagcacccacaggaagaaaaactaaatggaaaatgaatgagtaccacgccgttgacgaaacagtcaacgcttccacaatccctaagctgagacgtgagttcagtttatgtcgagtctacataacaacaggaagctccagagcttttgatagacgtcctgagggagttttgcagacagagagaatgctaacaagtgatgttgcagtagctgagacatcgttccgtgtggaaagctcactggaaacttcgatttcaggaggagaacatattgatgtctctatgaacacagagtttgttgatggactatcagaaccgatgtgggactgggaacagctgacttggccttgaagctatatagattttataatcaagcaaatttaaacttgtttcaattgcttattgttagtttgaattttatgacccgaaagattctttttctttctttaccttgtaacgtgagaatttgagaaataataaatgacctagacagtgacatttgaaaaaaaaaaaaaaaaaaaaaaaa

SEQ ID NO: 104 - protein, 235 aa, Arabidopsis thaliana
  G511
MADEVTIGFRFYPTEEELVSFYLRNQLEGRSDDSMHRVIPVLDVFEVEPSHLPNVAGVRCRGDAEQWFFFVPRQEREARGGRPSRTTGSGYWKATGSPGPVFSKDNKMIGAKKTMVFYTGKAPTGRKTKWKMNEYHAVDETVNASTIPKLRREFSLCRVYITTGSSRAFDRRPEGVLQTERMLTSDVAVAETSFRVESSLETSISGGEHIDVSMNTEFVDGLSEPMWDWEQLTWP

SEQ ID NO: 105 - DNA, 1285 bp, Arabidopsis thaliana
  G512
aaagctccctgagcaagtgagaagagaccacactgagaagaaaaatccttcaggttatcgaaaattcccggattttactctttgggcggtggccgatttcttacgcgctctttttgtggcgactgttgtatacgcgcgagccttcatggacacgaaggcggttggagtttctaaggatacggcggcgtcgatggaagcgtcgacggtgtttcctgggtttaaattctcgccgacggatgtggagttgatttcgtattacctgaagcggaagatggatggcttggagaggtccgttgaggttataccggaccttgagatttacaatttcgagccttgggatttacccgataagtcgattgtgaaatctgatagcgagtggttcttcttctgtgcgcgtgggaaaaagtatccacatggttcacagaacaggagagcaacgaagatgggatactggaaagcaactgggaaagagcgtgatgtgaagtctggttctgaggtcattggaacaaagaggacgcttgttttccatattggtcgtgcaccaaaaggcgaaagaactgactggattatgcacgagtactgcgtgaaaggagtatctctggatgatgctatggttgtttgccgggttaggaggaacaaagaatacaatagtggtacaagtcagaaggcaccaaagccaaattcatcagccgagaagcatgcgaaagtccaaaatggcgctacgagttcagggagcccgtctgattgggacaacttggttgatttttacctagcaggtgaatcaggggagaaactactcgctgagatggcagagtcatcagaaaatctacaggtggataatgacgaggatttctttgcggatatcctaagagacgaaatcatcaatctcgatgaagcggtgatgacagggaacacaccaaacgaagtgccaacactagaatcagcatcaatggagataagggtacttcctttaccaaacatgatagacaaacaaatgtcatcactgttagaggaaagaccatcacagaagaagaaaggaaaagacgccacggaatcattgtcgagctgcttcgtgggtttatactcgatcaaatcagtgaacaaggcacgatgggatgttattataggtgtagtggctctgatagcaatgttgttttatctagaataagaggcttatggaagtagcgaaaaacagtgtcctagctatgtttgtatcatcttttctcggacattgacaaggattatatgatgtttttgtgtaaaaaaaaaaaaaaaaaaaaaaaaa

SEQ ID NO: 106 - protein, 340 aa, Arabidopsis thaliana
  G512
MDTKAVGVSKDTAASMEASTVFPGFKFSPTDVELISYYLKRKMDGLERSVEVIPDLEIYNFEPWDLPDKSIVKSDSEWFFFCARGKKYPHGSQNRRATKMGYWKATGKERDVKSGSEVIGTKRTLVFHIGRAPKGERTDWIMHEYCVKGVSLDDAMVVCRVRRNKEYNSGTSQKAPKPNSSAEKHAKVQNGATSSGSPSDWDNLVDFYLAGESGEKLLAEMAESSENLQVDNDEDFFADILRDEIINLDEAVMTGNTPNEVPTLESASMEIRVLPLPNMIDKQMSSLLEERPSQKKKGKDATESLSSCFVGLYSIKSVNKARWDVIIGVVALIAMLFYLE

SEQ ID NO: 107 - DNA, 1179 bp, Arabidopsis thaliana
  G525
cttctctcttctcaaaaacccttccctcttcgtctccaaacaacaacaaacacaacaacaacaaaaatcttacaagaagatcatttttagaaaccctattaggataaaatggattacgaggcatcaagaatcgtcgaaatggtagaagatgaagaacatatagatctaccaccaggattcagatttcaccctactgatgaagaactcataactcactacctcaaaccaaaggttttcaacactttcttctctgctactgccattggtgaagttgatctcaacaagattgagccttgggacttaccatggaaggctaagatgggagaaaaagaatggtatttcttctgtgtgagagaccggaaatacccgaccggtttaaggacaaaccgggcgacagaagccggttattggaaagccacaggaaaagacaaagagatattcaagggaaaatcacttgtgggtatgaagaaaactttggttttctataaaggaagagctcctaaaggagttaaaaccaattgggttatgcatgaatatcgtttagaaggcaaatattgtattgaaaatcttccccaaacagctaagaacgaatgggttatatgtcgtgttttccaaaaacgtgccgatggtacaaaggttccaatgtcaatgcttgatccacacattaaccgaatggaaccagccggtttaccttcgttaatggattgttctcaacgagactccttcaccggttcgtcgtctcacgtgacctgcttctccgaccaagaaaccgaagacaaaagacttgtccacgagtccaaagacggttttggttctctgttttactcggatcctctgtttttacaagacaattattcgctaatgaagctgttgcttgacggtcaagaaactcaattctccggcaaacctttcgacggtcgtgattcgtccggtacagaagaattggattgcgtttggaatttctgagttgtataagttatgttgtagacttgtagtagtcatgtgttcgtgtgtgtgaatgaatattcttgttacatttttttgtaaaaaaggagaaaaaaatatgctagaaagtcaattgcttttgttatgtagcattagtgttttttatgtactcaatagacttcctaattaaataaaaatcttaatttatttgccaaaaaaaaaaaaaaaaaaaaa

SEQ ID NO: 108 - protein, 285 aa, Arabidopsis thaliana
  G525
MDYEASRIVEMVEDEEHIDLPPGFRFHPTDEELITHYLKPKVFNTFFSATAIGEVDLNKIEPWDLPWKAKMGEKEWYFFCVRDRKYPTGLRTNRATEAGYWKATGKDKEIFKGKSLVGMKKTLVFYKGRAPKGVKTNWVMHEYRLEGKYCIENLPQTAKNEWVICRVFQKRADGTKVPMSMLDPHINRMEPAGLPSLMDCSQRDSFTGSSSHVTCFSDQETEDKRLVHESKDGFGSLFYSDPLFLQDNYSLMKLLLDGQETQFSGKPFDGRDSSGTEELDCVWNF

SEQ ID NO: 109 - DNA, 1360 bp, Arabidopsis thaliana
  G555
caaaagtagtaacctttgttggtgattgatactatatctgttgtgggttttagacaaagaccacgtctttgcagttgtagattggaattttccggatctttctctaaatcgctttttctccgagcaacttttgtttggggttaagctcaaagaatccgttcttttcagtctttactccatctagggtaccacgattggatcggtttttatctgatgatttagtaacagagattttgaagaaaaagaaaaatgggagatactagtccaagaacatcagtctcaacagatggagacactgatcataataacctaatgttcgatgaagggcatttgggtatcggtgcttctgattctagtgaccgttcaaagagtaaaatggatcaaaagacgcttcgtaggctcgctcaaaaccgtgaggctgcaaggaaaagcagattgaggaagaaagcatatgttcagcagctagagaacagtcgattgaagctaacacaacttgagcaggagctacaaagagcacggcaacagggtgtctttatctcaagctctggagaccaagcccattctaccgctggagatggggcaatggcatttgatgtagaatacagacgatggcaggaagataaaaacagacagatgaaggagctgagttctgctatagattctcacgcgactgattctgagcttcggataattgtagatggagtaatagctcactatgaggagctttacaggataaaaggcaacgcagctaagagtgatgtcttccatttattatcagggatgtggaaaaccccagctgagagatgtttcttgtggctcggcggtttccgttcatcagaacttctcaagcttatagcgtgtcagttggagcccttgacagaacaacaatcgctagacataaataacttgcaacagtcaactcagcaagcagaagatgctttgtctcaagggatggacaacttacagcaatcactcgctgatactttatcgagtgggactctcggttcaagttcatcagggaatgtagctagctacatgggtcagatggccatggcgatggggaagttaggtacccttgaaggatttatccgccaggctgataacttaaggctacaaacatatcaacagatggtgagactattaacaacccgacaatcggctcgtgctctccttgcagtacacaattatacattgcggttacgtgctcttagctctctatggcttgccagaccaagagagtgaaccatgactctattatacttcaacgaaggtccagaaaatttgagattcttagcataagatttgacgactttagacacgtagctcgtatacaagattatgattatactgttttgtgttg

SEQ ID NO: 110 - protein, 330 aa, Arabidopsis thaliana
  G555
MGDTSPRTSVSTDGDTDHNNLMFDEGHLGIGASDSSDRSKSKMDQKTLRRLAQNREAARKSRLRKKAYVQQLENSRLKLTQLEQELQRARQQGVFISSSGDQAHSTAGDGAMAFDVEYRRWQEDKNRQMKELSSAIDSHATDSELRIIVDGVIAHYEELYRIKGNAAKSDVFHLLSGMWKTPAERCFLWLGGFRSSELLKLIACQLEPLTEQQSLDINNLQQSTQQAEDALSQGMDNLQQSLADTLSSGTLGSSSSGNVASYMGQMAMAMGKLGTLEGFIRQADNLRLQTYQQMVRLLTTRQSARALLAVHNYTLRLRALSSLWLARPRE

SEQ ID NO: 111 - DNA, 932 bp, Arabidopsis thaliana
  G350
ctttcttctcaatttagaacttagtagctagtcttcaagataatggcacttgaaactcttacttctccaagattatcttctccgatgccgactctgtttcaagattcagcactagggtttcatggaagcaaaggcaaacgatctaagcgatcaagatctgaattcgaccgtcagagtctcacggaggatgaatatatcgctttatgtctcatgcttcttgctcgcgacggagatagaaaccgtgaccttgacctgccttcttcttcgtcttcacctcctctgcttcctcctcttcctactccgatctacaagtgtagcgtctgtgacaaggcgttttcgtcttaccaggctcttggtggacacaaggcaagtcaccggaaaagcttttcgcttactcaatctgccggaggagatgagctgtcgacatcgtcggcgataaccacgtctggtatatccggtggcgggggaggaagtgtgaagtcgcacgtttgctctatctgtcataaatcgttcgccaccggtcaagctctcggcggccacaaacggtgccactacgaaggaaagaacggaggcggtgtgagtagtagcgtgtcgaattctgaagatgtggggtctacaagccacgtcagcagtggccaccgtgggtttgacctcaacataccgccgataccggaattctcgatggtcaacggagacgaagaggtgatgagtcctatgccggcgaagaaactccggtttgacttcccggagaaaccctaaacataaacctaggaaaaactttacagaattcattttataggaaattgttttactgtatatacaaatatcgattttgattgatgttcttcttcactgaaaaattatgattctttgttgtataattgatgtttctgaaaaagatataactttttattaaaaaaaaaaaaaaaaaa

SEQ ID NO: 112 - protein, 238 aa, Arabidopsis thaliana
  G350
MALETLTSPRLSSPMPTLFQDSALGFHGSKGKRSKRSRSEFDRQSLTEDEYIALCLMLLARDGDRNRDLDLPSSSSSPPLLPPLPTPIYKCSVCDKAFSSYQALGGHKASHRKSFSLTQSAGGDELSTSSAITTSGISGGGGGSVKSHVCSICHKSFATGQALGGHKRCHYEGKNGGGVSSSVSNSEDVGSTSHVSSGHRGFDLNIPPIPEFSMVNGDEEVMSPMPAKKLRFDFPEKP

SEQ ID NO: 113 - DNA, 1050 bp, Arabidopsis thaliana
  G220
atgggaagatcaccgtgttgcgatcaagacaaaggcgtgaagaaaggaccgtggctgccagaagaagatgataagctcactgcttatataaacgagaatggttatgggaattggcggtcgcttcctaagctcgctggacttaaccgctgtggcaagagctgtcggctccggtggatgaattatctccggcctgatatccggagaggcaaattttccgatggagaagagagtactatcgttagactccatgccctccttggcaacaaatggtcgaaaattgcgggtcatcttccaggaagaacagataatgaaattaaaaactattggaacactcatatgaggaagaagttattgcaaatggggattgatccagtgacgcacgagccaagaaccaacgatcttagccctatcctcgatgtttctcaaatgctcgcggcagcaatcaacaatggccaatttggtaacaataacctcctcaacaataacaccgctttggaagatattcttaaactccaattgatccataaaatgcttcaaatcataacccccaaagccataccaaacatcagtagcttcaagaccaatttactgaatcctaaaccagaaccggtagtcaatagcttcaataccaattcagtgaatcctaaaccggatcccccggctggtcttttcataaaccaaagtggaatcactcctgaggccgcctcggatttcattccatcttatgaaaatgtttgggatggttttgaagataaccagcttcctggtttggttacggtttctcaagagagtttaaatacagcaaaaccgggtaccagtacgaccaccaaggtaaatgatcatatcagaaccggtatgatgccgtgttactatggtgatcaactactagaaaccccatctactggttcggtatcggtctctcccgagacaaccagtttgaaccatcccagtacagctcaacactcatccggttcagatttcctagaggactgggagaagtttcttgatgatgaaacaagtgattcttgctggaaaagtttcttagagtaa

SEQ ID NO: 114 - protein, 349 aa, Arabidopsis thaliana
  G220
MGRSPCCDQDKGVKKGPWLPEEDDKLTAYINENGYGNWRSLPKLAGLNRCGKSCRLRWMNYLRPDIRRGKFSDGEESTIVRLHALLGNKWSKIAGHLPGRTDNEIKNYWNTHMRKKLLQMGIDPVTHEPRTNDLSPILDVSQMLAAAINNGQFGNNNLLNNNTALEDILKLQLIHKMLQIITPKAIPNISSFKTNLLNPKPEPVVNSFNTNSVNPKPDPPAGLFINQSGITPEAASDFIPSYENVWDGFEDNQLPGLVTVSQESLNTAKPGTSTTTKVNDHIRTGMMPCYYGDQLLETPSTGSVSVSPETTSLNHPSTAQHSSGSDFLEDWEKFLDDETSDSCWKSFLE

SEQ ID NO: 115 - DNA, 2292 bp, Arabidopsis thaliana
  G472
gacgatagaagctggttttacgggagagaaattagagaggttacagttacagagcaggaaggattgcgttgcgttacgattcagatctctctcgatcgttacttacatgcattagagaacaagaaaacgtggagacggaatccaagggaccttcttcttcaatttaattcctcactttattacgcacccttcttttattttcgactgtttcagcttccggttcttcatcggagagagatagagagactgtgtgttttggtgtgactgatagaagatttaatcagctgatggcaaatcgcggaggtgaatatctgtacgatgagttatggaaattatgcgcgggacctcttgttgatgttcctcaagctcaagaaagagtttattattttcctcaaggtcacatggaacaactcgaagcgtcaacgcaacaagtcgacttaaatacgatgaagcctctttttgttcttcctcctaagattctctgcaatgttatgaacgttagtcttcaggcggagaaagatacggatgaggtctatgctcagattactttgatccctgttggaactgaagttgatgaacctatgagtcctgatccctctcctcctgagttgcaaaggccgaaagttcactctttcagcaaggttttgacagcgtctgatacaagcacccatggtggcttttctgttctaaggaaacatgccacggaatgtcttcctccgctggatatgactcagcaaaccccgacccaggagttagtagccgaagatgtgcacggttatcagtggaaattcaagcatatttttagaggccaaccacggaggcatctattgacgacagggtggagcacctttgttacatcaaagagattggttgctggggacacctttgtattcctgagaggggagaacggagagttgcgagttggagtcagacgtgctaatcttcaacagagcagtatgccttcatccgttatatcaagtcatagtatgcatctgggagtgcttgctactgcacgccatgctactcaaacgaaaactatgttcattgtatactataaaccaaggacaagccaattcatcattagcttgaacaaatatctagaagccatgagcaataagttctctgtagggatgagatttaagatgcgttttgagggagaggattcccctgaaagaagatattctggcacggttattggtgtgaaagactgctcccctcactggaaagactcaaaatggcgatgcttagaagttcattgggatgagcctgcatcgatttcaagacccaataaggtttcaccatgggaaattgaaccgtttgtaaattcagaaaacgttcccaaatcagttatgctgaagaacaaaaggccccgtcaagttagtgaagtatctgcacttgatgtaggcataacagcttcaaacctttggagctctgttttgacgcaaccccatgagtttgcacaatcgtgcatcacctcacagtggagttctcctcagcaatgtcatcgtgatgcaaatgaggatgctaagaaatctgactggctaaataactcttactctgtgtcaaatgtagcaaaagactcaacactgaacgaccaaatggtttccccagtcgagcagaagaagcctgagacaaccgctaattatagattatttggaattgatctgatgagttcctccctagcggttcctgaggagaaaactgcacccatgcgaccaatcaacatatccaaaccgactatggacagccactcagacccaaaatcagagatttcaaaagtatcagaagagaaaaagcaggaacctgcggagggatcaccaaaagaggtccaaagcaagcaaagcagttctacaagaagccgtaccaaggtgcagatgcaaggcgtacctgtgggcagggctgtggatttaaatgcgctaaaggggtacaacgagctcatagatgacattgagaagctgtttgacataaaaggggaactgcggagtcgcaatcaatgggaaatagtgttcacagacgatgagggagatatgatgcttgtcggtgatgacccatggcctgagttctgcaacatggtgaagagaatattcatatggtcgaaagaggaagtgaagaaaatgacgcctgggaaccaactccggatgctgttaagggaagttgaaacaacactaacaacaacttccaaaacagataatcattccaactaatttttattctattctctatcagtcttcgtctccttcttatttgctttgtctttcggaattatcttgtttgtgtctgtgttttgttgcg

SEQ ID NO: 116 - protein, 638 aa, Arabidopsis thaliana
  G472
MANRGGEYLYDELWKLCAGPLVDVPQAQERVYYFPQGHMEQLEASTQQVDLNTMKPLFVLPPKILCNVMNVSLQAEKDTDEVYAQITLIPVGTEVDEPMSPDPSPPELQRPKVHSFSKVLTASDTSTHGGFSVLRKHATECLPPLDMTQQTPTQELVAEDVHGYQWKFKHIFRGQPRRHLLTTGWSTFVTSKRLVAGDTFVFLRGENGELRVGVRRANLQQSSMPSSVISSHSMHLGVLATARHATQTKTMFIVYYKPRTSQFIISLNKYLEAMSNKFSVGMRFKMRFEGEDSPERRYSGTVIGVKDCSPHWKDSKWRCLEVHWDEPASISRPNKVSPWEIEPFVNSENVPKSVMLKNKRPRQVSEVSALDVGITASNLWSSVLTQPHEFAQSCITSQWSSPQQCHRDANEDAKKSDWLNNSYSVSNVAKDSTLNDQMVSPVEQKKPETTANYRLFGIDLMSSSLAVPEEKTAPMRPINISKPTMDSHSDPKSEISKVSEEKKQEPAEGSPKEVQSKQSSSTRSRTKVQMQGVPVGRAVDLNALKGYNELIDDIEKLFDIKGELRSRNQWEIVFTDDEGDMMLVGDDPWPEFCNMVKRIFIWSKEEVKKMTPGNQLRMLLREVETTLTTTSKTDNHSN

SEQ ID NO: 117 - DNA, 989 bp, Arabidopsis thaliana
  G227
gtaccgtcgacgatccggcgatgtcaaacccgacccgtaagaatatggagaggattaaaggtccatggagtccagaagaagatgatctgttgcagaggcttgttcagaaacatggtccgaggaactggtctttgattagcaaatcaatccctggacgttccggcaaatcttgtcgtctccggtggtgtaaccagctatctccggaggtagagcaccgtgctttttcgcaggaagaagacgagacgattattcgagctcacgctcggtttggtaacaagtgggctacgatctctcgtcttctcaatggacgaaccgataacgctatcaagaatcattggaactcgacgctgaagcgaaaatgcagcgtcgaagggcaaagttgtgattttggtggtaatggagggtatgatggtaatttaggagaagagcaaccgttgaaacgtacggcgagtggtggtggtggtgtctcgactggcttgtatatgagtcccggaagtccatcgggatctgacgtcagcgagcaatctagtggtggtgcacacgtgtttaaaccaacggttagatctgaggttacagcgtcatcgtctggtgaagatcctccaacttatcttagtttgtctcttccttggactgacgagacggttcgagtcaacgagccggttcaacttaaccagaatacggttatggacggtggttatacggcggagctgtttccggttagaaaggaagagcaagtggaagtagaagaagaagaagcgaaggggatatctggtggattcggtggtgagttcatgacggtggttcaggagatgataaggacggaggtgaggagttacatggcggatttacagcgaggaaacgtcggtggtagtagttctggcggcggaggtggcggttcgtgtatgccacaaagtgtaaacagccgtcgtgttgggtttagagagtttatagtgaaccaaatcggaattgggaagatggagtaggcggcc

SEQ ID NO: 118 - protein, 320 aa, Arabidopsis thaliana
  G227
MSNPTRKNMERIKGPWSPEEDDLLQRLVQKHGPRNWSLISKSIPGRSGKSCRLRWCNQLSPEVEHRAFSQEEDETIIRAHARFGNKWATISRLLNGRTDNAIKNHWNSTLKRKCSVEGQSCDFGGNGGYDGNLGEEQPLKRTASGGGGVSTGLYMSPGSPSGSDVSEQSSGGAHVFKPTVRSEVTASSSGEDPPTYLSLSLPWTDETVRVNEPVQLNQNTVMDGGYTAELFPVRKEEQVEVEEEEAKGISGGFGGEFMTVVQEMIRTEVRSYMADLQRGNVGGSSSGGGGGGSCMPQSVNSRRVGFREFIVNQIGIGKME

SEQ ID NO: 119 - DNA, 1139 bp, Arabidopsis thaliana
  G234
cacaacatcatacccaccaacatatataatcttgatcatagagagataaacagaggccgctatcaagaacaagactaagaacaagacttcactaggagtacaagtatgggaagagcaccgtgttgtgacaaagcaaacgtgaagaaagggccttggtctcctgaggaagatgcaaaactcaaatcttacattgaaaatagtggcaccggaggcaattggatcgctttgcctcaaaagattggtttaaagagatgtggaaagagttgcaggctgaggtggcttaactatcttagaccaaacatcaaacatggtggcttctctgaggaagaagaaaacatcatttgtagcctttaccttacaattggtagcaggtggtctataatcgctgctcaattgccgggacgaacagacaacgatataaaaaactattggaacacgaggctcaagaagaaactcattaacaaacaacgcaaggagcttcaagaagcttgtatggagcagcaagagatgatggtgatgatgaagagacaacaccaacaacaacaaatccaaacttcttttatgatgagacaagaccaaacaatgttcacatggccactacatcatcataatgttcaagttccagctcttttcagaatcaaaccaactcgttttgcgaccaagaagatgttaagccagtgctcatcaagaacatggtcaagatcgaagatcaagaactggagaaaacaaacctcatcatcatcaagattcaatgacaacgcttttgatcatctctctttctctcaactcttgttagatcctaatcataaccacttaggatcaggagagggtttctccatgaactctatcttgagcgccaacacaaactctccattgcttaacacaagtaatgataatcagtggttcgggaatttccaggccgaaaccgtaaacttgttctcaggagcctccacaagtacttcggcagatcaaagcactataagttgggaagacataagctctcttgtttattctgattcaaagcaatttttttaattataataatatattattcttaagatgaaacgtacatcattattattaattgggggtacgtaacgtatatatggaataacgatctagtttgtttaaatttaaaa

SEQ ID NO: 120 - protein, 309 aa, Arabidopsis thaliana
  G234
MGRAPCCDKANVKKGPWSPEEDAKLKSYIENSGTGGNWIALPQKIGLKRCGKSCRLRWLNYLRPNIKHGGFSEEEENIICSLYLTIGSRWSIIAAQLPGRTDNDIKNYWNTRLKKKLINKQRKELQEACMEQQEMMVMMKRQHQQQQIQTSFMMRQDQTMFTWPLHHHNVQVPALFRIKPTRFATKKMLSQCSSRTWSRSKIKNWRKQTSSSSRFNDNAFDHLSFSQLLLDPNHNHLGSGEGFSMNSILSANTNSPLLNTSNDNQWFGNFQAETVNLFSGASTSTSADQSTISWEDISSLVYSDSKQFF

SEQ ID NO: 121 - DNA, 1339 bp, Arabidopsis thaliana
  G515
gtcgacaagaagggccgcataaatacaaaaccctatacgtctttcttctccatcattttaagttgcagaagaacatcaaccatggcgaaagctctccaatgcatatttttttgatcttttccttttcccttcttttttgatcgctgcagagaaatggaaactcctgtgggtttaagattctgtccgaccgacgaggagatcgtcgtcgattacctttggccgaaaaattccgatagagacacgagccatgtcgatcgattcattaacacagtccctgtctgtagactcgatccttgggagttaccttgccagtcaaggatcaaactgaaagatgtggcttggtgtttcttcagacctaaggagaacaaatatggcagaggtgatcagcagatgagaaaaacgaaatctgggttttggaagagtactggcagaccaaagcctatcatgcgtaatcgccaacagatcggtgagaaaaagattttgatgttttacacgagtaaggaatccaaatccgattgggttatacacgagtaccacggtttctctcataaccagatgatgatgacatacacactctgtaaagttatgtttaatggtggcatgagagagaagtcttcctcttctccttcttcttctggtgttagtggaattgagcagagtcgtcgtgactctttaatccctcagcttgtcaacaattctgagggatcctcacttcacagagaagatccaagtcagtttggtgatgtgctgcaagaagctccaatcgaggatgctaaactgaccgaggaattggtaaaatggctgatgaatgatgaggatgatgctcaaatcgaggatgctataccgattgaggaatgggaaacatggttgaatgatattgatgatgctaaggagaagagtatcatgtttatgcatgataatcgaagtgattacagacctccaaactcattaactggtgtcttcagtgatgatgttagcagtgatgataatgattctgatttgctaactccaaaaacaaactctattcaaacttcgagcacttgtgatagttttggtagctcaaatcatcgcatagaccagatcaaagacctgcaagaatctcctacctcaacaatcaacttagtgtcactaactcaagaggtgagcgcggccgctaataaccagtattgataccgccgagaagaagaagaatccttatgatgatgcacaagggactgagattggtgagcataaattgggtcaagagacgatcaagaagaaaagagctggtttctttcacaggatgatacaaaaattcgtcaagaaaattcacctatgttcttccatctcaagaacttga

SEQ ID NO: 122 - protein, 338 aa, Arabidopsis thaliana
  G515
METPVGLRFCPTDEEIVVDYLWPKNSDRDTSHVDRFINTVPVCRLDPWELPCQSRIKLKDVAWCFFRPKENKYGRGDQQMRKTKSGFWKSTGRPKPIMRNRQQIGEKKILMFYTSKESKSDWVIHEYHGFSHNQMMMTYTLCKVMFNGGMREKSSSSPSSSGVSGIEQSRRDSLIPQLVNNSEGSSLHREDPSQFGDVLQEAPIEDAKLTEELVKWLMNDEDDAQIEDAIPIEEWETWLNDIDDAKEKSIMFMHDNRSDYRPPNSLTGVFSDDVSSDDNDSDLLTPKTNSIQTSSTCDSFGSSNHRIDQIKDLQESPTSTINLVSLTQEVSAAANNQY

SEQ ID NO: 123 - DNA, 2371 bp, Arabidopsis thaliana
  G274
gacattattttaagtgtgttctctctctgtcacactcacaaagctttatactttctggctactgcaagctcatcagtgaaaagagcttaaaccagagagatctgataagagaaattttagagtctctctgcttcaacaagatctacatcgaccaggagattagaaagaatcatgggttctaagcataacccaccagggaataacagatcgagaagtacactatctctactcgttgtggttggtttatgttgtttcttctatcttcttggagcatggcaaaagagtgggtttggtaaaggagatagcatagctatggagattacaaagcaagcgcagtgtactgacattgtcactgatcttgattttgaacctcatcacaacacagtgaagatcccacataaagctgatcccaaacctgtttctttcaaaccgtgtgatgtgaagctcaaggattacacgccttgtcaagagcaagaccgagctatgaagttcccgagagagaacatgatttacagagagagacattgtcctcctgataatgagaagctgcgttgtcttgttccagctcctaaagggtatatgactcctttcccttggcctaaaagcagagattatgttcactatgctaatgctcctttcaagagcttgactgtcgaaaaagctggacagaattgggttcagtttcaagggaatgtgtttaaattccctggtggaggaactatgtttcctcaaggtgctgatgcgtatattgaagagctagcttctgttatccctatcaaagatggctctgttagaaccgcattggacactggatgtggggttgctagttggggtgcttatatgcttaagaggaatgttttgactatgtcgtttgcgccaagggataaccacgaagcacaagtccagtttgcgcttgagagaggtgttccagcgattatcgctgttcttggatcaatccttcttccttaccctgcaagagcctttgacatggctcaatgctctcgatgcttgataccatggaccgcaaacgagggaacatacttaatggaagtagatagagtcttgagacctggaggttactgggtcttatcgggtcctccaatcaactggaagacatggcacaagacgtggaaccgaactaaagcagagctaaatgccgagcaaaagagaatagagggaatcgcagagtccttatgctgggagaagaagtatgagaagggagacattgcaattttcagaaagaaaataaacgatagatcatgcgatagatcaacaccggttgacacctgcaaaagaaaggacactgacgatgtctggtacaaggagatagaaacgtgtgtaacaccattccctaaagtatcaaacgaagaagaagttgctggaggaaagctaaagaagttccccgagaggctattcgcagtgcctccaagtatctctaaaggtttgattaatggcgtcgacgaggaatcataccaagaagacatcaatctatggaagaagcgagtgaccggatacaagagaattaacagactgataggttccaccagataccgtaatgtgatggatatgaacgccggtcttggtggattcgctgctgcgcttgaatcgcctaaatcgtgggttatgaatgtgattccaaccattaacaagaacacattgagtgttgtttatgagagaggtctcattggtatctatcatgactggtgtgaaggcttttcaacttatccaagaacatacgatttcattcacgctagtggtgtcttcagcttgtatcagcacagctgcaaacttgaggatattcttcttgaaactgatcggattttacgaccggaagggattgtgattttccgggatgaggttgatgttttgaatgatgtgaggaagatcgttgatggaatgagatgggatactaagttaatggatcatgaagacggtcctctcgtgccggagaagattcttgtcgccacgaagcagtattgggtagccggcgacgatggaaacaattctccgtcgtcttctaatagtgaagaagaataaaacaaaaacaaaaaactcctcaggttactaagcttgaagtgtagatctattttacaacatctggaaaattcttatcaaaaaaggaaggaatcagaatttccattaaagaaaggtgtcaaaaaaaagttgtaaaactatatagtagtgatcaagacgaatatgtgcatttatgttttatttttgttccctagtttttaattttatttttttgaaggaagaaaaaattagttccatgtgtttttgcaagatagttgaaaccttggacgcttgttatgtatgatgcgatcttgacattttttaataacagttattttaaataaatttatgatataaa

SEQ ID NO: 124 - protein, 621 aa, Arabidopsis thaliana
  G274
MGSKHNPPGNNRSRSTLSLLVVVGLCCFFYLLGAWQKSGFGKGDSIAMEITKQAQCTDIVTDLDFEPHHNTVKIPHKADPKPVSFKPCDVKLKDYTPCQEQDRAMKFPRENMIYRERHCPPDNEKLRCLVPAPKGYMTPFPWPKSRDYVHYANAPFKSLTVEKAGQNWVQFQGNVFKFPGGGTMFPQGADAYIEELASVIPIKDGSVRTALDTGCGVASWGAYMLKRNVLTMSFAPRDNHEAQVQFALERGVPAIIAVLGSILLPYPARAFDMAQCSRCLIPWTANEGTYLMEVDRVLRPGGYWVLSGPPINWKTWHKTWNRTKAELNAEQKRIEGIAESLCWEKKYEKGDIAIFRKKINDRSCDRSTPVDTCKRKDTDDVWYKEIETCVTPFPKVSNEEEVAGGKLKKFPERLFAVPPSISKGLINGVDEESYQEDINLWKKRVTGYKRINRLIGSTRYRNVMDMNAGLGGFAAALESPKSWVMNVIPTINKNTLSVVYERGLIGIYHDWCEGFSTYPRTYDFIHASGVFSLYQHSCKLEDILLETDRILRPEGIVIFRDEVDVLNDVRKIVDGMRWDTKLMDHEDGPLVPEKILVATKQYWVAGDDGNNSPSSSNSEEE

SEQ ID NO: 125 - DNA, 759 bp, Arabidopsis thaliana
  G3
gtttgtcttttatcaatggaaagagaacaagaagagtctacgatgagaaagagaaggcagccacctcaagaagaagtgcctaaccacgtggctacaaggaagccgtacagagggatacggaggaggaagtggggcaagtgggtggctgagattcgtgagcctaacaaacgctcacggctttggcttggctcttacacaaccgatatcgccgccgctagagcctacgacgtggccgtcttctacctccgtggcccctccgcacgtctcaacttccctgatcttctcttgcaagaagaggaccatctctcagccgccaccaccgctgacatgcccgcagctcttataagggaaaaagcggcggaggtcggcgccagagtcgacgctcttctagcttctgccgctccttcgatggctcactccactccgccggtaataaaacccgacttgaatcaaatacccgaatccggagatatatagtcaatttatatacatgtagtttgttttgtttgattagaagattacatttacatacaagatacacatagatactggaaaatataggtatgtatacattcataaattatcttatgtatcaaagaattttatagattctgattagctttttgtttttgtttttgataagaactctgattagttgtccggagacaaaaccggctaagagcaatccatgagaagctagcgagtgttttttagttcaagttgtaatataaatgcatattaattctttagtaattttgt

SEQ ID NO: 126 - protein, 153 aa, Arabidopsis thaliana
  G3
MEREQEESTMRKRRQPPQEEVPNHVATRKPYRGIRRRKWGKWVAEIREPNKRSRLWLGSYTTDIAAARAYDVAVFYLRGPSARLNFPDLLLQEEDHLSAATTADMPAALIREKAAEVGARVDALLASAAPSMAHSTPPVIKPDLNQIPESGDI

SEQ ID NO: 127 - DNA, 1059 bp, Arabidopsis thaliana
  G1004
atggcgactcctaacgaagtatctgcactttggttcatcgagaaacatctactcgacgaggcttctcctgtggctacagatccatggatgaagcacgaatcatcatcagcaacagaatctagctctgactcttcttctatcatcttcggatcatcgtcctcttctttcgccccaattgatttctctgaatccgtatgcaaacctgaaatcatcgatctcgatactcccagatctatggaatttctatcgattccatttgaatttgactcagaagtttctgtttctgatttcgattttaaaccttctaatcaaaatcaaaatcagtttgaaccggagcttaaatctcaaattcgtaaaccgccattgaagatttcgcttccagctaaaacagagtggattcaattcgcagctgaaaacaccaaaccggaagttactaaaccggtttcggaagaagagaagaagcattacagaggagtaagacaaagaccgtgggggaaattcgcggcggagattcgtgacccgaataaacgcggatctcgcgtttggcttgggacgtttgatacagcgattgaagcggctagagcttatgacgaagcagcgtttagactacgaggatcgaaagcgattttgaatttccctcttgaagttgggaagtggaaaccacgcgccgatgaaggtgagaagaaacggaagagagacgatgatgagaaagtgactgtggttgagaaagtgttgaagacggaacagagcgttgacgttaacggtggagagacgtttccgtttgtaacgtcgaatttaacggaattatgtgactgggatttaacggggtttcttaactttccgcttctgtcgccgttatctcctcatccaccgtttggttattcccagttgaccgttgtttgattagttttttttgagtttttgaacgatgtgtatgctgacgtggacgtacacgtaggtgcatgcgatgaaaaaaacatctatttgttcatatttttgcgtttttctatttgttcattctttttcacaattcacaatacattatttcagttaatgatc

SEQ ID NO: 128 - protein, 300 aa, Arabidopsis thaliana
  G1004
MATPNEVSALWFIEKHLLDEASPVATDPWMKHESSSATESSSDSSSIIFGSSSSSFAPIDFSESVCKPEIIDLDTPRSMEFLSIPFEFDSEVSVSDFDFKPSNQNQNQFEPELKSQIRKPPLKISLPAKTEWIQFAAENTKPEVTKPVSEEEKKHYRGVRQRPWGKFAAEIRDPNKRGSRVWLGTFDTAIEAARAYDEAAFRLRGSKAILNFPLEVGKWKPRADEGEKKRKRDDDEKVTVVEKVLKTEQSVDVNGGETFPFVTSNLTELCDWDLTGFLNFPLLSPLSPHPPFGYSQLTVV

SEQ ID NO: 129 - DNA, 1099 bp, Arabidopsis thaliana
  G1043
aaataagataccactcaccaaaaacttcctcaaaactaacaaatggatactaataaagcaaaaaagcttaaagttatgaaccaactcgttgaaggccatgacttaacaactcagcttcagcaactcctctctcaacccgggtccggtctagaggatctagtggctaaaatcttagtgtgtttcaataacaccatctccgttcttgataccttcgaacccatctcctcctcctcatccctcgccgccgttgagggatctcaaaatgcttcatgtgataacgacggcaagtttgaagattccggcgatagtcggaaaagattgggacccgttaagggtaaaagaggatgctacaaaagaaaaaagagatcggagacgtgtactatagagtcgactatacttgaggacgcattttcttggaggaaatatggacaaaaggagattcttaatgccaaattcccaagaagttactttagatgcacacacaagtatacccaagggtgcaaggcaacaaagcaagtccagaaggttgagctcgaacccaagatgttcagtatcacatacataggaaaccacacgtgtaacaccaacgcagaaactcccaagagcaagacttgtgaccatcatgatgagatcttcatggattccgaagatcacaagagtcctagtttatctacctcaatgaaggaagaagacaatcctcatcgtcatcatggttcgtccacggagaatgacttgtcattggtgtggccagaaatggttttcgaagaagattatcatcatcaggccagttacgtcaatgggaaaacgagtacatctatcgatgttttgggttctcaggatctcatggtgtttggaggtggcggcgatttcgagtttagcgaaaatgagcacttctctatcttcagttcatgttcgaatctatcttgagtttaccactactataggactaagaccatgagttttaatcattaattaggccatgtagagtggaaaacatataatacatattttgcccttttctctaatgagtgtatgtactgtacatatagtactataaataaaactcttgctggattaaaacaaaaaaaaaaaaaaaaaa

SEQ ID NO: 130 - protein, 294 aa, Arabidopsis thaliana
  G1043
MDTNKAKKLKVMNQLVEGHDLTTQLQQLLSQPGSGLEDLVAKILVCFNNTISVLDTFEPISSSSSLAAVEGSQNASCDNDGKFEDSGDSRKRLGPVKGKRGCYKRKKRSETCTIESTILEDAFSWRKYGQKEILNAKFPRSYFRCTHKYTQGCKATKQVQKVELEPKMFSITYIGNHTCNTNAETPKSKTCDHHDEIFMDSEDHKSPSLSTSMKEEDNPHRHHGSSTENDLSLVWPEMVFEEDYHHQASYVNGKTSTSIDVLGSQDLMVFGGGGDFEFSENEHFSIFSSCSNLS

SEQ ID NO: 131 - DNA, 1351 bp, Arabidopsis thaliana
  G291
caagaacccaaagatctctctctatttgtttgccttcttctttctttctgactcaaaccctcaaatcaattctcgcgattaagcaaaaccctagatttattctactcttcgaagtcgatttcaatggaaggttcctcgtcagccatcgcgaggaagacatgggagctagagaacaacattctcccagtggaaccaaccgattcagcctccgacagtatattccactacgacgacgcttcacaagccaaaatccagcaggagaagccatgggcctccgatcctaactacttcaagcgcgttcacatctcagcccttgctcttctcaagatggtggttcacgctcgctccggtggcacaatcgagatcatgggtcttatgcagggtaaaaccgagggtgatacaatcatcgttatggatgcttttgctttgcctgttgaaggtactgagactagggttaatgctcagtctgatgcctatgagtatatggttgaatactctcagaccagcaagctggctgggaggttggagaacgttgttggatggtatcactctcaccctgggtatggatgttggctctcgggtattgatgtttcgacacagatgcttaaccaacagtatcaggagccattcttagctgttgttattgatccaacaaggactgtttcggctggtaaggttgagattggggcattcagaacatatccagagggacataagatctcggatgatcatgtttctgagtatcagactatccctcttaacaagattgaggactttggtgtacattgcaaacagtactactcattggacatcacttatttcaagtcatctctcgatagtcaccttctggatctcctttggaacaagtactgggtgaacactctttcttcttccccactgttgggcaatggagactatgttgccgggcaaatatcagacttggctgagaagctcgagcaagcggagagtcagctcgctaactcccggtatggaggaattgcgccagccggtcaccaaaggaggaaagaggatgagcctcaactcgcgaagataactcgggatagtgcaaagataactgtcgagcaggtccatggactaatgtcacaggttatcaaagacatcttgttcaattccgctcgtcagtccaagaagtctgctgacgactcatcagatccagagcccatgattacatcgtgaagttggtctattcttttgttttttggctgcggaaattgactatcggtttgacccggtttatgaggcaatgcccattgttccctatatctctagtgtagtatctgcttcaagacaaagatctttgggttattaaatgacattaacataaaaaaaa

SEQ ID NO: 132 - protein, 357 aa, Arabidopsis thaliana
  G291
MEGSSSAIARKTWELENNILPVEPTDSASDSIFHYDDASQAKIQQEKPWASDPNYFKRVHISALALLKMVVHARSGGTIEIMGLMQGKTEGDTIIVMDAFALPVEGTETRVNAQSDAYEYMVEYSQTSKLAGRLENVVGWYHSHPGYGCWLSGIDVSTQMLNQQYQEPFLAVVIDPTRTVSAGKVEIGAFRTYPEGHKISDDHVSEYQTIPLNKIEDFGVHCKQYYSLDITYFKSSLDSHLLDLLWNKYWVNTLSSSPLLGNGDYVAGQISDLAEKLEQAESQLANSRYGGIAPAGHQRRKEDEPQLAKITRDSAKITVEQVHGLMSQVIKDILFNSARQSKKSADDSSDPEPMITS

SEQ ID NO: 133 - DNA, 1380 bp, Arabidopsis thaliana
  G315
atggacgaacatgtcatgcgttcaatggattgggattccatcatgaaagaattggagcttgatgacgattctgctcctaactcacttaaaaccggtttcaccacaaccacaacagattctactattttgcctctttacgccgtcgattcaaatctccctggctttcccgatcagattcaaccgtcggatttcgaatcgtcttccgatgtttatcctggtcagaaccaaacaactgggtacggttttaattctcttgatagtgtcgacaatggaggatttgatttcattgaagatctcatccgagtcgtggattgtgttgaatcggacgagttacaactcgctcaggtggttttatcacggcttaatcaacgcttgagatctccggcgggtagaccgttacagagagctgcgttttactttaaggaagctctcggttcgtttttaaccggatcaaaccggaatccaatccggttatcttcttggtccgagattgttcagaggatccgagcgattaaggaatattccgggatttctccgatccctctcttctctcatttcacggcgaatcaagcgatactcgattcgttgagctcgcagtcgtcttctccgtttgttcacgtggtggattttgagattggattcggtggccaatacgcatcgcttatgagagaaatcactgagaaatctgtaagcggtggatttttaagagttacggcagtggtggcggaggagtgcgccgtagagacgcgactagtgaaagaaaacctaactcaattcgcggcggagatgaaaattcgtttccagattgagtttgtgctgatgaagacttttgagatgttatctttcaaagcgattaggtttgttgagggggagaggaccgtcgttttgatttccccggcgatatttcgtcgtctaagtggaatcactgattttgttaacaatttacggagagtatcacctaaggtcgttgtattcgtggatagtgaaggatggacggagatcgccggatctggatcattccggcgagagtttgttagcgctcttgagttctacacgatggtgctggagtcgctcgacgctgcagctcctcccggagatttggtgaagaagatagtggaagcgttcgttctacgaccgaagatctccgctgcggtagaaacggcggctgacaggagacacaccggcgaaatgacttggcgggaagcgttttgcgccgccgggatgaggccgatacagcaaagccagttcgccgactttcaagctgagtgtttattggagaaagcgcaagttagaggattccacgtggcgaaacgacaaggagaattggtgctgtgctggcatggaagagcacttgttgccacatcagcttggcggttttag

SEQ ID NO: 134 - protein, 459 aa, Arabidopsis thaliana
  G315
MDEHVMRSMDWDSIMKELELDDDSAPNSLKTGFTTTTTDSTILPLYAVDSNLPGFPDQIQPSDFESSSDVYPGQNQTTGYGFNSLDSVDNGGFDFIEDLIRVVDCVESDELQLAQVVLSRLNQRLRSPAGRPLQRAAFYFKEALGSFLTGSNRNPIRLSSWSEIVQRIRAIKEYSGISPIPLFSHFTANQAILDSLSSQSSSPFVHVVDFEIGFGGQYASLMREITEKSVSGGFLRVTAVVAEECAVETRLVKENLTQFAAEMKIRFQIEFVLMKTFEMLSFKAIRFVEGERTVVLISPAIFRRLSGITDFVNNLRRVSPKVVVFVDSEGWTEIAGSGSFRREFVSALEFYTMVLESLDAAAPPGDLVKKIVEAFVLRPKISAAVETAADRRHTGEMTWREAFCAAGMRPIQQSQFADFQAECLLEKAQVRGFHVAKRQGELVLCWHGRALVATSAWRF

SEQ ID NO: 135 - DNA, 1213 bp, Arabidopsis thaliana
  G398
agaaggtttctcttgtcctccatacacttagcacaactgataaatcttttgaggtaaaatcagctttagatcaaggtttttctagtcatctctactcataaagatcaaagcttttgctattctcattttctaccaagagacaatatcatgatgatgggtaaagaggatttgggtttaagtcttagcttgggatttgcacaaaaccatcctctccagctaaatcttaaacccacttcttcaccaatgtccaatctccagatgtttccatggaaccaaacccttgtttcttcctcagatcaacaaaagcaacagtttcttaggaaaatcgacgtgaacagcttgccaacaacggtggatttggaagaggagacaggagtttcgtctccaaacagtacgatctcgagcacagtgagtggaaagaggaggagtactgaaagagaaggtacctccggtggtggttgcggagatgaccttgacatcactctagatagatcttcctcacgtggaacctccgatgaagaggaagattacggaggtgagacttgtaggaagaagcttagactatccaaagatcaatccgcagttctcgaagacactttcaaagagcacaatactctcaatcccaaacagaagctggctttggctaagaagctaggtttaacagcaagacaagtggaagtgtggttccaaaacagaagagcaaggacaaagttaaagcagaccgaagtggattgcgagtatttgaaaagatgtgttgagaaattaacggaagagaatcggcggctcgagaaagaggcagcggaactaagagcattaaagctttcaccgcggttgtatggtcagatgagtccaccgaccacacttttgatgtgtccatcgtgtgaacgtgtggccggaccatcctcatctaaccacaaccagcgatctgtctcattgagtccatggctccaaatggcccatgggtcaacctttgatgtgatgcgtcctaggtcttaactttaatgctgcttctatgggttgtgtgtgggtcattgtactttttagattattgactctcagctaatgtatccttaaaagcctttttctacttttaaatttactttaatctaattaaattagttgtccatgtcttcttgataacaaaaaaatttataattataaaaaaaaaaaacaggataaaaaaaaaaaaaaaaaaaaaaaagctacgtcaggg

SEQ ID NO: 136 - protein, 282 aa, Arabidopsis thaliana
  G398
MMMGKEDLGLSLSLGFAQNHPLQLNLKPTSSPMSNLQMFPWNQTLVSSSDQQKQQFLRKIDVNSLPTTVDLEEETGVSSPNSTISSTVSGKRRSTEREGTSGGGCGDDLDITLDRSSSRGTSDEEEDYGGETCRKKLRLSKDQSAVLEDTFKEHNTLNPKQKLALAKKLGLTARQVEVWFQNRRARTKLKQTEVDCEYLKRCVEKLTEENRRLEKEAAELRALKLSPRLYGQMSPPTTLLMCPSCERVAGPSSSNHNQRSVSLSPWLQMAHGSTFDVMRPRS

SEQ ID NO: 137 - DNA, 1577 bp, Arabidopsis thaliana
  G4
aaagaatcgaatatttattatttcgccccgaagattctatttctgatcatttacacccctaaaaagagtagagctttcgtgaagccaccatgtgtggaggagctataatctccgatttcatacctccgccgaggtccctccgcgtcactaacgagtttatctggccggatctgaaaaacaaagtgaaagcttcaaagaagagatcgaataagcgatccgatttcttcgatcttgacgatgatttcgaagctgatttccaagggtttaaggatgactcggcttttgactgcgaagacgatgatgatgtcttcgtcaatgttaagcctttcgtcttcaccgcaactactaagcccgtagcttccgctttcgtctccactgtaggttcagcatatgccaagaaaactgtagagtccgctgagcaagctgagaaatcttctaagaggaagaggaagaatcaataccgagggattaggcagcgtccttggggaaaatgggctgcggagatccgtgatccgagaaaaggctcccgagaatggcttggaacattcgacactgctgaggaagcagcaagagcttatgatgctgcagcacgcagaatccgtggcacgaaagctaaggtgaattttcccgaggagaagaaccctagcgtcgtatcccagaaacgtcctagtgctaagactaataatcttcagaaatcagtggctaaaccaaacaaaagcgtaactttggttcagcagccaacacatctgagtcagcagtactgcaacaactcctttgacaactcttttggtgatatgagtttcatggaagagaagcctcagatgtacaacaatcagtttgggttaacaaactcgttcgatgctggaggtaacaatggataccagtatttcagttccgatcagggcagtaactccttcgactgttctgagttcgggtggagtgatcacggccctaaaacacccgagatctcttcaatgcttgtcaataacaacgaagcatcatttgttgaagaaaccaatgcagccaagaagctcaaaccaaactctgatgagtcagacgatctgatggcataccttgacaacgccttgtgggacaccccactagaagtggaagccatgcttggcgcagatgctggtgctgtgactcaggaagaggaaaacccagtggagctatggagcttagatgagatcaatttcatgctggaaggagacttttgaagtgatcgatggttccttagtttgtaaataaagctgtgttggattttgctgttgggggatggtacaagtcacacctcaagctctatgcattggtatctcatgagccttctcttccatagagagtttctcttttaattttgtcgaaataaaaaaggtgtgatgaagtaaatagaggtataataatatctatctattaagtcttgttttgttctttcatttttgtatttcttttctatttaaaagacagtttattagtcttctgagctctctttttgatctttgttatagcgtatcatcaccctcgaaagtgtaatgttttgtacccccaaacttgtttagcattataataaagtctctttg

SEQ ID NO: 138 - protein, 375 aa, Arabidopsis thaliana
  G4
MCGGAIISDFIPPPRSLRVTNEFIWPDLKNKVKASKKRSNKRSDFFDLDDDFEADFQGFKDDSAFDCEDDDDVFVNVKPFVFTATTKPVASAFVSTVGSAYAKKTVESAEQAEKSSKRKRKNQYRGIRQRPWGKWAAEIRDPRKGSREWLGTFDTAEEAARAYDAAARRIRGTKAKVNFPEEKNPSVVSQKRPSAKTNNLQKSVAKPNKSVTLVQQPTHLSQQYCNNSFDNSFGDMSFMEEKPQMYNNQFGLTNSFDAGGNNGYQYFSSDQGSNSFDCSEFGWSDHGPKTPEISSMLVNNNEASFVEETNAAKKLKPNSDESDDLMAYLDNALWDTPLEVEAMLGADAGAVTQEEENPVELWSLDEINFMLEGDF

SEQ ID NO: 139 - DNA, 1379 bp, Arabidopsis thaliana
  G428
ttacttttgtgtttcttcatattcttcagaagcaagcacaaggctagggatcgaagaagcggcgatcactgatcgtatctcactacgatcacattaatggatagaatgtgtggtttccgctcgacggaagactattcggagaaagcgacgttgatgatgccgtccgattatcagtctttgatttgttcaaccaccggagacaatcaaagactgtttggatccgacgaactcgctaccgctttgtcctcggagttgcttccgcgtattcgaaaagctgaggataatttctctcttagtgtcatcaaatccaaaatcgcttctcatcctttgtatcctcgcttactccaaacctacatcgattgccaaaaggtgggagcgcctatggaaatagcgtgtatattggaagagattcagcgagagaaccatgtgtacaagagagatgttgctccattatcttgctttggagctgatcctgagcttgatgaattcatggaaacctactgtgatatattggttaaatacaaaaccgatcttgcgaggccgttcgacgaggctacaactttcataaacaagattgaaatgcagcttcagaacttgtgcactggtccagcgtctgctacagctctttcagatgatggtgcggtttcatctgacgaggaactgagagaagatgatgacatagcagcggatgacagccaacaaagaagcaatgaccgcgatctgaaggaccagctactacgcaaatttggtagccatatcagttcattgaaactcgagttctctaaaaagaagaagaaagggaagctaccaagagaagcaagacaagcgttgctcgattggtggaatgttcataataaatggccttaccctactgaaggcgacaaaatagctctggctgaagaaacaggtttggatcaaaaacaaatcaacaattggtttataaaccaaaggaaacgccattggaagccttcggagaacatgccgtttgatatgatggacgattctaatgaaacattctttaccgaggaatgaaaagagagacatgggattgtgcattgtataatttttacactgttttcccaagaaaagaaaacagtaaaaagcttttggtaaatgggacatcatcgcgaatgaatggaaccagttagccaaaacggtcaagggcgtggcgtaacgagacattgtattggaaatagtggcaatattatgtcactaatcttccaatggtccaaaatgatagatttcttatttgtattgaaccttacttagatagctgatgtgtcaactaaataatttattttcatccttatactacttgtatcaatgtctctaattgatcaattgttgcttgctattcaaaaaaaaaaaaaaaaaaaaaa

SEQ ID NO: 140 - protein, 311 aa, Arabidopsis thaliana
  G428
MDRMCGFRSTEDYSEKATLMMPSDYQSLICSTTGDNQRLFGSDELATALSSELLPRIRKAEDNFSLSVIKSKIASHPLYPRLLQTYIDCQKVGAPMEIACILEEIQRENHVYKRDVAPLSCFGADPELDEFMETYCDILVKYKTDLARPFDEATTFINKIEMQLQNLCTGPASATALSDDGAVSSDEELREDDDIAADDSQQRSNDRDLKDQLLRKFGSHISSLKLEFSKKKKKGKLPREARQALLDWWNVHNKWPYPTEGDKIALAEETGLDQKQINNWFINQRKRHWKPSENMPFDMMDDSNETFFTEE

SEQ ID NO: 141 - DNA, 407 bp, Arabidopsis thaliana
  G226
ccagtagttatggataataccaaccgtcttcgtcttcgtcgcggtcccagtcttaggcaaactaagttcactcgatcccgatatgactctgaagaagtgagtagcatcgaatgggagtttatcagtatgaccgaacaagaagaagatctcatctctcgaatgtacagacttgtcggtaataggtgggatttaatagcaggaagagtcgtaggaagaaaggcaaatgagattgagagatactggattatgagaaactctgactatttttctcacaaacgacgacgtcttaataattctccctttttttctacttctcctcttaatctccaagaaaatctaaaattgtaaagaaatcaaaataaaagctttcaatcataaaagtagaacaaatcttgaatgtcttctca

SEQ ID NO: 142 - protein, 112 aa, Arabidopsis thaliana
  G226
MDNTNRLRLRRGPSLRQTKFTRSRYDSEEVSSIEWEFISMTEQEEDLISRMYRLVGNRWDLIAGRVVGRKANEIERYWIMRNSDYFSHKRRRLNNSPFFSTSPLNLQENLKL

SEQ ID NO: 143 - DNA, 1125 bp, Arabidopsis thaliana
  G464
ctccattctctctgtgtcatcaagcttcttttttgtgtgggttatttgaaagacactttctctgctggtatcattggagtctagggttttgttattgacatgcgtggtgtgtcagaattggaggtggggaagagtaatcttccggcggagagtgagctggaattgggattagggctcagcctcggtggtggcgcgtggaaagagcgtgggaggattcttactgctaaggattttccttccgttgggtctaaacgctctgctgaatcttcctctcaccaaggagcttctcctcctcgttcaagtcaagtggtaggatggccaccaattgggttacacaggatgaacagtttggttaataaccaagctatgaaggcagcaagagcggaagaaggagacggggagaagaaagttgtgaagaatgatgagctcaaagatgtgtcaatgaaggtgaatccgaaagttcagggcttagggtttgttaaggtgaatatggatggagttggtataggcagaaaagtggatatgagagctcattcgtcttacgaaaacttggctcagacgcttgaggaaatgttctttggaatgacaggtactacttgtcgagaaaaggttaaacctttaaggcttttagatggatcatcagactttgtactcacttatgaagataaggaaggggattggatgcttgttggagatgttccatggagaatgtttatcaactcggtgaaaaggcttcggatcatgggaacctcagaagctagtggactagctccaagacgtcaagagcagaaggatagacaaagaaacaaccctgtttagcttcccttccaaagctggcattgtttatgtattgtttgaggtttgcaatttactcgatactttttgaagaaagtattttggagaatatggataaaagcatgcagaagcttagatatgatttgaatccggttttcggatatggttttgcttaggtcattcaattcgtagttttccagtttgtttcttctttggctgtgtaccaattatctatgttctgtgagagaaagctcttgtttatttgttctctcagattgtaaatagttgaagttatctaattaatgtgataagagttatgtttatgattcc

SEQ ID NO: 144 - protein, 239 aa, Arabidopsis thaliana
  G464
MRGVSELEVGKSNLPAESELELGLGLSLGGGAWKERGRILTAKDFPSVGSKRSAESSSHQGASPPRSSQVVGWPPIGLHRMNSLVNNQAMKAARAEEGDGEKKVVKNDELKDVSMKVNPKVQGLGFVKVNMDGVGIGRKVDMRAHSSYENLAQTLEEMFFGMTGTTCREKVKPLRLLDGSSDFVLTYEDKEGDWMLVGDVPWRMFINSVKRLRIMGTSEASGLAPRRQEQKDRQRNNPV

SEQ ID NO: 145 - DNA, 1121 bp, Arabidopsis thaliana
  G263
tttttagttttatttttctgtggtaaaataaaaaaagttcgccggagatgacggctgtgacggcggcgcaaagatcagttccggcgccgtttttaagcaaaacgtatcagctagttgatgatcatagcacagacgacgtcgtttcatggaacgaagaaggaacagcttttgtcgtgtggaaaacagcagagtttgctaaagatcttcttcctcaatacttcaagcataataatttctcaagcttcattcgtcagctcaacacttacggatttcgtaaaactgtaccggataaatgggaatttgcaaacgattatttccggagaggcggggaggatctgttgacggacatacgacggcgtaaatcggtgattgcttcaacggcggggaaatgtgttgttgttggttcgccttctgagtctaattctggtggtggtgatgatcacggttcaagctccacgtcatcacccggttcgtcgaagaatcctggttcggtggagaacatggttgctgatttatcaggagagaacgagaagcttaaacgtgaaaacaataacttgagctcggagctcgcggcggcgaagaagcagcgcgatgagctagtgacgttcttgacgggtcatctgaaagtaagaccggaacaaatcgataaaatgatcaaaggagggaaatttaaaccggtggagtctgacgaagagagtgagtgcgaaggttgcgacggcggcggaggagcagaggagggggtaggtgaaggattgaaattgtttggggtgtggttgaaaggagagagaaaaaagagggaccgggatgaaaagaattatgtggtgagtgggtcccgtatgacggaaataaagaacgtggactttcacgcgccgttgtggaaaagcagcaaagtctgcaactaaaaaaagagtagaagactgttcaaaccagcgtgtgacacgtcatcgacgacgacgaaaaaaatgatttaaaaaactatttttttccgtaaggaagaaaagttatttttatgttttaaaaaggtgaagaaggtccagaaggatcaacgcaaatatataaatggattttcatgtattatataatttaattagtgtattaagaaaataaaacaaaaaaaaaaa

SEQ ID NO: 146 - protein, 284 aa, Arabidopsis thaliana
  G263
MTAVTAAQRSVPAPFLSKTYQLVDDHSTDDVVSWNEEGTAFVVWKTAEFAKDLLPQYFKHNNFSSFIRQLNTYGFRKTVPDKWEFANDYFRRGGEDLLTDIRRRKSVIASTAGKCVVVGSPSESNSGGGDDHGSSSTSSPGSSKNPGSVENMVADLSGENEKLKRENNNLSSELAAAKKQRDELVTFLTGHLKVRPEQIDKMIKGGKFKPVESDEESECEGCDGGGGAEEGVGEGLKLFGVWLKGERKKRDRDEKNYVVSGSRMTEIKNVDFHAPLWKSSKVCN

SEQ ID NO: 147 - DNA, 1409 bp, Arabidopsis thaliana
  G502
ttgatgccgctcaatcccactatccttcgcaaggacccttcctctatataaggaagttcatttcatttggagaggacacgctgacaagctgactctagcagatctgggaccgtcgacccacgcgtccgaattgattaggataggatcaggatcatcctcaacaacctcctcctaattcctcctccattcatagtaacaataatattaagaaagagggtaaactatgtcagaattattacagttgcctccaggtttccgatttcaccctaccgatgaagagcttgtcatgcactatctctgccgcaaatgtgcctctcagtccatcgccgttccgatcatcgctgagatcgatctctacaaatacgatccatgggagcttcctggtttagccttgtatggtgagaaggaatggtacttcttctctcccagggacagaaaatatcccaacggttcgcgtcctaaccggtccgctggttctggttactggaaagctaccggagctgataaaccgatcggactacctaaaccggtcggaattaagaaagctcttgttttctacgccggcaaagctccaaagggagagaaaaccaattggatcatgcacgagtaccgtctcgccgacgttgaccggtccgttcgcaagaagaagaatagtctcaggctggatgattgggttctctgccggatttacaacaaaaaaggagctaccgagaggcggggaccaccgcctccggttgtttacggcgacgaaatcatggaggagaagccgaaggtgacggagatggttatgcctccgccgccgcaacagacaagtgagttcgcgtatttcgacacgtcggattcggtgccgaagctgcatactacggattcgagttgctcggagcaggtggtgtcgccggagttcacgagcgaggttcagagcgagcccaagtggaaagattggtcggccgtaagtaatgacaataacaatacccttgattttgggtttaattacattgatgccaccgtggataacgcgtttggaggaggagggagtagtaatcagatgtttccgctacaggatatgttcatgtacatgcagaagccttactagaagggaattcctttcctgccgccgaaacgcaacgcaaaacgaccctcgtttttgcgtttatggcaacacgagaccgttttatatggtcaatgagtgtgccgattcggccattagatttctgttcagtcttcgtttattctatagaccgtccgatttcagatcatccctaatcggacggtggtcgttggatgtatcagtagtgtattactgtgttaggtagaagaaaatccacttgttcttaaattggcataaaagtcagaagctaatatttatatgtgccgcaatcaatttaatattttctgtctaaaaaaaaaaa

SEQ ID NO: 148 - protein, 319 aa, Arabidopsis thaliana
  G502
MSELLQLPPGFRFHPTDEELVMHYLCRKCASQSIAVPIIAEIDLYKYDPWELPGLALYGEKEWYFFSPRDRKYPNGSRPNRSAGSGYWKATGADKPIGLPKPVGIKKALVFYAGKAPKGEKTNWIMHEYRLADVDRSVRKKKNSLRLDDWVLCRIYNKKGATERRGPPPPVVYGDEIMEEKPKVTEMVMPPPPQQTSEFAYFDTSDSVPKLHTTDSSCSEQVVSPEFTSEVQSEPKWKDWSAVSNDNNNTLDFGFNYIDATVDNAFGGGGSSNQMFPLQDMFMYMQKPYKGIPFLPPKRNAKRPSFLRLWQHETVLYGQ

SEQ ID NO: 149 - DNA, 1454 bp, Arabidopsis thaliana
  G509
cttcctcctttgctaataaacttttctttgaaccttacacgccttgttgatattactctcttaaatatatattttcgtacattaacacagacatatataaagctaaagatttcttcacgtaatgggtttgaaagatattgggtccaaattgccaccggggtttcgatttcatccaagtgatgaagagttggtttgtcattatctttgcaacaagattagggccaaatctgatcatggtgatgttgatgatgatgatgatgatgttgatgaagctttgaagggttctactgatcttgtggagattgacttgcatatctgtgagccatgggagcttcctgatgtggcaaagttaaacgcaaaggaatggtacttcttcagtttccgtgatcgaaagtatgctactggatatcgcacgaacagagcgacagtaagcggatactggaaagcaacaggaaaagatcgaacggtgatggatccacgtacaaggcaattggtagggatgagaaaaacactagtgttctacagaaacagagcaccaaatgggatcaaaactacttggatcatgcacgagttccgtcttgagtgtcctaacatcccacataaggaagactgggtcttgtgcagagtgttcaacaaaggcagagactcatcgctacaagacaataattattataacaatgataatcagacgcaaaggcttgaagttaatgacgctccggatcttaattacaacaatcagttgccacctttgctatcatcccctcctcataatcatcaacatgagaagatgaaaatccaagtttgtgatcagtgggagcagctaatgaagcagccttcaaggaccaccggccacccctatcatcaccattgtcatcatcaaaccatagcatgtggttgggagcagatgatgatcggttcgctgtcatcaccttcgagtcatggccctgatcacgagtcctttgctaaatttgctttaccgtcgacaataacaacagtgtcaacatcagtggtgatcatcatcagaattatgagaagattttgttgtcatcactagacatgacgagtttggatcacgacaagacatgtatgggatcatcatcggatggtggtatggtctctgatcttcacatggaatgtggtggattgagttttgagaccgaaaatatcctcgctttccaatgaacataattcaaggggttcgccaatttgttgattcgtgaattatacaaacattttatctatagatttatcacattatcaaacatgtaagttgtgtggcatttgggtatagggtttgtgtgattctaggtttttaggacgatgtatgttgttatatttagcgtgtttttaggatttattctcattttaaaattatatgaaaacccattactatgaatacaattagttttctttgttgtaaataatattttagattatcaaaaaaaaaaaaaaaa

SEQ ID NO: 150 - protein, 310 aa, Arabidopsis thaliana
  G509
MGLKDIGSKLPPGFRFHPSDEELVCHYLCNKIRAKSDHGDVDDDDDDVDEALKGSTDLVEIDLHICEPWELPDVAKLNAKEWYFFSFRDRKYATGYRTNRATVSGYWKATGKDRTVMDPRTRQLVGMRKTLVFYRNRAPNGIKTTWIMHEFRLECPNIPHKEDWVLCRVFNKGRDSSLQDNNYYNNDNQTQRLEVNDAPDLNYNNQLPPLLSSPPHNHQHEKMKIQVCDQWEQLMKQPSRTTGHPYHHHCHHQTIACGWEQMMIGSLSSPSSHGPDHESFAKFALPSTITTVSTSVVIIIRIMRRFCCHH

SEQ ID NO: 151 - DNA, 1217 bp, Arabidopsis thaliana
  G514
tccaagatcaaatcaagaaacccattaaaaaaaaaaatcaggttttggtttcagttttaagggtttaaggtttcttggggaagaaacgatggagactttttgtgggtttcaaaaggaggaagagcagatggatttacctcctgggttcaggtttcatccaacagatgaagaactcataactcactatctccataataaggttcttgacaccagcttctcagctaaagctatcggtgaagttgatttaaacaaatcagagccatgggagttaccatggatggcaaaaatgggtgagaaagaatggtattttttctgtgtgagagacagaaagtatcccaccggtttaagaactaaccgagcaactgaagccggttattggaaggcgaccgggaaggataaagagatataccgaggcaaatcacttgttgggatgaagaagacacttgttttctatagaggaagagctcctaaaggtcagaaaaccaactgggtgatgcatgagtacaggcttgaaggaaaattctctgcccataacttgccgaaaaccgcaaagaagtgggtatgcagggtgttccaaaagagtgctggagggaagaagatcccgatttcgagtctaatccgaatcggttcactcggaaccgactttaacccttcgcttttgccctctttaaccgattcttcgccttacaacgataaaaccaaaacagaaccggtctacgtgccctgcttctccaaccaaacggatcaaaaccaaggaaccacactcaattgcttcagcagccctgttcttaactcgatccaagccgacatttttcacaggattccactctatcaaactcagtccctccaggtttctatgaatctacagagcccggttctcacgcaagaacactcagttctacatgctatgatcgagaacaacagaagacaaagtctcaaaacgatgagtgtctcacaagaaaccggagtttcaactgacatgaacactgatatctcatcggattttgaatttggtaagagacggtttgattctcaagaagatccgtcttcctctactggaccggttgatcttgaacctttctggaattactgaagatgattcaagattctcatgtccattaatttactgtggtgtgttaaagtttgtataggctattgtcatatactctcatatcaacttccactatatattataacaaattaaagaaacttaaaaa

SEQ ID NO: 152 - protein, 334 aa, Arabidopsis thaliana
  G514
METFCGFQKEEEQMDLPPGFRFHPTDEELITHYLHNKVLDTSFSAKAIGEVDLNKSEPWELPWMAKMGEKEWYFFCVRDRKYPTGLRTNRATEAGYWKATGKDKEIYRGKSLVGMKKTLVFYRGRAPKGQKTNWVMHEYRLEGKFSAHNLPKTAKKWVCRVFQKSAGGKKIPISSLIRIGSLGTDFNPSLLPSLTDSSPYNDKTKTEPVYVPCFSNQTDQNQGTTLNCFSSPVLNSIQADIFHRIPLYQTQSLQVSMNLQSPVLTQEHSVLHAMIENNRRQSLKTMSVSQETGVSTDMNTDISSDFEFGKRRFDSQEDPSSSTGPVDLEPFWNY

SEQ ID NO: 153 - DNA, 1529 bp, Arabidopsis thaliana
  G553
ttgatcggaatattccttttagaatgccaagattcattcttcatctctcgagctcctcatgaatttcttctctgcttatcaatggagatgatgagctcttcttcttctactactcaagttgtatcattcagagacatggggatgtatgaaccatttcaacagttatctggttgggagagtcctttcaaatcagatatcaacaatattactagtaatcagaataacaatcagagttcttcaacaacacttgaggttgatgctagaccagaagcagatgataacaatagagtgaattatacttctgtgtataataactctcttgaagcagaaccgtcgagtaataatgatcaggacgaagaccggatcaatgataagatgaaacggcgtttggctcagaaccgagaagctgctcgcaaaagtcgtttgagaaagaaggcccatgttcaacagttagaagaaagccggttgaagttgtcacagctcgagcaggaacttgtaagagctaggcagcagggattatgcgtacgcaattcttcagatactagttatctaggaccagctgggaatatgaactcaggtattgctgcatttgagatggaatacacacactggctagaagagcaaaacaggagagttagtgagattcgaacagcgctccaagctcatataggtgacattgagctcaaaatgttggtagatagttgcttgaaccactacgcaaatctcttccgcatgaaagctgatgctgcaaaggctgatgtgttcttcttgatgtcgggaatgtggcgaacttcaactgaacgcttcttccaatggattggaggtttccgcccatccgagcttttaaatgttgtgatgccatacgttgagcccttaaccgatcagcaactgttggaggtgcgtaacctgcaacagtcgtctcagcaagcagaggaggctctctctcaaggcttagataaacttcagcagggtttggtcgaaagcatagcaattcagataaaagttgttgagtctgtgaatcacggggctccaatggcttcagccatggagaatcttcaagcattggagagttttgtgaaccaggcggatcatctgagacaacagaccctgcagcaaatgagtaagatattgacgacaagacaggctgctcgagggttgctcgctctaggagaatacttccacaggctgcgtgctcttagttctctctgggcagctcgtccacgagaacacacttgacaagttaggaggcaaacaaaacaaagagaaagaagcacaaggcagatgatgttagctatagggacttgctttatctctcagaaagtgttggctgatattttctccattagagagcatcatcttcctcattgatgattttgtttacttgaaaggaataagagatgtgtaaatttgggtggaaaacatgtaatgtctttgatgcattaggctctttatttgtaaaatatatagggtttgttgtcactcatctcctcgtatatgaaaatttgagcccacaatcaaattttttgtct

SEQ ID NO: 154 - protein, 384 aa, Arabidopsis thaliana
  G553
MEMMSSSSSTTQVVSFRDMGMYEPFQQLSGWESPFKSDINNITSNQNNNQSSSTTLEVDARPEADDNNRVNYTSVYNNSLEAEPSSNNDQDEDRINDKMKRRLAQNREAARKSRLRKKAHVQQLEESRLKLSQLEQELVRARQQGLCVRNSSDTSYLGPAGNMNSGIAAFEMEYTHWLEEQNRRVSEIRTALQAHIGDIELKMLVDSCLNHYANLFRMKADAAKADVFFLMSGMWRTSTERFFQWIGGFRPSELLNVVMPYVEPLTDQQLLEVRNLQQSSQQAEEALSQGLDKLQQGLVESIAIQIKVVESVNHGAPMASAMENLQALESFVNQADHLRQQTLQQMSKILTTRQAARGLLALGEYFHRLRALSSLWAARPREHT

SEQ ID NO: 155 - DNA, 1104 bp, Arabidopsis thaliana
  G572
aggtaaccatggtccacaattgtccccctactagtctatatgaacacctcttctcaccacacacaccaaacattcatcacaattccctaaatcctacaatctttcttttcttccatcacctaagagtaaaccctaccttgcgtcccaagtctccaccgataatgcaaccatccaccaacatttttagcctccatggttgtccaccttcgtacctttcccatattcctacatcctcaccattttgcggacaaaaccctaatccattcttcagcttcgaaactggcgtgaacacttcacagttcatgagtttgatcagcagcaacaactcgacctcagatgaagcagaagagaatcataaggagataataaatgagaggaagcaaaagaggaagatatctaacagagaatcggcaagaagatcgcgtatgaggaagcagagacaagtggatgagctttggtcacaggtgatgtggcttagggatgagaatcatcagctgcttcgtaagcttaactgcgttttagagtctcaagagaaggttattgaagagaatgttcagctcaaagaggaaaccactgagcttaagcaaatgattagtgatatgcagcttcagaatcagagtcctttctcttgcatcagagacgacgacgacgttgtatagatgatttttatttttatttttttggggctctagtatgtatcaacaaatgtatatccagagatctgtctcagcttaagatttaattaagactctaaattagtaattgttaaagcagagttcttcaaacttgtgttgcccgtgaatagaagtaagaaattgtttcacaattgtttgcataacaaacgataaaaattcacattgctgaagtataattttaaaaaaaattacatgcataaatatttttgcaaaatgtaaatacaagcatcaattactaacagtagactttcttctttttctctttaaaattttacgtttataatgaccaattttgttctttctaaattggcttaagtagtgtctttgaaaaaaatatacaccagccgtaaacggtttattttcttccatttccatttgcatttttatgttgaaaaattattaagaaaag

SEQ ID NO: 156 - protein, 216 aa, Arabidopsis thaliana
  G572
MVHNCPPTSLYEHLFSPHTPNIHHNSLNPTIFLFFHHLRVNPTLRPKSPPIMQPSTNIFSLHGCPPSYLSHIPTSSPFCGQNPNPFFSFETGVNTSQFMSLISSNNSTSDEAEENHKEIINERKQKRKISNRESARRSRMRKQRQVDELWSQVMWLRDENHQLLRKLNCVLESQEKVIEENVQLKEETTELKQMISDMQLQNQSPFSCIRDDDDVV

SEQ ID NO: 157 - DNA, 1553 bp, Arabidopsis thaliana
  G632
ctttttttcattttgttctgagggggcttttgtgttgacggtggtgggaacaaaggaacatcgtatccattctgtccgggaaacacagcaatgttcatttccgacaaatctcgtcctactgatttctacaaagacgatcatcacaattcctccaccaccagcaccacacgcgatatgatgatcgatgtactcaccactaccaacgaatcagtagatctacaatctcaccaccaccacaatcaccacaatcatcatctccaccaatctcagccacaacaacagattctcctcggagaaagcagtggagaagatcacgaagttaaagcaccaaagaaacgagcggagacatgggttcaagacgaaactcgtagcttaatcatgttccgtagaggtatggatggtttattcaatacatccaaatctaataaacatctctgggaacagatttcgtctaagatgagagaaaaagggtttgatcgatctccgactatgtgtactgataaatggaggaatctgttgaaagagtttaagaaagctaagcatcatgatagaggaaatggatcggcgaagatgtcgtattacaaagagattgaagatattcttagagagaggagcaaaaaagtgacaccaccacagtataataagagccctaatacaccacctacatcagccaaagttgattcctttatgcaatttactgataaaggttttgatgatacgagcatttcttttggatccgttgaagctaatggcaggccagccttaaaccttgaaaggcgtcttgatcatgatggtcatcctcttgcaatcactacagcagttgatgctgttgcagcaaatggagttactccttggaattggagagagactcctggaaacggtgatgatagtcatggtcagccttttggtggtagggtcataacagtgaaatttggtgactatacaagaagaatcggtgttgatggtagtgcagaagcaatcaaagaggtaatcagatctgcttttgggttaagaactcgaagggctttttggttagaagatgaagatcagattattcgctgtcttgaccgagacatgcccttagggaactatctactccgtctggatgatggactggccattagggtttgccattatgatgaatccaaccaattaccagtccattcagaagagaaaatcttctacactgaagaagactaccgcgagtttctggctctacagggatggtcaagcctgcaagttgatggttttaggaacatagaaaacatggatgatcttcaacctggtgctgtgtatcgaggtgtgagatgaggaaatgagatcacaaaacttcttctccaattctatcaacagttatctgaacagaaaacagtcccctgtagatatgatctcattctctttatacatttcttctttttttcatgtacttgctcaaatatgaaatatcataactggcatttaccgtacagaccaagaagacctcaaaatagatttttggtctgttacatttgtaagaacagaacataagagaagtgaataaattgttcacaatc

SEQ ID NO: 158 - protein, 406 aa, Arabidopsis thaliana
  G632
MFISDKSRPTDFYKDDHHNSSTTSTTRDMMIDVLTTTNESVDLQSHHHHNHHNHHLHQSQPQQQILLGESSGEDHEVKAPKKRAETWVQDETRSLIMFRRGMDGLFNTSKSNKHLWEQISSKMREKGFDRSPTMCTDKWRNLLKEFKKAKHHDRGNGSAKMSYYKEIEDILRERSKKVTPPQYNKSPNTPPTSAKVDSFMQFTDKGFDDTSISFGSVEANGRPALNLERRLDHDGHPLAITTAVDAVAANGVTPWNWRETPGNGDDSHGQPFGGRVITVKFGDYTRRIGVDGSAEAIKEVIRSAFGLRTRRAFWLEDEDQIIRCLDRDMPLGNYLLRLDDGLAIRVCHYDESNQLPVHSEEKIFYTEEDYREFLALQGWSSLQVDGFRNIENMDDLQPGAVYRGVR

SEQ ID NO: 159 - DNA, 859 bp, Arabidopsis thaliana
  G7
gtgactctaacgaagaaaccggcaatggccagtatcactacaatgccgaagaaataacaagaatcataaacgagccagaatattatcccccgggttacaacttgtctaccaccgcaatttcaaacatggtgtctatgctgactaatgttgtctctggtgagaccgaaccctcggcatctgcgacatggacgatgggtcataagagagaaagagaagagttttctttgcctcctcaaccattgattaccggttcagctgtgactaaagaatgtgaaagctcaatgtccttggagaggccaaaaaaatatagaggagtaaggcaacgaccatggggaaaatgggcggcggagattcgagacccacacaaggcgacacgtgtatggcttgggacattcgagacagccgaggccgccgcaagagcctatgatgcggcagcacttcgctttagaggaagcaaagcaaagcttaatttccccgaaaatgttggaactcagacgattcaacgaaattctcatttcttgcaaaactcaatgcaaccttctctaacatacatcgatcaatgtccaactctattatcttactctcgatgtatggagcaacaacaaccattagtaggcatgttgcagccaacagaagaggaaaatcactttttcgaaaaaccatggaccgaatatgatcaatacaattactcctcttttggttaactaacatatcgtcaacgctttgtatttctacttattcgatctaccaattttttctctcccaatacaacttcagtctgattattgccttcttagatatgtcttcgaatgttatgactatacatgggtgtatataaaatttgtgatcaaagtcttgt

SEQ ID NO: 160 - protein, 192 aa, Arabidopsis thaliana
  G7
MVSMLTNVVSGETEPSASATWTMGHKREREEFSLPPQPLITGSAVTKECESSMSLERPKKYRGVRQRPWGKWAAEIRDPHKATRVWLGTFETAEAAARAYDAAALRFRGSKAKLNFPENVGTQTIQRNSHFLQNSMQPSLTYIDQCPTLLSYSRCMEQQQPLVGMLQPTEEENHFFEKPWTEYDQYNYSSFG

SEQ ID NO: 161 - DNA, 1221 bp, Arabidopsis thaliana
  G725
cctctttcagagagagaaagagagtcagagagagagagagagagaatgttccatgctaagaaaccttcaagtatgaatggttcatatgagaacagagctatgtgcgttcaaggcgattcaggccttgtcctcaccaccgaccctaaaccgcgtttgcgttggaccgtcgaactccacgagcgttttgtggacgccgtcgctcagctcggcggccccgacaaagcgaccccaaagacgattatgagagttatgggtgtgaagggtcttactctttaccacctaaagagccatcttcagaaattcaggcttggaaagcagccgcacaaggagtacggagatcactccacaaaggaaggttcaagagcttctgccatggatattcagcgcaacgtagcttcttcttctggcatgatgagtcgcaacatgaatgagatgcaaatggaagtgcagagaaggttgcatgaacagctagaggtgcaaagacatctgcaactgaggattgaagcacaaggaaagtacatgcaatctatcttggagagagcttgccaaaccctagccggtgagaacatggcagccgccaccgcagcagccgccgtcggaggaggatacaagggtaatctgggaagttcgagtctttcagcagcggtgggcccacctcctcatcctcttagtttcccgccgtttcaagacctaaacatctatggaaacacaaccgaccaagtcctcgaccatcacaacttccatcatcaaaacatagagaaccatttcacgggtaacaatgctgcagacaccaacatttacttggggaagaagcgacctaatcctaattttggtaacgatgtaaggaaaggactattgatgtggtctgatcaagatcacgatctttccgcaaaccaatcgatcgatgatgagcatagaattcagatacagatggctacacatgtctccacggatttggattctttgtcggagatctacgaaaggaaatcaggtttatcaggtgatgaagggaataatggtgggaaattactggaaaggccatcgcctaggagatcaccattgagtcctatgatgaaccctaatggtggattaatacaaggaagaaactcgccatttgggtgatacaatttattaatttttatctatgagtgatgcatgggaatgtaagaacgagatatatatgttttgtcattgtgagtttgacgtagggtttagagaaaa

SEQ ID NO: 162 - protein, 358 aa, Arabidopsis thaliana
  G725
MFHAKKPSSMNGSYENRAMCVQGDSGLVLTTDPKPRLRWTVELHERFVDAVAQLGGPDKATPKTIMRVMGVKGLTLYHLKSHLQKFRLGKQPHKEYGDHSTKEGSRASAMDIQRNVASSSGMMSRNMNEMQMEVQRRLHEQLEVQRHLQLRIEAQGKYMQSILERACQTLAGENMAAATAAAAVGGGYKGNLGSSSLSAAVGPPPHPLSFPPFQDLNIYGNTTDQVLDHHNFHHQNIENHFTGNNAADTNIYLGKKRPNPNFGNDVRKGLLMWSDQDHDLSANQSIDDEHRIQIQMATHVSTDLDSLSEIYERKSGLSGDEGNNGGKLLERPSPRRSPLSPMMNPNGGLIQGRNSPFG

SEQ ID NO: 163 - DNA, 2001 bp, Arabidopsis thaliana
  G760
tgcttaattccaatgccatcgtgatcgattcatctctctctctctcttccaattttcccaattcttttttaaaaccctaatttttcagatatctgattatctcttgtatttcttctactcgatttgctcccataaaaacccttactttcttcaagttctggttttcaccgattgatgggtcgtggctcagtgacgtcgcttgctcctgggttccgttttcacccgacggatgaggaacttgttcgctactaccttaagcgtaaggtctgcaacaaaccctttaagttcgatgctatttccgtcaccgacatatacaagtctgagccttgggatctaccagataagtcgaagctgaaaagtagagacttggaatggtacttctttagtatgctggataagaagtacagtaatggttccaagacgaatcgtgctacggagaaagggtattggaagacgactgggaaagatcgggagattcgtaatggttcaagagtcgttgggatgaagaagacacttgtttatcacaagggtcgagctcctcgtggtgaaaggaccaattgggttatgcatgagtatcggctttctgatgaggacttgaagaaagctggtgtgccacaagaagcatatgtgttatgtaggatattccagaaaagtggtacgggtcctaagaatggggagcagtatggtgctccttatcttgaggaggagtgggaagaagatggaatgacttatgtacctgctcaagatgctttcagtgaaggattggctttgaatgatgatgtttatgtcgatattgatgacattgacgagaagcccgaaaatctggtggtctatgatgccgttcctattctacctaactattgtcatggggaatcaagtaacaatgttgaatcaggcaattactcagactctggaaattacattcaaccaggaaacaatgttgtcgactctggtgggtactttgaacaaccaattgaaacttttgaggaagatcggaagcctattatacgggagggtagcattcagccttgttctctgtttccagaggaacaaattggctgtggtgtgcaagacgaaaatgtggtgaatctggaatcttccaacaataatgtgtttgtagctgatacatgctacagtgacattcctattgatcataactatttacccgatgagccattcatggatcctaataacaatcttccactcaacgatggtctgtacctggaaacgaatgatctcagctgtgctcaacaagatgattttaacttcgaagattatctcagcttctttgatgatgagggtttgacttttgacgattctctattaatgggacctgaagattttcttcccaaccaagaagcccttgaccagaaacctgcccctaaagaattggagaaggaggtcgcaggaggcaaagaggcagtggaggaaaaggaaagtggcgaaggatcttcttcaaaacaagatacagatttcaaggactttgattcagctccgaagtacccatttctcaaaaagacgagccacatgcttggagccattcctactccatcttcatttgcttcacagttccaaacaaaggacgcaatgcgtctacacgcagcacaatcttctggttcagttcacgtgactgcaggtatgatgagaatatcaaacatgactctagcagcggacagcggtatgggctggtcatatgacaagaacggtaacctcaacgtagtcctttcattcggggtagtccaacaggatgatgcgatgactgcctcgggaagcaagacaggaattacggcgacaagagctatgttagtcttcatgtgtttatgggttctcctactctctgttagcttcaaaatagtaaccatggtgtctgctcggtaataggatcaaagttgaatcgtctcaaagactttttttggtgtttgtacctctccaatcatatagcctttaactttggcagtgctttgctgctcaatatttaaattttaaaaaaaaaaaaaaaaa

SEQ ID NO: 164 - protein, 567 aa, Arabidopsis thaliana
  G760
MGRGSVTSLAPGFRFHPTDEELVRYYLKRKVCNKPFKFDAISVTDIYKSEPWDLPDKSKLKSRDLEWYFFSMLDKKYSNGSKTNRATEKGYWKTTGKDREIRNGSRVVGMKKTLVYHKGRAPRGERTNWVMHEYRLSDEDLKKAGVPQEAYVLCRIFQKSGTGPKNGEQYGAPYLEEEWEEDGMTYVPAQDAFSEGLALNDDVYVDIDDIDEKPENLVVYDAVPILPNYCHGESSNNVESGNYSDSGNYIQPGNNVVDSGGYFEQPIETFEEDRKPIIREGSIQPCSLFPEEQIGCGVQDENVVNLESSNNNVFVADTCYSDIPIDHNYLPDEPFMDPNNNLPLNDGLYLETNDLSCAQQDDFNFEDYLSFFDDEGLTFDDSLLMGPEDFLPNQEALDQKPAPKELEKEVAGGKEAVEEKESGEGSSSKQDTDFKDFDSAPKYPFLKKTSHMLGAIPTPSSFASQFQTKDAMRLHAAQSSGSVHVTAGMMRISNMTLAADSGMGWSYDKNGNLNVVLSFGVVQQDDAMTASGSKTGITATRAMLVFMCLWVLLLSVSFKIVTMVSAR

SEQ ID NO: 165 - DNA, 1213 bp, Arabidopsis thaliana
  G770
ccttcctctatataaggaagttcatttcatttggagaggacacgctgacaagctgactctagcagatctggtaccgtcgacggttcttggatttggagtaaactaaagatcatataaaatggaacaaggagatcatcagcagcataagaaagaagaagaagctttgccaccgggtttcagatttcatccgacggatgaggagctaatctcatattacttggttaataagattgccgatcaaaacttcaccgggaaagcaatcgctgacgttgatcttaacaagtccgagccatgggagcttcctgagaaggcgaaaatgggaggaaaagaatggtacttttttagcctccgggaccggaagtacccgacgggagtgaggacgaatagggcgacgaatacaggatattggaaaaccacaggaaaagacaaagagatattcaatagcacaacctcggagttggtcgggatgaagaagactttggtcttttacagaggacgagctcctcgtggggagaagacttgttgggtcatgcatgagtatcgacttcactccaagtcctcatatagaacctccaagcaagacgagtgggtagtgtgtagagtgttcaagaaaacagaagcaaccaagaaatacataagcaccagtagcagcagcacaagtcatcaccacaacaaccacacaagagcctcaatactatcaaccaacaacaataatcctaattactcatcagacctccttcaactcccaccgcatctacaaccacacccgagcctcaatattaaccaatccctcatggcaaacgccgttcacctagctgagctctcaagagtcttccgtgcctctacaagcaccaccatggactcttctcatcagcagctaatgaactacacccacatgcctgtctcagggctcaacctcaaccttggcggtgcactggtccagccgcctcctgttgtgtctcttgaggatgttgccgcggttagtgcttcgtacaatggcgaaaacgggtttggaaatgtggagatgagccagtgcatggacttggatggatactggccatcttattgattggtaattgtcagtttaagttatggtttttatattgtttccatttacttgttggtaaaacgattttggttgttcttgcgaacgctctagacaggcctcgtaccggatcctctagctagagctttcgttcgtatcatcggtttc

SEQ ID NO: 166 - protein, 316 aa, Arabidopsis thaliana
  G770
MEQGDHQQHKKEEEALPPGFRFHPTDEELISYYLVNKIADQNFTGKAIADVDLNKSEPWELPEKAKMGGKEWYFFSLRDRKYPTGVRTNRATNTGYWKTTGKDKEIFNSTTSELVGMKKTLVFYRGRAPRGEKTCWVMHEYRLHSKSSYRTSKQDEWVVCRVFKKTEATKKYISTSSSSTSHHHNNHTRASILSTNNNNPNYSSDLLQLPPHLQPHPSLNINQSLMANAVHLAELSRVFRASTSTTMDSSHQQLMNYTHMPVSGLNLNLGGALVQPPPVVSLEDVAAVSASYNGENGFGNVEMSQCMDLDGYWPSY

SEQ ID NO: 167 - DNA, 1389 bp, Arabidopsis thaliana
  G778
tctcaataacacaaaaccttttaaactagtaaaatacacagattttaggatgagccaatgtgttccaaactgtcacatcgatgatactccggcagcagccaccaccaccgtccgctccaccacagccgcagacatccccatattagactacgaggtagccgagctgacgtgggagaacgggcaactaggcttgcacggcttaggtccaccgcgagtgacggcttcgtcgaccaagtactccacaggcgccggtggaacgttggagtcgatagtggaccaagctactcgcctccctaaccctaagcccacggatgagctcgtcccgtggttccatcatcgctcctccagggccgcgatggcaatggacgcgcttgtcccttgctccaacctagtacacgagcagcagagcaagcctggtggcgttggctccacccgggtggggtcatgtagcgatggtcgtaccatgggcggtggaaaacgagcaagagtggcaccggagtggagcggcggcgggagtcagcggctgaccatggacacttacgacgtaggtttcacctcaacatcaatgggctcgcacgataacacaatcgacgatcatgactccgtctgccacagccgcccacagatggaggacgaagaagagaagaaagccggaggaaaatcatcagtttcaaccaagagaagcagagctgctgctattcataaccaatccgaacgtaagaggagagataaaatcaatcaaaggatgaagactttgcaaaaactggttcccaattccagcaagacggataaagcatctatgttggatgaagtgatagagtatttgaagcaacttcaagcacaagtgagcatgatgagcagaatgaatatgccttctatgatgcttcctatggccatgcagcaacaacaacaactacaaatgtctctcatgtccaatcccatgggtttagggatgggcatggggatgcccggtctcggtctcctcgaccttaattctatgaaccgagctgctgcaagcgctcctaatatccatgccaacatgatgccaaacccatttttgcccatgaattgtccatcgtgggatgcttcttccaatgactctcgatttcagtctcctctcatccccgatcctatgtctgcctttcttgcatgctctactcagccaacgacgatggaagcgtatagcaggatggctacattatatcagcaaatgcaacaacaacttcctcctccttcgaatccaaaatgattattactcaaacacctctatatagtttacgtctatatatgtgttagtcacatacatacatatatatattccatcataattatttatttatatgtataggcttctcatgaattatgatattatacgtattacgtaaaaaa

SEQ ID NO: 168 - protein, 399 aa, Arabidopsis thaliana
  G778
MSQCVPNCHIDDTPAAATTTVRSTTAADIPILDYEVAELTWENGQLGLHGLGPPRVTASSTKYSTGAGGTLESIVDQATRLPNPKPTDELVPWFHHRSSRAAMAMDALVPCSNLVHEQQSKPGGVGSTRVGSCSDGRTMGGGKRARVAPEWSGGGSQRLTMDTYDVGFTSTSMGSHDNTIDDHDSVCHSRPQMEDEEEKKAGGKSSVSTKRSRAAAIHNQSERKRRDKINQRMKTLQKLVPNSSKTDKASMLDEVIEYLKQLQAQVSMMSRMNMPSMMLPMAMQQQQQLQMSLMSNPMGLGMGMGMPGLGLLDLNSMNRAAASAPNIHANMMPNPFLPMNCPSWDASSNDSRFQSPLIPDPMSAFLACSTQPTTMEAYSRMATLYQQMQQQLPPPSNPK

SEQ ID NO: 169 - DNA, 1152 bp, Arabidopsis thaliana
  G881
gtgaccgaagaaagcaaattgagactacgcaccaactagtcctttggtttgtatcttaagataaaggtttcttttatggacggttcttcgtttctcgacatctctctcgatctcaacaccaatcctttctccgcaaaacttccgaagaaggaggtctcagttttggcttctactcacttaaagaggaaatggttggagcaagacgagagcgcaagtgagttacgagaggagctaaacagagttaattcagagaacaagaagctaacagagatgttagctagagtctgtgagagctacaacgaactacataatcatttggagaagcttcagagtcgccagagccctgaaatcgagcagaccgatataccgataaagaaaagaaaacaagacccggatgagttcttaggctttcctattggactcagtagtggaaaaactgagaacagctccagcaacgaagatcatcatcatcatcatcagcaacatgagcagaaaaatcagcttctttcatgtaaaagaccagtcactgatagcttcaacaaagcaaaagtttcgactgtctacgtgcctactgaaacatcggacacaagcttgacagttaaagatggatttcaatggaggaaatacggacaaaaggttacaagagacaacccgtcacctagagcttactttagatgctcgtttgcaccgtcttgtccagtaaaaaagaaggtacaacgcagcgcagaggatccatctttacttgtagcgacatacgaagggacgcataaccacttgggtccaaatgcttctgaaggggatgctacaagccagggtgggtcaagcacagtgactttggatctggttaatggctgtcatagactagcgttggagaaaaacgaaagggataatacgatgcaagaggttctgattcaacaaatggcgtcatcgttaacaaaagattcgaaatttacagctgctcttgctgctgctatatctgggaggttaatggagcaatctagaacatgaacgtttttagtgaatgtattgtttttgtttgtttagaatgattcttcgttttcgaattgtgtctttcgattaggagataaaagatgtatataaatattataagtagatgaagaaatcgtataagtaaaaaaaaaaaaaaaaaaa

SEQ ID NO: 170 - protein, 310 aa, Arabidopsis thaliana
  G881
MDGSSFLDISLDLNTNPFSAKLPKKEVSVLASTHLKRKWLEQDESASELREELNRVNSENKKLTEMLARVCESYNELHNHLEKLQSRQSPEIEQTDIPIKKRKQDPDEFLGFPIGLSSGKTENSSSNEDHHHHHQQHEQKNQLLSCKRPVTDSFNKAKVSTVYVPTETSDTSLTVKDGFQWRKYGQKVTRDNPSPRAYFRCSFAPSCPVKKKVQRSAEDPSLLVATYEGTHNHLGPNASEGDATSQGGSSTVTLDLVNGCHRLALEKNERDNTMQEVLIQQMASSLTKDSKFTAALAAAISGRLMEQSRT

SEQ ID NO: 171 - DNA, 1246 bp, Arabidopsis thaliana
  G9
gtgtttcttctttctgctaaaaggttataatttttgtttcttggtttggtgagaatcttcaagaaactgaaacaaagaaaatggattctagttgcatagacgagataagttcctccacttcagaatctttctccgccaccaccgccaagaagctctctcctcctcccgcggcggcgttacgcctctaccggatgggaagcggcgggagcagcgtcgtgttggatcccgagaacggcctagagacggagtcacgaaagctaccatcttcaaaatacaaaggtgttgttcctcagcctaacggaagatggggagctcagatctacgagaagcaccaacgagtatggctcgggactttcaacgagcaagaagaagctgctcgttcctacgacatcgcagcttgtagattccgtggccgcgacgccgtcgtcaacttcaagaacgttctggaagacggcgatttagcttttcttgaagctcactcaaaggccgagatcgtcgacatgttgagaaaacacacttacgccgacgagcttgaacagaacaataaacggcagttgtttctctccgtcgacgctaacggaaaacgtaacggatcgagtactactcaaaacgacaaagttttaaagacgtgtgaagttcttttcgagaaggctgttacacctagcgacgttgggaagctaaaccgtctcgtgatacctaaacaacacgccgagaaacactttccgttaccgtcaccgtcaccggcagtgactaaaggagttttgatcaacttcgaagacgttaacggtaaagtgtggaggttccgttactcatactggaacagtagtcaaagttacgtgttgaccaagggatggagtcgattcgtcaaggagaagaatcttcgagccggtgatgttgttactttcgagagatcgaccggactagagcggcagttatatattgattggaaagttcggtctggtccgagagaaaacccggttcaggtggtggttcggcttttcggagttgatatctttaatgtgaccaccgtgaagccaaacgacgtcgtggccgtttgcggtggaaagagatctcgagatgttgatgatatgtttgcgttacggtgttccaagaagcaggcgataatcaatgctttgtgacatatttccttttccgattttatgctttcgttttttaatttttttttttgtcaagttgtgtaggttgtgattcatgctaggttgtatttaggaaaagagataagacc

SEQ ID NO: 172 - protein, 352 aa, Arabidopsis thaliana
  G9
MDSSCIDEISSSTSESFSATTAKKLSPPPAAALRLYRMGSGGSSVVLDPENGLETESRKLPSSKYKGVVPQPNGRWGAQIYEKHQRVWLGTFNEQEEAARSYDIAACRFRGRDAVVNFKNVLEDGDLAFLEAHSKAEIVDMLRKHTYADELEQNNKRQLFLSVDANGKRNGSSTTQNDKVLKTCEVLFEKAVTPSDVGKLNRLVIPKQHAEKHFPLPSPSPAVTKGVLINFEDVNGKVWRFRYSYWNSSQSYVLTKGWSRFVKEKNLRAGDVVTFERSTGLERQLYIDWKVRSGPRENPVQVVVRLFGVDIFNVTTVKPNDVVAVCGGKRSRDVDDMFALRCSKKQAIINAL

SEQ ID NO: 173 - DNA, 534 bp, Arabidopsis thaliana
  G437
atggaaggtgggtctgaaaaaacaacacctgaaggttgtggtggtgagagtaagtctaaacggaaaatgaaaactgctgctcaacttgaagttcttgaaaacacttattcagctgagccttatccttcggaagctataagagcggatctctcagtgaaactgaatctttccgatagacagttacagatgtggttttgtcaccggcggcttaaagaacggaaatctactacgccgagcaaacgtcagcgtaaggagttagtaactccaacggctatggaatcctgggaaccacccgtcaatgccggtgatttagtggcgggaaatgagcttgattctagaagagctgctcgaggcagtggcggtagtggtgtgacggttgtgaggcggtttaatgaaccgtcttctgctgaggttagagctattggttatgttgaagctcaattgggagagcggttgagagataacggaccggttcttggaatggagtttgatcctttacctcctggtgcatttggcatgcctattggtatataa

SEQ ID NO: 174 - protein, 177 aa, Arabidopsis thaliana
  G437
MEGGSEKTTPEGCGGESKSKRKMKTAAQLEVLENTYSAEPYPSEAIRADLSVKLNLSDRQLQMWFCHRRLKERKSTTPSKRQRKELVTPTAMESWEPPVNAGDLVAGNELDSRRAARGSGGSGVTVVRRFNEPSSAEVRAIGYVEAQLGERLRDNGPVLGMEFDPLPPGAFGMPIGI

SEQ ID NO: 175 - DNA, 1324 bp, Arabidopsis thaliana
  G773
tccgggcaggtcttcaatctcagaaggcaaaaaagaaaatcaaagaagatagttttgtgattgttttctataaaaagtgcagatattttctttgtcctagagaaagaggtgataggaaaaatgggtctccaagagcttgacccgttagcccaattgagcttaccgccgggttttcggttttatccgactgacgaagagctgatggttgaatatctctgtagaaaagccgccggtcacgacttctctctccagctcatagctgaaatcgatctctacaagtttgatccatgggttttaccaagtaaggcgttattcggtgaaaaagaatggtattttttcagcccgagggataggaagtatccaaacgggtcaagacctaatcgggttgccgggtcgggttattggaaagccaccggtacggataaagttatctcgacggagggaagaagagttggtatcaagaaagctttggtgttttacattggaaaagctccaaaaggaaccaaaaccaattggattatgcatgagtaccgtctcatcgaaccctctcgtcgaaatggaagcaccaagcttgatgattgggttttatgtcgaatatacaaaaagcaaacaagcgcacaaaaacaagcttacaataatctaatgacgagtggtcgtgaatacagcaacaatggttcgtcgacatcttcttcgtctcatcaatacgacgacgttctcgagtcgttgcatgagattgacaacagaagtttggggtttgccgccggttcatcaaacgcgctgcctcatagtcatagaccggttttaaccaatcataaaaccgggtttcagggtttagccagggagccaagttttgattgggcgaatttgattggacagaactcggtcccggaactcggactgagtcataacgttccgagtattcgttacggtgacggtggaacgcagcaacaaactgaggggattcctcggtttaataataactcggacgtctcggctaatcagggttttagtgttgacccggttaacggatttgggtactcgggtcaacaatctagtgggttcgggtttatttgattgtgtaatggtaacgtaataagaaaaacatatttttattttttgtccgtgtcagattagttaattaatatagcgtagaattcgaactctagggttagatttaggttctacgacttgtattgtatattcgtcgtcatttgtcctgacatttacatttttgtaaacttttatagctggaacttttgtattgatcaattatttattagaaaaacagatattgcattggatctataaaaaaaaaaaaaaaaaa

SEQ ID NO: 176 - protein, 317 aa, Arabidopsis thaliana
  G773
MGLQELDPLAQLSLPPGFRFYPTDEELMVEYLCRKAAGHDFSLQLIAEIDLYKFDPWVLPSKALFGEKEWYFFSPRDRKYPNGSRPNRVAGSGYWKATGTDKVISTEGRRVGIKKALVFYIGKAPKGTKTNWIMHEYRLIEPSRRNGSTKLDDWVLCRIYKKQTSAQKQAYNNLMTSGREYSNNGSSTSSSSHQYDDVLESLHEIDNRSLGFAAGSSNALPHSHRPVLTNHKTGFQGLAREPSFDWANLIGQNSVPELGLSHNVPSIRYGDGGTQQQTEGIPRFNNNSDVSANQGFSVDPVNGFGYSGQQSSGFGFI

SEQ ID NO: 177 - DNA, 1281 bp, Arabidopsis thaliana
  G1005
gctttttgtgttgaagagagagtttcctatcttctccattcctcccaccatctccctcatcttcatcttcctctctctttctctctttctcaacaatctctattagatctttctccattaccattacctctggctttctcttaaatccaccatcatgaggagaggaagaggctcttccgccgtcgccggacctaccgtcgttgccgccatcaacggatctgtaaaagaaatcagattcagaggcgtaaggaagagaccttggggacgattcgcagctgagatccgtgatccatggaaaaaagctcgtgtttggttaggtactttcgattccgccgaagaagctgctcgcgcttacgactccgccgctcgtaacctccgtggtcctaaagccaaaactaatttccccatcgattcttcttctcctcctcctcctaatctccgatttaatcagattcgtaatcaaaatcaaaaccaagtcgatccgtttatggaccaccggttattcaccgaccatcaacaacagttcccgattgttaaccggcctactagtagcagcatgagcagcaccgttgaatcgtttagcggacccagacctacgacgatgaaaccggccacgacgaagagatatcctagaactccaccggttgttccggaggattgtcacagcgattgcgattcgtcgtcgtctgtaatcgacgacgacgacgatatcgcatcgtcttcacggcgacggaatccgccgtttcaattcgatcttaattttccaccgttggattgtgttgacttgttcaatggcgctgatgatcttcactgtaccgatctacgtctctaatgaattggtaaaatcaaactcaaaatcacagatccgtgatcggtttgattttaatcgaaaacacacaacaaaatccttttttttttttttttaaattttctgtttcgttgatctcatataatttttactatgcgggagaaatagaaagacaaagaaacgaagaagaagaagaagatggtgatgagcttgagagagcttgagctggttctgtgtttcttctgtgatgatattgtaagagtattattattttactattattactaaatcttcaaaaccaagaagaagaagaccgaacacgatgatctgttgtgtctgtttgttttactgtaagaaaaacgcagatctgggtttcgtttttttcttgagatagatcaaacaacccccatctttgtaacatatacatttggaacactcatgattctaaataaaaaatctagaatctttttttc

SEQ ID NO: 178 - protein, 225 aa, Arabidopsis thaliana
  G1005
MRRGRGSSAVAGPTVVAAINGSVKEIRFRGVRKRPWGRFAAEIRDPWKKARVWLGTFDSAEEAARAYDSAARNLRGPKAKTNFPIDSSSPPPPNLRFNQIRNQNQNQVDPFMDHRLFTDHQQQFPIVNRPTSSSMSSTVESFSGPRPTTMKPATTKRYPRTPPVVPEDCHSDCDSSSSVIDDDDDIASSSRRRNPPFQFDLNFPPLDCVDLFNGADDLHCTDLRL

SEQ ID NO: 179 - DNA, 1349 bp, Arabidopsis thaliana
  G1232
gcatctctttcgcaaaaggcattgattttgctatagcgaatagtgataggggtttttttttctaagcgcagctctctcttctcaatttccccaaatgaaataaattcctccgaaagtttatagatttcactctcccccgaactatacaaaattccctcaaaaggaaaaactaaaaagccgatcttggcttttttgtttctgagtgcatctcgatacttggaagcattctttggtgaggctgattcattctcatcttgtgaaagtctttggcttttgaagcaatactatggaaggaattcagcatcctatacctcgcactgtcgaagaggtttttagcgactttcgtggtcgtagagctggtctcatcaaggctctctctactgatgtacagaagttttaccatcagtgtgaccctgagaaggaaaacttgtgtctttatggacttccaaatgagacatgggaggttaatcttcccgttgaggaggtccctcctgagcttcctgaaccagcgttaggcatcaatttcgcaagggacggaatgcaagaaaaagactggatttctttggttgcagttcacagtgattcatggcttatctctgttgcattttactttggtgcacgtttcggatttggcaagaatgagaggaagaggctcttccagatgataaatgatctcccaaccattttcgaggttgtaactggcaatgcaaagcaatccaaggatcaatctgctaaccacaacagtagcagaagcaaatctagtggtggcaagcctcgtcattctgaatctcacactaaggcttcaaagatgtcccctccaccaagaaaagaagatgagagcggagatgaggatgaagatgacgagcaaggtgctgtttgtggtgcgtgtggcgacaactatggaggagatgagttctggatatgttgtgatgcttgcgagaaatggttccatgggaaatgcgtgaagatcacaccagcaaaggctgagcatatcaaacattacaaatgcccaagttgcactaccagcaagaaaatgaaagcctgaaagtgataataacattagagcctgtgaaagaggaaaacgttttcacagtttaagaagacagtacaagagtttagctgtgggaagaagaagaaacaaaaggcttctgattgcgtagttttaatgggattgagacagcttacagttagtaatgtctctttctcttttcttttctttttttttgctgttaatgttaaaatgttatgactaatttgtcttaagtttctgagaaaattctgtcttttaaccactctctagtctatgtcaagtggtttcttcttctcggtaaaaaaaaaaaaaaaaaaaa

SEQ ID NO: 180 - protein, 252 aa, Arabidopsis thaliana
  G1232
MEGIQHPIPRTVEEVFSDFRGRRAGLIKALSTDVQKFYHQCDPEKENLCLYGLPNETWEVNLPVEEVPPELPEPALGINFARDGMQEKDWISLVAVHSDSWLISVAFYFGARFGFGKNERKRLFQMINDLPTIFEVVTGNAKQSKDQSANHNSSRSKSSGGKPRHSESHTKASKMSPPPRKEDESGDEDEDDEQGAVCGACGDNYGGDEFWICCDACEKWFHGKCVKITPAKAEHIKHYKCPSCTTSKKMKA

SEQ ID NO: 181 - DNA, 1892 bp, Arabidopsis thaliana
  G915
atcacttctctcattttcttcttctccaagcccccctcctttctatatatatctgaaattggtcttttataaaacaaacttccatttttcgtatggctgcttcttttcttacaatggacaatagcagaaccagacaaaacatgaatggttctgctaattggtcacaacaatccggaagaacatctacttcctctttggaagatcttgagataccaaagttcagatcttttgctccttcttcaatctctatctctccttctcttgtctctccttccacttgtttcagtccctctctttttctcgattcccctgcttttgtctcctcctctgctaacgttctagcttctccaaccacaggagctttaatcacaaacgtaactaaccagaaaggtataaatgaaggagacaagagcaacaacaacaactttaacttattcgatttctcattccacacacaatcatcaggagtttctgctccgaccacaactacaactacaactacaactacaacaacaacaaacagttctatctttcaatctcaggaacaacagaagaagaaccagtcagaacaatggagccaaaccgagactcgtccaaacaatcaagctgtatcttacaatggaagagagcaaaggaaaggagaggatggttacaattggagaaagtacggacaaaaacaggtgaaaggaagtgagaatcctcggagttactataagtgtactttccctaattgtccaacgaagaagaaagtggagagatctttggaaggtcagatcacagagattgtgtataaaggaagccacaaccatcctaaacctcagtctactagaagatcttcttcgtcttcttcgacttttcattcagctgtgtacaatgccagtttggatcataatcgtcaagcttcttctgatcagcctaattccaataatagctttcatcagtctgattcctttgggatgcaacaagaggataatactacttctgattctgttggtgacgatgagttcgaacaaggctcatcgattgtcagcagagacgaagaagattgtgggagtgaacctgaagcaaagagatggaaaggggacaatgaaacaaatggtgggaatggtggtggaagcaagacagtgagagagccgagaatcgtagtgcagacaacgagtgatattgacattcttgacgacggttacagatggagaaaatacggccagaaagtcgttaagggaaacccaaatccaagaagctactacaagtgcacaaccatcggttgtccagtgaggaaacatgtggagagagcatcacacgacatgagagcagtaatcacaacctacgaagggaaacacaaccacgatgttcctgcagctcgtggtagcggttacgccacaaacagagcaccacaggattcgtcttcagtcccgattagaccagctgctattgctggtcactccaactacactacttcttctcaagcaccatatacacttcagatgctgcacaacaacaacactaataccgggccttttggttacgccatgaacaacaataacaacaacagcaaccttcaaacgcaacaaaactttgttggtggtggattctctagagcaaaggaagaaccaaacgaggagacctcatttttcgattcgtttatgccctgaagaaaaaaggaaccagttgttccttttttatgttccttttgtacatttctgccaccaaaggattttactacttactagttatcctgcaggatagtaagtcagacttctatagtccatagagaaattttttcatttgttttttcacacgcctgtaatatgtttaatgtttgtactttgtaccatagaactagaacacggaataaaaccaatcaattttcagtttcaaaaaaaaaaaaaaaa

SEQ ID NO: 182 - protein, 519 aa, Arabidopsis thaliana
  G915
MAASFLTMDNSRTRQNMNGSANWSQQSGRTSTSSLEDLEIPKFRSFAPSSISISPSLVSPSTCFSPSLFLDSPAFVSSSANVLASPTTGALITNVTNQKGINEGDKSNNNNFNLFDFSFHTQSSGVSAPTTTTTTTTTTTTTNSSIFQSQEQQKKNQSEQWSQTETRPNNQAVSYNGREQRKGEDGYNWRKYGQKQVKGSENPRSYYKCTFPNCPTKKKVERSLEGQITEIVYKGSHNHPKPQSTRRSSSSSSTFHSAVYNASLDHNRQASSDQPNSNNSFHQSDSFGMQQEDNTTSDSVGDDEFEQGSSIVSRDEEDCGSEPEAKRWKGDNETNGGNGGGSKTVREPRIVVQTTSDIDILDDGYRWRKYGQKVVKGNPNPRSYYKCTTIGCPVRKHVERASHDMRAVITTYEGKHNHDVPAARGSGYATNRAPQDSSSVPIRPAAIAGHSNYTTSSQAPYTLQMLHNNNTNTGPFGYAMNNNNNNSNLQTQQNFVGGGFSRAKEEPNEETSFFDSFMP

SEQ ID NO: 183 - DNA, 1003 bp, Arabidopsis thaliana
  G21
tgtggaggaatattaatacagcccacttcacatctatttttgtgcaaccatctctctaaagcttcttctctcataacaatggcaagacaaatcaacatagagagtagtgtttctcaagttacctttatctcctccgccatccccgccgtatcttcctcctcctccatcaccgcttccgcctcattgtcctcttcacctactacatcttcctcttcttcgtcatcaacaaattctaacttcattgaggaagacaactctaaaagaaaagcatctcgaagatcattgtcatcgttagtctccgttgaagacgatgatgatcaaaacggtggaggtgggaaacggcgaaagaccaacggtggagataaacatccgacgtatagaggagtgaggatgaggagttggggaaaatgggtgtcggagattagagagccgagaaagaaatcaagaatctggctcgggacttatccaacggctgagatggcagctcgagctcatgacgtagcggctttagccattaaaggtacaacggcttacctcaattttcccaagttagccggcgagcttcctcgtccagtcacaaattctcctaaagacattcaagccgccgcctctttagcggccgttaactggcaagattcggtcaacgatgtgagtaattctgaagtggctgaaatagttgaagccgagccgagtcgagccgtggtggctcagttgttttcttcggacacaagcacgacgacgacgactcagagtcaagagtattcggaagcttcgtgtgcttcgacttcggcgtgtacggacaaagacagtgaggaagagaagctgtttgatttgccggatttgtttaccgatgagaatgagatgatgatacgaaacgatgcgttttgctactactcgtccacgtggcagctttgtggagccgatgctgggtttcggcttgaagagccgttttttctatctgaatgactaaagtacccctctcgagagagctctcactaacact

SEQ ID NO: 184 - protein, 295 aa, Arabidopsis thaliana
  G21
MARQINIESSVSQVTFISSAIPAVSSSSSITASASLSSSPTTSSSSSSSTNSNFIEEDNSKRKASRRSLSSLVSVEDDDDQNGGGGKRRKTNGGDKHPTYRGVRMRSWGKWVSEIREPRKKSRIWLGTYPTAEMAARAHDVAALAIKGTTAYLNFPKLAGELPRPVTNSPKDIQAAASLAAVNWQDSVNDVSNSEVAEIVEAEPSRAVVAQLFSSDTSTTTTTQSQEYSEASCASTSACTDKDSEEEKLFDLPDLFTDENEMMIRNDAFCYYSSTWQLCGADAGFRLEEPFFLSE

SEQ ID NO: 185 - DNA, 1713 bp, Arabidopsis thaliana
  G8
aaaaaaaaatatccgtctcactctctcgccgccggtaacatttcccggcgacaaaacttctctactctcaccattcctccatcgtaatctctaaattcttctccattctcttcttcctcccgatcatctcgagctcttcgtgagagattatgtgattatgtaatcgttgttgctgtagaagacgatctctaacaactgattccttcatcatcaccttcgctagatttgtaattttcagagcttgagatgttggatcttaacctcaacgctgattctcccgagtcgactcagtacggtggtgactcatacttagatcggcagacatcagacaactccgccgggaatcgagtggaagagtccggtacatcgacgtcgtcagttatcaatgccgatggagacgaagactcttgctctactcgagctttcactctcagtttcgatattttaaaagtcggaagtagtagcggcggagacgaaagccccgccgcttcagcttccgttactaaagagttttttccggtgagtggagactgtggacatctacgagatgttgaaggatcatcaagctctagaaactggatagatctttcttttgaccgtattggtgacggagaaacgaaattggtaactccggttccgactccggctccggttccggctcaggttaaaaagagtcggagaggaccaaggtctagaagttcacagtatagaggagttactttttatagaagaactggtcgatgggagtcacatatttgggattgtgggaaacaagtttatttaggtggtttcgacactgctcatgctgcagctagagcttatgatcgagctgctattaaatttagaggtgttgatgctgatatcaactttactcttggtgattatgaggaagatatgaaacaggtacaaaacttgagtaaggaagagtttgtgcatatactgcgtagacagagcacggggttttcgcgggggagttcgaagtatcgaggggttacgttacacaaatgtggtagatgggaagctaggatggggcagtttcttggtaaaaaggcttatgacaaggctgcaatcaacactaatggtagagaagcagtcacgaacttcgagatgagttcataccaaaatgagattaactctgagagcaataactctgagattgacctcaacttgggaatctctttatcgaccggtaatgcgccaaagcaaaatgggaggctctttcacttcccttctaatacttatgaaactcagcgtggagttagcttgaggatagataacgaatacatgggaaagccggtgaatacacctcttccttatggatcctcggatcatcgcctttactggaacggagcatgcccgagttataataatcccgccgagggaagagcaacagaaaagagaagtgaagctgaagggatgatgagtaactggggatggcagagaccggggcaaacaagcgccgtgagaccgcagccaccgggaccacaaccaccaccattgttctcagttgcagcagcatcatcaggattctcacatttccggccacaacctcccaatgacaatgcaacacgtggttacttttatccacacccttaacttgtaaggggacatatgagagtttttttaccatctctctctctctcaacactctagtcccctttcaaaaatgtcatttgggttttagatttttcacatacaatgatcaatttttcc

SEQ ID NO: 186 - protein, 449 aa, Arabidopsis thaliana
  G8
MLDLNLNADSPESTQYGGDSYLDRQTSDNSAGNRVEESGTSTSSVINADGDEDSCSTRAFTLSFDILKVGSSSGGDESPAASASVTKEFFPVSGDCGHLRDVEGSSSSRNWIDLSFDRIGDGETKLVTPVPTPAPVPAQVKKSRRGPRSRSSQYRGVTFYRRTGRWESHIWDCGKQVYLGGFDTAHAAARAYDRAAIKFRGVDADINFTLGDYEEDMKQVQNLSKEEFVHILRRQSTGFSRGSSKYRGVTLHKCGRWEARMGQFLGKKAYDKAAINTNGREAVTNFEMSSYQNEINSESNNSEIDLNLGISLSTGNAPKQNGRLFHFPSNTYETQRGVSLRIDNEYMGKPVNTPLPYGSSDHRLYWNGACPSYNNPAEGRATEKRSEAEGMMSNWGWQRPGQTSAVRPQPPGPQPPPLFSVAAASSGFSHFRPQPPNDNATRGYFYPHP

SEQ ID NO: 187 - DNA, 1432 bp, Arabidopsis thaliana
  G1362
aggtagaatctctattcgagtccccaagattctctttatatccctagtttagttgtatggttgggttttgattggaatatcaaggggtagtttttagctaggttcacttgatacttggaagatctgagttctttgggattctgttaagtttgtggagatctaaaagacacgaaatttgtagaaatctggttgatatcccagacttttagagggattagggtagattctatagaatttgaggcgggtttgattggaattatgacggtggaggaagttagtgatggttctgtgtggagtagggaggatgatattgcctttgagagacctctagccaataataccgatgaatcagaggaacggtgggagaagattgctgcagacgttccaggcaaaagtgttgaacagattaaagaacattacgagcttttagttgaagatgttactaggattgaatcaggatgtgtgcctcttcctgcctatgggtctcctgaaggatcgaatggccatgctggtgatgaaggagcaagtagtaagaaaggaggtaacagtcatgcgggagagtctaaccaagcaggtaaatcaaagtccgatcaagaacgacgaaagggtatcgcgtggacagaagatgagcacaggttatttcttcttggtttggataagtacgggaaaggtgattggcgtagcatttctcgcaactttgtagtaacaagaacaccgacccaagttgcgagccatgctcaaaagtatttcattcgtctaaattcaatgaacaaagacagaaggcgatcaagcattcacgacatcactagtgttggcaacgcagatgtctcaaccgccacaaggccaatcactggtcagaacaacagcaataacaacaacaacaacaacaacaacaacagttctcctgctgttgctggaggaggaaacaaatcagccaagcaagccgtctctcaagcaccacctggacctcctatgtatggaacacccgccataggtcagccagcagttggaacaccagtgaacctcccagctccacctcacatggcttatggagttcatgcggctccagtccctggctcagtggttcctggtgcagcaatgaacattggtcaaatgccgtacaccatgccgcgtacaccaacggctcataggtaactcgaaagcacctttgctgtcatagtgcactttgtttttaggtgtaagaaagaagatgtgtaaaggatttagtgaatattcaagcttgttccttgagtgagttttttttattacttagtttgtggggattttgtatgaggtccgaataagatatgaagatgacatgattagtttccagactcgagaagcaaaaatactcctgtttgtatgtgaacacaataaagcctctgttatgagacttacaacaaagcaacattgtatatcttgttctcacaaa

SEQ ID NO: 188 - protein, 298 aa, Arabidopsis thaliana
  G1362
MTVEEVSDGSVWSREDDIAFERPLANNTDESEERWEKIAADVPGKSVEQIKEHYELLVEDVTRIESGCVPLPAYGSPEGSNGHAGDEGASSKKGGNSHAGESNQAGKSKSDQERRKGIAWTEDEHRLFLLGLDKYGKGDWRSISRNFVVTRTPTQVASHAQKYFIRLNSMNKDRRRSSIHDITSVGNADVSTATRPITGQNNSNNNNNNNNNNSSPAVAGGGNKSAKQAVSQAPPGPPMYGTPAIGQPAVGTPVNLPAPPHMAYGVHAAPVPGSVVPGAAMNIGQMPYTMPRTPTAHR

SEQ ID NO: 189 - DNA, 1093 bp, Arabidopsis thaliana
  G142
atttatcgtgtacgatactttattccttttatctattcttgaaaaaaagttaccaattcttgagaagaagaagaaatcagaatcaagagaaggagagagaaagatgggaagagggagagtggagatgaagaggatagagaacaagattaatagacaagtgaccttctcaaaaagaagaaacggtttgctgaagaaagcttatgagctttctgttctttgcgatgccgaagttgctctcatcatcttctcaagccgtggcaagctctacgagtttggtagtgttggaattgaaagcacaatcgaacggtataatcgttgttacaactgctctctaagcaataataagcctgaagagactacacagagttggtgtcaggaggtgacaaagcttaaatccaaatacgaatctcttgttcgtactaacaggaatttgcttggagaagatcttggagaaatgggtgtgaaggaactgcaagcgctcgagaggcagctcgaagccgctcttaccgcgactcgacagcgcaagacacaagttatgatggaagaaatggaagaccttaggaaaaaggagaggcaactaggagacataaacaaacaactcaagattaagtttgaaacggaaggccatgctttcaaaacctttcaagacttatgggcaaactcggcggcatcggtggccggggatccaaacaattctgaatttccggtagagccttctcatcctaatgtattggattgcaacaccgaaccctttttacaaatagggtttcaacaacattactacgtgcaaggtgaagggtcttcggtatcaaagagtaacgtggcaggtgagactaatttcgtccaaggttgggttctttgactctctgttgattagcccacgatgccacggtcaggccaatttcagctctctacagttgttcttttttcaaattagatttctggttttttttttcctataagaaaaacttttgcactagatgtttgtcatttaatttccagctcgtgtgaatctatattcgcatgtatgtgctttgaagaatttctcctcttactcctacttgatctaaaacattatttttcttttgggttt

SEQ ID NO: 190 - protein, 252 aa, Arabidopsis thaliana
  G142
MGRGRVEMKRIENKINRQVTFSKRRNGLLKKAYELSVLCDAEVALIIFSSRGKLYEFGSVGIESTIERYNRCYNCSLSNNKPEETTQSWCQEVTKLKSKYESLVRTNRNLLGEDLGEMGVKELQALERQLEAALTATRQRKTQVMMEEMEDLRKKERQLGDINKQLKIKFETEGHAFKTFQDLWANSAASVAGDPNNSEFPVEPSHPNVLDCNTEPFLQIGFQQHYYVQGEGSSVSKSNVAGETNFVQGWVL

SEQ ID NO: 191 - DNA, 1164 bp, Arabidopsis thaliana
  G1425
actctctcaaaccataaaaaatattctccgatcatcattttaatggagagtacagattcttccggtggtcctccgccgccgcaaccaaacctccctccaggattccggtttcatccaacagacgaagaacttgtaattcattacctcaaacgcaaagcagattctgttcctttaccagtcgcgatcatcgccgacgttgatctttacaaatttgatccatgggaacttcccgcgaaagcttcgtttggagaacaagaatggtattttttcagtccaagagatcggaaatatcccaacggagctagacctaaccgagctgcgacttccggttattggaaagcgactggtacagataaaccggtgatttcaaccggcggtggtggtagtaaaaaagtgggagttaaaaaggctctagtgttttacagtggtaaaccaccaaaaggagttaaatcagattggattatgcatgaatatcggttaactgataataaacctactcacatttgtgacttcggcaacaagaaaaactctctcaggcttgatgattgggtgttgtgtcgtatctacaagaaaaacaatagtacagcatctagacatcatcatcatcttcatcatattcatctagataatgatcatcatcgtcatgatatgatgattgatgatgatcgattccgtcatgttcctcctggtcttcacttcccggcgattttttctgacaataatgatccgacggctatatatgatggtggcggcggcggatacggaggtggaagttactcgatgaatcattgtttcgcatctggatcaaagcaggagcagttgtttccaccggtgatgatgatgactagtctaaatcaagattccggtattggatcgtcgtcgtcacctagcaagagatttaacggcggcggcgttggagattgttcgacttctatggcggcgacgccgttaatgcagaaccaaggtgggatttaccaattgcctggtttgaattggtattcttgaaaacaatttacgatgaagaatttttaaaatttgtgtatatatatacggtttgagtgattagggggcattgggggatttatttacggttgattattattgtagtgttatagaactaaggagattaaattaaatagattggaggaaaaaaaaaaaaaaaaa

SEQ ID NO: 192 - protein, 320 aa, Arabidopsis thaliana
  G1425
MESTDSSGGPPPPQPNLPPGFRFHPTDEELVIHYLKRKADSVPLPVAIIADVDLYKFDPWELPAKASFGEQEWYFFSPRDRKYPNGARPNRAATSGYWKATGTDKPVISTGGGGSKKVGVKKALVFYSGKPPKGVKSDWIMHEYRLTDNKPTHICDFGNKKNSLRLDDWVLCRIYKKNNSTASRHHHHLHHIHLDNDHHRHDMMIDDDRFRHVPPGLHFPAIFSDNNDPTAIYDGGGGGYGGGSYSMNHCFASGSKQEQLFPPVMMMTSLNQDSGIGSSSSPSKRFNGGGVGDCSTSMAATPLMQNQGGIYQLPGLNWYS

SEQ ID NO: 193 - DNA, 1090 bp, Arabidopsis thaliana
  G1448
atgtctgtatctgtagcagcagagcatgattacataggtttgtcagagtttccaaccatggaagcaacaacaatgtctgacaaaaccaaaaccagagacaataacaacggtctcaatttcaaggctaccgagttaagactcggtttacccggttctgagtcgccggagcgagtcgactcaagattcttggctctcaacaagagtagctgtcccgtgtcaggtgccaaaagggtgttctccgacgctattaacgactctaacaaatgggtcttctctcctggatccactactgctactggtgatgtcggctcgggttctggtccccgtacctccgtcgttaaagatggaaagtcgacaactttcactaaaccggctgttccggttaaggagaagaagagctctgctacagctccagcttcaaaagcacaagtggtgggatggccaccaataagatcattcaggaagaactcaatggcttcttctcaatctcagaaacctggtaataactcagagactgaagaagcagaagctaagtctggaccagaacaaccttgcttgtatgtcaaagtgagtatggaaggtgctccttacttgaggaaaatcgatctcaagacttacaaaagctaccttgagctctcttctgctcttgagaagatgttcagttgcttcaccattggtcagtttggttctcatggagggtgtggcagagatgggttaaacgagagtcgcttgactgatctcttgcgtggttctgagtatgttgtaacctatgaagataaagacagtgactggatgctggtcggagatgtcccttgggaaatgtttatatgctcctgcaagaagctgagaatcatgaagagctctgaggctatcggcttagctccaagggtgatggagaagtgcagaagcaggaactaggaaaagcccttttatgtgataatatatgatttgaaaaaatatgtttttaagaaattgaatagccattttgttttgaagtattcttgtgtgtttgtgtgctaggagtgatgtttgatctttcttaaaccagcctctactaagctctaataaggcctcttcctcttaaaaaaaa

SEQ ID NO: 194 - protein, 305 aa, Arabidopsis thaliana
  G1448
MSVSVAAEHDYIGLSEFPTMEATTMSDKTKTRDNNNGLNFKATELRLGLPGSESPERVDSRFLALNKSSCPVSGAKRVFSDAINDSNKWVFSPGSTTATGDVGSGSGPRTSVVKDGKSTTFTKPAVPVKEKKSSATAPASKAQVVGWPPIRSFRKNSMASSQSQKPGNNSETEEAEAKSGPEQPCLYVKVSMEGAPYLRKIDLKTYKSYLELSSALEKMFSCFTIGQFGSHGGCGRDGLNESRLTDLLRGSEYVVTYEDKDSDWMLVGDVPWEMFICSCKKLRIMKSSEAIGLAPRVMEKCRSRN

SEQ ID NO: 195 - DNA, 906 bp, Arabidopsis thaliana
  G145
aagaaaatgggaagagggagagtagaattgaagaggatagagaacaagatcaataggcaagtgacgtttgcaaagagaaggaatggtcttttgaagaaagcatacgagctttcagttctatgtgatgcagaagttgctctcatcatcttctcaaatagaggaaagctgtacgagttttgcagtagttcgagcatgcttcggacactggagaggtaccaaaagtgtaactatggagcaccagaacccaatgtgccttcaagagaggccttagcagttgaacttagtagccagcaggagtatctcaagcttaaggagcgttatgacgccttacaaagaacccaaaggaatctgttgggagaagatcttggacctctaagtacaaaggagcttgagtcacttgagagacagcttgattcttccttgaagcagatcagagctctcaggacacagtttatgcttgaccagctcaacgatcttcagagtaaggaacgcatgctgactgagacaaataaaactctaagactaaggttagctgatgggtatcagatgccactccagctgaaccctaaccaagaagaggttgatcactacggtcgtcatcatcatcaacaacaacaacactcccaagctttcttccagcctttggaatgtgaacccattcttcagatcgggtatcaggggcagcaagatggaatgggagcaggaccaagtgtgaataattacatgttgggttggttaccttatgacaccaactctatttgaatctttctcacttaattaatctctcttttttttgacatttttaagatgatgtttctattttattacctctctcacgttttctgtcttgtgtgcatgtgtctgtgtaatgtttattgcccttctattattcaatgatttctcgac

SEQ ID NO: 196 - protein, 251 aa, Arabidopsis thaliana
  G145
MGRGRVELKRIENKINRQVTFAKRRNGLLKKAYELSVLCDAEVALIIFSNRGKLYEFCSSSSMLRTLERYQKCNYGAPEPNVPSREALAVELSSQQEYLKLKERYDALQRTQRNLLGEDLGPLSTKELESLERQLDSSLKQIRALRTQFMLDQLNDLQSKERMLTETNKTLRLRLADGYQMPLQLNPNQEEVDHYGRHHHQQQQHSQAFFQPLECEPILQIGYQGQQDGMGAGPSVNNYMLGWLPYDTNSI

SEQ ID NO: 197 - DNA, 826 bp, Arabidopsis thaliana
  G162
agacatacaacaccaaaatcttcttcttcaccaacatattcactttcacagcaaaaaaaaacgagaggttctctcttattcgtaccgttttagcaaacaaatgggtcggagaaagatcaagatggagatggttcaggacatgaacacacgacaggttaccttttcaaaacggaggactggtttgttcaagaaggcgagcgagttagccacgctctgcaacgctgagttgggcatcgttgtcttttcaccaggaggcaagcctttctcctacgggaaaccgaatcttgattctgttgcagagcgattcatgagagaatatgatgattcagacagtggcgatgaagaaaaaagtggtaattacaggcctaaactgaagaggctgagtgaacgtctcgatttgctcaaccaagaggttgaagctgagaaggaacgaggcgagaagagtcaggagaagcttgaatctgctggggatgagagattcaaggagtccattgagacgcttaccctcgatgaactcaatgaatacaaagataggcttcagacagtccatggtaggattgaaggtcaagtcaatcacttgcaggcttcgtcttgcctcatgcttctctccagaaaatagctagaccgacttgttagagttacattctattttttgtatcagcctacagaacttaccaacacatgaaagttattgctggtgtagaattttctgtcatctatggggtgtgactttctatttgacatcaaatgaaaatgtacctggaaatttgtctgtattaatctcaagtgtacttgctaaacttgatcagctttttcgcaaaaaaaa

SEQ ID NO: 198 - protein, 172 aa, Arabidopsis thaliana
  G162
MGRRKIKMEMVQDMNTRQVTFSKRRTGLFKKASELATLCNAELGIVVFSPGGKPFSYGKPNLDSVAERFMREYDDSDSGDEEKSGNYRPKLKRLSERLDLLNQEVEAEKERGEKSQEKLESAGDERFKESIETLTLDELNEYKDRLQTVHGRIEGQVNHLQASSCLMLLSRK

SEQ ID NO: 199 - DNA, 2526 bp, Arabidopsis thaliana
  G680
cagttatcttcttccttcttctctctgttttttaaatttatttttagagaattttttttgttttgcttccgatttgattatttccgggaacgatgacttctccggggagttcccggtgagatgataagtcagattgcatacttgtctcctccatggctactctcaagggttttggctgcggtggattcgtttggtttctctagaatctaaagaggttatcacaacggctttgcaatttgaaaactttcatgtttggggagatcaaagatggtttcttttttatactttacttgttagagaggatttgaagcagcgaatagctgcaaccggtcctgttatggatactaatacatctggagaagaattattagctaaggcaagaaagccatatacaataacaaagcagcgagagcgatggactgaggatgagcatgagaggtttctagaagccttgaggctttatggaagagcttggcaacgaattgaagaacatattgggacaaagactgctgttcagatcagaagtcatgcacaaaagttcttcacaaagttggagaaagaggctgaagttaaaggcatccctgtttgccaagctttggacatagaaattccgcctcctcgtcctaaacgaaaacccaatactccttatcctcgaaaacctgggaacaacggtacatcttcctctcaagtatcatcagcaaaagatgcaaaacttgtttcatcggcctcttcttcacagttgaatcaggcgttcttggatttggaaaaaatgccgttctctgagaaaacatcaactggaaaagaaaatcaagatgagaattgctcgggtgtttctactgtgaacaagtatcccttaccaacgaaacaggtaagtggcgacattgaaacaagtaagacctcaactgtggacaacgcggttcaagatgttcccaagaagaacaaagacaaagatggtaacgatggtactactgtgcacagcatgcaaaactacccttggcatttccacgcagatattgtgaacgggaatatagcaaaatgccctcaaaatcatccctcaggtatggtatctcaagacttcatgtttcatcctatgagagaagaaactcacgggcacgcaaatcttcaagctacaacagcatctgctactactacagcttctcatcaagcgtttccagcttgtcattcacaggatgattaccgttcgtttctccagatatcatctactttctccaatcttattatgtcaactctcctacagaatcctgcagctcatgctgcagctacattcgctgcttcggtctggccttatgcgagtgtcgggaattctggtgattcatcaaccccaatgagctcttctcctccaagtataactgccattgccgctgctacagtagctgctgcaactgcttggtgggcttctcatggacttcttcctgtatgcgctccagctccaataacatgtgttccattctcaactgttgcagttccaactccagcaatgactgaaatggataccgttgaaaatactcaaccgtttgagaaacaaaacacagctctgcaagatcaaaccttggcttcgaaatctccagcttcatcatctgatgattcagatgagactggagtaaccaagctaaatgccgactcaaaaaccaatgatgataaaattgaggaggttgttgttactgccgctgtgcatgactcaaacactgcccagaagaaaaatcttgtggaccgctcatcgtgtggctcaaatacaccttcagggagtgacgcagaaactgatgcattagataaaatggagaaagataaagaggatgtgaaggagacagatgagaatcagccagatgttattgagttaaataaccgtaagattaaaatgagagacaacaacagcaacaacaatgcaactactgattcgtggaaggaagtctccgaagagggtcgtatagcgtttcaggctctctttgcaagagaaagattgcctcaaagcttttcgcctcctcaagtggcagagaatgtgaatagaaaacaaagtgacacgtcaatgccattggctcctaatttcaaaagccaggattcttgtgctgcagaccaagaaggagtagtaatgatcggtgttggaacatgcaagagtcttaaaacgagacagacaggatttaagccatacaagagatgttcaatggaagtgaaagagagccaagttgggaacataaacaatcaaagtgatgaaaaagtctgcaaaaggcttcgattggaaggagaagcttctacatgacagacttggaggtaaaaaaaaaacatccacatttttatcaatatctttaaatctagtgttagtagtttgcttctccaatctttatgaaagagacttttaattttccttccgaacatttctttggtcatgtcaggttctgtaccatattaccccatgtcttgtctcttgtctctgtttgtgtatgctacttgtggtctatatgtcatctgctactactgttaattaaccattaagcaatggatttgtcttta

SEQ ID NO: 200 - protein, 645 aa, Arabidopsis thaliana
  G680
MDTNTSGEELLAKARKPYTITKQRERWTEDEHERFLEALRLYGRAWQRIEEHIGTKTAVQIRSHAQKFFTKLEKEAEVKGIPVCQALDIEIPPPRPKRKPNTPYPRKPGNNGTSSSQVSSAKDAKLVSSASSSQLNQAFLDLEKMPFSEKTSTGKENQDENCSGVSTVNKYPLPTKQVSGDIETSKTSTVDNAVQDVPKKNKDKDGNDGTTVHSMQNYPWHFHADIVNGNIAKCPQNHPSGMVSQDFMFHPMREETHGHANLQATTASATTTASHQAFPACHSQDDYRSFLQISSTFSNLIMSTLLQNPAAHAAATFAASVWPYASVGNSGDSSTPMSSSPPSITAIAAATVAAATAWWASHGLLPVCAPAPITCVPFSTVAVPTPAMTEMDTVENTQPFEKQNTALQDQTLASKSPASSSDDSDETGVTKLNADSKTNDDKIEEVVVTAAVHDSNTAQKKNLVDRSSCGSNTPSGSDAETDALDKMEKDKEDVKETDENQPDVIELNNRKIKMRDNNSNNNATTDSWKEVSEEGRIAFQALFARERLPQSFSPPQVAENVNRKQSDTSMPLAPNFKSQDSCAADQEGVVMIGVGTCKSLKTRQTGFKPYKRCSMEVKESQVGNINNQSDEKVCKRLRLEGEAST

SEQ ID NO: 201 - DNA, 1023 bp, Arabidopsis thaliana
  G6
tatctatccgagaatggccaagatgggcttgaaacccgacccggctactactaaccagacccacaataatgccaaggagattcgttacagaggcgttaggaagcgtccttggggccgttatgccgccgagatccgagatccgggcaagaaaacccgcgtctggcttggcactttcgatacggctgaagaggcggcgcgtgcttacgatacggcggcgcgtgattttcgtggtgctaaggctaagaccaatttcccaacttttctcgagctgagtgaccagaaggtccctaccggtttcgcgcgtagccctagccagagcagcacgctcgactgtgcttctcctccgacgttagttgtgccttcagcgacggctgggaatgttcccccgcagctcgagcttagtctcggcggaggaggcggcggctcgtgttatcagatcccgatgtcgcgtcctgtctactttttggacctgatggggatcggtaacgtaggtcgtggtcagcctcctcctgtgacatcggcgtttagatcgccggtggtgcatgttgcgacgaagatggcttgtggtgcccaaagcgactctgattcgtcatcggtcgttgatttcgaaggtgggatggagaagagatctcagctgttagatctagatcttaatttgcctcctccatcggaacaggcctgagcttttaacggtgtcgtttcaattcgaagcgcatgcgtttcttcttctttttgagctgtgaaaattcgttttctcatagtttttcctctctctctctctcagtctaaatttattaccagtttttagaaagaaaaaacagattaaatctgagagagaaaaatataattttagctgacatggatcgttatgtacatattattacataaccggagatctgaacttttgttgtgtgcttttaattttttgcgacttggtttcaccccatgttgtttctctattttttttactactttttttttttttgttcttccaaattttcaatcaataatttggtaatcttc

SEQ ID NO: 202 - protein, 222 aa, Arabidopsis thaliana
  G6
MAKMGLKPDPATTNQTHNNAKEIRYRGVRKRPWGRYAAEIRDPGKKTRVWLGTFDTAEEAARAYDTAARDFRGAKAKTNFPTFLELSDQKVPTGFARSPSQSSTLDCASPPTLVVPSATAGNVPPQLELSLGGGGGGSCYQIPMSRPVYFLDLMGIGNVGRGQPPPVTSAFRSPVVHVATKMACGAQSDSDSSSVVDFEGGMEKRSQLLDLDLNLPPPSEQA

SEQ ID NO: 203 - DNA, 1009 bp, Arabidopsis thaliana
  G801
gatagtgataacgaaatcctaattccatggccgacaacgacggagcagtgagtaacggcatcatagtcgagcagacgtcaaacaaaggacctcttaacgccgttaagaaaccaccgtctaaagatcgacacagcaaagttgacggaagaggaagaaggattcgtatgccaatcatttgcgcagctcgagtttttcaattgaccagagagttaggtcacaagtccgatggtcaaaccatagagtggcttctccgtcaagctgagccttctatcatagccgccactggaactggcactactccggcgagtttctccactgcttctctctccacttcttctccgtttactctcgggaaacgtgtcgtcagagcggaggaaggagaatccggcggcggaggaggaggagggttaacagtgggacacacaatggggacttcgttaatgggtggtggtggttctggtgggttttgggctgttccggcgaggccggatttcggacaagtctggagctttgcaaccggagctccaccggaaatggtttttgcgcagcagcagcaaccagctacactcttcgtccgccaccagcagcaacagcaagcttccgccgccgcagcagctgcaatgggtgaggcttcagcagctagagttgggaattatcttccgggtcatcatctcaatttgcttgcttctttgtctggtggagctaacgggtcgggtcggagggaagacgaccacgaaccacgttgagaaatggtattgtctttttggtaatgtatagaaaaattcctatgttttatgtcatcgaaagtgtttagaaagtacctctaatttgcggtttcttttgctccttttttacttaatttaagcttattgcttgtttgattagggttttagggtttaagaatatttggtctcgttaatttgtttcggagagtgatagaaagagagagagattgattgattgttgtacctaaaacgctataaaagctctgtttttactagcgaaaaaa

SEQ ID NO: 204 - protein, 239 aa, Arabidopsis thaliana
  G801
MADNDGAVSNGIIVEQTSNKGPLNAVKKPPSKDRHSKVDGRGRRIRMPIICAARVFQLTRELGHKSDGQTIEWLLRQAEPSIIAATGTGTTPASFSTASLSTSSPFTLGKRVVRAEEGESGGGGGGGLTVGHTMGTSLMGGGGSGGFWAVPARPDFGQVWSFATGAPPEMVFAQQQQPATLFVRHQQQQQASAAAAAAMGEASAARVGNYLPGHHLNLLASLSGGANGSGRREDDHEPR

SEQ ID NO: 205 - DNA, 1535 bp, Arabidopsis thaliana
  G216
ttagctcaaagcccttaaatcttgtctcaccatctgacttgatacaccattgtttaccattccactaaatttcgagtgagcatactcacgacgaaactgatcatcagccttcaaactctgttcagcttccttcccactcctaatccaagcagtattgttcaggtcatttgatatagagatcaattctttatctaactctgcagaagaatgttcctcataatccacaaactctgatcccagcaccgacgatgatgacagtgttgtgctgtcttgatggttgcagcaaccgtgaccgcattttgaagggataataggatcataacaaagagactgaagaaatttaccagccaatgagtcaggtcttgatgcttgaaccaaaggtccctcacatggagccatgtatcctggtttacagacactgaaagcacccattcgagccactggacgataaacaccagttttactattttgctcccgaggaggctcctttgcttcatcatccattttatcatcacttaatacaccggtttcttcttcttgaggtagatgttctttgggggatgcgtttgatatccttctgaccattgatggatacagttgatatactatctctgtattagacatcaatagaggaatcttccattgttctgctggtttacgtctaagattagagttccaatgattcttaatggcattatctgttctcccaggtaagagtttagcaatcacagaccatttgttcccaagaaccgcctgtgcagacattatcatatgctcctcctcatctgcatttcaaatcattttcataaaaacattatacaaacatgtgaaaacacataactcacacactaatcatcaaaaaccacatctctaaagaattacaacataaacatcttgaatccaagagaatgctaaaacctattaaaccatctccatatacataagaatctgattctgaagaaaagttcatatccagtaaaatcaccagcaaaacagaccaatctacaaatctctcacaaatttttcaaaaataagctaaaacaccgcttctcattagcactactgagaaaatcatatgaatcaagtaccacaacaaagatcctgaatcaacaatagctaacactagacatagataaagagaaatccagagaaatcgaccaaagcaggaatctggaatcaaggagaatcaaaaagaagatcgagaatcgtgttttaccagagaaaggttttcgtttgaggataggatcgagctgattacaccaacgtaatcggcaagatttaccagagcgacctgggattccacgggagattagattccagttcctcggcccacacattttcacaagcctcgtgagagcctcatcttgctccggcaaccaaggtcctttcactttactctttccgccgccgttcgcgtcgcttttcgctagctcggcgagctctgcctcaactgctttgtgtattgcattttcaaaactcttgcaaggattccgggacgatattagctccggcggacgagagatttctccgttcat

SEQ ID NO: 206 - protein, 367 aa, Arabidopsis thaliana
  G216
MNGEISRPPELISSRNPCKSFENAIHKAVEAELAELAKSDANGGGKSKVKGPWLPEQDEALTRLVKMCGPRNWNLISRGIPGRSGKSCRLRWCNQLDPILKRKPFSDEEEHMIMSAQAVLGNKWSVIAKLLPGRTDNAIKNHWNSNLRRKPAEQWKIPLLMSNTEIVYQLYPSMVRRISNASPKEHLPQEEETGVLSDDKMDDEAKEPPREQNSKTGVYRPVARMGAFSVCKPGYMAPCEGPLVQASRPDSLAGKFLQSLCYDPIIPSKCGHGCCNHQDSTTLSSSSVLGSEFVDYEEHSSAELDKELISISNDLNNTAWIRSGKEAEQSLKADDQFRREYAHSKFSGMVNNGVSSQMVRQDLRALS

SEQ ID NO: 207 - DNA, 930 bp, Arabidopsis thaliana
  G230
atgtctggttcgacccggaaagaaatggatcggatcaaaggaccatggagtcctgaagaagacgatctgttacaatcgttggttcagaagcacggaccaaggaactggtctctgataagcaaatcaatccctggacgttccggtaaatcttgccgtctccgttggtgtaatcagctttctccggaggtagagcaccgtggattcacggcggaggaagatgatacgattatactagcgcacgctcggtttggtaacaagtgggcgacgattgcacggcttctcaatggtcgcactgataacgcgattaagaatcactggaactcaacgctgaagcggaaatgtagcggcggaggcggcggaggagaggaagggcagagttgtgatttcggtggtaatggagggtatgatggtaatttaactgatgaaaaaccgttaaaacggagggcgagtggtggaggaggagttgttgtggtgacggcgttaagtccaacgggatctgacgtcagcgagcaatcgcaatctagtggatctgttttaccggtttcttcttcttgtcatgtttttaaaccgacggcgagagctggtggagtggtgattgagtcatcatcgccggaggaggaggagaaagatccgatgacttgtttgaggttgtctttgccttgggtcaatgagtcaacaactccaccggagttgtttccggtgaagagagaagaagaagaagagaaggaaagagagatttctggacttggtggagattttatgacggtggtgcaggagatgattaagacggaggttaggagttacatggcggatttacagctaggaaacggcggaggagctggaggaggagcaagttcgtgtatggtgcaaggaactaatggtcgtaatgtagggtttagagagtttattggattaggtaggatcgagtag

SEQ ID NO: 208 - protein, 309 aa, Arabidopsis thaliana
  G230
MSGSTRKEMDRIKGPWSPEEDDLLQSLVQKHGPRNWSLISKSIPGRSGKSCRLRWCNQLSPEVEHRGFTAEEDDTIILAHARFGNKWATIARLLNGRTDNAIKNHWNSTLKRKCSGGGGGGEEGQSCDFGGNGGYDGNLTDEKPLKRRASGGGGVVVVTALSPTGSDVSEQSQSSGSVLPVSSSCHVFKPTARAGGVVIESSSPEEEEKDPMTCLRLSLPWVNESTTPPELFPVKREEEEEKEREISGLGGDFMTVVQEMIKTEVRSYMADLQLGNGGGAGGGASSCMVQGTNGRNVGFREFIGLGRIE

SEQ ID NO: 209 - DNA, 1040 bp, Arabidopsis thaliana
  G528
ccacgcgtccgccattaaacaaaaaaaaatcaaatctctctctttctctctctaatggcggcgacattaggcagagaccagtatgtgtacatggcgaagctcgccgagcaggcggagcgttacgaagagatggttcaattcatggaacagctcgttacaggcgctactccagcggaagagctcaccgttgaagagaggaatctcctctctgttgcttacaaaaacgtgatcggatctctacgcgccgcctggaggatcgtgtcttcgattgagcagaaggaagagagtaggaagaacgacgagcacgtgtcgcttgtcaaggattacagatctaaagttgagtctgagctttcttctgtttgctctggaatccttaagctccttgactcgcatctgatcccatctgctggagcgagtgagtctaaggtcttttacttgaagatgaaaggtgattatcatcggtacatggctgagtttaagtctggtgatgagaggaaaactgctgctgaagataccatgctcgcttacaaagcagctcaggatatcgcagctgcggatatggcacctactcatccgataaggcttggtctggccctgaatttctcagtgttctactatgagattctcaattcttcagacaaagcttgtaacatggccaaacaggcttttgaggaggccatagctgagcttgacactctgggagaggaatcctacaaagacagcactctcataatgcagttgctgagggacaatttaaccctttggacctccgatatgcaggagcagatggacgaggcctgaggatctagatgaagggggggagggttgttacgcgatgtttctgccaccaaatcgatctcaaaatccccataacctttgctcaaaaactgtgaaaaaagattgaagtgtttatgatgattatgattgtgcacagcttgatgatttatctactctactaaacctctgtgctcttaatatttattgtctcgactctgctcaagccttaaaaacatctttctccttaaaaaaaaaaaaaaaaa

SEQ ID NO: 210 - protein, 248 aa, Arabidopsis thaliana
  G528
MAATLGRDQYVYMAKLAEQAERYEEMVQFMEQLVTGATPAEELTVEERNLLSVAYKNVIGSLRAAWRIVSSIEQKEESRKNDEHVSLVKDYRSKVESELSSVCSGILKLLDSHLIPSAGASESKVFYLKMKGDYHRYMAEFKSGDERKTAAEDTMLAYKAAQDIAAADMAPTHPIRLGLALNFSVFYYEILNSSDKACNMAKQAFEEAIAELDTLGEESYKDSTLIMQLLRDNLTLWTSDMQEQMDEA

SEQ ID NO: 211 - DNA, 740 bp, Arabidopsis thaliana
  G228
tcaaaaacataaccatgagaaaatgagataaagtaacaaaagcggaagtagaaagataaaaggtttaaggttgccacatttaggagctttttgtggatatgggtgattggctttcctctttggtcgaggaggtgggagatgttctttagtcccattcttctgaactttgagaaagtacttctgtgcgtggcttcgtatctacaaaaccaatattctagttactatccaacatacatagaacttatatctccaattccaccccaaagaacagagttcataaaattacttatatgtaacttcagacaaaagtatggaatgcatatgtttgattgattacctgtatcactgtttttgatccaacaaaggcctttattttcttccaatcccggtcaaatctgcaatcaaatagacatgatcatatcagaagaagaagaacaaatctcaaagattctatatatcattaatcagataaatgaaggcagagaagtgaaatccgtgtcaacatacagatgaagggcttcaaggaacttgtcgtgttcttgctccgtccagttctctctagatttggtgatagtgtaaggctttctaaccttctttgttggatcctcaaaaaacgacatcgtcctattgctccgtatggaaaccggtatcgttgttgccgtgtggggaagagtattgaaacccggtagactcattttcataggcaaacaatgagcttggctagggtttacagtcaccat

SEQ ID NO: 212 - protein, 162 aa, Arabidopsis thaliana
  G228
MVTVNPSQAHCLPMKMSLPGFNTLPHTATTIPVSIRSNRTMSFFEDPTKKVRKPYTITKSRENWTEQEHDKFLEALHLFDRDWKKIKAFVGSKTVIQIRSHAQKYFLKVQKNGTKEHLPPPRPKRKANHPYPQKAPKCGNLKPFIFLLPLLLLYLIFSWLCF

SEQ ID NO: 213 - DNA, 646 bp, Arabidopsis thaliana
  G241
atgggaagagctccatgctgtgagaagatggggttgaagagaggaccatggacacctgaagaagatcaaatcttggtctcttttatcctcaaccatggacatagtaactggcgagccctccctaagcaagctggtatgtaaaaattataatatcaaatttcttaattttgatcaaatttcttacattaattattggtaattattatttacaggtcttttgagatgtggaaaaagctgtagacttaggtggatgaactatttaaagcctgatattaaacgtggcaatttcaccaaagaagaggaagatgctatcatcagcttacaccaaatacttggcaataggtattttacttcaacatataagtatataaccgacacacaagttttattttgttttcttactatatataaatcaccaaaagaaagtaactttattacacacgtctaggtcgcactactattcttttaactacacaatgatcggctctttaattacttttgtattgatgttcttcgttttccttttggtatcatctttagatggtcagcgattgcagcaaaactgcctggaagaaccgataacgagatcaagaacgtatggcacactcacttgaagaagagactcgaagatttatcaaccagctaa

SEQ ID NO: 214 - protein, 122 aa, Arabidopsis thaliana
  G241
MGRAPCCEKMGLKRGPWTPEEDQILVSFILNHGHSNWRALPKQAGLLRCGKSCRLRWMNYLKPDIKRGNFTKEEEDAIISLHQILGNRWSAIAAKLPGRTDNEIKNVWHTHLKKRLEDLSTS

SEQ ID NO: 215 - DNA, 2206 bp, Arabidopsis thaliana
  G681
tcaaaagagatgatcaagtatgtgactactctcgtacatgacagcgtcgtacgaagtcatctcgtacatatccattgcatcaatctccattaaacgcctaacttcctcctcgccccaaatctgaatatcttctccttcactaccgtcaatcttctgacccatcattgaatctctttcttcttcttcttcttctttttcttcttcctcttttgcttgatcactgctttcgattattgaattcgacaagatgtacttgatcctatcgagactatgttgtctagatgagatacctgcggcgagtttgttaagaagacgagacgagccggaggaaggagagctttccgtcgtcgacggggacgtcgtggtggaagagttaccacaatctgcgttagtggtattgacggtgaggtgtttgatgatgggctcgtgggtcatagggtcgattccctttctcgaaagtcttttcttgagacaagagttccaatggttcttgatatcattgtctgttcgattctgcatcttcttcgccatggctgcccacctttattttttcaagaataacaagatttagtatgtcctttatcaaaaaaaaaaaaaaaaaagataaattaatattgtttccgaagatcccaaataaaaaaattcaaattaatctccaaactttgtggaaagatcacttaacatataaattaacgtcagagtggttaatcattactgaagtatatatgtggactctctaccaaagtatataatgttacatatatataacttgataattaacctgtttccgagaacagcatgaagttggatgatttcttcttcttcttgaggagtgaatttgcctcttctaatcccaggctttagatagttaagccaccttaatctgcagctctttccacatctctgcaaacctgtttttcataataaattattcgaagaaataattgtgcttttgtagtggcaaacaaaagaatctgccaagaattaaactttttattaacactggaggatttgaaagcaaggaagttgtaaaagcttcaacttttcgagattgggtcttaaaaaaacaaggaattttccaaccactatgtatatggacaaagcacagggaacacctgtcgctcttgagcaccgttaaaagatggaaatcattttttatagtatcatatggatataattggtatgagtttctttttttgtgaaagaattggtatgagtttctactaaatagaaataagcatctacaaaaattataatcaatcatggactaaaaagtattataaaacatgggtgtatgtgtaagtacgtactttcaatattcattcttgatttggttgcaaaaatcattataagaaaaaaaaatcatcagtgattttgaattcaaagaacatggattatactattcacttcagcatgtagaagagaatctctacatatgtaacaaatttccaatcccgaaaatttcggtgtgtatattaatatacccacttatgatttaactcatagtcctctaataatacaaatgagaactttcagcaacaaataggggaaagaaagtacaaagaaagtatcttcaatattgtcttaatcattttaaaacattttaaagatatatatacgaaccaaaaacaatgttaaatattctatatacttaacacatgctttttagatgttcttaatccaactatgttgctacatacctacgtgcaatatattaataggcagcaattcgaatttatactttctgtaaactcgttgttcccaaattagtttattaagaaaacaaaaaaaaaagtgagaagaagaaaatatgttgtaacacagttttgcaaaaacacaacatatagatgcatacacttctctatatgtttatggaatataaatataaatatcccaagtgttttgctacatatatacacgcatattttcatagggcagcaactagaatttatctagacttcaacaaatcccttgttccaaatacatatctaatgaaaaagagtagaagaagacacaatttcatatgagcacaaactaaaatgcacttttatgaatgacatgtttatattataccagctcttttggggagggaacgccaatcacaaacgccatgctcgttgatgtaagcgccgagcttctggtcttcctctgccgtccactctcctttcttcatcccgtcgacgtcgaaccatgtcgtcctccccat

SEQ ID NO: 216 - protein, 314 aa, Arabidopsis thaliana
  G681
MIDYNFCRCLFLFSRNSYQFFHKKRNSYQLYPYDTIKNDFHLLTVLKSDRCSLCFVHIHSGWKIPCFFKPQTRKVEAFTTSLLSNPPVLIKSLQRCGKSCRLRWLNYLKPGIRRGKFTPQEEEEIIQLHAVLGNRWAAMAKKMQNRTDNDIKNHWNSCLKKRLSRKGIDPMTHEPIIKHLTVNTTNADCGNSSTTTSPSTTESSPSSGSSRLLNKLAAGISSRQHSLDRIKYILSNSIIESSDQAKEEEEKEEEEEERDSMMGQKIDGSEGEDIQIWGEEEVRRLMEIDAMDMYEMTSYDAVMYESSHILDHLF

SEQ ID NO: 217 - DNA, 1232 bp, Arabidopsis thaliana
  G1261
atggaagcagaagaaggtcatcagcgtgaccgcctctgcgactattgcgactcctccgtggctcttgtctactgcaaagctgactccgccaagctctgcctcgcctgcgacaagcaagtccacgtcgccaaccaactcttcgccaaacacttcaggtcacttctctgcgactcctgcaacgaatctccctcttcccttttctgcgagactgaaaggtctgttctttgccagaactgcgactggcaacaccacaccgcctcttcctcccttcatagccgcagaccctttgaaggatttaccggctgtccctccgtgcctgagttgctggccatcgttggcctcgatgacctcactctcgattccggattgctttgggagtcacctgagatcgttagcctcaacgaccttattgtttcgggcgggtcgggtactcataacttccgggccacggatgttcctcctctgcctaaggtataacttgctctgttttttttctcgagccaattgctcggtttttatatttctctgaaatgttttgaaatgctccagaatcgtcacgccacctgcgggaaatacaaagatgagatgatccgacagctccgtggactatccagatctgagcctggttgtctgaaatttgaaaccccagatgctgagatcgatgccgggttccaattcctagcgccggatttgttttctacatgcgagctggtaatagagactgctattggttgtgttttcttgtgcttgtcaaatgattttccagcttataaatctctcttgtcaatatgaaggagagtggactgaaatggttcgatcagcaagatcatgaggactttccatattgctctctgctaaagaacttgtcggagtcagatgagaaacctgagaatgtagaccgagagtcatcggtgatggttcccgtttccggctgcttaaaccgatgtgaggaagagactgtgatggttccggttatcactagtacaaggtcgatgacacatgagatcaacagtcttgagaggaactctgctctctctcgctacaaagaaaagaagaagtctcgaaggtaaacaagctaatttaaaaagtgagacaccaccaataggattgggatttgaatcatgtgttgatgaaacaggtacgagaaacacatcaggtatgaatcacgcaaggttcgtgcagaaagcaggacaagaatcaggggacgtttcgccaaggcagcagatccatga

SEQ ID NO: 218 - protein, 332 aa, Arabidopsis thaliana
  G1261
MEAEEGHQRDRLCDYCDSSVALVYCKADSAKLCLACDKQVHVANQLFAKHFRSLLCDSCNESPSSLFCETERSVLCQNCDWQHHTASSSLHSRRPFEGFTGCPSVPELLAIVGLDDLTLDSGLLWESPEIVSLNDLIVSGGSGTHNFRATDVPPLPKNRHATCGKYKDEMIRQLRGLSRSEPGCLKFETPDAEIDAGFQFLAPDLFSTCELESGLKWFDQQDHEDFPYCSLLKNLSESDEKPENVDRESSVMVPVSGCLNRCEEETVMVPVITSTRSMTHEINSLERNSALSRYKEKKKSRRYEKHIRYESRKVRAESRTRIRGRFAKAADP

SEQ ID NO: 219 - DNA, 2774 bp, Arabidopsis thaliana
  G313
tcattctgaccctccatttcccagtacctcgtgtctctctactctctctgactcctcacatgctatcatgttcactatgtctcttgctacacagtgttgttctgcgctgattctctgcttatcatcccgtggccgtgctgcatctatcgactcaaacatcgctgtgtagtaatccagtgtttccacaaaccgtgacaggaatggcgaggtgtttgtgttggattcttgctctactagagtaaccagttttggggagaggctcttgatcagatgcagcagcctgtctctgtgattttcaacacttacgctctcgtctggcatgtggtgtaatacatatgggaagttcacaacaacagcaaagccaggttccaacccgagatgttcccgctgcaccttgcacccagacatgatggcatcgtgaaactcaaacgggacaccacatgactgcgccaaagttgcaagcctctcacctaccaagctgagtcctcccccacgagcataggtggactgtgaatcatccacacccgtcacacgcagcaacggcggcccaccagggtgtttcgcaagctcctgaatcaaaaacatgtattgtgatccctgtgcaatctgaaaatcgataatgtggactctggtttccccagctattgcttccaagatctcaacattcgcagttgtgtacgcgaatttccagtaggggcagatttcatagagaacactcatgtaagacatcagttcccttcccgttggttcattgcatttcaaggatttgtatatattgctcccagacccctcaagcctcgctctaagcccttctgccatgtaagtgcctagccgttggatcggactgcctgagactgacaccatctgttccaagacatcaaggaatccatacgctgtagcgaaatccccatcagcgactgcccgagcagcctccaccagcacctctttcaagtccaactgtggagtcaaagccaaaagttcatcccagttccacgacttcccagcagcggggctaaaacctgagaattcttcaacttttgtatcaccactcagcagcgagacctcaagttcccgaatcttactcttgactccagcttcatcataggcaagagatgacaccccactcaaaggtgatccatagacgttgtcaggggaatgatggagatccgagatgcaagactgagagccctgaggagaaacgggctccggccagatgtaatgctcacagaaggagagtcatacgaaggaagactaccagaagcagtggaggattcgagggtaaaaaagttttctttggacgggatatcagagaagcctttgttgtctttcgcttgaaactgaggtgaacagtagacttgaggatacagcatgtgcagtccagccgcactgtgatgtttctgagacgtttgcattggaaaaaccccagcaatacactacacagctcctgaatcaatcttcttttagccaaatcactctctacagtgtaacttgttcaaaccaaggagaataaaaacctgcctccatgaaaacatccaaaaacaacaagtgagctacaacagagtaaagcaactaaatacagacactgtgatttgtgatagaagaagaaaaaaaaggtctagttgtgaatttttctctaaacaaactcaaacatatgcgacaaagtctgctctcacaatcaatcactcaagtcatcatagggttatcagaaaacagatctgacatctttcagaaatcatatatcggtttttgctactgccttcagatttctattgccagataaaaggaaccccaaaaaatggaagctttacactctctatatatctctctctacagaagaaattacatctattcgaaggttggagtcataccctaaagcagggttttttaccagaatcaggttaaactaagggatttcaaagctttaaaacctaggctccaagtcaagatttttacacaatcatcaggataaagtttagtacttttgaatcaacacaaggcaaacaaagggacaggtagacaaatcgatgttgggttacagggaataatcgctttttgcgccaaaaccaaatcaaacaagtcatatcctttcaaaaacaaacaatcaaaaaaggcacatataagctcagattcctaacccctaggaccaaaagcttaaaaccagatgagagcagagctaaggaagaagcaattgttcaatttagaaacaaatgaaattgaatcgttttgacaagaaaataaaaaggagaaagtgactacagagatcaccatgaaagctaaccttttgaaggagagaagctgaagaaattttgttgaatgggagatttttgttgacaaggggacaaaagagaagttttccttgcatactcaagaatcagcttgaggaagaagaaggagattgagatataataaaagtgcaaattgtcatgactctcttggagctaataaaatgcccccatttatgtggattatttacttgaatgccactctgaccattgacttttggacggcagtagctgcactaccactgtttatttattaattactctgttaacgtctaattgcttaccattaatgaaatgcttaagtagagtaatcataatgtgaagttttgccgaaatagcgtgtggacataaaacttattaacttagggtagactaatatactcttgtaatgtgttttctactcttgtcgctttcgttttttttatttactaaagaaaatacatttttaaaaaatttacttccataatagaatattttgactgtggcttccat

SEQ ID NO: 220 - protein, 375 aa, Arabidopsis thaliana
  G313
MEATVKIFYYGTGVKSKIRELEVSLLSGDTKVEEFSGFSPAAGKSWNWDELLALTPQLDLKEVLVEAARAVADGDFATAYGFLDVLEQMVSVSGSPIQRLGTYMAEGLRARLEGSGSNIYKSLKCNEPTGRELMSYMSVLYEICPYWKFAYTTANVEILEAIAGETRVHIIDFQIAQGSQYMFLIQELAKHPGGPPLLRVTGVDDSQSTYARGGGLSLVGERLATLAQSCGVPFEFHDAIMSGCKVQREHLGLEPGFAVVVNFPYVLHHMPDESVSVENHRDRLLHLIKSLSPKLVTLVEQESNTNTSPFLSRFVETLDYYTAMFESIDAARPRDDKQRISAEQHCVARDIVNMIACEESERVERHEVLGNGGSE

SEQ ID NO: 221 - DNA, 1104 bp, Arabidopsis thaliana
  G335
atgtcggatgagttgtggaatgagatacaaaacctagaattaggtcaagaagatccagccctttttatccctcatgaggcttatgtgatggtggaagctaccaatagacttagtatgatagctagacctctgaacccaagggtccagaacctcaattctgttgtggtagcattaccaagaacatggggtttaacaaatcaagttcatggcagaatacttgatgctacttatgttcagtttctttttcaaaatgaaattgatctaatgatggttcaacgcaaggagccttggctttttaacaattggtttgttgcagctactcgatgggaggtggcaccggctcataacttcgttactacaattgatctgtgggtgcagattcgtggaatccctctgccatatgtttctgaggagacggttatggagattgctcaagatctgggggaagttcttatgctagactaccatgatacaacatctattcagattgcttacatacgggtgagagtgcgctttggtattacggacaggctaaggttctttcagcgaattgtctttgactccggggaaactgcaacaataagatttcagtatgaacgtctccggagaatctgcagtagctgcttcagattcactcacaacagagcttattgtccataccgaccaagacctctcagcattgcaagggaaagagctctgtttagggatagtgtgcatcgttcctccatgaactctcagtctcagatgactgatagttcatttccaattccccagactccaccaccaagaatatcccacccaccactgaatcatgatgagtttgtagctgcttatcctcacttggactcaggtaggaatgaccacatcagatgtgagggagaatcaagcaatttcagacaagacttgtcttcagcttcaaactccataactccaagaagggaaccacaatacctaactgatagaagacactttgaacctggtcaatcctcaagaagacatgatgttcgggatttaagaagagggtcagagaggattgggaatctcaatcaacagaactatgtgcaaagatcaggagggatactgaaacctccgaagaaacgctaa

SEQ ID NO: 222 - protein, 248 aa, Arabidopsis thaliana
  G335
MSDELWNEIQNLELGQEDPALFIPHEAYVMVEATNRLSMIARPLNPRVQNLNSVVVALPRTWGLTNQVHGRILDATYVQFLFQNEIDLMMVQRKEPWLFNNWFVAATRWEVAPAHNFVTTIDLWVQIRGIPLPYVSEETVMEIAQDLGEVLMLDYHDTTSIQIAYIRVRVRFGITDRLRFFQRIVFDSGETATIRFQYERLRRICSSCFRFTHNRAYCPYRPRPLSIARERALFRDSVHRSSMNSQSQ

SEQ ID NO: 223 - DNA, 5394 bp, Arabidopsis thaliana
  G624
gggttttatatatgctttcctcttcatccatgtcatcgtcttcgttatctgctagattttgcttcaaccacgaatgttttgagttcaaactcgaccattgccgtcctggttggcgtctccgttctggcgatttcgtcgacctctgtgatcgttgcgcgtaagcaaccttttccgtacttcttttcttcgcttatttttactctccctgttccatttttttgagttgatcatttttttttttttcaagtgtgcttgtctttgattttggaaaacttacttggttgtttttttcctacttctttctttgtgtaatttgggtctgtcttagtaagatgtggcagcctgtggtaatttctgagtttttttgttgtttgctttgttttggagtatagagtccccatgtgttgactacaagccgagaaattaggagccactcttttcggatgttttggaattgtttaacagtgttgaatttaagtgactggaatatagagttttaagcattttgatgggcgtgtgaggactaattcgtttcacctgaattctttatgtggcatcgatgttgtgtaacaagtttagagtctagaggtccaaagtccaagtgacacgaaagagatagaagaagtgatactaggagacaaaataattaaagcaataggaagctactttgcattctgatccattagattcgagaatttgttcactagtggccggttttctgaatttttatattctgcttcatcaaagttagaaattagacacatgattagtttcttaggcttactatgatattttgtgaaataataaattgggtatggtaatggtttctatgtaaacaataaggtcaccttcatgggattgcaaagccttttgagtatcaggaattaactaggaatgagatacttttgctcttactacttcttgattgtaaacatgcatacatgtgttctgtctttgatgggtttatttttggaaaacatgtatcagtttgatgatataatcttcaataattttcgttcctgtatgtagatcttttcttaaaaatgctttgattctttaactatggatgcgtggagactatatagtgtatagtttccttaggcttctgtttttgttctgaaacataaacttcatcagagctgaaaaagtatagttaaagcttacaagttctagttgcattaggcattaaccaacacaaatagtctggtttttctttgtttgatttcagtactctgcagacacgattcttttacctgctctttgcatcaatttcattaacttgttagtgtattatcgattttgcaaatatctaatgattgtattctggttccagctctgcgtatgaacaaggaaagttttgtgacgtatttcaccaaagggcttctggatggaggtgttgtgagtcatgtggaaaggtaactatatgatttactagttaaaattttctatgtctccttggcatcccaaaatcatacatggggtttccagatgtgaagtctctcactaatagcatttcgtgcagcgaattcattgtggatgtattgcgtctgcttctgcttatacgttaatggatgctggaggaattgagtgtttggcttgcgcaagaaaaaagtttgctttggtgagggacgatttcacttttttatttcctgtcaccaaattccatccatattttttctcggaggcatgatgttacctgattcaatgtctctcagtaattatatatcttaagctagcaagcaaacttcaactgtacaaagatcttttcaagtttaagatcgatgttgagctgtagtctgtgtgctataacagtctgtattcatgtgtttcagggacccaatttttcgccatcaccgtcctttcttttccagtctccaatttctgagaaattcaaggacttgtcgatcaactggagttcttcaactagatcaaatcagataagctaccagcctcctagttgtttagatccttcagttctgcagtttgattttcgtaatcgtggaggtaataatgaatttagtcaacctgcttctaaagaaagggtcactgcatgtaccatggagaaaaaaaggggaatgaatgacatgatagggaaattaatgagtgaaaactcgaaacattatagagtatctccttttccaaatgtcaatgtatatcacccactgatctcactaaaggagggtccatgtgggacacagcttgcgtttcctgtgccaattacaacaccaattgagaaaactggtcactctagactagatggaagtaatttatggcacacacgtaattcttctccgctaagtcgcttacacaatgacttgaatggtggagcagactcgccattcgaaagcaaaagtcgtaatgtgatggctcatcttgagacgccaggaaaatatcaggtggttccacgattttggcccaaggtttcatataaaaaccaagttctgcagaatcaatcaaaagagtatccttcttctcttatagatactacattggaatataactttgtatgtttaccagaagcatatgaatcatgctttttttcatacttctgtgtccttttactcttgcttttagtttgtcgttctagcttttgatatgaataagtctatactaggttgtagaatagggtggatgtaaggctatcagataaatggttttagggataggttttactttggatacttctaagttaccagaaccttaacgatgtgtaagatctgaatccgttgtcactcctctctttgagaagatcctgagtgcaactgatactggaaagcggctggtcctgccaaagaaatatgcagaggtaaataactgtttgcttatgcaagtacatattctcgttttcttatttaaatggttaatcatgtgaatagaatttgggatgtggctctctgttgcttatagcaaatacttcactgcttttgtatgttgaatctagtagtcataatatctgaaaactttacagcgtctcaaattatatataagatgccaaatgaacaaggcatctgctatgtaacaggctttcttgccccaactttcccacaccaaaggtgtgcctctcacagtacaggacccaatgggtaaagagtggagatttcagtttcgcttttggcccagcagcaaaggcagaatatatgttctagagggagtaactccattcatacagaccttgcagttgcaggctggtgatacaggtatatcgtttgtgatatagtaatttggtttacaggaatatatgttttatgtagtagcatttgctgttcacaattccatatgtttaatgcggaagcaactttttttctcactcaattactatttttaaatgatatcttttatgaagcatattacacttcttctgagttaataaaatttttctagagcttatgcctgggagctgatttccatttactttttagtcatattcagtcgattagatccagaaaggaagttaattttgggattcagaaaggcttcgattactcaatcgtctgatcaggtgactttccaaattaactagatagcttttaattgtaaatacttatggttcactatagttacaatcttaacttagtagtaaactagcggctagttttaaaagctttcctgaatgtcagctgcactctagagtatagtcaaagcacaaccaggaataacacgctgaagtataatgagtgaatacgcattgtctacttctctattctttgttcaatttgtgttaagtgattaaataggtcggacaagattcacaagtatgcagttcgaatatggttttttttatcatcatctcttctgtctcccattttggttagttctcaaagtgctgagtagtcatgtttattcgggcatggaaatgagttacaaagtatgcgaagtaactaaaatcaaactacaccttttcttgacaagttagcttgacaaatatattatatctcttctccatttccattagcaggctgatccggctgatatgcattcaccgtttgaagttaagaagtctgcttatataactaaagaaaccccaggagtagaatgttcctctggtaagaagaaaagcagcatgatgattacaagaagtaaacgtcaaaaagttgaaaagggagacgacaacttgctgaagctaacatgggaagaagctcaaggatttctcctgcctcctcccaacctcactccatctagagttgtgatagaggattacgagtttgaggagtatgaggtacatccatatgtttttgctctaatttgcataagtggtctatgccgattaagaatcttattctcttttcgtttctttaggatttggctgtagtttctcaagtttcttctggaatacaagattatttcatgatattaaaaataacctgttgttcatatacacattgggtgctagaagttgtttgtctctagcttttaaaatattgtgtggttaatcggcttagagtgctcttacctaaaacagttgataccattgatgtgccgtgttattttgttatcaaccatcgttttttttttttttttttttgtgtttttcaaatcctgttttacttgttttggaacatatcaattcactgagttatttctttaatgcttctcgtgtcaaaataggaggctccaatcatcgggaagcctactgatgttgcagggtaaaccacacatctttgcttttattggcttttctctttgcttttaggtgtttgaaatggtgaatcttatcgtagataatggcggtagaatcaaattagggtactggtactattcattgtccgagagtgtgttgccaagtgttgaaaagctcattgattttcttctgcttcttgtttttttcttttggatcgtgttgtctctagtttcagatcaacgtgcactgaagttgaaggactgttgatatcaccaacaaccacaaagcacccacgacatagagatggctgtacttgcatcatttgcatacaatccccaagtgggataggtcctaaacacgaccgatgctgttcttgtgcagtctgcgacactaacaaacgccgtaggcgttccctgttgctacggagggaaaagaagcagatggagaaagaagacaatgcgcgcaagttgctcgagcaactgaattctgataatggacttcaccaatctgctaacaacagtgagaatcatgagcgtcatgcttcgcctttgaaagttcaattagatttgaacttcaaaccagaaaaggacgaagaatctctccctggttctaacaaaactaccaagagcgagactctgcctcatgatgacacagtaaagtctagcttcacgtcaccaagctcatcaagtgctcatagccaaaataacaaggaagatgaaggaaaactcaagacaaccacagaaattgcagacaccaccaccacaagctccatgtag

SEQ ID NO: 224 - DNA, 1216 bp, Arabidopsis thaliana
  G501
aattgaattttcaaccaacgaagaagagatttttccaagagcaacagacaagaagaagagaatgaagtcggagctaaatttaccagctgggttccgattccatccaacggacgaggagcttgtgaaattctacttgtgccggaaatgtgcttccgagcagatctcggctccggttatcgccgagattgatctctacaagttcaatccttgggagcttccagagatgtctctgtacggagagaaagagtggtacttcttctcacctagagatcggaaatacccaaacggttcgcgtcctaaccgggcagcaggaaccggttattggaaagctaccggagcagataaaccgattggtaaaccgaagacgttgggtatcaagaaagcactcgtcttctacgcagggaaagctccaaaagggattaagaccaattggataatgcatgagtatcgtctcgctaatgttgatagatcagcttctgttaacaaaaagaacaacctacgacttgatgattgggttttatgtcgaatatacaacaagaaaggaaccatggagaagtatttccccgcggatgagaagccgaggaccacgacaatggctgaacagtcatcatcaccttttgatacatcagactcgacttacccgacattgcaagaggatgattccagcagctcaggtggtcacggtcacgtggtgtcaccggatgttttggaggttcagagcgagcctaaatggggagagcttgaggatgctttggaagcttttgatacttcaatgttggtagttccatggagttgttgcagcctgacgcttttgtccctcagttcttgtatcagtctgattatttcacttccttccaggatccgcctgagcagaaaccattcttgaattggagttttgctccacaggggtaaaaacggaagagaccaaaaaaggtgtttgctagtagtactgtgatgtgccagagagaagagtctcatctcaactcatccctggctcttagtagtaaaagaagattgtagaatgttaatagcttttagcatcaatgtctcattagcaggcacattcttgttctttcatgagaagtttatatgaaaactaaaaatttatattcaaattcttcaagatgttgcacttatgtagatactgatattaaataacaacctaacctttatgagaaaaaaaaaaaaaaaaaaaaaaaaaaaaaaaaaaaaccct

SEQ ID NO: 225 - DNA, 434 bp, Arabidopsis thaliana
  G682
tcaatttttcatgacccaaaacctctcaatttctccagcggttcttcctgggatcctcccagctatcagttcccacctttcatcaaataataacacacaaaattcagcttttactatggtgttacaattaaattattttcctacgaaatagtattcattattagttaaaagatcaaacctgtcaccgacaagcttatgcattcgagagaccaaatcttcttcttcttgactcatgttcacaacttcccactcaagactactcacttctgttccttgtcatcaccaaaattcagatttctcattatatatagataagtataaaaaaacatggaaaaatgagaaaacgaaggtgtttaagttttcagcttaccttcagaagaagaagtaacgatggagttggtcttgggttgcttagtcctgcgatggttatccat

SEQ ID NO: 226 - DNA, 878 bp, Arabidopsis thaliana
  G1274
ggtcatcgagttgcatttagaacgagatcgaagattgatgtgatggatgatggttttaaatggaggaagtatggcaagaaatctgtcaaaaacaacattaacaagaggtaacttattcatttacaaattactcatttgatctaattaatatgttttactagtacttgtttttaacataacatatggttgcttgataattttctataaccatcttgtaatcctgttgttatagaatatatagccactagctagctagaccatatagtgatttgaatactatttaatggacccgtattatctaccaacccacttggaataaaatttagcttctatatacatcatgcatgtgtgttttgtttatacaaatatataaaaggttcctatagcaaaattgattgattagtggtatatggatcaatgaatccatggttgataatcagtcatcgtatagatttattttgactcatatatcataagagaaacaaatataatgaattcatcatctaaacatatcactttcattcaaagcatggtttgaaagcaatttacacactctgtaattggttaatgatattcttatatatatacatacatatatatatatatatatatatatatataacttggtgcgatatattataaaagtgattacaatttaacatgtggtggatgaatttataatataaaggaattactacaaatgctcaagtgaaggttgctcggtgaagaagagggtagagagagatggtgacgatgcagcttatgtaattacaacatatgaaggagtccataaccatgagagtctctctaatgtctattacaatgaaatggttttatcttatgatcatgataactggaaccaacactctcttcttcga

SEQ ID NO: 227 - DNA, 953 bp, Arabidopsis thaliana
  G767
atgatgaaatctggggctgatttgcaatttccaccaggatttagatttcatcctacggatgaggagctagtcctcatgtatctctgtcgtaaatgcgcgtcgcagccgatccctgctccgattatcaccgaactcgatttgtaccgatatgatccttgggaccttcccggtacgtttctttcttttcccctttaaatcacactatacactaacagtaacaggattctataaaatcggtctaagaattaataaaccggtttacaatttacagacatggctttgtacggtgaaaaggagtggtattttttctcaccaagagatcgaaagtatccaaacggttcaagacccaaccgtgcagctggtactggatattggaaagctaccggagctgataaaccaataggtcgtcctaaaccggttggtattaagaaggctctagtgttttactcgggaaaacctccaaatggagagaaaaccaattggattatgcacgaataccggctcgctgacgttgaccggtcggttcgtaagaaaaacagtctaagagtgagtgaatttatcagttcaaaccgaactttgaatatgattttgaatcggtttagttaaccggacgtgtttttgatattcttgaacagttggacgattgggtattgtgtcgtatatataacaagaaaggtgtcatcgagaagcgacgaagcgatatcgaggacgggttaaagcctgtgactgacacgtgtccaccggaatctgtggcgagattgatctccggctcggagcaagcggtgtcaccggaattcacgtgtagcaacggtcggttgagtaatgcccttgattttccgtttaattacgtagatgccatcgccgataacgagattgtgtcacggctattgggcgggaatcagatgtggtcgacgacgcttgatccacttgtggttaggcagggaactttctga

SEQ ID NO: 228 - DNA, 2522 bp, Arabidopsis thaliana
  G961
tcaatggagagagatctgcatctgaggcgatgaaagatcgaaatgaacctgcctctgccatggttggaacggccagaactgaagctgcagacgaaggagacaaacaagcagcaaaactggaagagcaaaggatttagacttcacagatctagatctgtgaatttagaacagagaccgttggaagaaaataggattcagatcacattaaacccacaagaaaacgaaactaaagagattacaacaaagaaattccggttttggtgctgaaaaagccaccggaactggaaaccctaggcggggcggcgaagcttctcgtaatatgttttttttttcttctagaaggaaaactataaaattaaatggaagacattttgattattataatgaaatgtaatgctctccccaacaataacgaaatatctcctttttgagatctttattctcctcttttaaattcttatattaatctattttacaatatttcacataaccactgtaattatcacaaatctgactttcttttatatgtttcaacaactatatggtatgattatatctttttacacaaatgtgaaatgtcttatgtgtgtgtgcctctctctctttccctccatttccgttatccactaccattcgacacgtgacaaaatgggccaggcgatgacgatagagacgacgtcgtcgtattccatagctccatctccggtgtatattcctgtgttaaaccggcgtggtacgaagcggaccggtttaggctggggtacggagatcggacggaaggcaaagcgagatgatcatggcccacgtggctctcattgacggctgtaattgaatatgatgtgggtccgttaagctgcgaggccacgaggcggtctaaagccgcccagctagtaacaaagctagtgtcgaccacgttgctgacgtggatattatgattagtgtcgggagagcttacgtggcagttgttgattgcctgactgttagggctttcgaggtttgggagtttcatgaaagggtcaagatttagctcttctttacaagatctccccataagttcaagaaattggtcgagagtatcctcgttgaagatctgagatgatgtcgtcttcggcgtatctccgccgccgctcagggaagctcctccgacgggactgtttagggttttgtgaagattcttcttcttgaaaatacgacacaccacccatccttcgtcttgtgatgcttcccctataatactcacgaccttaaatattcaaaaatcaactaagatatattagcaaagttcatgttttgcactaatcccatttaggttaaaaccaaaaagaaaaagaaaattcatatttcagtttcatgtgcgtctgtatatgacaaaggagaaaagaaatatttgtgggaagaagtaactaaaactattatcattaaatattgggaatttaagaaaaaaataataacgaaaacgaatatgtgatagttttcataatgttattaaagaaaattacgatcatactttgcataaatattgttttttaaaatatattaattacctttatatatatattaattacatgatattttttacttgtacatcttatcatcagtaacaacttttataatggtcaatgaaataataattaatcaaaacaaaatcgagattgtgatgtaaaatgagatattcaaaaaagagaccaaatcctttgattgaataactcgtatttaggttatgatggaattaatacatacctcatgaacggtgacatcctcgggggaaataatgttgtcatcgagtctatattcatgcatgatccaatcagatttttggccgtgaggagctcggcctttgtagaaaacaagagtctttctcatcccaattctacggccattgctatatatgatcttgtcgcggccagttgctttccaaaatccagccgcagtggctctgttagttctcgttcccgtcggatattttttgtccttgtggctaaagaaataccagtcgttttgtggcgttgttcctattttacacatctctgcataacgacaaaaataatcagaagaaaaaaacttaaatagtaaattgtataataaccaaacctatgttggtctttgcagtaactaactccgaatatgaacaaacaacatcaacaactaagtgtagattctctgtactctagtcatattctcgtaaaaggatacatgagatcacgtcatgaatcgaaagtagtatagacaagacagcactttcatatatatggaaaaattaataaattcggaaaattggattatgagagtgtgaaagagagaaaattaattcataaaaaatttctttatggaaataaaccttgaatgtcccaaggctcgagcttgttgagatcaacgtcgcgaatgacatcaagatcgatctcgatgctattaactttcttccggagataatactgcaacagctcttcctcggtcggatgaaacctaaacccaggaggcacttgagattgtccgttcactgatatgctcatagattttgacat

SEQ ID NO: 229 - DNA, 1580 bp, Arabidopsis thaliana
  G206
atggggagaagaccatgctgtgagaagataggattgaagaaagggccatggagtgctgaagaagatcgaatcttgatcaattatattagtctccatggccatcccaattggagagctctccctaaactagccggtaaataagccattttatatttgctatttgccatatgtatatattagtaacaaaataattttgtacctagctagcatgtgtattattgttctatagtatatagtagtgtatatttgttgtgtaaagttaataatggtgtttgtgaaatgattagggctacttcggtgcggaaaaagttgcaggcttcgttggattaattatttgagaccagacatcaaacgtggcaatttcactcctcatgaagaagatactatcatcagcttacatcaactcttaggcaacaggtttctcctccttttccattttctgtttttttacttcttcctttgattatatgcgaaattttagattttttggtcctatattctttgaaaccagaaattaaacaagaatttgagaaatcactcacaaatttttctttatttttaatctttgagaagtcaaatcacaagtttttttctttcttttaaagtaagtcaatgcaatttctgtttagtccataactccatatatattacagtgaaaatgatataatgttcatttgtagatgaaggattttgggacatcaaattgtgaaaagtatcattattttggaagtttagtttgttttaactgtggagactagctagctagattactttacaattttctctttgtatatgtgttgcttttgtcaatatgcgagagtttatatctttgtggggcagagaactgagaagattccaacccgtgattgcctttgtccattaaataatttcaattttttactttcctctaattatactttttattcactagtagacaaagataggcactgtgatcagtgtaagacatgtgcaaattcttttaaccatacgcatgaacttcaataacattccttttgttttactcgctctttgttctcataacaatgtagacgacgtattatgatgattgatgagtcattcatgtgacatttgttttgagtagatggtctgcgatagctgcaaaattgcctggacgaacagacaacgaaattaaaaatgtttggcacactcatttaaagaaaagactccaccacagtcaagatcaaaacaacaaggaagatttcgtctctactacagctgcggagatgccaacctctccgcaacaacaatctagtagtagtgccgacatttcagcaattacaacattgggaaacaacaatgacatctccaatagcaacaaagactccgcgacgtcatccgaagatgttcttgcaattatagatgagagcttttggtcagaagtggtattgacggactgtgacatttcaggaaatgagaagaatgagaaaaagatagagaattgggagggctcactagatagaaacgataagggatataaccatgacatggagttttggtttgaccatctcactagtagtagttgtataattggagaaatgtccgacatttctgagttttga

SEQ ID NO: 230 - DNA, 1080 bp, Arabidopsis thaliana
  G907
atgatgatcggagaatctcatcgtggttttaatccaacggttcatattcctccatggccactttctgaagatctaacggtgtctgatatttacggaagtccagacggaggaagtagtatgatggaagctttggctgagttacaacgttatcttccgtcaaacgaaccggatccggattcagatccggatctctcgggtccggattcaccaatcgatgcttatacatgcgatcattttcggatgtatgagtttaaagtgagacgttgtgctcgtggccggagccatgattggacggagtgtccttacgctcatcccggagaaaaagctcgccgtcgtgatccgaggaagtttcattactccggtacggcgtgtcctgagtttcgtaaaggttgttgcaagagaggagacgcatgtgagttttctcatggtgtttttgagtgttggcttcacccggcgcgttaccggactcagccgtgtaaagacggtggtaactgtcgccgtcgtgtttgtttctttgctcattcgccggatcagattagggttttgcctaatcaaagccctgatcgtgttgattcgttcgacgttttgtctcctacgattcgtagagcgtttcagttttcgatttctccgtcgtctaactcgccgccggtgagtccacgaggtgactcggactcgtcgtgttcgttactgagtcgttcactcgggtctaatctgggaaacgacgtcgttgcgtctctcaggaatctgcaacttaataaagtgaagtcttctctttcgtcgtcatacaacaatcaaatcggaggatacggatccggattcgggtcgcctcgtggatcggttttgggtcctggtttccgtagcttaccaactacaccgacccgacctggttttatgaacatttgggagaatggcttggaggaagaaccggcgatggaacgggtcgagtcgggtcgtgaactgcgagcgcagctgttcgagaagctgagcaaagagaattgcatgggtcggattgaaccggatccggatcagggagcgggtgatactcctgatgtcgggtgggtctctgacttggttatgtga

SEQ ID NO: 231 - DNA, 861 bp, Arabidopsis thaliana
  G171
tcaaacggaagaaacaaatgacatttctcacatagaaagtttacttgaatcctccaaatcattaacttgtgcaaacctgaagggataatcttggaactgcttcacattgttgaacatactgtttcctaaaccccaatgtattggctccatataagctgtcctctgtaccatcggaccatatggaccatccgaacaactcccgtaaaacaagctttgcccgttcgctatcatcggctctgtcacattcgagaaggaagacacttccgcagtatttgaggtcatctggttattaaaagggaagaaagagagcccgttgtaatccatagtaggcagaggaaacaactgatactgctcaaaacattgccgattgtaaccgcataaaccaagttggtcattagaccaacaactagtgtcaggaaatgtctgattcctattcgtagcctcttggatcttgttttctactgccatgaaaaccgcatagaggtcatttaaagaacactggtcgagctttttgtcccatgtaggatactttgtcgcaatcgtcggtttctccaccttagtgttgtttttctccaagcattcttgcacggtgtatgtcttggtgcagctgcttgacgcagtgtctctgtacttggttaagatttcacggactttgctcccatcctttggccataactcgggctccatgaccatttcgtcccctgctctgctcgggccgtacacaatgacacaagtgtccacaccgcagagtgttgagaactcactggctttcttatacaaacatgctttcctcttcttgtaagtcgttatcctcgttttctcgttcgttatcctcgtcatctttaccatctttcgacccat

SEQ ID NO: 232 - DNA, 375 bp, Arabidopsis thaliana
  G348
atggatccaaggaagctactatcttgttcatcctcttacgtgtcagtgagaatgaaagaagagaaggggacaattaggtgttgcagtgagtgtaagaccaccaagacaccaatgtggagaggtggaccaactggtcctaaggtctcttcttctacctttaattactatattcataactttgtttgatcttaagataattcatcaagtgttcttaagttgtttattttgatttgtggtgggatttgcagtcactttgcaatgcatgtggaattagacacagaaaacagagacgatcagagttattgggtattcatattattcgcagccacaaaagcttagcctccaagaagataaacctattatcatcatcacacg

SEQ ID NO: 233 - DNA, 1187 bp, Arabidopsis thaliana
  G1237
cgggttgacagtgagatgggatctgctgtgaccttgggtcagaagtggttcaatggcaagtgtgtgtagatcactccagctagagctgagcatatcaaacaggacaagtgcccttcatgcagcaacaaaagagctcgagcttaaaaatgttgtattgtagagaatagcctgtagtgacaactctcactaccctcgatctgtctttatttggctagtcttttagctcgttattggccataacagcttgggcctatttctctgtataattggtttgattttgcaactgagaaaaaaaaggaaactctttaactgaatttgtggttagcttcaaaccaagaggaaacataacaattcttaattcggatacagcaatatactctagaagtctcgaaatcatcactaaccctatctggaacaattatgttagggttctcagaacctgaggtaacttgtgatgcttctctagcaatctagcaatagtctcaactctcaattgggagtgtaacgaaactgaagatcacatcaacaaaatcttgacacattttatagtaaattcaaaataaaacataaacatttcggacacatgaaaacaaacaaaaaacaataacaagaagtcttgaagcaaacaaatttagaaaggatagccaacacttgagagcnccnnatttaaagagaagaaatgaacnccgatccctagttcactatgcccggtctctcacgaactgaatccggcgtagttttcataacgaaaccttaatcccttaacaattccattaccccttaccccccttacccgagaagacactacccttccctaagtcagttcccatctcccctgcgaaaagaaatagcccgttactcgcttaacttcccggttcctcctggcgagaatcatccccccccaatttttatgagactacaacttcctaaggcaggtttttatcttcgacaaaattgttttcaagaaaattttgaatactccaaaatctcgatcttccccctccctggcgccgcaccgggcgtttttcctccggcggatcctccatacccaccccctgcgaagaccaactcttgcaccgcgttcgccgcggtggcccctatatccggggaaactcgctcccgctattatatccacagagtggcctcctctatcccgagacacaataaacctcttctgcgcgcccccc

SEQ ID NO: 234 - DNA, 1113 bp, Arabidopsis thaliana
  G516
ctatagctttgtgagaaaaaactgaaaacagaggagatagtaaaggtaaaataggaaaattttgcttacagttgttgcagaaatcggatcagaatcactaccatcggtgctatgatcactgaagacaccggtcaatggcttttgaggtgtgtggttgttgcgatcatcttgcatagaaacaatcttactctgctcatcgtcatcattaaagaagcctttgaaatcatcactcaaaatattgcagaagtccctatctagtgcatccacatcaagaaagccagaaaactgacttggattctgtgattcttggctgtgaaacaacgatccctgaaaaaaggaacaacgtaacacagatgaaaaaactatgtgtacgagatatgattttgaagatttaaaagagaaacagggaaagtttttgagattgactcacctctgtctctacactcaaaactccagaattgttggtaggagggattaaagagtgagtatgcttaattccactaccagtagcaactgaaaattctctcctttcacccttaaacttaactttgcagagtgtgtatgtcattatctacagatcaactcagcataaatgattaccataaactcagatttttcccgtttacgagaaataaaatcacggtaagaaaacaacaacaacaagttaaagaagcctaaacaagagaatcgaaaaatgacctgattaggagacgagaacgtagcgtgatactcatgcatagcccaatcggacttggaaccaccgtgattcttaaacaccaaaacccttttctcaccgatcttctcacgatctcccgttttgcgcattatatccatggtaacgccagttttcttccaaaaaccagacttggtttttcttatctgtcgatcacctttaccatatttgttctccttacggccgaagaaataccaaacctgatctcttgttttcatcctagaatgggctgggaaagacagaacatgatcagaaattaccgggatgatacaacagttgggaaccaaagaaaaacaaacactcgcgttagaaagacttacaaggtaagtcccaagggtcgaagctacaaatatcaactgtgctaatgacttcatcgacat

SEQ ID NO: 235 - DNA, 651 bp, Arabidopsis thaliana
  G1222
atgctggctggcctagcaacatgtggtttcatgatgaacatagtctcgagagcttcagatctgacacaagacttcaagacaggttacctcactttatcatctccaaagtcaatgtttgtgagccaagtgattggaacagcaatgggttgtgtggtatctccttgcgttttctggctattctataaagcatttgatgatttaggcctcccaaacactgaataccatactccatttgctaccgtatatcgaagcatggctaaactaggagtaccaagagaatgtctcgttctatgctacgtgttcttcggtgtggcgattctcgtaaacatagtaaaagatagtctccatagcaaatggggaaggttcattccactttccatggcaatggctataccgtttttcttgggactttactttgcaattgaaatgtgtgtgggaagtttgatactttttatctgggaaagagtagatgcagccaaggctgaagcttttgggacagcggtggcttcttgtttgatatgcagagatggcacttggtctaagccaagctcagtgctcgctgtagccgcagttaatcctcctgtttgcatgaagtttctctcttctcaaacaaattcaaaagtcgacaacattctgcaaggatccacttaa

SEQ ID NO: 236 - DNA, 2610 bp, Arabidopsis thaliana
  G1218
atggagagatctagatccaagagaaactaccactatgaccaagactatgacggagattccatgcctaggtctaaacctcgctacaacaataattaccactttggtggtggcggcggaggaaataacagataccgtggcggcggcggtggtggaggtggaaatggccggccatcgaagtctcatcctgaaacgatggcaacaacgacttatcgaatcctctgtcacgacgctaaagcaggaggtgttattggtaaatccgggacaattatcaaatctataaggcaacacaccggcgcgtggattaacgtgcacgagctagttcctggagatgcagagcgtatcattgagatttctgataatcgtaggcgtgaccctgatgggagaatgccgtctttctctcctgctcaagaggctttgtttagtgttcatgacaggattttggagagtgaagctcagtttggttatggtggacctccgccggaagaggaggaggattacggtggagttagacctggtggagggagagttgtgactagattagtggtttctaggatgcatgttggttgtttgttgggtaaaggtgggaagattattgagcagatgaggattgaaactaagactcatattcggattcttccaagagaaagtaacttacctcgttgtgtttcactctctgaagagattgttcaggtctgaaaaattgctattttgagaacatatttgaaatggtgaatcttgttgagattttgatatgaaatgtagtcagtttattgaaatgctagataatttaaatgtgtgattgtgaggttgaggttgttgacaagagttgaatcactcttatgtagttagcttggtcattatttgttatgtttaagctttctttggtgttttctacctatgggaaatgcatttataatgttggttctttcagattgtaggtgaactaaatgcagtgaaaaacgctttagccattgtatcatctcgtctaagggagagtcagcatcgtgatcggagtaatttccaagggcgttcacattcaccagaacggtcgtttgcagctgcgggggatgattacatgccacagctaagaagacaatcatccgataggtttcctcgtggtaattttagaaataacaacttcagttctcgccagtccaactatgcagaagaagctccagcagttccagttggtgagaatgtctactccgaagagcttgtgttccagattctctgcccagctgacaagattgttcgggttgttggggaatcgcaaggaataattgatttgcttcagaacgagatcggcgtggacgttagagtatctgacccagttgctggatctgatgaacagataatcaccatttcttcggaagaggtactctaaataccctcagatcttcttcaatattatctgttacatttacaattgtgaacttgaagttttgtattcttactttgcttaggcacctgatgatccatttttccctgctcaagaagccttgttgcacattcaaactcagatcatagatcttattccggataaagataatttaataacaacaagattacttgtcccatctagagactctatatgtttggaagggaaagcaggatcagtatctgagatatcacgattaactggaacaagcgtacaaattctagctagggaagaaattccccgatgtgcttctatcaatgatgttgttatacaggtatgtttgtctatttttgaggagtcgagcattttctaaacaacttctggtctcaaaagagaattcttatgtgaatgtcgatgtagattactggcgagatcagagctgctcgggaagcacttgttgaattaacattactattgaggagtcatatgttcaaggagctctctcaaaaggagacaccaccagcttccacatccacaactggccctctcgaaggtgttgcaggagtaatggaggtagcttcatcaaataacacaattcaatctcgtgaaggtcctactagttcgaatctgaacttgcagcaagttagcacaatcttgccacagtttaaggtttttgttctcatcaacaatcttttcaatataaaggcatctgtaatcagttagggtcagacttatttttgccatcgaaacaatgtaatattactatcaggctatgcataattacctgaatacttggaaagtagtctatggttcttgtccattcgtgcaatgtccctatatttacatatatttttctcttggtttttataggaaggtttcggatcagttgcaaaggcgggtgaaagtgaacaccgggaagaggttcctgtcacgacaagcaggtagagaaactcccaaatttgatatattttcccaaaaccgtacaagtgcttgtttcacacctttccttcctgctttttgtttttttctctagaatggcggtaccacttgttactagaagcactcttgaagttgtcttaccagaggcggttgttcctaagctcgttacgaaatcaagaaacaagcttgcacaaatcagtgaggtagaactctttttaactggaactagattggtgcaaatcatgactctaatcaaaatctttgacgctttgtcttatctctttcccacttga

SEQ ID NO: 237 - DNA, 923 bp, Arabidopsis thaliana
  G251
atgaaacttgtgcaagaagaataccgtaaaggaccgtggacagaacaggaggacatcctcttggtcaactttgtccacttgttcggagatcgaagatgggattttgtagcgaaagtttcaggtttgaaggtggagggagaaacataagaataggtataggtttgttttggaaaaatgggcggtctttggaaaggaccttccatttaaagaaatgacctggttttggttgtaggtttaaacagaacaggaaagagttgcaggttaaggtgggttaattacctgcatcctggtctcaaacgtggtaagatgactccacaagaagagcgtttagtccttgagcttcacgccaaatggggaaacaggtcagaagaatcttcaagaaacagagaaaccctaaaaatgtttttttttttaaccctaaaaatgttctttgtttgttgtttgagcttttggatactttcatatgcaggtggtcaaaaattgcccggaaattaccggggagaacagataatgagataaagaactactggaggactcatatgaggaagaaggctcaagagaagaagcgacctatgtctcctacttcctcatcttcaaactgttgctcatcatctatgaccactactactagtcaagacactggaggctccaacgggaaaatgaatcaagaatgcgaagacgggtactactccatggatgacatatggagagagattgatcagtctggagcaaacgttattaaaccggtaaaagacaactactactcagagcaaagctgttacttgaatttccctcctctggcttctccaacatgggaaagttccttggaatctatatggaacatggatgcagatgaaagtaagatgtcttcttttgctattgatcagtttcctctaagttttgaacatggtagtggtcgcctt

SEQ ID NO: 238 - DNA, 896 bp, Arabidopsis thaliana
  G134
gagaaagagatagagagagaaagatgggtagaggaaagatcgagataaagaggatagagaacgcaaacaacagagtggtgacgttctcaaagaggaggaatggattggtgaagaaggctaaagagatcacagttctttgtgatgcaaaagttgccctcataatctttgcaagtaatggtaagatgattgattactgttgtccttccatggatcttggtgctatgttggaccaataccagaagttatctggcaagaaactatgggatgctaagcatgagaaccttagcaatgagattgataggatcaagaaagagaatgatagcttacaactggagctcaggcatttgaagggagaagatatacagtctctcaacttgaaaaatctgatggctgtcgagcacgccattgaacatggcctcgacaaagtccgagaccaccagatggagatccttatatcaaagaggagaaatgagaagatgatggcggaggagcaacggcaactcactttccagctgcaacaacaggagatggctatagcaagcaacgcaagaggaatgatgatgagagatcatgatgggcagtttggatatagagtgcaaccgattcagccaaatcttcaggaaaagattatgtctttggtcatcgattgatcatcgagattttataatctcatcctgatcaactcctatctatactatcgtggtctttagtttgtctttatcaatctgtgtgtcttaatctcgagcaacatatatactcgctatcagactttttacttaagctatatgtgtgttgtttgcttatgacctctatgtattggttgtgttgtgtgcttaaacgtttcgaactaattgatctgtgtgaaacttattattctgtgataagctatttgtgat